(19) United States
(12) Patent Application Publication
(10) Pub. No.: US 2025/0282877 A1
(43) Pub. Date: Sep. 11, 2025

(54) METHODS OF USING ALK2 AND ALK3 ANTIBODIES

(71) Applicant: Keros Therapeutics, Inc., Lexington, MA (US)

(72) Inventors: Jasbir S. SEEHRA, Lexington, MA (US); Jennifer LACHEY, Lincoln, MA (US); Ffolliott Martin FISHER, Norfolk, MA (US)

(21) Appl. No.: 18/289,039

(22) PCT Filed: May 11, 2022
(86) PCT No.: PCT/US22/28712

§ 371 Date: Oct. 31, 2023

Related U.S. Application Data
(60) Provisional application No. 63/187,350, filed on May 11, 2021.

(51) Int. Cl. C07K 16/28

(52) U.S. Cl. CPC C07K 16/2863; C07K 2317/565; C07K 2317/76; C07K 2317/94

(57) ABSTRACT

The invention features methods of treating a disease or condition that can be treated with an ALK2 or ALK3 inhibitor including a bone disease or bone damage, low red blood cell levels (e.g., anemia or blood loss), heterotopic ossification (e.g., heterotopic ossification resulting from fibrodysplasia ossificans progressiva), Sjogren's syndrome (e.g., dry eye associated with Sjogren's syndrome), multiple osteochondroma, diffuse intrinsic pontine glioma, posterior capsule opacification, or cardiac hypertrophy and/or cardiac fibrosis by administering to a subject an isolated ALK2 or ALK3 antibody, or antigen binding fragment thereof, in an amount of 0.002 mg/kg to 3 mg/kg.

### BACKGROUND OF THE INVENTION

Healthy bone undergoes a constant remodeling that involves both bone breakdown and bone growth. Bone growth is mediated by the osteoblast cell type whereas the osteoclasts resorb the bone. Pathology occurs when these systems fall out of balance either through downregulation of the anabolic program, upregulation of the catabolic system or a combination of both, resulting in a net bone loss. Therefore, controlling the balance in bone remodeling can be useful for promoting the healing of damage to bone as well as the treatment of disorders, such as osteoporosis, associated with loss of bone mass and bone demineralization.

Bone damage can result from a range of root causes, including age- or cancer-related bone loss, genetic conditions, or adverse side effects of drug treatment. The World Health Organization estimates that osteoporosis alone affects 75 million people in the U.S., Europe, and Japan, and is a significant risk factor in bone damage. In general, the whole of bone loss represents pathological states for which there are few effective treatments. Treatment instead focuses on immobilization, exercise, and dietary modifications rather than agents that directly promote bone growth and increase bone density. With respect to osteoporosis, estrogen, calcitonin, osteocalcin with vitamin K, or high doses of dietary calcium are all used as therapeutic interventions. Other therapeutic approaches to osteoporosis include bisphosphonates, parathyroid hormone, parathyroid hormone related protein, calcimimetics, statins, anabolic steroids, lanthanum and strontium salts, and sodium fluoride. Such therapeutics, however, are often associated with undesirable side effects.

Anemia is a global health problem with health implications that affect both morbidity and mortality. In the United States alone, the prevalence of anemia nearly doubled from 2003 to 2012. Symptoms of anemia include fatigue, weakness, shortness of breath, heart palpitations, and reduced cognitive performance, and children, pregnant women, women of reproductive age, and the elderly have been found to have the highest risk of developing anemia. The most common form of anemia is iron deficiency anemia, but anemia can also be caused by chronic diseases, blood loss, and red blood cell destruction. While iron deficiency anemia can be treated with iron supplements, many other forms of anemia, such as aplastic anemia, anemia of chronic disease, and hemolytic anemia may require blood transfusions.

Heterotopic ossification (HO) is the pathologic formation of extra-skeletal bone in soft tissues. This process occurs in patient populations with severe trauma, including large-surface area burns, musculoskeletal injury, orthopedic operations, and spinal cord injury, and in patient populations with a genetic disease known as fibrodysplasia ossificans progressiva (FOP). FOP is caused by a hyper-activating mutation in the type I bone morphogenetic protein (BMP) receptor activin A receptor type 1 (ACVR1), and patients with FOP can develop ectopic bone lesions in muscles, tendons, ligaments, and other connective tissues in the absence of any substantial trauma. The clinical sequela of these pathologic ectopic bone formations, whether in the setting of trauma or genetic mutations, include non-healing wounds, chronic pain, and joint immobility. In the case of FOP, the formation of ectopic bone can lead to bridges of bone that develop across joints and progressively restrict movement and progressive ossification may lead to death due to loss of thoracic cage compliance.

Treatment options for HO are limited as bone often recurs following surgical resection and attempts to surgically remove extra bone can result in further bone growth. Moreover, some patients may have non-resectable HO due to its sensitive location. The risk of an operation may outweigh the benefits of excision, especially in the face of recurrence. There is a need for therapeutic options that can prevent HO before its initial occurrence in at-risk patients and/or reduce the amount of HO, reduce the recurrence of HO, or prevent additional ectopic bone formation in patients with FOP. There is currently no approved treatment for FOP.

Sjogren's syndrome is a systemic autoimmune disorder identified by its two most common symptoms—dry eye and dry mouth. It may also feature joint pain, swelling, and stiffness, swollen salivary glands, skin rashes or dry skin, vaginal dryness, persistent dry cough, and prolonged fatigue. Sjogren's can also cause dysfunction of organs such as the kidneys, gastrointestinal system, blood vessels, lungs, liver, pancreas, and the central nervous system. It is one of the most prevalent autoimmune disorders with over four million Americans suffering from the disorder, nine out of ten of which are women. Dry eye associated with Sjogren's syndrome may be treated with immunosuppressants and steroid eye drops; however, due to side effects such as increased intraocular pressure and susceptibility to infection, steroid eye drops should not be used for an extended period of time.

Diffuse intrinsic pontine glioma (DIPG) is a pediatric brain tumor that originates in the pons and accounts for approximately 20% of all pediatric brain tumors. DIPG remains incurable with a median survival of 10-11 months. Failure to identify a successful therapy for DIPG likely stems from the lack of biological understanding of the disease, as biopsies were not commonly performed until recently due to the sensitive location of the tumor. As a result, many trials have been based upon genetic alterations found in adult glioblastomas, which have recently been found to be molecularly distinct from DIPG.

Multiple osteochondroma (MO), also called hereditary multiple exostoses, is a rare genetic disorder characterized by the development of multiple benign (noncancerous) bone tumors called osteochondromas, often on the growing end (metaphysis) of the long bones of the legs, arms, and digits and on flat bones such as the hip and shoulder blade. The number of osteochondromas and the bones on which they are located vary greatly among affected individuals. The osteochondromas are not present at birth, but approximately 96% of affected individuals develop multiple osteochondromas by the time they are 12 years old. These osteochondromas usually continue to grow until shortly after puberty and may lead to bone deformities, skeletal abnormalities, differences in limb length, short stature, pain, decreased range of motion, and pressure on nerves, blood vessels, the spinal cord, and tissues surrounding the osteochondromas. Hereditary multiple osteochondromas is inherited as an autosomal dominant genetic condition and is associated with mutations in the exostosin-1 (EXT1) or exostosin-2 (EXT2) gene. Current treatment options include a “watch and wait” approach (when no symptoms are present), surgical removal of the osteochondroma, corrective osteotomy, and growth plate arrest or limb-lengthening procedures.

Posterior capsule opacification (PCO) is the most frequent complication associated with decreased vision after cataract surgery and intraocular lens implantation. During cataract surgery, the natural lens of the eye is replaced with an artificial intraocular lens, which is placed inside the lens capsule. PCO occurs when lens epithelial cells migrate toward the posterior capsule, proliferate, and differentiate, growing over the back of the capsule and causing it to thicken and become slightly opaque. This reduces the amount of light that can reach the retina and may lead to blurred or cloudy vision or problems with bright lights and glare. Although PCO can be treated using Nd:YAG laser capsulotomy, the potential complications and significant cost of treatment underscore the importance of identifying new PCO therapies.

Cardiac hypertrophy is the abnormal enlargement or thickening of the heart muscle resulting from increases in cardiomyocyte size and changes in other heart muscle components, such as extracellular matrix. Cardiac hypertrophy may be physiological, occurring in response to exercise or pregnancy, or pathological, occurring in response to stress (e.g., hemodynamic stress), such as hypertension or valvular disease. Cardiac fibrosis may also feature an abnormal thickening of the heart valves due to inappropriate proliferation of cardiac fibroblasts or excess deposition of extracellular matrix in the cardiac muscle, which may result in stiffer, less compliant muscle and occur during the progression to heart failure. There is a lack of efficacious therapies for reducing or reversing cardiac fibrosis, and existing therapies for cardiac hypertrophy, such as adrenergic antagonists, renin-angiotensin system modulators such as angiotensin-converting enzyme (ACE) inhibitors, and angiotensin receptor blockers may have a ceiling effect, characterized by a lack of efficacy, and even regression, in some patients.

There exists a need for novel and effective treatments for bone diseases, anemia, HO (e.g., HO resulting from FOP), Sjogren's syndrome (e.g., dry eye associated with Sjogren's syndrome), DIPG, MO, PCO, and cardiac hypertrophy and/or cardiac fibrosis.

### SUMMARY OF THE INVENTION

Described herein are methods of treating a subject having a disease or condition that can be treated with an ALK2 or ALK3 inhibitor by administering to the subject an activin receptor-like kinase-2 (ALK2, also known as activin A receptor type I (ACVR1)) antibody, such as an ALK2 neutralizing antibody or an ALK2 binding fragment thereof, or an activin receptor-like kinase-3 (ALK3) antibody, such as an ALK3 neutralizing antibody or an ALK3 binding fragment thereof, in an amount of 0.002 mg/kg to 3 mg/kg. The methods described herein can be used to treat a subject, such as a human subject, that has a bone disease (e.g., a disease or condition involving bone damage, such as primary osteoporosis, secondary osteoporosis, osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, or immobility-related bone loss.), low red blood cell levels (e.g., low hemoglobin levels or low red blood cell count, e.g., anemia), heterotopic ossification (HO), such as heterotopic ossification resulting from Fibrodysplasia ossificans progressiva (FOP), Sjogren's syndrome (e.g., dry eye associated with Sjogren's syndrome), diffuse intrinsic pontine glioma (DIPG), multiple osteochondroma (MO), posterior capsule opacification (PCO), or cardiac hypertrophy and/or cardiac fibrosis.

In a first aspect, the disclosure provides a method of treating a subject having a disease or condition that can be treated with an ALK2 or ALK3 inhibitor, the method comprising administering to the subject an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the subject is administered an ALK2 antibody, or an antigen-binding fragment thereof. In some embodiments, the ALK2 antibody, or antigen-binding fragment thereof, is a composition of Table 2. In some embodiments, the ALK2 antibody, or antigen-binding fragment thereof, has the CDR sequences of an antibody described in Table 3 (e.g., any of the light chain CDR1, CDR2, and CDR3 sequences and heavy chain CDR1, CDR2, and CDR3 sequences described in Table 3). In some embodiments, the ALK2 antibody or antigen binding fragment thereof includes a light chain CDR1, CDR2, and CDR3 sequence and a heavy chain CDR1, CDR2, and CDR3 sequence from the same row of Table 3 (e.g., a light chain CDR1, CDR2, and CDR3 sequence and a heavy chain CDR1, CDR2, and CDR3 sequence of any one of Antibodies 1-38). In some embodiments, the ALK2 antibody, or antigen-binding fragment thereof, has a variable light chain (VL) sequence and a variable heavy chain (VH) sequence of an antibody described in Table 4. In some embodiments, the ALK2 antibody, or antigen-binding fragment thereof, has a VL sequence and a VH sequence having at least 90% sequence identity (e.g., 90%, 91%, 92%, 93%, 94%, 95%, 95%, 97%, 98%, 99% or 100% sequence identity) to a VL sequence and a VH sequence of Table 5 (e.g., any VL sequence and any VH sequence of Table 5). In some embodiments, the ALK2 antibody, or an antigen binding fragment thereof, has a VL sequence and a VH sequence from the same row of Table 5. In some embodiments, the VH and VL have the sequence of SEQ ID NOs: 107 and 108; 109 and 110; 109 and 115; 111 and 112; 113 and 114; 116 and 118; 116 and 119; 117 and 118; 131 and 118; 117 and 119; 131 and 119; 117 and 115; 131 and 115; 117 and 120; 131 and 120; 121 and 115; 122 and 126; 132 and 126; 122 and 127; 132 and 127; 122 and 128; 132 and 128; 123 and 126; 133 and 126; 123 and 127; 133 and 127; 123 and 128; 133 and 128; 124 and 126; 124 and 127; 124 and 128; 124 and 129; 125 and 128; 125 and 129; 125 and 130; 134 and 129; 135 and 127; 139 and 129; 140 and 136; 140 and 137; 140 and 138; 201 and 202; 203 and 204; 205 and 206; 207 and 208; 304 and 305; 306 and 307; 308 and 309; 310 and 311; 306 and 312; 313 and 314; 315 and 316; 317 and 318; 319 and 320; 321 and 322; 323 and 324; 325 and 326; 327 and 328; 329 and 330; 331 and 332; 333 and 334; 335 and 336; 337 and 338; 339 and 340; or 341 and 342. In some embodiments, the ALK2 antibody, or an antigen-binding fragment thereof, has a heavy chain sequence and light chain sequence having at least 90% sequence identity (e.g., 90%, 91%, 92%, 93%, 94%, 95%, 95%, 97%, 98%, 99% or 100% sequence identity) to a heavy chain and light chain sequence of Table 6 (e.g., any heavy chain sequence and any light chain sequence of Table 6). In some embodiments, the ALK2 antibody or an antigen binding fragment thereof, has a heavy chain sequence and light chain sequence from the same row of Table 6. In some embodiments, the subject is administered an ALK3 antibody, or an antigen-binding fragment thereof. In some embodiments, the ALK3 antibody, or antigen-binding fragment thereof, contains an antigen binding fragment (Fab) described in Harth et al., PLoS ONE 5: e13049, 2010, such as AbD1556 or AbD1564. In some embodiments, the ALK3 antibody, or antigen binding fragment thereof, has a heavy chain CDR1 including TGYYMK (SEQ ID NO: 79); a heavy chain CDR2 including RINPDNGGRTYNQIFKDK (SEQ ID NO: 80); and a heavy chain CDR3 including RERGQYGNYGGFSD (SEQ ID NO: 81). In some embodiments, the ALK3 antibody, or antigen binding fragment thereof, contains a heavy chain variable region having at least 90% (e.g., at least 91%, 92%, 93%, 94%, 95%, 96%, 97%, 98%, 99%, or more) sequence identity to SEQ ID NO: 77 or SEQ ID NO: 78.

In some embodiments, the antibody is administered in an amount of 0.01 mg/kg to 2 mg/kg (e.g., 0.05 mg/kg to 2 mg/kg, 0.1 mg/kg to 2 mg/kg, 0.5 mg/kg to 2 mg/kg, 1 mg/kg to 2 mg/kg, 1.5 mg/kg to 2 mg/kg, 0.01 mg/kg to 1.5 mg/kg, 0.01 mg/kg to 1 mg/kg, 0.01 mg/kg to 0.5 mg/kg, 0.01 mg/kg to 0.25 mg/kg, 0.01 mg/kg to 0.1 mg/kg, or 0.01 mg/kg to 0.05 mg/kg). In some embodiments, the antibody is administered in an amount of 0.05 mg/kg to 1 mg/kg (e.g., 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.05 mg/kg to 0.9 mg/kg, 0.05 mg/kg to 0.08 mg/kg, 0.05 mg/kg to 0.8 mg/kg, 0.05 mg/kg to 0.7 mg/kg, 0.05 mg/kg to 0.6 mg/kg, 0.05 mg/kg to 0.5 mg/kg, 0.05 mg/kg to 0.4 mg/kg, 0.05 mg/kg to 0.3 mg/kg, 0.05 mg/kg to 0.2 mg/kg, or 0.05 mg/kg to 0.1 mg/kg). In some embodiments, the antibody is administered in an amount of 0.01 mg/kg to 1 mg/kg (e.g., 0.05 mg/kg to 1 mg/kg, 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.01 mg/kg to 0.9 mg/kg, 0.01 mg/kg to 0.8 mg/kg, 0.01 mg/kg to 0.7 mg/kg, 0.01 mg/kg to 0.6 mg/kg, 0.01 mg/kg to 0.5 mg/kg, 0.01 mg/kg to 0.4 mg/kg, 0.01 mg/kg to 0.3 mg/kg, 0.01 mg/kg to 0.2 mg/kg, 0.01 mg/kg to 0.1 mg/kg, or 0.01 mg/kg to 0.05 mg/kg). In some embodiments, the antibody is administered in an amount of 0.01 mg/kg to 0.3 mg/kg (e.g., 0.01 mg/kg to 3 mg/kg, 0.025 mg/kg to 0.3 mg/kg, 0.05 mg/kg to 0.3 mg/kg, 0.075 mg/kg to 0.3 mg/kg, 0.1 mg/kg to 0.3 mg/kg, 0.125 mg/kg to 0.3 mg/kg, 0.15 mg/kg to 0.3 mg/kg, 0.175 mg/kg to 0.3 mg/kg, 0.2 mg/kg to 0.3 mg/kg, 0.225 mg/kg to 0.3 mg/kg, 0.25 mg/kg to 0.3 mg/kg, 0.275 mg/kg to 0.3 mg/kg, 0.01 mg/kg to 0.275 mg/kg, 0.01 mg/kg to 0.25 mg/kg, 0.01 mg/kg to 0.225 mg/kg, 0.01 mg/kg to 0.2 mg/kg, 0.01 mg/kg to 0.225 mg/kg, 0.01 mg/kg to 0.2 mg/kg, 0.01 mg/kg to 0.175 mg/kg, 0.01 mg/kg to 0.15 mg/kg, 0.01 mg/kg to 0.125 mg/kg, 0.01 mg/kg to 0.1 mg/kg, 0.01 mg/kg to 0.075 mg/kg, 0.01 mg/kg to 0.05 mg/kg, or 0.01 mg/kg to 0.025 mg/kg). In some embodiments, the antibody is administered in amount of 0.1 mg/kg to 0.3 mg/kg (e.g., 0.1 mg/kg to 0.25 mg/kg, 0.1 mg/kg to 0.2 mg/kg, 0.1 mg/kg to 0.15 mg/kg, 0.15 mg/kg to 0.3 mg/kg, 0.2 mg/kg to 0.3 mg/kg, or 0.25 mg/kg to 0.3 mg/kg).

In some embodiments, the antibody is administered to the subject at a frequency that is weekly to semi-annually. For example, the antibody may be administered once a week, once every 14 days, once a month, once every two months, once every three months, once every four months, once every five months, or once every six months. In some embodiments, the antibody may be administered to the subject at a frequency of once every 6 months. In some embodiments, the antibody may be administered to the subject at a frequency of once every 4 months. In some embodiments, the antibody may be administered to the subject at a frequency of once every 2 months. In some embodiments, the antibody may be administered to the subject at a frequency of once every month. In some embodiments, the antibody may be administered to the subject at a frequency of once biweekly. In some embodiments, the antibody is administered to the subject at a frequency of once every 10 to 14 days (e.g., once every 10 days, once every 11 days, once every 12 days, once every 13 days, or once every 14 days). In some embodiments, the antibody is administered to the subject at a frequency of once a week.

In some embodiments, the antibody, or antigen-binding fragment thereof, is administered subcutaneously. In some embodiments, the antibody, or antigen-binding fragment thereof, is administered intravenously.

In some embodiments, the disease or condition that can be treated with an ALK2 or ALK3 inhibitor is anemia. In some embodiments, the subject being treated for anemia is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 6 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 4 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 3 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 2 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every month. In some embodiments, the subject being treated for anemia is administered an isolated ALK2 or ALK3 antibody, or an antigen-binding fragment thereof, in an amount of 0.01 mg/kg to 2 mg/kg (e.g., 0.05 mg/kg to 2 mg/kg, 0.1 mg/kg to 2 mg/kg, 0.5 mg/kg to 2 mg/kg, 1 mg/kg to 2 mg/kg, 1.5 mg/kg to 2 mg/kg, 0.01 mg/kg to 1.5 mg/kg, 0.01 mg/kg to 1 mg/kg, 0.01 mg/kg to 0.5 mg/kg, 0.01 mg/kg to 0.25 mg/kg, 0.01 mg/kg to 0.1 mg/kg, or 0.01 mg/kg to 0.05 mg/kg). In some embodiments, the subject being treated for anemia is administered an isolated ALK2 or ALK3 antibody, or an antigen-binding fragment thereof, in an amount of 0.05 mg/kg to 1 mg/kg (e.g., 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.05 mg/kg to 0.9 mg/kg, 0.05 mg/kg to 0.8 mg/kg, 0.05 mg/kg to 0.08 mg/kg, 0.05 mg/kg to 0.7 mg/kg, 0.05 mg/kg to 0.6 mg/kg, 0.05 mg/kg to 0.5 mg/kg, 0.05 mg/kg to 0.4 mg/kg, 0.05 mg/kg to 0.3 mg/kg, 0.05 mg/kg to 0.2 mg/kg, or 0.05 mg/kg to 0.1 mg/kg). In some embodiments, the subject being treated for anemia is administered an isolated ALK2 or ALK3 antibody, or an antigen-binding fragment thereof, in an amount of 0.01 mg/kg to 1 mg/kg (e.g., 0.05 mg/kg to 1 mg/kg, 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.01 mg/kg to 0.9 mg/kg, 0.01 mg/kg to 0.8 mg/kg, 0.01 mg/kg to 0.7 mg/kg, 0.01 mg/kg to 0.6 mg/kg, 0.01 mg/kg to 0.5 mg/kg, 0.01 mg/kg to 0.4 mg/kg, 0.01 mg/kg to 0.3 mg/kg, 0.01 mg/kg to 0.2 mg/kg, 0.01 mg/kg to 0.1 mg/kg, or 0.01 mg/kg to 0.05 mg/kg). In some embodiments, the subject being treated for anemia is administered an isolated ALK2 or ALK3 antibody, or an antigen-binding fragment thereof, in an amount of 0.01 mg/kg to 0.3 mg/kg (e.g., 0.01 mg/kg to 3 mg/kg, 0.025 mg/kg to 0.3 mg/kg, 0.05 mg/kg to 0.3 mg/kg, 0.075 mg/kg to 0.3 mg/kg, 0.1 mg/kg to 0.3 mg/kg, 0.125 mg/kg to 0.3 mg/kg, 0.15 mg/kg to 0.3 mg/kg, 0.175 mg/kg to 0.3 mg/kg, 0.2 mg/kg to 0.3 mg/kg, 0.225 mg/kg to 0.3 mg/kg, 0.25 mg/kg to 0.3 mg/kg, 0.275 mg/kg to 0.3 mg/kg, 0.01 mg/kg to 0.275 mg/kg, 0.01 mg/kg to 0.25 mg/kg, 0.01 mg/kg to 0.225 mg/kg, 0.01 mg/kg to 0.2 mg/kg, 0.01 mg/kg to 0.225 mg/kg, 0.01 mg/kg to 0.2 mg/kg, 0.01 mg/kg to 0.175 mg/kg, 0.01 mg/kg to 0.15 mg/kg, 0.01 mg/kg to 0.125 mg/kg, 0.01 mg/kg to 0.1 mg/kg, 0.01 mg/kg to 0.075 mg/kg, 0.01 mg/kg to 0.05 mg/kg, or 0.01 mg/kg to 0.025 mg/kg). In some embodiments, the subject being treated for anemia is administered an isolated ALK2 or ALK3 antibody, or an antigen-binding fragment thereof, in an amount of 0.1 mg/kg to 0.3 mg/kg (e.g., 0.1 mg/kg to 0.25 mg/kg, 0.1 mg/kg to 0.2 mg/kg, 0.1 mg/kg to 0.15 mg/kg, 0.15 mg/kg to 0.3 mg/kg, 0.2 mg/kg to 0.3 mg/kg, or 0.25 mg/kg to 0.3 mg/kg). In some embodiments, the anemia is associated with cancer, cancer treatment, chronic kidney disease, acute renal disease or failure, chronic renal disease or failure, myelofibrosis treatment, a myelodysplastic syndrome, thalassemia, a nutritional deficit, adverse reaction to medication, ineffective hematopoiesis, an inflammatory or autoimmune disease, splenomegaly, porphyria, vasculitis, hemolysis, a bone marrow defect, bone marrow transplantation, myelofibrosis, diabetes, acute liver disease, chronic liver disease, acute bleeding, chronic bleeding, infection, hemoglobinopathy, drug use, alcohol abuse, Churg-Strauss syndrome, Felty syndrome, Pearson syndrome, dyskeratosis congenita, graft versus host disease, hematopoietic stem cell transplantation, osteomyelofibrosis, pancytopenia, pure red-cell aplasia, purpura Schoenlein-Henoch, Shwachman syndrome, advanced age, contraindication to transfusion, surgery, trauma, a wound, an ulcer, urinary tract bleeding, digestive tract bleeding, frequent blood donation, or heavy menstrual bleeding. In some embodiments, the anemia is aplastic anemia, iron deficiency anemia, vitamin deficiency anemia, anemia of inflammation, IRIDA, anemia associated with bone marrow disease, hemolytic anemia, sickle cell anemia, microcytic anemia, hypochromic anemia, sideroblastic anemia, congenital dyserythropoietic anemia, Diamond Blackfan anemia, Fanconi anemia, or refractory anemia with excess of blasts. In some embodiments, the anemia is aplastic anemia, iron deficiency anemia, vitamin deficiency anemia, anemia of inflammation, IRIDA, anemia associated with bone marrow disease, hemolytic anemia, sickle cell anemia, microcytic anemia, hypochromic anemia, sideroblastic anemia, Diamond Blackfan anemia, Fanconi's anemia, or refractory anemia with excess of blasts. In some embodiments, the anemia is IRIDA. In some embodiments, the anemia is anemia of inflammation. In some embodiments, the anemia is associated with elevated hepcidin levels.

In some embodiments, the disease or condition that can be treated with an ALK2 or ALK3 inhibitor is fibrodysplasia ossificans progressiva (FOP). In some embodiments, the subject being treated for FOP is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every month. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once biweekly. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 10 to 14 days (e.g., once every 10 days, once every 11 days, once every 12 days, once every 13 days, or once every 14 days). In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once a week. In some embodiments, the subject being treated for FOP is administered an isolated ALK2 or ALK3 antibody, or an antigen-binding fragment thereof, in an amount of 0.01 mg/kg to 2 mg/kg e.g., 0.05 mg/kg to 2 mg/kg, 0.1 mg/kg to 2 mg/kg, 0.5 mg/kg to 2 mg/kg, 1 mg/kg to 2 mg/kg, 1.5 mg/kg to 2 mg/kg, 0.01 mg/kg to 1.5 mg/kg, 0.01 mg/kg to 1 mg/kg, 0.01 mg/kg to 0.5 mg/kg, 0.01 mg/kg to 0.25 mg/kg, 0.01 mg/kg to 0.1 mg/kg, or 0.01 mg/kg to 0.05 mg/kg). In some embodiments, the subject being treated for FOP is administered an isolated ALK2 or ALK3 antibody, or an antigen-binding fragment thereof, in an amount of 0.05 mg/kg to 1 mg/kg (e.g., 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.05 mg/kg to 0.9 mg/kg, 0.05 mg/kg to 0.8 mg/kg, 0.05 mg/kg to 0.08 mg/kg, 0.05 mg/kg to 0.7 mg/kg, 0.05 mg/kg to 0.6 mg/kg, 0.05 mg/kg to 0.5 mg/kg, 0.05 mg/kg to 0.4 mg/kg, 0.05 mg/kg to 0.3 mg/kg, 0.05 mg/kg to 0.2 mg/kg, or 0.05 mg/kg to 0.1 mg/kg). In some embodiments, the subject being treated for FOP is administered an isolated ALK2 or ALK3 antibody, or an antigen-binding fragment thereof, in an amount of 0.01 mg/kg to 1 mg/kg (e.g., 0.05 mg/kg to 1 mg/kg, 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.01 mg/kg to 0.9 mg/kg, 0.01 mg/kg to 0.8 mg/kg, 0.01 mg/kg to 0.7 mg/kg, 0.01 mg/kg to 0.6 mg/kg, 0.01 mg/kg to 0.5 mg/kg, 0.01 mg/kg to 0.4 mg/kg, 0.01 mg/kg to 0.3 mg/kg, 0.01 mg/kg to 0.2 mg/kg, 0.01 mg/kg to 0.1 mg/kg, or 0.01 mg/kg to 0.05 mg/kg). In some embodiments, the subject being treated for FOP is administered an isolated ALK2 or ALK3 antibody, or an antigen-binding fragment thereof, in an amount of 0.01 mg/kg to 0.3 mg/kg (e.g., 0.01 mg/kg to 3 mg/kg, 0.025 mg/kg to 0.3 mg/kg, 0.05 mg/kg to 0.3 mg/kg, 0.075 mg/kg to 0.3 mg/kg, 0.1 mg/kg to 0.3 mg/kg, 0.125 mg/kg to 0.3 mg/kg, 0.15 mg/kg to 0.3 mg/kg, 0.175 mg/kg to 0.3 mg/kg, 0.2 mg/kg to 0.3 mg/kg, 0.225 mg/kg to 0.3 mg/kg, 0.25 mg/kg to 0.3 mg/kg, 0.275 mg/kg to 0.3 mg/kg, 0.01 mg/kg to 0.275 mg/kg, 0.01 mg/kg to 0.25 mg/kg, 0.01 mg/kg to 0.225 mg/kg, 0.01 mg/kg to 0.2 mg/kg, 0.01 mg/kg to 0.225 mg/kg, 0.01 mg/kg to 0.2 mg/kg, 0.01 mg/kg to 0.175 mg/kg, 0.01 mg/kg to 0.15 mg/kg, 0.01 mg/kg to 0.125 mg/kg, 0.01 mg/kg to 0.1 mg/kg, 0.01 mg/kg to 0.075 mg/kg, 0.01 mg/kg to 0.05 mg/kg, or 0.01 mg/kg to 0.025 mg/kg). In some embodiments, the subject being treated for FOP is administered an isolated ALK2 or ALK3 antibody, or an antigen-binding fragment thereof, in an amount of 0.1 mg/kg to 0.3 mg/kg (e.g., 0.1 mg/kg to 0.25 mg/kg, 0.1 mg/kg to 0.2 mg/kg, 0.1 mg/kg to 0.15 mg/kg, 0.15 mg/kg to 0.3 mg/kg, 0.2 mg/kg to 0.3 mg/kg, or 0.25 mg/kg to 0.3 mg/kg). In some embodiments, the FOP is inherited FOP or sporadic FOP.

In some embodiments, the disease or condition is a bone disease. In some embodiments, the subject being treated for a bone disease is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 6 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 4 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 3 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 2 months. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every month. In some embodiments, the subject being treated for a bone disease is administered an isolated ALK2 or ALK3 antibody, or an antigen-binding fragment thereof, in an amount of 0.01 mg/kg to 2 mg/kg (e.g., 0.05 mg/kg to 2 mg/kg, 0.1 mg/kg to 2 mg/kg, 0.5 mg/kg to 2 mg/kg, 1 mg/kg to 2 mg/kg, 1.5 mg/kg to 2 mg/kg, 0.01 mg/kg to 1.5 mg/kg, 0.01 mg/kg to 1 mg/kg, 0.01 mg/kg to 0.5 mg/kg, 0.01 mg/kg to 0.25 mg/kg, 0.01 mg/kg to 0.1 mg/kg, or 0.01 mg/kg to 0.05 mg/kg). In some embodiments, the subject being treated for a bone disease is administered an isolated ALK2 or ALK3 antibody, or an antigen-binding fragment thereof, in an amount of 0.05 mg/kg to 1 mg/kg (e.g., 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.05 mg/kg to 0.9 mg/kg, 0.05 mg/kg to 0.8 mg/kg, 0.05 mg/kg to 0.08 mg/kg, 0.05 mg/kg to 0.7 mg/kg, 0.05 mg/kg to 0.6 mg/kg, 0.05 mg/kg to 0.5 mg/kg, 0.05 mg/kg to 0.4 mg/kg, 0.05 mg/kg to 0.3 mg/kg, 0.05 mg/kg to 0.2 mg/kg, or 0.05 mg/kg to 0.1 mg/kg). In some embodiments, the subject being treated for a bone disease is administered an isolated ALK2 or ALK3 antibody, or an antigen-binding fragment thereof, in an amount of 0.01 mg/kg to 1 mg/kg (e.g., 0.05 mg/kg to 1 mg/kg, 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.01 mg/kg to 0.9 mg/kg, 0.01 mg/kg to 0.8 mg/kg, 0.01 mg/kg to 0.7 mg/kg, 0.01 mg/kg to 0.6 mg/kg, 0.01 mg/kg to 0.5 mg/kg, 0.01 mg/kg to 0.4 mg/kg, 0.01 mg/kg to 0.3 mg/kg, 0.01 mg/kg to 0.2 mg/kg, 0.01 mg/kg to 0.1 mg/kg, or 0.01 mg/kg to 0.05 mg/kg). In some embodiments, the subject being treated for a bone disease is administered an isolated ALK2 or ALK3 antibody, or an antigen-binding fragment thereof, in an amount of 0.01 mg/kg to 0.3 mg/kg (e.g., 0.01 mg/kg to 3 mg/kg, 0.025 mg/kg to 0.3 mg/kg, 0.05 mg/kg to 0.3 mg/kg, 0.075 mg/kg to 0.3 mg/kg, 0.1 mg/kg to 0.3 mg/kg, 0.125 mg/kg to 0.3 mg/kg, 0.15 mg/kg to 0.3 mg/kg, 0.175 mg/kg to 0.3 mg/kg, 0.2 mg/kg to 0.3 mg/kg, 0.225 mg/kg to 0.3 mg/kg, 0.25 mg/kg to 0.3 mg/kg, 0.275 mg/kg to 0.3 mg/kg, 0.01 mg/kg to 0.275 mg/kg, 0.01 mg/kg to 0.25 mg/kg, 0.01 mg/kg to 0.225 mg/kg, 0.01 mg/kg to 0.2 mg/kg, 0.01 mg/kg to 0.225 mg/kg, 0.01 mg/kg to 0.2 mg/kg, 0.01 mg/kg to 0.175 mg/kg, 0.01 mg/kg to 0.15 mg/kg, 0.01 mg/kg to 0.125 mg/kg, 0.01 mg/kg to 0.1 mg/kg, 0.01 mg/kg to 0.075 mg/kg, 0.01 mg/kg to 0.05 mg/kg, or 0.01 mg/kg to 0.025 mg/kg). In some embodiments, the subject being treated for a bone disease is administered an isolated ALK2 or ALK3 antibody, or an antigen-binding fragment thereof, in an amount of 0.1 mg/kg to 0.3 mg/kg (e.g., 0.1 mg/kg to 0.25 mg/kg, 0.1 mg/kg to 0.2 mg/kg, 0.1 mg/kg to 0.15 mg/kg, 0.15 mg/kg to 0.3 mg/kg, 0.2 mg/kg to 0.3 mg/kg, or 0.25 mg/kg to 0.3 mg/kg). In some embodiments, the bone disease is osteoporosis (e.g., primary or secondary osteoporosis), osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, or immobility-related bone loss.

In some embodiments, the disease or condition that can be treated with an ALK2 or ALK3 inhibitor is Sjogren's syndrome, multiple osteochondroma, diffuse intrinsic pontine glioma, posterior capsule opacification, cardiac hypertrophy, or cardiac fibrosis. In some embodiments, the subject being treated for Sjogren's syndrome, multiple osteochondroma, diffuse intrinsic pontine glioma, posterior capsule opacification, cardiac hypertrophy, or cardiac fibrosis is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every month. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once biweekly. In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once every 10 to 14 days (e.g., once every 10 days, once every 11 days, once every 12 days, once every 13 days, or once every 14 days). In some embodiments, the subject is administered an isolated ALK2 or ALK3 antibody, or an antigen-binding fragment thereof, in an amount of 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg) once a week. In some embodiments, the subject being treated for Sjogren's syndrome, multiple osteochondroma, diffuse intrinsic pontine glioma, posterior capsule opacification, cardiac hypertrophy, or cardiac fibrosis is administered an isolated ALK2 or ALK3 antibody, or an antigen-binding fragment thereof, in an amount of 0.01 mg/kg to 2 mg/kg (e.g., 0.05 mg/kg to 2 mg/kg, 0.1 mg/kg to 2 mg/kg, 0.5 mg/kg to 2 mg/kg, 1 mg/kg to 2 mg/kg, 1.5 mg/kg to 2 mg/kg, 0.01 mg/kg to 1.5 mg/kg, 0.01 mg/kg to 1 mg/kg, 0.01 mg/kg to 0.5 mg/kg, 0.01 mg/kg to 0.25 mg/kg, 0.01 mg/kg to 0.1 mg/kg, or 0.01 mg/kg to 0.05 mg/kg). In some embodiments, the subject being treated for Sjogren's syndrome, multiple osteochondroma, diffuse intrinsic pontine glioma, posterior capsule opacification, cardiac hypertrophy, or cardiac fibrosis is administered an isolated ALK2 or ALK3 antibody, or an antigen-binding fragment thereof, in an amount of 0.05 mg/kg to 1 mg/kg (e.g., 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.05 mg/kg to 0.9 mg/kg, 0.05 mg/kg to 0.08 mg/kg, 0.05 mg/kg to 0.8 mg/kg, 0.05 mg/kg to 0.7 mg/kg, 0.05 mg/kg to 0.6 mg/kg, 0.05 mg/kg to 0.5 mg/kg, 0.05 mg/kg to 0.4 mg/kg, 0.05 mg/kg to 0.3 mg/kg, 0.05 mg/kg to 0.2 mg/kg, or 0.05 mg/kg to 0.1 mg/kg). In some embodiments, the subject being treated for Sjogren's syndrome, multiple osteochondroma, diffuse intrinsic pontine glioma, posterior capsule opacification, cardiac hypertrophy, or cardiac fibrosis is administered an isolated ALK2 or ALK3 antibody, or an antigen-binding fragment thereof, in an amount of 0.01 mg/kg to 1 mg/kg (e.g., 0.05 mg/kg to 1 mg/kg, 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.01 mg/kg to 0.9 mg/kg, 0.01 mg/kg to 0.8 mg/kg, 0.01 mg/kg to 0.7 mg/kg, 0.01 mg/kg to 0.6 mg/kg, 0.01 mg/kg to 0.5 mg/kg, 0.01 mg/kg to 0.4 mg/kg, 0.01 mg/kg to 0.3 mg/kg, 0.01 mg/kg to 0.2 mg/kg, 0.01 mg/kg to 0.1 mg/kg, or 0.01 mg/kg to 0.05 mg/kg). In some embodiments, the subject being treated for Sjogren's syndrome, multiple osteochondroma, diffuse intrinsic pontine glioma, posterior capsule opacification, cardiac hypertrophy, or cardiac fibrosis is administered an isolated ALK2 or ALK3 antibody, or an antigen-binding fragment thereof, in an amount of 0.01 mg/kg to 0.3 mg/kg (e.g., 0.01 mg/kg to 3 mg/kg, 0.025 mg/kg to 0.3 mg/kg, 0.05 mg/kg to 0.3 mg/kg, 0.075 mg/kg to 0.3 mg/kg, 0.1 mg/kg to 0.3 mg/kg, 0.125 mg/kg to 0.3 mg/kg, 0.15 mg/kg to 0.3 mg/kg, 0.175 mg/kg to 0.3 mg/kg, 0.2 mg/kg to 0.3 mg/kg, 0.225 mg/kg to 0.3 mg/kg, 0.25 mg/kg to 0.3 mg/kg, 0.275 mg/kg to 0.3 mg/kg, 0.01 mg/kg to 0.275 mg/kg, 0.01 mg/kg to 0.25 mg/kg, 0.01 mg/kg to 0.225 mg/kg, 0.01 mg/kg to 0.2 mg/kg, 0.01 mg/kg to 0.225 mg/kg, 0.01 mg/kg to 0.2 mg/kg, 0.01 mg/kg to 0.175 mg/kg, 0.01 mg/kg to 0.15 mg/kg, 0.01 mg/kg to 0.125 mg/kg, 0.01 mg/kg to 0.1 mg/kg, 0.01 mg/kg to 0.075 mg/kg, 0.01 mg/kg to 0.05 mg/kg, or 0.01 mg/kg to 0.025 mg/kg). In some embodiments, the subject being treated for Sjogren's syndrome, multiple osteochondroma, diffuse intrinsic pontine glioma, posterior capsule opacification, cardiac hypertrophy, or cardiac fibrosis is administered an isolated ALK2 or ALK3 antibody, or an antigen-binding fragment thereof, in an amount of 0.1 mg/kg to 0.3 mg/kg (e.g., 0.1 mg/kg to 0.25 mg/kg, 0.1 mg/kg to 0.2 mg/kg, 0.1 mg/kg to 0.15 mg/kg, 0.15 mg/kg to 0.3 mg/kg, 0.2 mg/kg to 0.3 mg/kg, or 0.25 mg/kg to 0.3 mg/kg).

In some embodiments of any of the foregoing aspects, the method includes administering an ALK2 antibody or an antigen binding fragment thereof. In some embodiments, the ALK2 antibody or antigen binding fragment thereof, is an antibody of Table 2. In some embodiments, the ALK2 antibody, or antigen-binding fragment thereof, has the CDR sequences of an antibody described in Table 3 (e.g., any of the light chain CDR1, CDR2, and CDR3 sequences and heavy chain CDR1, CDR2, and CDR3 sequences described in Table 3). In some embodiments, the ALK2 antibody or antigen binding fragment thereof includes a light chain CDR1, CDR2, and CDR3 sequence and a heavy chain CDR1, CDR2, and CDR3 sequence from the same row of Table 3 (e.g., a light chain CDR1, CDR2, and CDR3 sequence and a heavy chain CDR1, CDR2, and CDR3 sequence of any one of Antibodies 1-38). In some embodiments, the ALK2 antibody, or antigen-binding fragment thereof, has a variable light chain (VL) sequence and a variable heavy chain (VH) sequence of an antibody described in Table 4. In some embodiments, the ALK2 antibody, or antigen-binding fragment thereof, has a VL sequence and a VH sequence having at least 90% sequence identity (e.g., 90%, 91%, 92%, 93%, 94%, 95%, 95%, 97%, 98%, 99% or 100% sequence identity) to a VL sequence and a VH sequence of Table 5 (e.g., any VL sequence and any VH sequence of Table 5). In some embodiments, the ALK2 antibody, or an antigen binding fragment thereof, has a VL sequence and a VH sequence from the same row of Table 5. In some embodiments, the VH and VL have the sequence of SEQ ID NOs: 107 and 108; 109 and 110; 109 and 115; 111 and 112; 113 and 114; 116 and 118; 116 and 119; 117 and 118; 131 and 118; 117 and 119; 131 and 119; 117 and 115; 131 and 115; 117 and 120; 131 and 120; 121 and 115; 122 and 126; 132 and 126; 122 and 127; 132 and 127; 122 and 128; 132 and 128; 123 and 126; 133 and 126; 123 and 127; 133 and 127; 123 and 128; 133 and 128; 124 and 126; 124 and 127; 124 and 128; 124 and 129; 125 and 128; 125 and 129; 125 and 130; 134 and 129; 135 and 127; 139 and 129; 140 and 136; 140 and 137; 140 and 138; 201 and 202; 203 and 204; 205 and 206; 207 and 208; 304 and 305; 306 and 307; 308 and 309; 310 and 311; 306 and 312; 313 and 314; 315 and 316; 317 and 318; 319 and 320; 321 and 322; 323 and 324; 325 and 326; 327 and 328; 329 and 330; 331 and 332; 333 and 334; 335 and 336; 337 and 338; 339 and 340; or 341 and 342. In some embodiments, the ALK2 antibody, or an antigen-binding fragment thereof, has a heavy chain sequence and light chain sequence having at least 90% sequence identity (e.g., 90%, 91%, 92%, 93%, 94%, 95%, 95%, 97%, 98%, 99% or 100% sequence identity) to a heavy chain and light chain sequence of Table 6 (e.g., any heavy chain sequence and any light chain sequence of Table 6). In some embodiments, the ALK2 antibody or an antigen binding fragment thereof, has a heavy chain sequence and light chain sequence from the same row of Table 6. In some embodiments of any of the foregoing aspects, the method includes administering an ALK3 antibody or an antigen binding fragment thereof. In some embodiments, the ALK3 antibody, or antigen-binding fragment thereof, contains an antigen binding fragment (Fab) described in Harth et al., PLoS ONE 5: e13049, 2010, such as AbD1556 or AbD1564. In some embodiments, the ALK3 antibody, or antigen binding fragment thereof, has a heavy chain CDR1 including TGYYMK (SEQ ID NO: 79); a heavy chain CDR2 including RINPDNGGRTYNQIFKDK (SEQ ID NO: 80); and a heavy chain CDR3 including RERGQYGNYGGFSD (SEQ ID NO: 81). In some embodiments, the ALK3 antibody, or antigen binding fragment thereof, contains a heavy chain variable region having at least 90% (e.g., at least 91%, 92%, 93%, 94%, 95%, 96%, 97%, 98%, 99%, or more) sequence identity to SEQ ID NO: 77 or SEQ ID NO: 78.

In some embodiments, the subject is a human.

### Definitions

As used herein, the term “about” refers to a value that is within 10% above or below the value being described.

As used herein, “administration” refers to providing or giving a subject a therapeutic agent (e.g., an ALK2 or ALK3 antibody or antigen binding fragment thereof (e.g., an ALK2 or ALK3 binding fragment) described herein), by any effective route. Exemplary routes of administration are described herein below.

The term “antibody” is used in the broadest sense and specifically covers intact monoclonal antibodies, polyclonal antibodies, multispecific antibodies (e.g., bispecific antibodies) formed from at least two intact antibodies, and antibody fragments so long as they exhibit the desired biological activity.

“Antibody fragments” include a portion of an intact antibody, preferably the antigen binding or variable region of the intact antibody. Examples of antibody fragments include Fab, Fab′, F(ab′)₂, and Fv fragments; diabodies; linear antibodies (Zapata et al. Protein Eng. 8(10):1057-1062 (1995)); single-chain antibody molecules; and multispecific antibodies formed from antibody fragments.

The term “monoclonal antibody” as used herein refers to an antibody obtained from a population of substantially homogeneous antibodies, i.e., the individual antibodies included in the population are identical except for possible naturally occurring mutations that may be present in minor amounts. Monoclonal antibodies are highly specific, being directed against a single antigenic site.

The term “monoclonal antibody” as used herein specifically includes “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

“Humanized” forms of non-human (e.g., murine) antibodies are chimeric antibodies, antibody chains or fragments thereof, (such as Fv, Fab, Fab′, F(ab′)2 or other antigen-binding subsequences of antibodies) which contain minimal sequence derived from non-human antibody. For the most part, humanized antibodies are human antibodies (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human antibody are replaced by corresponding non-human residues. Further, humanized antibodies may include residues which are found neither in the recipient antibody nor in the imported CDR or framework sequences.

As used herein, the terms “bone mineral density (BMD),” “bone density,” and “bone mass” refer to a measure of the amount of bone mineral (e.g., calcium) in bone tissue. BMD may be measured by well-established clinical techniques known to one of skill in the art (e.g., by single-1 or dual-energy photon or X-ray absorptiometry (DEXA)). The concept of BMD relates to the mass of mineral per volume of bone, although clinically it is measured by proxy according to optical density per square centimeter of bone surface upon imaging. BMD measurement is used in clinical medicine as an indirect indicator of osteoporosis and fracture risk. In some embodiments, BMD test results are provided as a T-score, where the T-score represents the BMD of a subject compared to the ideal or peak bone mineral density of a healthy 30-year-old adult. A score of 0 indicates that the BMD is equal to the normal reference value for a healthy young adult. Differences between the measured BMD of subject and that of the reference value for a healthy young adult are measured in standard deviations units (SDs). Accordingly, a T-score of between +1 SD and −1 SD may indicate a normal BMD, a T-score of between −1 SD and −2.5 SD may indicate low bone mass (e.g., osteopenia), and a T-score lower than −2.5 SD may indicate osteoporosis or severe osteoporosis. In some embodiments, an ALK2 or ALK3 antibody or an antigen binding fragment thereof (e.g., an ALK2 or ALK3 binding fragment), of the invention is administered to a subject in need thereof, wherein the patient has low bone mass (e.g., a T-Score of between −1 SD and −2.5 SD). In some embodiments, an ALK2 or ALK3 antibody or an antigen binding fragment thereof (e.g., an ALK2 or ALK3 binding fragment), of the invention is administered to a subject in need thereof, wherein the patient has osteoporosis (e.g., a T-Score of less than −2.5 SD). In some embodiments, administration of an ALK2 or ALK3 antibody or an antigen binding fragment thereof (e.g., an ALK2 or ALK3 binding fragment), of the invention treats the subject by increasing their BMD (e.g., increasing the T-Score of the subject).

As used herein, the term “bone strength” refers to a measurement of bone that is determined by bone quality in addition to bone mineral density. Bone quality is influenced by bone geometry, microarchitecture, and the properties of constituent tissues. Bone strength can be used to assess the bone's risk of fracture.

As used herein, the term “bone disease” refers to a condition characterized by bone damage (e.g., decreased bone mineral density, decreased bone strength, and/or bone loss). Such diseases or conditions may be caused by an imbalance in osteoblast and/or osteoclast activity (e.g., increased bone resorption or reduced bone formation). Bone diseases include primary osteoporosis, secondary osteoporosis, osteopenia, osteopetrosis, osteogenesis imperfecta, bone fracture, bone cancer or cancer metastasis-related bone loss (e.g., bone loss associated with multiple myeloma), Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, and immobility-related bone loss.

As used herein, the term “neuromuscular disease-related bone loss” refers to bone loss that occurs in a subject having a neuromuscular disease. Poor bone health is often a significant problem for patients with neuromuscular disease. Deficiency of bone mineral density and increased incidence of bone fractures, for example, are a well-recognized clinical consequence of diseases such as DMD, ALS, and SMA.

As used herein, the terms “bone remodeling” or “bone metabolism” refer to the process for maintaining bone strength and ion homeostasis by replacing discrete parts of old bone with newly synthesized packets of proteinaceous matrix. Bone is resorbed by osteoclasts and is deposited by osteoblasts in a process called ossification. Osteocyte activity plays a key role in this process. Conditions that result in a decrease in bone mass, can either be caused by an increase in resorption, or a decrease in ossification. In a healthy individual, during childhood, bone formation exceeds resorption. As the aging process occurs, resorption exceeds formation. Bone resorption rates are also typically much higher in post-menopausal older women due to estrogen deficiency related to menopause.

As used herein, the terms “bone resorption” or “bone catabolic activity” refer to a process by which osteoclasts break down the tissue in bones and release the minerals, resulting in a transfer of the mineral (e.g., calcium) from bone tissue to the blood. Increased rates of bone resorption are associated with aging, including in post-menopausal women. High rates of bone resorption, or rates of bone resorption that exceed the rate of ossification, are associated with bone disorders, such as decreased bone mineral density, including osteopenia and osteoporosis, and result in bone loss. In some embodiments, an ALK2 or ALK3 antibody or an antigen binding fragment thereof (e.g., an ALK2 or ALK3 binding fragment), of the invention is administered to a subject in need thereof, to decrease bone resorption in the subject (e.g., the rate of bone resorption in the subject).

As used herein, the terms “bone formation,” “ossification,” “osteogenesis,” or “bone anabolic activity” refer to the process of forming new bone tissue by osteoblasts. In some embodiments, an ALK2 or ALK3 antibody or an antigen binding fragment thereof (e.g., an ALK2 or ALK3 binding fragment), of the invention is administered to a subject in need thereof, to increase bone formation (e.g., increase the rate of bone formation or osteogenesis in the subject).

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, bone tissue, or muscle tissue) and/or those that are isolated from a common organ, tissue system, blood vessel, or other structure and/or region in an organism.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term a “disease or condition that can be treated with an ALK2 or ALK3 inhibitor” refers to any disease or condition that could be expected to benefit from ALK2 or ALK3 inhibition based on studies performed in cell culture conditions, animal models, or human trials. Such diseases and conditions include anemia, bone diseases, fibrodysplasia ossificans progressiva; Sjogren's syndrome, multiple osteochondroma, diffuse intrinsic pontine glioma, posterior capsule opacification, cardiac hypertrophy, and cardiac fibrosis.

As used herein, the term an “isolated antibody” refers to an antibody that is substantially free of other antibodies having different antigenic specificities (e.g., an isolated antibody that binds to ALK2 or ALK3 is substantially free of contaminants, e.g., antibodies that do not bind to ALK2 or ALK3). In addition, an “isolated” antibody is one that has been identified and separated and/or recovered from a component of its natural environment. Contaminant components of its natural environment are materials that could interfere with diagnostic or therapeutic uses for the antibody, and may include enzymes, hormones, and other proteinaceous or non-proteinaceous solutes.

As used herein, the terms “increasing” and “decreasing” refer to modulating resulting in, respectively, greater or lesser amounts, of function, expression, or activity of a metric relative to a reference. For example, subsequent to administration of an ALK2 or ALK3 antibody or an antigen binding fragment thereof (e.g., an ALK2 or ALK3 binding fragment), of the invention in a method described herein, the amount of a marker of a metric (e.g., red blood cell levels, bone mineral density, tear production, survival time) as described herein may be increased in a subject relative to the amount of the marker prior to administration or relative to an untreated subject, or the amount of a marker of a metric (e.g., heterotopic ossification, or osteochondroma size, number, or formation) as described herein may be decreased in a subject relative to the amount of the marker prior to administration or relative to an untreated subject. Generally, the metric is measured subsequent to administration at a time that the administration has had the recited effect, e.g., at least one week, one month, 3 months, or 6 months, after a treatment regimen has begun.

As used herein, the terms “increase red blood cell levels” and “promote red blood cell formation” refer to clinically observable metrics, such as increases in hematocrit, red blood cell counts, and hemoglobin measurements, and are intended to be neutral as to the mechanism by which such changes occur. The term “low red blood cell levels” as used herein refers to red blood cell counts, hematocrit, and/or hemoglobin measurements that are below the range of values that is considered normal for the subject's age and gender. In some embodiments, an ALK2 or ALK3 antibody or an antigen binding fragment thereof (e.g., an ALK2 or ALK3 binding fragment), of the invention is administered to a subject (e.g., a subject having anemia) to increase red blood cell levels (e.g., increase hemoglobin levels or red blood cell counts).

As used herein, the terms “red blood cell formation” and “red blood cell production” refer to the generation of red blood cells, such as the process of erythropoiesis in which red blood cells are produced in the bone marrow.

As used herein, the term “anemia” refers to any abnormality in hemoglobin or red blood cells that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels.

As used herein the term “anemia of inflammation” refers to a type of anemia driven by inflammatory cytokines and characterized by hypoferremia despite adequate iron stores, which is largely due to elevated hepcidin. Bone morphogenetic proteins (BMP) upregulate hepcidin by activating the SMAD signaling pathway through ALK2. Anemia of inflammation is also called anemia of chronic disease. Chronic conditions that cause anemia of inflammation include infection (e.g., chronic infection, such as HIV/AIDS or tuberculosis), autoimmune disease (e.g., rheumatoid arthritis or lupus), cancer (e.g., cancer or cancer treatment), inflammatory bowel disease (e.g., Crohn's disease or ulcerative colitis), and chronic kidney disease.

As used herein the terms “iron refractory iron deficiency anemia” and “IRIDA” refer to an inherited form of iron deficiency anemia. Key features of IRIDA include lifelong anemia (hemoglobin 6-9 g/dL); very low red blood cell size (microcytic), with a mean corpuscular volume (MCV) of 45-65 fL; very low iron levels in the blood (transferrin saturation <5%); abnormal oral iron absorption—no response to oral iron supplements or failure of an “oral iron challenge;” abnormal iron utilization—a slow, incomplete, and transient response to parenteral iron (iron injected intravenously); and other affected family members with an autosomal recessive inheritance pattern. A diagnosis of IRIDA can be confirmed by measuring the level of hepcidin in the blood. Mutations in TMPRSS6 have been found to be associated with IRIDA.

As used herein, the term “low transfusion burden” refers to a condition of a subject that has received less than four units of red blood cells (RBCs) within eight weeks (e.g., 3, 2, 1, or 0 units of RBCs within eight weeks) prior to treatment with an antibody described herein. A subject with a low transfusion burden can be identified as having anemia based on measurements of mean hemoglobin concentration. A subject with a low transfusion burden and a mean hemoglobin concentration of less than 10.0 g/dL of two measurements performed at least one week apart prior to treatment with an antibody described herein (e.g., one measurement performed within one day prior to treatment and the other performed 7-28 days prior, not influenced by RBC transfusion within seven days of measurement) is defined as having anemia. In some embodiments, a subject with a low transfusion burden receives 1-3 units of RBCs (1-3 RBC transfusions) within eight weeks prior to treatment with an antibody described herein. In some embodiments, a subject with a low transfusion burden does not receive any units of RBCs (0 RBC transfusions) within eight weeks prior to treatment with an antibody described herein.

As used herein, the term “high transfusion burden” refers to a condition of a subject requiring greater than or equal to four units of RBCs (e.g., 4, 5, 6, 7, 8, or more units) within eight weeks prior to treatment with an antibody described herein. A subject with a high transfusion burden can be identified as having anemia based on measurements of mean hemoglobin concentration. A subject with a high transfusion burden and a mean hemoglobin concentration of less than or equal to 9.0 g/dL is defined as having anemia.

As used herein, the term “ineffective hematopoiesis” refers to the failure to produce fully mature hematopoietic cells (e.g., the failure to produce red blood cells, platelets, and neutrophils). Ineffective hematopoiesis may be due to single or multiple defects, such as abnormal proliferation and/or differentiation of progenitor cells (e.g., an excessive production of progenitors that are unable to complete differentiation), that can lead to a hyperproliferation or a shortage of progenitor cells.

As used herein, the terms “erythropoiesis stimulating agent” and “ESA” refer to a class of drugs that act on the proliferation stage of red blood cell development by expanding the pool of early-stage progenitor cells. Examples of erythropoiesis-stimulating agents are epoetin alfa and darbepoetin alfa.

As used herein, the terms “heterotopic ossification,” “heterotopic bone formation,” and “ectopic bone formation” refer to the abnormal growth of bone in non-skeletal tissues, such as muscles, tendons, and other soft tissue. “Ectopic bone” refers to bone that has formed in non-skeletal tissues. Heterotopic ossification can occur in subjects with FOP.

As used herein, the terms “fibrodysplasia ossificans progressiva” and “FOP” refer to a disorder in which skeletal muscle and connective tissue, such as tendons and ligaments, are gradually replaced by bone (ossified). This condition leads to bone formation outside the skeleton (extra-skeletal or heterotopic bone) that restricts movement. This process generally becomes noticeable in early childhood, starting with the neck and shoulders and moving down the body and into the limbs. People with FOP are born with abnormal big toes (hallux valgus) which can be helpful in making the diagnosis. Trauma, such as a fall or invasive medical procedure, or a viral illness may trigger episodes of muscle swelling and inflammation (myositis). These flare-ups last for several days to months and often result in permanent bone growth in the injured area. FOP is caused by mutation of the ACVR1 gene and is inherited in an autosomal dominant manner.

As used herein, the term “fibrosis” refers to the pathological process of excess formation of fibrous connective tissue. Fibrosis is characterized by fibroblast accumulation and collagen deposition in excess of normal deposition in any particular tissue. In response to inflammation or an injury to a tissue, nearby fibroblasts can migrate into the wound, proliferate, and produce large amounts of collagenous extracellular matrix. When fibrosis occurs in response to injury, the term “scarring” can be used as synonym. Fibrosis may occur in many tissues of the body, including, e.g., lungs, skin, liver, kidney, heart, eye, lacrimal gland, tendon, cartilage, pancreatic tissue, uterine tissue, neural tissue, testis, ovary, adrenal gland, artery, vein, colon, small and large intestine, biliary tract, and gut.

As used herein, the term “cardiac hypertrophy” refers to the abnormal enlargement, or thickening, of the heart muscle resulting from a process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality and has been found to be associated with fibrous tissue deposition in the cardiac interstitium accompanied by alterations in the extracellular matrix scaffold (e.g., cardiac fibrosis).

As used herein, the term “cardiac fibrosis,” also known as myocardial fibrosis, refers to fibrosis of the myocardium and is characterized by wall stiffening, reduced contractility, and impaired overall heart performance. Cardiac myofibroblasts mediate fibrosis by the excessive deposition of connective tissue (e.g., extracellular matrix) in the interstitial space. Cardiac fibrosis is often found in association with cardiac hypertrophy.

As used herein, the term “dry eye” refers to a chronic condition that occurs when the eyes do not produce enough tears (e.g., reduced tear production or tear volume) or when the tears evaporate too quickly. Dry eye may be accompanied by eye discomfort and abnormal visual function.

The term “Sjogren's syndrome” as used herein refers to a systemic inflammatory disorder characterized by dry mouth, decreased tearing, and other dry mucous membranes. Dryness of the eyes and mouth are the most common symptoms of this syndrome.

As used herein, “dry eye associated with Sjogren's syndrome” refers to dry eye associated with primary Sjogren's syndrome and dry eye associated with secondary Sjogren's syndrome. Dry eye associated with Sjogren's syndrome is generally classified as dry eye with reduced tear secretion.

As used herein, the term “multiple osteochondroma” or “MO” refers to a condition or disease associated with formation of osteochondromas on bones, e.g., at the ends of long bones or on flat bones. Subjects with MO often carry a loss-of-function mutation in an exostosin gene, e.g., EXT1 or EXT2. MO is also known as multiple hereditary exostoses, Bessel-Hagen disease, diaphyseal aclasis, multiple cartilaginous exostoses, multiple congenital exostosis, and hereditary multiple osteochondroma.

As used herein, the term “osteochondroma” refers to a benign (noncancerous) tumor that develops during childhood or adolescence. It is an abnormal overgrowth of cartilage and bone that typically forms on the surface of a bone near the growth plate. Osteochondromas most often form on the long bones of the leg and arm and on flat bones such as the pelvis and shoulder blade (scapula).

As used herein, the term “diffuse intrinsic pontine glioma” refers to a highly aggressive and difficult to treat brain tumor that originates in the pons and accounts for approximately 20% of all pediatric brain tumors. The median overall survival for children with diffuse intrinsic pontine glioma (DIPG) is 9-11 months, with one- and two-year survival rates of approximately 30% and less than 10%, respectively. DIPG has been found to be molecularly distinct from adult gliomas, and frequently harbors mutations in genes encoding histone variants. Mutations in ALK2 (ACVR1) have also been observed in DIPG.

As used herein, the term “posterior capsule opacification” refers to the most common postoperative complication of cataract surgery that occurs in 20% to 40% of patients. In posterior capsule opacification (PCO), the posterior capsule undergoes secondary opacification due to the migration, proliferation, and differentiation of lens epithelial cells. PCO can cause significant visual symptoms, particularly when it involves the central visual axis, and can be identified by the formation of fibrosis (e.g., scar tissue) on the posterior capsule (e.g., behind a lens implant). Symptoms of PCO include a gradual decrease of vision, blurred vision, sensitivity to sunlight, an observation of a glare or halo around lights.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity over the length of comparison, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. In certain embodiments, the present amino acid sequence identity is at least 80%, 90%, 95%, 98%, or 99%.

As used herein, the term “affinity” or “binding affinity” refers to the strength of the binding interaction between two molecules. Generally, binding affinity refers to the strength of the sum total of non-covalent interactions between a molecule and its binding partner, such as an ALK2 or ALK3 antibody or an antigen binding fragment thereof (e.g., an ALK2 or ALK3 binding fragment), and ALK2 or ALK3 protein. Unless indicated otherwise, binding affinity refers to intrinsic binding affinity, which reflects a 1:1 interaction between members of a binding pair. The binding affinity between two molecules is commonly described by the dissociation constant (K_{D}) or the affinity constant (K_{A}). Two molecules that have low binding affinity for each other generally bind slowly, tend to dissociate easily, and exhibit a large K_{D}. Two molecules that have high affinity for each other generally bind readily, tend to remain bound longer, and exhibit a small K_{D}. The K_{D} of two interacting molecules may be determined using methods and techniques well known in the art, e.g., surface plasmon resonance. K_{D} is calculated as the ratio of k_{off}/kₒₙ.

As used herein, the term “specific binding” of an antibody or ALK2 binding fragment thereof (e.g., a Fab, Fab′, F(ab′)₂, or Fv fragment), is binding to a target molecule that is measurably different from binding to molecules that are not target molecules. As used herein, specific binding refers to a greater than 95% preference for binding a particular antigen versus background (“non-specific”) binding. “Substantially specific” binding refers to a greater than about 80% preference for binding a particular antigen versus background. Binding can be measured using a variety of methods including, but not limited to, Western blot, immunoblot, enzyme-linked immunosorbent assay (“ELISA”), radioimmunoassay (“RIA”), immunoprecipitation, surface plasmon resonance, bio-layer interferometry, chemiluminescence, fluorescent polarization, phosphorescence, immunohistochemical analysis, matrix-assisted laser desorption/ionization time-of-flight (“MALDI-TOF”) mass spectrometry, microcytometry, microarray, microscopy, fluorescence activated cell sorting (“FACS”) and flow cytometry. In certain embodiments, an antibody that specifically binds to a target (e.g., ALK2 or ALK3) has a dissociation constant (K_{D}) no more than 0.1 nM, 0.15 nM, 0.2 nM, 0.3 nM, 0.5 nM, 0.7 nM, 1.0 nM, 2.0 nM, 4.0 nM, 5.0 nM, 7.0 nM, 10.0 nM, 14.0 nM, or 15.0 nM.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the terms “effective amount,” “therapeutically effective amount,” and “sufficient amount” of a composition or ALK2 or ALK3 antibody or an antigen binding fragment thereof (e.g., an ALK2 or ALK3 binding fragment), described herein refer to a quantity sufficient to, when administered to the subject effect beneficial or desired results, including clinical results, and, as such, an “effective amount” or synonym thereto depends upon the context in which it is being applied. For example, in the context of treating patient having a or at risk of developing a disease, such as bone disease (e.g., osteoporosis, or a condition involving bone damage, e.g., primary osteoporosis, secondary osteoporosis, osteopenia, osteopetrosis, osteogenesis imperfecta, bone fracture, bone cancer or cancer metastasis-related bone loss (e.g., bone loss associated with multiple myeloma), Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, and immobility-related bone loss), a disease or condition involving low red blood cell levels (e.g., anemia or blood loss), heterotopic ossification (e.g., heterotopic ossification resulting from FOP), Sjogren's syndrome (e.g., dry eye associated with Sjogren's syndrome), DIPG, MO, PCO, or cardiac hypertrophy and/or fibrosis, it is an amount of the composition or ALK2 or ALK3 antibody or an antigen binding fragment thereof (e.g., an ALK2 or ALK3 binding fragment), sufficient to achieve a treatment response as compared to the response obtained without administration of the composition or ALK2 or ALK3 antibody or an antigen binding fragment thereof (e.g., an ALK2 or ALK3 binding fragment). The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art by routine methods known in the art. Dosage regimen may be adjusted to provide the optimum therapeutic response.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, and administration to a mucous membrane of the subject, wherein the administration is intended to have a local and not a systemic effect.

As used herein, the term “pharmaceutical composition” refers to a mixture containing a therapeutic agent, optionally in combination with one or more pharmaceutically acceptable excipients, diluents, and/or carriers, to be administered to a subject in order to prevent, treat or control a particular disease or condition affecting or that may affect the subject. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and suitable for contact with the tissues of a subject without excessive toxicity, irritation, allergic response, and other problem complications commensurate with a reasonable benefit/risk ratio. In the present invention, the pharmaceutically acceptable carrier or excipient must provide adequate pharmaceutical stability to the ALK2 or ALK3 antibody or an antigen binding fragment thereof (e.g., an ALK2 or ALK3 binding fragment) of the invention. The nature of the carrier or excipient differs with the mode of administration. For example, for intravenous administration, an aqueous solution carrier is generally used; for oral administration, a solid carrier is preferred.

As used herein, the term “sample” refers to a specimen (e.g., blood, blood component (e.g., serum or plasma), urine, saliva, amniotic fluid, cerebrospinal fluid, tissue (e.g., neural tissue, placental tissue, or dermal tissue), pancreatic fluid, chorionic villus sample, and cells (e.g., blood cells or bone cells)) isolated from a subject.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the terms “subject” and “patient” refer to a mammal, e.g., a human. Mammals include, but are not limited to, humans and domestic and farm animals, such as monkeys (e.g., a cynomolgus monkey), mice, dogs, cats, horses, and cows, etc. A subject to be treated according to the methods described herein may be one who has been diagnosed with bone disease (e.g., a disease or condition involving bone damage, e.g., osteoporosis, osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, and immobility-related bone loss), a disease involving low blood cell levels (e.g., anemia or blood loss), heterotopic ossification (e.g., heterotopic ossification resulting from FOP), Sjogren's syndrome (e.g., dry eye associated with Sjogren's syndrome), DIPG, MO, PCO, or cardiac hypertrophy and/or fibrosis or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

### DESCRIPTION OF THE DRAWINGS

FIGS. 1A-1B are a series of graphs showing the pharmacokinetic properties of ALK2 antibodies after administration to cynomolgus monkeys. FIG. 1A is a graph of the pharmacokinetic properties of Antibody 2 over a 14-day period. FIG. 1B is a graph of the pharmacokinetic properties of Antibody 4 over a 17-day period. Error bars represent SEM, and the pharmacokinetic values were calculated using a standard non-compartmentalized model.

FIGS. 2A-2B are a series of a graphs showing the effect of treatment with an ALK2 antibody on serum hepcidin after administration to cynomolgus monkeys. FIG. 2A is a graph of hepcidin serum concentration after administration of Antibody 2. FIG. 2B is a graph of hepcidin serum concentration after administration of Antibody 4. Error bars represent SEM.

FIGS. 3A-3B are a series of graphs showing the effect of treatment with an ALK2 antibody on serum iron levels after administration to cynomolgus monkeys. FIG. 3A is a graph of serum iron levels after administration of Antibody 2. FIG. 3B is a graph of serum iron levels after administration of Antibody 4. Error bars represent SEM.

FIGS. 4A-4B are a series of graphs showing the effect of treatment with an ALK2 antibody on transferrin saturation after administration to cynomolgus monkeys. FIG. 4A is a graph of percent transferrin levels after administration of Antibody 2. FIG. 4B is a graph of percent transferrin saturation after administration of Antibody 4. The serum from three monkeys was combined for each time point before the assay.

FIGS. 5A-5C are a series of graphs showing the effect of treatment with an ALK2 antibody on reticulocyte hemoglobin, red blood cell hemoglobin, and mean corpuscular hemoglobin content in cynomolgus monkeys. The graphs show the percent change in reticulocyte hemoglobin (FIG. 5A), percent change in red blood cell (RBC) hemoglobin (FIG. 5B), and percent change in mean corpuscular hemoglobin concentration (MCHC) (FIG. 5C) over time after administration of Antibody 2 or Antibody 4. Data from Antibody 2 and Antibody 4 were combined for each metric. Error bars represent SEM.

FIGS. 6A-6C are a series of graphs showing the effect of single dose administration of an ALK2 antibody on CD1 mice. The graphs show drug exposure (FIG. 6A), hepcidin levels (FIG. 6B), and iron levels (FIG. 6C) from mice treated with 0.5 mg/kg, 1.0 mg/kg, or 3.0 mg/kg of an IgG1 isotype control, Antibody 2, or Antibody 4. ns—not significant; *p<0.05; **p<0.01; ***p<0.001. Error bars represent SEM.

### DETAILED DESCRIPTION OF THE INVENTION

Described herein are methods for treating for a disease or condition that can be treated with an ALK2 or ALK3 inhibitor, including bone disease, anemia, heterotopic ossification (e.g., heterotopic ossification resulting from FOP), Sjogren's syndrome (e.g., dry eye associated with Sjogren's syndrome), DIPG, MO, PCO, or cardiac hypertrophy and/or cardiac fibrosis. The invention features methods of treating these diseases and conditions by administering an ALK2 or ALK3 antibody (e.g., an ALK2 or ALK3 neutralizing antibody) or an antigen binding fragment thereof (e.g., an ALK2 or ALK3 binding fragment), in an amount of 0.002 mg/kg to 3 mg/kg.

### ALK2 and ALK3 Antibodies

ALK2 (also known as ACVR1) and ALK3 (also known as BMPR1A) are bone morphogenetic protein (BMP) receptors in the transforming growth factor β (TGF-β) superfamily. ALK2 and ALK3 are widely expressed in many tissues during embryonic development and highly expressed in bones during postnatal development and are thought to have essential roles in both osteogenesis and chondrogenesis. Gain of function mutations in ALK2, including c.617G>A (p.R206H), c.619C>G (p.Q207E), c.1067G>A (p.G356D), c.982G>T(p.G328W), c.983G>A(p.G328E), c.982G>A (p.G328R), c.774G>C/c.774G>T (P.R258S), c.1124G>C (p.R375P), c.587T>C (p.L196P), c.590-592deICTT (p.P197_F198delinsL), and c.605G>T (p.R2021), have been found in subjects with FOP, and studies using ALK2^{R206H} mutant mice suggest that ALK2 regulates the osteogenic differentiation of mesenchymal stem cells. Activating mutations in ALK2 have also been observed in approximately 25% of DIPG patients and small molecule ALK2 inhibitors have been found to extend survival and reduce cellularity in orthotopic DIPG xenograft models. ALK2 may also play a role in anemia, as BMP activation of the SMAD signaling pathway through ALK2 induces the upregulation of hepcidin, a master regulator of iron homeostasis that is implicated in anemia of inflammation (also known as anemia of chronic disease) and iron refractory iron deficiency anemia (IRIDA). ALK2 also promotes the proliferation of lens epithelial cells during development; therefore, inhibition of ALK2 may prevent or reduce the aberrant proliferation of lens epithelial cells that leads to PCO. In addition, small molecule ALK2 inhibitors have been found to decrease inflammation and treat symptoms of Sjogren's syndrome in an established mouse model, and to reduce osteochondroma formation, growth, and size in multiple studies using mouse models of MO. Furthermore, both treatment with a small molecule ALK2 inhibitor and cardiomyocyte-specific deletion of ALK2 mitigated cardiac hypertrophy and left ventricular fibrosis in mice. Moreover, the effect of ALK2 ligands in promoting fibrosis suggests that inhibiting ALK2 can be used to treat diseases or conditions in which fibrosis contributes to the pathology (e.g., fibrosis of the posterior capsule in PCO, fibrosis of the lacrimal gland in Sjogren's syndrome, and fibrosis in cardiac fibrosis and/or cardiac hypertrophy). Downregulation of ALK3 signaling in young and old mice has been found to increase bone mass and/or prevent bone loss, suggesting that inhibiting ALK3 could be used to treat bone diseases. In addition, knockout of ALK3 has been observed to partly protect from anemia of inflammation, suggesting that ALK3 may be a possible therapeutic target for treatment. In addition, inhibition of ALK3 may also disrupt ALK2 signaling.

The present invention is based, in part, on the discovery that administration of an ALK2 antibody described herein to non-human primates in an amount of 3 mg/kg resulted in reduced serum hepcidin levels within six hours of administration that was sustained for at least 10 days and increased serum iron and increased transferrin saturation for at least 10 days after administration. Increased reticulocyte hemoglobin and red blood cell hemoglobin were also observed.

The ALK2 antibody administered to the subject may be an ALK2 antibody or an antigen-binding fragment thereof as described in Table 2. In some embodiments, the ALK2 antibody administered to the subject is an ALK2 antibody or antigen-binding fragment thereof, as described in International Patent Application Publication Nos. WO2020086730, WO2020118011, and WO2021163170, US Patent Application Publication Nos. US20210253720, US20220098310, and US20210253716, and U.S. Pat. No. 10,428,148, which are incorporated herein by reference. In some embodiments, the ALK2 antibody administered to the subject is a Mab or Fab described in Aykul et al., J Clin Invest., e-publication doi: 10.1172/JCI153792, 2022.

TABLE 2

| Row | Composition |
|---|---|
| 1 | An isolated antibody, or ALK2 binding fragment thereof, comprising (1) a light chain variable |
| | domain comprising a light chain complementarity determining region (CDR)1 comprising an |
| | amino acid sequence selected from the group consisting of SGSSSNIGSNYVS (SEQ ID NO: |
| | 1) and SGDX₁X₂X₃X₄X₅X₆X₇X₈ (SEQ ID NO: 2, wherein X₁ is S or N, X₂ is I or L, X₃ is P, G, |
| | or R, X₄ is S, T, or K, X₅ is F, K, or Y, X₆ is F, Y, or S, X₇ is A or V, and X₈ is S, Y, or H); a |
| | light chain CDR2 comprising the amino acid sequence X₁X₂IYX₃X₄X₅X₆RPS (SEQ ID NO: 3, |
| | wherein X₁ is V or L, X₂ is V or L, X₃ is K, R, G or Y, X₄ is N or D, X₅ is N or S, and X₆ is H, N, |
| | D, or K); and a light chain CDR3 comprising an amino acid sequence selected from the |
| | group consisting of ASWDHSDRFYV (SEQ ID NO: 4), YVTAPWKSIW (SEQ ID NO: 5), |
| | YSADAQQMKA (SEQ ID NO: 6), QVYASVHRM (SEQ ID NO: 7), and QTYDWSHFGW (SEQ |
| | ID NO: 8); and (2) a heavy chain variable domain comprising a heavy chain CDR1 |
| | comprising the amino acid sequence GX₁TFX₂SX₃X₄X₅X₆ (SEQ ID NO: 9, wherein X₁ is G or |
| | F, X₂ is S or N, X₃ is Y, H, S, or A, X₄ is G or A, X₅ is V, M, or I, and X₆ is S or H); a heavy |
| | chain CDR2 comprising an amino acid sequence selected from the group consisting of |
| | WMGX₁IIPX₂FGX₃ANYAQKFQG (SEQ ID NO: 10, wherein X₁ is G or R, X₂ is H or D, and X₃ |
| | is I or T), WVGRIKSKX₁DX₂X₃TTDYAAPVKG (SEQ ID NO: 11, wherein X₁ is A or R, X₂ is S |
| | or G, and X₃ is G or Y), and WVSVISSDGGSTYYADSVKG (SEQ ID NO: 12); and a heavy |
| | chain CDR3 comprising an amino acid sequence selected from the group consisting of |
| | EIGSLDI (SEQ ID NO: 13), DYGVAFAY (SEQ ID NO: 14), DYGGLKFDY (SEQ ID NO: 15), |
| | GPTQAIHYFAY (SEQ ID NO: 16), and AGFILGSLGVAWMDV (SEQ ID NO: 17). |
| 2 | The isolated antibody, or ALK2 binding fragment thereof, of row 1, wherein the light chain |
| | CDR1 comprises the sequence SGSSSNIGSNYVS (SEQ ID NO: 1). |
| 3 | The isolated antibody, or ALK2 binding fragment thereof, of row 1, wherein the light chain |
| | CDR1 comprises the sequence SGDSIPSFFAS (SEQ ID NO: 18). |
| 4 | The isolated antibody, or ALK2 binding fragment thereof, of row 1, wherein the light chain |
| | CDR1 comprises the sequence SGDNIGTKYAY (SEQ ID NO: 19). |
| 5 | The isolated antibody, or ALK2 binding fragment thereof, of row 1, wherein the light chain |
| | CDR1 comprises the sequence SGDNLRKYSAH (SEQ ID NO: 20). |
| 6 | The isolated antibody, or ALK2 binding fragment thereof, of row 1, wherein the light chain |
| | CDR1 comprises the sequence SGDSLGSKSVH (SEQ ID NO: 21). |
| 7 | The isolated antibody, or ALK2 binding fragment thereof, of any one of rows 1 to 6, wherein |
| | the light chain CDR2 comprises the sequence VLIYKNNHRPS (SEQ ID NO: 24). |
| 8 | The isolated antibody, or ALK2 binding fragment thereof, of any one of rows 1 to 6, wherein |
| | the light chain CDR2 comprises the sequence LVIYRDSNRPS (SEQ ID NO: 25). |
| 9 | The isolated antibody, or ALK2 binding fragment thereof, any one of rows 1 to 6, wherein the |
| | light chain CDR2 comprises the sequence LVIYGDSDRPS (SEQ ID NO: 26). |
| 10 | The isolated antibody, or ALK2 binding fragment thereof, any one of rows 1 to 6, wherein the |
| | light chain CDR2 comprises the sequence LVIYYDNKRPS (SEQ ID NO: 27). |
| 11 | The isolated antibody, or ALK2 binding fragment thereof, of any one of rows 1 to 6, wherein |
| | the light chain CDR2 comprises the sequence LVIYRDSKRPS (SEQ ID NO: 28). |
| 12 | The isolated antibody, or ALK2 binding fragment thereof, of any one of rows 1 to 11, wherein |
| | the light chain CDR3 comprises the sequence ASWDHSDRFYV (SEQ ID NO: 4). |
| 13 | The isolated antibody, or ALK2 binding fragment thereof, of any one of rows 1 to 11, wherein |
| | the light chain CDR3 comprises the sequence YVTAPWKSIW (SEQ ID NO: 5). |
| 14 | The isolated antibody, or ALK2 binding fragment thereof, of any one of rows 1 to 11, wherein |
| | the light chain CDR3 comprises the sequence YSADAQQMKA (SEQ ID NO: 6). |
| 15 | The isolated antibody, or ALK2 binding fragment thereof, of any one of rows 1 to 11, wherein |
| | the light chain CDR3 comprises the sequence QVYASVHRM (SEQ ID NO: 7). |
| 16 | The isolated antibody, or ALK2 binding fragment thereof, of any one of rows 1 to 11, wherein |
| | the light chain CDR3 comprises the QTYDWSHFGW (SEQ ID NO: 8). |
| 17 | The isolated antibody, or ALK2 binding fragment thereof, of any one of rows 1 to 16, wherein |
| | the heavy chain CDR1 comprises the sequence GGTFSSYGVS (SEQ ID NO: 31). |
| 18 | The isolated antibody, or ALK2 binding fragment thereof, of any one of rows 1 to 16, wherein |
| | the heavy chain CDR1 comprises the sequence GFTFSSHAMS (SEQ ID NO: 32). |
| 19 | The isolated antibody, or ALK2 binding fragment thereof, of any one of rows 1 to 16, wherein |
| | the heavy chain CDR1 comprises the sequence GFTFNSSAMS (SEQ ID NO: 33). |
| 20 | The isolated antibody, or ALK2 binding fragment thereof, of any one of rows 1 to 16, wherein |
| | the heavy chain CDR1 comprises the sequence GGTFSSYAIH (SEQ ID NO: 34). |
| 21 | The isolated antibody, or ALK2 binding fragment thereof, of any one of rows 1 to 16, wherein |
| | the heavy chain CDR1 comprises the sequence GFTFSSAAMH (SEQ ID NO: 35). |
| 22 | The isolated antibody, or ALK2 binding fragment thereof, of any one of rows 1 to 21, wherein |
| | the heavy chain CDR2 comprises the sequence WMGGIIPHFGIANYAQKFQG (SEQ ID NO: |
| | 36) |
| 23 | The isolated antibody, or ALK2 binding fragment thereof, of any one of rows 1 to 21, wherein |
| | the heavy chain CDR2 comprises the sequence WVGRIKSKADSGTTDYAAPVKG (SEQ ID |
| | NO: 37). |
| 24 | The isolated antibody, or ALK2 binding fragment thereof, of any one of row s1 to 21, wherein |
| | the heavy chain CDR2 comprises the sequence WVGRIKSKRDGYTTDYAAPVKG (SEQ ID |
| | NO: 38). |
| 25 | The isolated antibody, or ALK2 binding fragment thereof, of any one of rows 1 to 21, wherein |
| | the heavy chain CDR2 comprises the sequence WMGRIIPDFGTANYAQKFQG (SEQ ID NO: |
| | 39). |
| 26 | The isolated antibody, or ALK2 binding fragment thereof, of any one of rows 1 to 21, wherein |
| | the heavy chain CDR2 comprises the sequence WVSVISSDGGSTYYADSVKG (SEQ ID NO: |
| | 12). |
| 27 | The isolated antibody, or ALK2 binding fragment thereof, of any one of rows 1 to 26, wherein |
| | the heavy chain CDR3 comprises the sequence EIGSLDI (SEQ ID NO: 13). |
| 28 | The isolated antibody, or ALK2 binding fragment thereof, of any one of row 1 to 26, wherein |
| | the heavy chain CDR3 comprises the sequence DYGVAFAY (SEQ ID NO: 14). |
| 29 | The isolated antibody, or ALK2 binding fragment thereof, of any one of row 1 to 26, wherein |
| | the heavy chain CDR3 comprises the sequence DYGGLKFDY (SEQ ID NO: 15). |
| 30 | The isolated antibody, or ALK2 binding fragment thereof, of any one of row 1 to 26, wherein |
| | the heavy chain CDR3 comprises the sequence GPTQAIHYFAY (SEQ ID NO: 16). |
| 31 | The isolated antibody, or ALK2 binding fragment thereof, of any one of row 1 to 26, wherein |
| | the heavy chain CDR3 comprises the sequence AGFILGSLGVAWMDV (SEQ ID NO: 17). |
| 32 | The isolated antibody, or ALK2 binding fragment thereof, of row 1, wherein the light chain |
| | CDR2 comprises the sequence LVIYX₁DX₂X₃RPS (SEQ ID NO: 22, wherein X₁ is R, G, or Y, |
| | X₂ is S or N, and X₃ is N, D, or K). |
| 33 | The isolated antibody, or ALK2 binding fragment thereof, of row 1, wherein the light chain |
| | CDR2 comprises the sequence LVIYRDSX₁RPS (SEQ ID NO: 23, wherein X₁ is N or K). |
| 34 | The isolated antibody, or ALK2 binding fragment thereof, of row 1, wherein the heavy chain |
| | CDR1 comprises the sequence GFTFSSX₁AMX₂ (SEQ ID NO: 29, wherein X₁ is H or A, and |
| | X₂ is S or H). |
| 35 | The isolated antibody, or ALK2 binding fragment thereof, of row 1, wherein the heavy chain |
| | CDR1 comprises the sequence GFTFX₁SX₂AMS (SEQ ID NO: 30, wherein X₁ is S or N, and |
| | X₂ is H or S). |
| 36 | The isolated antibody, or ALK2 binding fragment thereof, of row 1, wherein the light chain |
| | CDR1 comprises the amino acid sequence SGSSSNIGSNYVS (SEQ ID NO: 1); the light |
| | chain CDR2 comprises the amino acid sequence VLIYKNNHRPS (SEQ ID NO: 24); and the |
| | light chain CDR3 comprises the amino acid sequence ASWDHSDRFYV (SEQ ID NO: 4). |
| 37 | The isolated antibody, or ALK2 binding fragment thereof, of row 1 or 36, wherein the heavy |
| | chain CDR1 comprises the amino acid sequence GGTFSSYGVS (SEQ ID NO: 31); the |
| | heavy chain CDR2 comprises the amino acid sequence WMGGIIPHFGIANYAQKFQG (SEQ |
| | ID NO: 36); and the heavy chain CDR3 comprises the amino acid sequence EIGSLDI (SEQ |
| | ID NO: 13). |
| 38 | The isolated antibody, or ALK2 binding fragment thereof, of row 1, wherein the light chain |
| | CDR1 comprises the amino acid sequence SGDSIPSFFAS (SEQ ID NO: 18); the light chain |
| | CDR2 comprises the amino acid sequence LVIYRDSNRPS (SEQ ID NO: 25); and the light |
| | chain CDR3 comprises the amino acid sequence YVTAPWKSIW (SEQ ID NO: 5). |
| 39 | The isolated antibody, or ALK2 binding fragment thereof, of row 1 or 38, wherein the heavy |
| | chain CDR1 comprises the amino acid sequence GFTFSSHAMS (SEQ ID NO: 32); the |
| | heavy chain CDR2 comprises the amino acid sequence WVGRIKSKADSGTTDYAAPVKG |
| | (SEQ ID NO: 37); and the heavy chain CDR3 comprises the amino acid sequence |
| | DYGVAFAY (SEQ ID NO: 14). |
| 40 | The isolated antibody, or ALK2 binding fragment thereof, of row 1, wherein the light chain |
| | CDR1 comprises the amino acid sequence SGDNIGTKYAY (SEQ ID NO: 19); the light chain |
| | CDR2 comprises the amino acid sequence LVIYGDSDRPS (SEQ ID NO: 26); and the light |
| | chain CDR3 comprises the amino acid sequence YSADAQQMKA (SEQ ID NO: 6). |
| 41 | The isolated antibody, or ALK2 binding fragment thereof, of row 1 or 40, wherein the heavy |
| | chain CDR1 comprises the amino acid sequence GFTFNSSAMS (SEQ ID NO: 33); the |
| | heavy chain CDR2 comprises the amino acid sequence WVGRIKSKRDGYTTDYAAPVKG |
| | (SEQ ID NO: 38); and the heavy chain CDR3 comprises the amino acid sequence |
| | DYGGLKFDY (SEQ ID NO: 15). |
| 42 | The isolated antibody, or ALK2 binding fragment thereof, of row 1, wherein the light chain |
| | CDR1 comprises the amino acid sequence SGDNLRKYSAH (SEQ ID NO: 20); the light |
| | chain CDR2 comprises the amino acid sequence LVIYYDNKRPS (SEQ ID NO: 27); and the |
| | light chain CDR3 comprises the amino acid sequence QVYASVHRM (SEQ ID NO: 7). |
| 43 | The isolated antibody, or ALK2 binding fragment thereof, of row 1 or 42 wherein the heavy |
| | chain CDR1 comprises the amino acid sequence GGTFSSYAIH (SEQ ID NO: 34); the heavy |
| | chain CDR2 comprises the amino acid sequence WMGRIIPDFGTANYAQKFQG (SEQ ID |
| | NO: 39); and the heavy chain CDR3 comprises the amino acid sequence GPTQAIHYFAY |
| | (SEQ ID NO: 16). |
| 44 | The isolated antibody, or ALK2 binding fragment thereof, of row 1, wherein the light chain |
| | CDR1 comprises the amino acid sequence SGDSLGSKSVH (SEQ ID NO: 21); the light |
| | chain CDR2 comprises the amino acid sequence LVIYRDSKRPS (SEQ ID NO: 28); and the |
| | light chain CDR3 comprises the amino acid sequence QTYDWSHFGW (SEQ ID NO: 8). |
| 45 | The isolated antibody, or ALK2 binding fragment thereof, of row 1 or 44, wherein the heavy |
| | chain CDR1 comprises the amino acid sequence GFTFSSAAMH (SEQ ID NO: 35); the |
| | heavy chain CDR2 comprises the amino acid sequence WVSVISSDGGSTYYADSVKG |
| | (SEQ ID NO: 12); and the heavy chain CDR3 comprises the amino acid sequence |
| | AGFILGSLGVAWMDV (SEQ ID NO: 17). |
| 46 | The isolated antibody of row 1, wherein the antibody, apart from the light chain CDR1, |
| | CDR2, and CDR3 and the heavy chain CDR1, CDR2, and CDR3, has at least 90% |
| | sequence identity (e.g., 90%, 91%, 92%, 93%, 94%, 95%, 95%, 97%, 98%, 99% or more |
| | sequence identity) to amino acids 1 to 331 of the sequence of SEQ ID NO: 67, amino acids 1 |
| | to 332 of the sequence of SEQ ID NO: 68, amino acids 1 to 333 of the sequence of SEQ ID |
| | NO: 69, amino acids 1 to 332 of the sequence of SEQ ID NO: 70, or amino acids 1 to 337 of |
| | the sequence of SEQ ID NO: 71. |
| 47 | The isolated antibody of row 1, wherein the antibody comprises amino acids 1 to 433 of the |
| | sequence of SEQ ID NO: 67, amino acids 1 to 434 of the sequence of SEQ ID NO: 68, |
| | amino acids 1 to 435 of the sequence of SEQ ID NO: 69, amino acids 1 to 434 of the |
| | sequence of SEQ ID NO: 70, or amino acids 1 to 439 of the sequence of SEQ ID NO: 71. |
| 48 | An isolated antibody, or ALK2 binding fragment thereof, comprising (1) a light chain variable |
| | domain comprising a light chain complementarity determining region (CDR)1 comprising an |
| | amino acid sequence selected from the group consisting of RASQGISGNWLT (SEQ ID NO: |
| | 40), SGDX₁X₂RX₃X₄X₅X₆H (SEQ ID NO: 64, wherein X₁ is N or A, X₂ is I or L, X₃ is K or Y, X₄ |
| | is K or Y, X₅ is Y or I, and X₆ is V or A), and SGSSSNIGQNYVS (SEQ ID NO: 58); a light |
| | chain CDR2 comprising the amino acid sequence LX₁IYX₂X₃X₄X₅X₆X₇S (SEQ ID NO: 65, |
| | where X₁ is V or L, X₂ is D, R, or Y, X₃ is A, D, or N, X₄ is S or N, X₅ is K or N, X₆ is L or R, |
| | and X₇ is Q or P); and a light chain CDR3 comprising an amino acid sequence selected from |
| | the group consisting of HQSYRGPM (SEQ ID NO: 42), SSAGRDNY (SEQ ID NO: 48), |
| | QSYGPGSV (SEQ ID NO: 54), and SSWDLLSKSR (SEQ ID NO: 60); and (2) a heavy chain |
| | variable domain comprising a heavy chain CDR1 comprising the amino acid sequence |
| | GX₁TFX₂X₃X₄X₅X₆X₇ (SEQ ID NO: 66, wherein X₁ is F or G, X₂ is G or S, X₃ is R, S, D, or T, |
| | X₄ is F, S, Y, or H, X₅ is V or A, and X₆ is M or I, and X₇ is H or S); a heavy chain CDR2 |
| | comprising an amino acid sequence selected from the group consisting of |
| | WVSX₁IX₂YX₃X₄SX₅TYYADSVKG (SEQ ID NO: 76, wherein X₁ is V or S, X₂ is G, H, or F, X₃ |
| | is S or D, X₄ is G or S, and X₅ is S, E, or N), and WMGLIQPRFGTANYAQKFQR (SEQ ID |
| | NO: 62,; and a heavy chain CDR3 comprising an amino acid sequence selected from the |
| | group consisting of EPGYYYPSGYYRGPGYWMDV (SEEQ ID NO: 45), DRYFFDV (SEQ ID |
| | NO: 51), PKSYASGPFAY (SEQ ID NO: 57), and DYYGGMAY (SEQ ID NO: 63). |
| 49 | The isolated antibody, or ALK2 binding fragment thereof, of row 48, wherein the light chain |
| | CDR1 comprises the sequence RASQGISGNWLT (SEQ ID NO: 40). |
| 50 | The isolated antibody, or ALK2 binding fragment thereof, of row 48, wherein the light chain |
| | CDR1 comprises the sequence SGDNIRKKYVH (SEQ ID NO: 46). |
| 51 | The isolated antibody, or ALK2 binding fragment thereof, of row 48, wherein the light chain |
| | CDR1 comprises the sequence SGDALRYYIAH (SEQ ID NO: 52). |
| 52 | The isolated antibody, or ALK2 binding fragment thereof, of row 48, wherein the light chain |
| | CDR1 comprises the sequence SGSSSNIGQNYVS (SEQ ID NO: 58). |
| 53 | The isolated antibody, or ALK2 binding fragment thereof, of any one of rows 48 to 52, |
| | wherein the light chain CDR2 comprises the sequence LLIYDASNLQS (SEQ ID NO: 41). |
| 54 | The isolated antibody, or ALK2 binding fragment thereof, of any one of rows 48 to 52, |
| | wherein the light chain CDR2 comprises the sequence LVIYRDSNRPS (SEQ ID NO: 47). |
| 55 | The isolated antibody, or ALK2 binding fragment thereof, of any one of rows 48 to 52, |
| | wherein the light chain CDR2 comprises the sequence LVIYYNNNRPS (SEQ ID NO: 53). |
| 56 | The isolated antibody, or ALK2 binding fragment thereof, of any one of rows 48 to 52, |
| | wherein the light chain CDR2 comprises the sequence LLIYDNSKRPS (SEQ ID NO: 59). |
| 57 | The isolated antibody, or ALK2 binding fragment thereof, of any one of rows 48 to 56, |
| | wherein the light chain CDR3 comprises the sequence HQSYRGPM (SEQ ID NO: 42). |
| 58 | The isolated antibody, or ALK2 binding fragment thereof, of any one of rows 48 to 56, |
| | wherein the light chain CDR3 comprises the sequence SSAGRDNY (SEQ ID NO: 48). |
| 59 | The isolated antibody, or ALK2 binding fragment thereof, of any one of rows 48 to 56, |
| | wherein the light chain CDR3 comprises the sequence QSYGPGSV (SEQ ID NO: 54). |
| 60 | The isolated antibody, or ALK2 binding fragment thereof, of any one of rows 48 to 56, |
| | wherein the light chain CDR3 comprises the sequence SSWDLLSKSR (SEQ ID NO: 60). |
| 61 | The isolated antibody, or ALK2 binding fragment thereof, of any one of rows 48 to 60, |
| | wherein the heavy chain CDR1 comprises the sequence GFTFGRFVMH (SEQ ID NO: 43). |
| 62 | The isolated antibody, or ALK2 binding fragment thereof, of any one of rows 48 to 60, |
| | wherein the heavy chain CDR1 comprises the sequence GFTFSSSAMH (SEQ ID NO: 49). |
| 63 | The isolated antibody, or ALK2 binding fragment thereof, of any one of rows 48 to 60, |
| | wherein the heavy chain CDR1 comprises the sequence GFTFSDYAMH (SEQ ID NO: 55). |
| 64 | The isolated antibody, or ALK2 binding fragment thereof, of any one of rows 48 to 60, |
| | wherein the heavy chain CDR1 comprises the sequence GGTFSTHAIS (SEQ ID NO: 61). |
| 65 | The isolated antibody, or ALK2 binding fragment thereof, of any one of rows 48 to 64, |
| | wherein the heavy chain CDR2 comprises the sequence WVSVIGYSGSSTYYADSVKG |
| | (SEQ ID NO: 44). |
| 66 | The isolated antibody, or ALK2 binding fragment thereof, of any one of rows 48 to 64, |
| | wherein the heavy chain CDR2 comprises the sequence WVSVIHYDSSETYYADSVKG |
| | (SEQ ID NO: 50). |
| 67 | The isolated antibody, or ALK2 binding fragment thereof, of any one of rows 48 to 64, |
| | wherein the heavy chain CDR2 comprises the sequence WVSSIFYSGSNTYYADSVKG |
| | (SEQ ID NO: 56). |
| 68 | The isolated antibody, or ALK2 binding fragment thereof, of any one of rows 48 to 64, |
| | wherein the heavy chain CDR2 comprises the sequence WMGLIQPRFGTANYAQKFQR |
| | (SEQ ID NO: 62). |
| 69 | The isolated antibody, or ALK2 binding fragment thereof, of any one of rows 48 to 68, |
| | wherein the heavy chain CDR3 comprises the sequence EPGYYYPSGYYRGPGYWMDV |
| | (SEQ ID NO: 45). |
| 70 | The isolated antibody, or ALK2 binding fragment thereof, of any one of rows 48 to 68, |
| | wherein the heavy chain CDR3 comprises the sequence DRYFFDV (SEQ ID NO: 51). |
| 71 | The isolated antibody, or ALK2 binding fragment thereof, of any one of rows 48 to 68, |
| | wherein the heavy chain CDR3 comprises the sequence PKSYASGPFAY (SEQ ID NO: 57). |
| 72 | The isolated antibody, or ALK2 binding fragment thereof, of any one of rows 48 to 68, |
| | wherein the heavy chain CDR3 comprises the sequence DYYGGMAY (SEQ ID NO: 63). |
| 73 | The isolated antibody, or ALK2 binding fragment thereof, of row 48, wherein the light chain |
| | CDR1 comprises the amino acid sequence RASQGISGNWLT (SEQ ID NO: 40); the light |
| | chain CDR2 comprises the amino acid sequence LLIYDASNLQS (SEQ ID NO: 41); and the |
| | light chain CDR3 comprises the amino acid sequence HQSYRGPM (SEQ ID NO: 42). |
| 74 | The isolated antibody, or ALK2 binding fragment thereof, of row 48 or 73, wherein the heavy |
| | chain CDR1 comprises the amino acid sequence GFTFGRFVMH (SEQ ID NO: 43); the |
| | heavy chain CDR2 comprises the amino acid sequence WVSVIGYSGSSTYYADSVKG (SEQ |
| | ID NO: 44); and the heavy chain CDR3 comprises the amino acid sequence |
| | EPGYYYPSGYYRGPGYWMDV (SEQ ID NO: 45). |
| 75 | The isolated antibody, or ALK2 binding fragment thereof, of row 48, wherein the light chain |
| | CDR1 comprises the amino acid sequence SGDNIRKKYVH (SEQ ID NO: 46); the light chain |
| | CDR2 comprises the amino acid sequence LVIYRDSNRPS (SEQ ID NO: 47); and the light |
| | chain CDR3 comprises the amino acid sequence SSAGRDNY (SEQ ID NO: 48). |
| 76 | The isolated antibody, or ALK2 binding fragment thereof, of row 48 or 75, wherein the heavy |
| | chain CDR1 comprises the amino acid sequence GFTFSSSAMH (SEQ ID NO: 49); the |
| | heavy chain CDR2 comprises the amino acid sequence WVSVIHYDSSETYYADSVKG (SEQ |
| | ID NO: 50); and the heavy chain CDR3 comprises the amino acid sequence DRYFFDV |
| | (SEQ ID NO: 51). |
| 77 | The isolated antibody, or ALK2 binding fragment thereof, of row 48, wherein the light chain |
| | CDR1 comprises the amino acid sequence SGDALRYYIAH (SEQ ID NO: 52); the light chain |
| | CDR2 comprises the amino acid sequence LVIYYNNNRPS (SEQ ID NO: 53); and the light |
| | chain CDR3 comprises the amino acid sequence QSYGPGSV (SEQ ID NO: 54). |
| 78 | The isolated antibody, or ALK2 binding fragment thereof, of row 48 or 77, wherein the heavy |
| | chain CDR1 comprises the amino acid sequence GFTFSDYAMH (SEQ ID NO: 55); the |
| | heavy chain CDR2 comprises the amino acid sequence WVSSIFYSGSNTYYADSVKG (SEQ |
| | ID NO: 56); and the heavy chain CDR3 comprises the amino acid sequence |
| | PKSYASGPFAY (SEQ ID NO: 57). |
| 79 | The isolated antibody, or ALK2 binding fragment thereof, of row 48, wherein the light chain |
| | CDR1 comprises the amino acid sequence SGSSSNIGQNYVS (SEQ ID NO: 58); the light |
| | chain CDR2 comprises the amino acid sequence LLIYDNSKRPS (SEQ ID NO: 59); and the |
| | light chain CDR3 comprises the amino acid sequence SSWDLLSKSR (SEQ ID NO: 60). |
| 80 | The isolated antibody, or ALK2 binding fragment thereof, of row 48 or 79, wherein the heavy |
| | chain CDR1 comprises the amino acid sequence GGTFSTHAIS (SEQ ID NO: 61); the heavy |
| | chain CDR2 comprises the amino acid sequence WMGLIQPRFGTANYAQKFQR (SEQ ID |
| | NO: 62); and the heavy chain CDR3 comprises the amino acid sequence DYYGGMAY (SEQ |
| | ID NO: 63). |
| 81 | The isolated antibody of row 48, wherein the antibody, apart from the light chain CDR1, |
| | CDR2, and CDR3 and the heavy chain CDR1, CDR2, and CDR3, has at least 90% |
| | sequence identity (e.g., 90%, 91%, 92%, 93%, 94%, 95%, 95%, 97%, 98%, 99% or more |
| | sequence identity) to amino acids 1 to 344 of the sequence of SEQ ID NO: 72, amino acids 1 |
| | to 327 of the sequence of SEQ ID NO: 73, amino acids 1 to 331 of the sequence of SEQ ID |
| | NO: 74, or amino acids 1 to 332 of the sequence of SEQ ID NO: 75. |
| 82 | The isolated antibody of row 48, wherein the antibody comprises amino acids 1 to 446 of the |
| | sequence of SEQ ID NO: 72, amino acids 1 to 429 of the sequence of SEQ ID NO: 73, |
| | amino acids 1 to 433 of the sequence of SEQ ID NO: 74, or amino acids 1 to 434 of the |
| | sequence of SEQ ID NO: 75. |

In some embodiments, the ALK2 antibody administered to the subject is an ALK2 antibody or an antigen-binding fragment thereof, having CDR sequences described in Table 3 (i.e., a light chain CDR1, CDR2, and CDR3 and a heavy chain CDR1, CDR2, and CDR3). In some embodiments, the ALK2 antibody or antigen binding fragment thereof includes a light chain variable CDR1 sequence having at least 90% (e.g., at least 91%, 92%, 93%, 94%, 95%, 96%, 97%, 98%, 99%, or 100%) sequence identity to a light chain variable CDR1 sequence in Table 3, such as any one of SEQ ID NOs: 1, 18-21, 40, 46, 52, 58, 98, 85, 104, 91, 180, 186, 192, 198, 220, 226, 231, 239, 245, 252, 257, 262, 267, 278, 286, 289, 295, and 301; a light chain variable CDR2 sequence having at least 90% (e.g., at least 91%, 92%, 93%, 94%, 95%, 96%, 97%, 98%, 99%, or 100%) sequence identity to a light chain variable CDR2 sequence in Table 3, such as any one of SEQ ID NOs: 24-28, 41, 47, 53, 59, 99, 86, 105, 92, 94, 181, 187, 193, 199, 221, 227, 232, 234, 240, 246, 248, 253, 263, 273, 281, 290, 296, and 302; a light chain variable CDR3 sequence having at least 90% (e.g., at least 91%, 92%, 93%, 94%, 95%, 96%, 97%, 98%, 99%, or 100%) sequence identity to a light chain variable CDR3 sequence in Table 3, such as any one of SEQ ID NOs: 4-8, 42, 48, 54, 60, 100, 87, 106, 93, 87, 182, 188, 194, 200, 222, 228, 235, 241, 247, 254, 258, 264, 268, 274, 279, 282, 287, 291, 297, and 303; a heavy chain variable CDR1 sequence having at least 90% (e.g., at least 91%, 92%, 93%, 94%, 95%, 96%, 97%, 98%, 99%, or 100%) sequence identity to a heavy chain variable CDR1 sequence in Table 3, such as any one of SEQ ID NOs: 31-35, 43, 49, 55, 61, 95, 82, 101, 88,177, 183, 189, 195, 217, 223, 229, 236, 242, 249, 255, 259, 265, 269, 275, 283, and 298; a heavy chain variable CDR2 sequence having at least 90% (e.g., at least 91%, 92%, 93%, 94%, 95%, 96%, 97%, 98%, 99%, or more) sequence identity to a heavy chain variable CDR2 sequence in Table 3, such as any one of SEQ ID NOs: 36-39, 12, 44, 50, 56, 62, 96, 83, 102, 89, 178, 184, 190, 196, 218, 224, 230, 237, 243, 250, 256, 260, 270, 276, 280, 284, 288, 292, 293, and 299; and a heavy chain variable CDR3 sequence having at least 90% (e.g., at least 91%, 92%, 93%, 94%, 95%, 96%, 97%, 98%, 99%, or more) sequence identity to a heavy chain variable CDR3 sequence in Table 3, such as any one of SEQ ID NOs: 13-17, 45, 51, 57, 63, 97, 84, 103, 90, 179, 185, 191, 197, 219, 225, 233, 238, 244, 251, 382, 261, 266, 271, 277, 285, 294, and 300. In some embodiments, the ALK2 antibody or antigen binding fragment thereof includes a light chain CDR1, CDR2, and CDR3 sequence and a heavy chain CDR1, CDR2, and CDR3 sequence from the same row of Table 3 (e.g., a light chain CDR1, CDR2, and CDR3 sequence and a heavy chain CDR1, CDR2, and CDR3 sequence from any one of Antibodies 1-38).

TABLE 3
ALK2 Antibody CDR sequences

| Anti- | VL | | | VH | | |
|---|---|---|---|---|---|---|
| body | CDR1 | CDR2 | CDR3 | CDR1 | CDR2 | CDR3 |
| 1 | SGSSSNIGS | VLIYKNNHR | ASWDHSDR | GGTFSSYG | WMGGIIPHF | EIGSLDI |
| | NYVS (SEQ | PS (SEQ ID | FYV (SEQ ID | VS (SEQ ID | GIANYAQKF | (SEQ ID NO: |
| | ID NO: 1) | NO: 24) | NO: 4) | NO: 31) | QG (SEQ ID | 13) |
| | | | | | NO: 36) | |
| 2 | SGDSIPSFF | LVIYRDSNR | YVTAPWKSI | GFTFSSHA | WVGRIKSKA | DYGVAFAY |
| | AS (SEQ ID | PS (SEQ ID | W (SEQ ID | MS (SEQ ID | DSGTTDYA | (SEQ ID NO: |
| | NO: 18) | NO: 25) | NO: 5) | NO: 32) | APVKG (SEQ | 14) |
| | | | | | ID NO: 37) | |
| 3 | SGDNIGTKY | LVIYGDSDR | YSADAQQM | GFTFNSSA | WVGRIKSK | DYGGLKFD |
| | AY (SEQ ID | PS (SEQ ID | KA (SEQ ID | MS (SEQ ID | RDGYTTDY | Y (SEQ ID |
| | NO: 19) | NO: 26) | NO: 6) | NO: 33) | AAPVKG | NO: 15) |
| | | | | | (SEQ ID NO: | |
| | | | | | 38) | |
| 4 | SGDNLRKY | LVIYYDNKR | QVYASVHR | GGTFSSYAI | WMGRIIPDF | GPTQAIHYF |
| | SAH (SEQ ID | PS (SEQ ID | M (SEQ ID | H (SEQ ID | GTANYAQK | AY (SEQ ID |
| | NO: 20) | NO: 27) | NO: 7) | NO: 34) | FQG (SEQ | NO: 16) |
| | | | | | ID NO: 39) | |
| 5 | SGDSLGSK | LVIYRDSKR | QTYDWSHF | GFTFSSAA | WVSVISSDG | AGFILGSLG |
| | SVH (SEQ ID | PS (SEQ ID | GW (SEQ ID | MH (SEQ ID | GSTYYADS | VAWMDV |
| | NO: 21) | NO: 28) | NO: 8) | NO: 35) | VKG (SEQ | (SEQ ID NO: |
| | | | | | ID NO: 12) | 17) |
| 6 | RASQGISGN | LLIYDASNL | HQSYRGPM | GFTFGRFV | WVSVIGYS | EPGYYYPS |
| | WLT (SEQ | QS (SEQ ID | (SEQ ID NO: | MH (SEQ ID | GSSTYYAD | GYYRGPGY |
| | ID NO: 40) | NO: 41) | 42) | NO: 43) | SVKG (SEQ | WMDV (SEQ |
| | | | | | ID NO: 44) | ID NO: 45) |
| 7 | SGDNIRKKY | LVIYRDSNR | SSAGRDNY | GFTFSSSA | WVSVIHYDS | DRYFFDV |
| | VH (SEQ ID | PS (SEQ ID | (SEQ ID NO: | MH (SEQ ID | SETYYADSV | (SEQ ID NO: |
| | NO: 46) | NO: 47) | 48) | NO: 49) | KG (SEQ ID | 51) |
| | | | | | NO: 50) | |
| 8 | SGDALRYYI | LVIYYNNNR | QSYGPGSV | GFTFSDYA | WVSSIFYSG | PKSYASGPF |
| | AH (SEQ ID | PS (SEQ ID | (SEQ ID NO: | MH (SEQ ID | SNTYYADSV | AY (SEQ ID |
| | NO: 52) | NO: 53) | 54) | NO: 55) | KG (SEQ ID | NO: 57) |
| | | | | | NO: 56) | |
| 9 | SGSSSNIGQ | LLIYDNSKR | SSWDLLSKS | GGTFSTHAI | WMGLIQPR | DYYGGMAY |
| | NYVS (SEQ | PS (SEQ ID | R (SEQ ID | S (SEQ ID | FGTANYAQ | (SEQ ID NO: |
| | ID NO: 58) | NO: 59) | NO: 60) | NO: 61) | KFQR (SEQ | 63) |
| | | | | | ID NO: 62) | |
| 10 | KASQNIYKY | YSNSLQT | FQYSSGPT | GFTFSNYY | SINTDGGST | STPNIPLAY |
| | LN (SEQ ID | (SEQ ID NO: | (SEQ ID NO: | MY (SEQ ID | YYPDSVKG | (SEQ ID NO: |
| | NO: 98) | 99) | 100) | NO: 95) | (SEQ ID NO: | 97) |
| | | | | | 96) | |
| 11 | RANQGVSL | RSSNLAS | QQSRESPFT | GFTFSHYY | SITNSGGSI | EGGENYGG |
| | SRYNLMH | (SEQ ID NO: | (SEQ ID NO: | MA (SEQ ID | NYRDSVKG | YPPFAY |
| | (SEQ ID NO: | 86) | 87) | NO: 82) | (SEQ ID NO: | (SEQ ID NO: |
| | 85) | | | | 83) | 84) |
| 12 | QASQDIGN | GATSLAD | LQAYSAPFT | GFTFSYYA | SISRGGDNT | LNYNNYFDY |
| | WLS (SEQ | (SEQ ID NO: | (SEQ ID NO: | MS (SEQ ID | YYRDTVKG | (SEQ ID NO: |
| | ID NO: 104) | 105) | 106) | NO: 101) | (SEQ ID NO: | 103) |
| | | | | | 102) | |
| 13 | LASSSVSYM | GTSNLAS | LHLTSYPPY | GSTFSNYG | SISRSSTYIY | AISTPFYWY |
| | T (SEQ ID | (SEQ ID NO: | T (SEQ ID | MK (SEQ ID | YADTVKG | FDF (SEQ ID |
| | NO: 91) | 92) | NO: 93) | NO: 88) | (SEQ ID NO: | NO: 90) |
| | | | | | 89) | |
| 14 | RANQGVSL | RSSNLAQ | QQSRESPFT | GFTFSHYY | SITNSGGSI | EGGENYGG |
| | SRYNLMH | (SEQ ID NO: | (SEQ ID NO: | MA (SEQ ID | NYRDSVKG | YPPFAY |
| | (SEQ ID NO: | 94) | 87) | NO: 82) | (SEQ ID NO: | (SEQ ID NO: |
| | 85) | | | | 83) | 84) |
| 15 | LASQTIGTW | AATSLAD | QQLYSTPW | GFNIKDSLM | IDPEDGETK | YTSDYYTM |
| | LA (SEQ ID | (SEQ ID NO: | T (SEQ ID | H (SEQ ID | YAPNFQD | DY (SEQ ID |
| | NO: 180) | 181) | NO: 182) | NO: 177) | (SEQ ID NO: | NO: 179) |
| | | | | | 178) | |
| 16 | LASQTIGTW | AATSLAD | QQLYSTPW | GFNIKDSLM | IDPEDGETK | YTSDYYTM |
| | LA (SEQ ID | (SEQ ID NO: | T (SEQ ID | H (SEQ ID | YAPNFQD | DY (SEQ ID |
| | NO: 186) | 187) | NO: 188) | NO: 183) | (SEQ ID NO: | NO: 185) |
| | | | | | 184) | |
| 17 | LASQTIGTW | AATSLAD | QQVYSTPW | GFNIKDSLM | IDPEDGETK | YTSPYYTM |
| | LA (SEQ ID | (SEQ ID NO: | T (SEQ ID | H (SEQ ID | YAPNFQS | DY (SEQ ID |
| | NO: 192) | 193) | NO: 194) | NO: 189) | (SEQ ID NO: | NO: 191) |
| | | | | | 190) | |
| 18 | LASQTIGTW | AATSLAD | QQLYWTPW | GFNIKDSLM | IDPEDGETR | YTSRYYTM |
| | LA (SEQ ID | (SEQ ID NO: | T (SEQ ID | H (SEQ ID | YAPNFQD | EY (SEQ ID |
| | NO: 198) | 199) | NO: 200) | NO: 195) | (SEQ ID NO: | NO: 197) |
| | | | | | 196) | |
| 19 | QNISSK | GAS (SEQ | LQYNNWWT | GFTFSNYG | ISYDGNNK | AKDRGLDT |
| | (SEQ ID NO: | ID NO: 221) | (SEQ ID NO: | (SEQ ID NO: | (SEQ ID NO: | AGDYFDY |
| | 220) | | 222) | 217) | 218) | (SEQ ID NO: |
| | | | | | | 219) |
| 20 | QDINIY | DVS (SEQ ID | QHYDDLPLT | NGSFSSYY | INHRGST | TAHTRGFLY |
| | (SEQ ID NO: | NO: 227) | (SEQ ID NO: | (SEQ ID NO: | (SEQ ID NO: | (SEQ ID NO: |
| | 226) | | 228) | 223) | 224) | 225) |
| 21 | QDINNY | DVF (SEQ ID | QHYDDLPLT | NGSFSSFY | INHRGRT | TAHTRGFLY |
| | (SEQ ID NO: | NO: 232) | (SEQ ID NO: | (SEQ ID NO: | (SEQ ID NO: | (SEQ ID NO: |
| | 231) | | 228) | 229) | 230) | 225) |
| 22 | QDINNY | DAS (SEQ ID | QHYYDLPLT | NGSFSSYY | INHRGRT | SAHTRGFLY |
| | (SEQ ID NO: | NO: 234) | (SEQ ID NO: | (SEQ ID NO: | (SEQ ID NO: | (SEQ ID NO: |
| | 231) | | 235) | 223) | 230) | 233) |
| 23 | QDINNY | DAS (SEQ ID | QHYYDLPLT | NGSFSSYY | INHRGST | TAHTRGFLY |
| | (SEQ ID NO: | NO: 234) | (SEQ ID NO: | (SEQ ID NO: | (SEQ ID NO: | (SEQ ID NO: |
| | 231) | | 235) | 223) | 224) | 225) |
| 24 | QSLVYSDGI | QVS (SEQ | MEGTHWPW | GFTFSDSA | IRNKANTYA | TSDQFD |
| | TY (SEQ ID | ID NO: 240) | T (SEQ ID | (SEQ ID NO: | T (SEQ ID | (SEQ ID NO: |
| | NO: 239) | | NO: 241) | 236) | NO: 237) | 238) |
| 25 | QSLVHSDG | TVS (SEQ ID | MQGTQFPA | GFTFSDYY | ISRTGSTK | AREPLQH |
| | DTY (SEQ ID | NO: 246) | LT (SEQ ID | (SEQ ID NO: | (SEQ ID NO: | (SEQ ID NO: |
| | NO: 245) | | NO: 247) | 242) | 243) | 244) |
| 26 | QSLVHSDG | TIS (SEQ ID | MQGTQFPA | GFTFSDYY | ISRTGSTK | AREPLQH |
| | DTY (SEQ ID | NO: 248) | LT (SEQ ID | (SEQ ID NO: | (SEQ ID NO: | (SEQ ID NO: |
| | NO: 245) | | NO: 247) | 242) | 243) | 244) |
| 27 | QSISTW | KAS (SEQ ID | QQYNTYWT | GFTLSDSS | IRSKPYSYA | TGGDDF |
| | (SEQ ID NO: | NO: 253) | (SEQ ID NO: | (SEQ ID NO: | T (SEQ ID | (SEQ ID NO: |
| | 252) | | 254) | 249) | NO: 250) | 251) |
| 28 | QSVSSN | GAS (SEQ | QQYNNWPP | GFTFSSYA | ISGSGGST | AIPYNWNY |
| | (SEQ ID NO: | ID NO: 221) | YT (SEQ ID | (SEQ ID NO: | (SEQ ID NO: | GGAFDI |
| | 257) | | NO: 258) | 255) | 256) | (SEQ ID NO: |
| | | | | | | 382) |
| 29 | QGINNY | AAS (SEQ ID | QKFNSAPLT | GYTFTGYY | INPNSVGT | TRDGAAAG |
| | (SEQ ID NO: | NO: 263) | (SEQ ID NO: | (SEQ ID NO: | (SEQ ID NO: | LFDY (SEQ |
| | 262) | | 264) | 259) | 260) | ID NO: 261) |
| 30 | QSISSY | AAS (SEQ ID | QQSYSTPPI | GFTFSTYA | ISGSGGST | ANSPSWFD |
| | (SEQ ID NO: | | T (SEQ ID | (SEQ ID NO: | (SEQ ID NO: | P (SEQ ID |
| | 267) | NO: 263) | NO: 268) | 265) | 256) | NO: 266) |
| 31 | QSVLYSSN | WAS (SEQ | QQYYSTPLT | GFTFDDYG | ISWNSATI | AKKNSLGW |
| | DNNY (SEQ | ID NO: 273) | (SEQ ID NO: | (SEQ ID NO: | (SEQ ID NO: | FFDY (SEQ |
| | ID NO: 272) | | 274) | 269) | 270) | ID NO: 271) |
| 32 | QGIRND | AAS (SEQ ID | LQHNSYPW | GYTFTSYG | ISAYNGNT | ARDINWYFD |
| | (SEQ ID NO: | NO: 263) | T (SEQ ID | (SEQ ID NO: | (SEQ ID NO: | L (SEQ ID |
| | 278) | | NO: 279) | 275) | 276) | NO: 277) |
| 33 | QGIRND | TAS (SEQ ID | LOHNNYPYT | GFTFDDYG | ISWNGATI | AKKNSLGW |
| | (SEQ ID NO: | NO: 281) | (SEQ ID NO: | (SEQ ID NO: | (SEQ ID NO: | FFDY (SEQ |
| | 278) | | 282) | 269) | 280) | ID NO: 271) |
| 34 | QSVISY | GAS (SEQ | QQNYLPPLT | GFTFSNYA | ITGNGVNT | VKERGHSW |
| | (SEQ ID NO: | ID NO: 221) | (SEQ ID NO: | (SEQ ID NO: | (SEQ ID NO: | FGDWFDP |
| | 286) | | 287) | 283) | 284) | (SEQ ID NO: |
| | | | | | | 285) |
| 35 | QGLVNSDG | EIS (SEQ ID | MQSTQFPLT | GFTFSNYA | ITGNGIKT | VKERGHSW |
| | NTY (SEQ ID | NO: 290) | (SEQ ID NO: | (SEQ ID NO: | (SEQ ID NO: | FGDWFDP |
| | NO: 289) | | 291) | 283) | 288) | (SEQ ID NO: |
| | | | | | | 285) |
| 36 | QGIRND | AAS (SEQ ID | LQHNSYPW | GFTFSNYA | ITGNGINT | VKERGHSW |
| | (SEQ ID NO: | NO: 263) | T (SEQ ID | (SEQ ID NO: | (SEQ ID NO: | FGDWFDP |
| | 278) | | NO: 279) | 283) | 292) | (SEQ ID NO: |
| | | | | | | 285) |
| 37 | QGISSH | VAS (SEQ ID | QQYYSYPPT | GFTFSNYG | VSYDGSNK | AKDRGITGT |
| | (SEQ ID NO: | NO: 296) | (SEQ ID NO: | (SEQ ID NO: | (SEQ ID NO: | SGGVFD |
| | 295) | | 297) | 217) | 293) | (SEQ ID NO: |
| | | | | | | 294) |
| 38 | QSLVYSDG | KVS (SEQ ID | MQGTHWPP | GFTSSRYW | INQDGSEK | ARDEEGY |
| | DTY (SEQ ID | NO: 302) | T (SEQ ID | (SEQ ID NO: | (SEQ ID NO: | (SEQ ID NO: |
| | NO: 301) | | NO: 303) | 298) | 299) | 300) |

In some embodiments, the ALK2 antibody administered to the subject is an ALK2 antibody or an antigen-binding fragment thereof, having a variable light chain (VL) sequence and a variable heavy chain (VH) sequence of an antibody described in Table 4 (in which the CDR sequences are in bold), or an ALK2 antibody or an antigen-binding fragment thereof, having a VL and VH sequence having at least 90% sequence identity (e.g., 90%, 91%, 92%, 93%, 94%, 95%, 95%, 97%, 98%, 99% or more sequence identity) to a VH and VL sequence of an antibody listed in Table 4. In some embodiments, the ALK2 antibody or an antigen binding fragment thereof, apart from the light chain CDR1, CDR2, and CDR3 and the heavy chain CDR1, CDR2, and CDR3, has a VL and VH sequence having at least 90% sequence identity (e.g., 90%, 91%, 92%, 93%, 94%, 95%, 95%, 97%, 98%, 99% or more sequence identity) to a VH and VL sequence of an antibody listed in Table 4.

TABLE 4
ALK2 antibody sequences

| SEQ ID | | |
|---|---|---|
| NO: | Antibody | Sequence |
| 67 | Antibody 1 | DIVLTQPPSVSGAPGQRVTISCSGSSSNIGSNYVSWYQQLPGTAPKVLIY |
| | VL: | KNNHRPSGVPDRFSGSKSGTSASLAITGLQAEDEADYYCASWDHSDRFYV |
| | Positions | FGGGTKLTVLGQPKAAPSVTLFPPSSEELQANKATLVCLISDFYPGAVTV |
| | 1-112 | AWKADSSPVKAGVETTTPSKQSNNKYAASSYLSLTPEQWKSHRSYSCQVT |
| | VH: | HEGSTVEKTVAPTEAQVQLVQSGAEVKKPGSSVKVSCKASGGTFSSYGVS |
| | Positions | WVRQAPGQGLEWMGGIIPHFGIANYAQKFQGRVTITADESTSTAYMELSS |
| | 216-331 | LRSEDTAVYYCAREIGSLDIWGQGTLVTVSSASTKGPSVFPLAPSSKSTS |
| | | GGTAALGCLVKDYFPEPVTVSWNSGALTSGVHTFPAVLQSSGLYSLSSVV |
| | | TVPSSSLGTQTYICNVNHKPSNTKVDKKVEPKSEFDYKDDDDKGAPHHHH |
| | | HH |
| 68 | Antibody 2 | DIELTQPPSVSVSPGQTASITCSGDSIPSFFASWYQQKPGQAPVLVIYRD |
| | VL: | SNRPSGIPERFSGSNSGNTATLTISGTQAEDEADYYCYVTAPWKSIWVFG |
| | Positions | GGTKLTVLGQPKAAPSVTLFPPSSEELQANKATLVCLISDFYPGAVTVAW |
| | 1-110 | KADSSPVKAGVETTTPSKQSNNKYAASSYLSLTPEQWKSHRSYSCQVTHE |
| | VH: | GSTVEKTVAPTEAEVQLVESGGGLVKPGGSLRLSCAASGFTFSSHAMSWV |
| | Positions | RQAPGKGLEWVGRIKSKADSGTTDYAAPVKGRFTISRDDSKNTLYLQMNS |
| | 214-332 | LKTEDTAVYYCARDYGVAFAYWGQGTLVTVSSASTKGPSVFPLAPSSKST |
| | | SGGTAALGCLVKDYFPEPVTVSWNSGALTSGVHTFPAVLQSSGLYSLSSV |
| | | VTVPSSSLGTQTYICNVNHKPSNTKVDKKVEPKSEFDYKDDDDKGAPHHH |
| | | HHH |
| 69 | Antibody 3 | DIELTQPPSVSVSPGQTASITCSGDNIGTKYAYWYQQKPGQAPVLVIYGD |
| | VL: | SDRPSGIPERFSGSNSGNTATLTISGTQAEDEADYYCYSADAQQMKAVFG |
| | Positions | GGTKLTVLGQPKAAPSVTLFPPSSEELQANKATLVCLISDFYPGAVTVAW |
| | 1-110 | KADSSPVKAGVETTTPSKQSNNKYAASSYLSLTPEQWKSHRSYSCQVTHE |
| | VH: | GSTVEKTVAPTEAEVQLVESGGGLVKPGGSLRLSCAASGFTFNSSAMSWV |
| | Positions | RQAPGKGLEWVGRIKSKRDGYTTDYAAPVKGRFTISRDDSKNTLYLQMNS |
| | 214-333 | LKTEDTAVYYCARDYGGLKFDYWGQGTLVTVSSASTKGPSVFPLAPSSKS |
| | | TSGGTAALGCLVKDYFPEPVTVSWNSGALTSGVHTFPAVLQSSGLYSLSS |
| | | VVTVPSSSLGTQTYICNVNHKPSNTKVDKKVEPKSEFDYKDDDDKGAPHH |
| | | HHHH |
| 70 | Antibody 4 | DIELTQPPSVSVSPGQTASITCSGDNLRKYSAHWYQQKPGQAPVLVIYYD |
| | VL: | NKRPSGIPERFSGSNSGNTATLTISGTQAEDEADYYCQVYASVHRMVFGG |
| | Positions | GTKLTVLGQPKAAPSVTLFPPSSEELQANKATLVCLISDFYPGAVTVAWK |
| | 1-109 | ADSSPVKAGVETTTPSKQSNNKYAASSYLSLTPEQWKSHRSYSCQVTHEG |
| | VH: | STVEKTVAPTEAQVQLVQSGAEVKKPGSSVKVSCKASGGTFSSYAIHWVR |
| | Positions | QAPGQGLEWMGRIIPDFGTANYAQKFQGRVTITADESTSTAYMELSSLRS |
| | 213-332 | EDTAVYYCARGPTQAIHYFAYWGQGTLVTVSSASTKGPSVFPLAPSSKST |
| | | SGGTAALGCLVKDYFPEPVTVSWNSGALTSGVHTFPAVLQSSGLYSLSSV |
| | | VTVPSSSLGTQTYICNVNHKPSNTKVDKKVEPKSEFDYKDDDDKGAPHHH |
| | | HHH |
| 71 | Antibody 5 | DIELTQPPSVSVSPGQTASITCSGDSLGSKSVHWYQQKPGQAPVLVIYRD |
| | VL: | SKRPSGIPERFSGSNSGNTATLTISGTQAEDEADYYCQTYDWSHFGWVFG |
| | Positions | GGTKLTVLGQPKAAPSVTLFPPSSEELQANKATLVCLISDFYPGAVTVAW |
| | 1-110 | KADSSPVKAGVETTTPSKQSNNKYAASSYLSLTPEQWKSHRSYSCQVTHE |
| | VH: | GSTVEKTVAPTEAEVQLLESGGGLVQPGGSLRLSCAASGFTFSSAAMHWV |
| | Positions | RQAPGKGLEWVSVISSDGGSTYYADSVKGRFTISRDNSKNTLYLQMNSLR |
| | 214-337 | AEDTAVYYCARAGFILGSLGVAWMDVWGQGTLVTVSSASTKGPSVFPLAP |
| | | SSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSEFDYKDDDDKG |
| | | APHHHHHH |
| 72 | Antibody 6 | DIQMTQSPSSLSASVGDRVTITCRASQGISGNWLTWYQQKPGKAPKLLIY |
| | VL: | DASNLQSGVPSRFSGSGSGTDFTLTISSLQPEDFATYYCHQSYRGPMTFG |
| | Positions | QGTKVEIKRTVAAPSVFIFPPSDEQLKSGTASVVCLLNNFYPREAKVQWK |
| | 1-110 | VDNALQSGNSQESVTEQDSKDSTYSLSSTLTLSKADYEKHKVYACEVTHQ |
| | VH: | GLSSPVTKSFNRGEAEVQLLESGGGLVQPGGSLRLSCAASGFTFGRFVMH |
| | Positions | WVRQAPGKGLEWVSVIGYSGSSTYYADSVKGRFTISRDNSKNTLYLQMNS |
| | 216-344 | LRAEDTAVYYCAREPGYYYPSGYYRGPGYWMDVWGQGTLVTVSSASTKGP |
| | | SVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKSEFDY |
| | | KDDDDKGAPHHHHHH |
| 73 | Antibody 7 | DIELTQPPSVSVSPGQTASITCSGDNIRKKYVHWYQQKPGQAPVLVIYRD |
| | VL: | SNRPSGIPERFSGSNSGNTATLTISGTQAEDEADYYCSSAGRDNYVFGGG |
| | Positions | TKLTVLGQPKAAPSVTLFPPSSEELQANKATLVCLISDFYPGAVTVAWKA |
| | 1-108 | DSSPVKAGVETTTPSKQSNNKYAASSYLSLTPEQWKSHRSYSCQVTHEGS |
| | VH: | TVEKTVAPTEAEVQLLESGGGLVQPGGSLRLSCAASGFTFSSSAMHWVRQ |
| | Positions | APGKGLEWVSVIHYDSSETYYADSVKGRFTISRDNSKNTLYLQMNSLRAE |
| | 212-327 | DTAVYYCARDRYFFDVWGQGTLVTVSSASTKGPSVFPLAPSSKSTSGGTA |
| | | ALGCLVKDYFPEPVTVSWNSGALTSGVHTFPAVLQSSGLYSLSSVVTVPS |
| | | SSLGTQTYICNVNHKPSNTKVDKKVEPKSEFDYKDDDDKGAPHHHHHH |
| 74 | Antibody 8 | DIELTQPPSVSVSPGQTASITCSGDALRYYIAHWYQQKPGQAPVLVIYYN |
| | VL: | NNRPSGIPERFSGSNSGNTATLTISGTQAEDEADYYCQSYGPGSVVFGGG |
| | Positions | TKLTVLGQPKAAPSVTLFPPSSEELQANKATLVCLISDFYPGAVTVAWKA |
| | 1-108 | DSSPVKAGVETTTPSKQSNNKYAASSYLSLTPEQWKSHRSYSCQVTHEGS |
| | VH: | TVEKTVAPTEAEVQLLESGGGLVQPGGSLRLSCAASGFTFSDYAMHWVRQ |
| | Positions | APGKGLEWVSSIFYSGSNTYYADSVKGRFTISRDNSKNTLYLQMNSLRAE |
| | 212-331 | DTAVYYCARPKSYASGPFAYWGQGTLVTVSSASTKGPSVFPLAPSSKSTS |
| | | GGTAALGCLVKDYFPEPVTVSWNSGALTSGVHTFPAVLQSSGLYSLSSVV |
| | | TVPSSSLGTQTYICNVNHKPSNTKVDKKVEPKSEFDYKDDDDKGAPHHHH |
| | | HH |
| 75 | Antibody 9 | DIVLTQPPSVSGAPGQRVTISCSGSSSNIGQNYVSWYQQLPGTAPKLLIY |
| | VL: | DNSKRPSGVPDRFSGSKSGTSASLAITGLQAEDEADYYCSSWDLLSKSRV |
| | Positions | FGGGTKLTVLGQPKAAPSVTLFPPSSEELQANKATLVCLISDFYPGAVTV |
| | 1-112 | AWKADSSPVKAGVETTTPSKQSNNKYAASSYLSLTPEQWKSHRSYSCQVT |
| | VH: | HEGSTVEKTVAPTEAQVQLVQSGAEVKKPGSSVKVSCKASGGTFSTHAIS |
| | Positions | WVRQAPGQGLEWMGLIQPRFGTANYAQKFQGRVTITADESTSTAYMELSS |
| | 216-332 | LRSEDTAVYYCARDYYGGMAYWGQGTLVTVSSASTKGPSVFPLAPSSKST |
| | | SGGTAALGCLVKDYFPEPVTVSWNSGALTSGVHTFPAVLQSSGLYSLSSV |
| | | VTVPSSSLGTQTYICNVNHKPSNTKVDKKVEPKSEFDYKDDDDKGAPHHH |
| | | HHH |

In some embodiments, the ALK2 antibody administered to the subject is an ALK2 antibody or an antigen-binding fragment thereof, having a VL and VH sequence having at least 90% sequence identity (e.g., 90%, 91%, 92%, 93%, 94%, 95%, 95%, 97%, 98%, 99% or 100% sequence identity) to a VH and VL sequence provided in Table 5. In some embodiments, the ALK2 antibody or an antigen binding fragment thereof, has a VL and VH sequence from the same row of Table 5. In some embodiments, the VH and VL have the sequence of SEQ ID NOs: 107 and 108; 109 and 110; 109 and 115; 111 and 112; 113 and 114; 116 and 118; 116 and 119; 117 and 118; 131 and 118; 117 and 119; 131 and 119; 117 and 115; 131 and 115; 117 and 120; 131 and 120; 121 and 115; 122 and 126; 132 and 126; 122 and 127; 132 and 127; 122 and 128; 132 and 128; 123 and 126; 133 and 126; 123 and 127; 133 and 127; 123 and 128; 133 and 128; 124 and 126; 124 and 127; 124 and 128; 124 and 129; 125 and 128; 125 and 129; 125 and 130; 134 and 129; 135 and 127; 139 and 129; 140 and 136; 140 and 137; 140 and 138; 201 and 202; 203 and 204; 205 and 206; 207 and 208; 304 and 305; 306 and 307; 308 and 309; 310 and 311; 306 and 312; 313 and 314; 315 and 316; 317 and 318; 319 and 320; 321 and 322; 323 and 324; 325 and 326; 327 and 328; 329 and 330; 331 and 332; 333 and 334; 335 and 336; 337 and 338; 339 and 340; or 341 and 342 (e.g., the VH has at least 90% sequence identity (e.g., 90%, 91%, 92%, 93%, 94%, 95%, 95%, 97%, 98%, 99% or 100% sequence identity) to the sequence of the first SEQ ID NO: in each pair and the VL has at least 90% sequence identity (e.g., 90%, 91%, 92%, 93%, 94%, 95%, 95%, 97%, 98%, 99% or 100% sequence identity) to the sequence of the second SEQ ID NO: in each pair). In some embodiments, the VH has the sequence of any one of SEQ ID NOs: 116, 117, 121-125, 131-135, 139, and 140 and the VL has the sequence of SEQ ID NO: 136. In some embodiments, the VH has the sequence of any one of SEQ ID NOs: 116, 117, 121-125, 131-135, 139, and 140 and the VL has the sequence of SEQ ID NO: 137. In some embodiments, the VH has the sequence of any one of SEQ ID NOs: 116, 117, 121-125, 131-135, 139, and 140 and the VL has the sequence of SEQ ID NO: 138. In some embodiments, the VH has the sequence of any one of SEQ ID NOs: 116, 117, 121-125, 131-135, 139, and 140 and the VL has the sequence of SEQ ID NO: 141. In some embodiments, the VH has the sequence of any one of SEQ ID NOs: 116, 117, 121-125, 131-135, 139, and 140 and the VL has the sequence of SEQ ID NO: 142. In some embodiments, the VH has the sequence of any one of SEQ ID NOs: 116, 117, 121-125, 131-135, 139, and 140 and the VL has the sequence of SEQ ID NO: 143.

TABLE 5
Variable heavy and light chain sequences of
exemplary ALK2 antibodies

| Heavy chain variable | Light chain variable |
|---|---|
| region sequence | region sequence |
| EVQLVETGGGLVQPGRSLKLSCVASGFTFSNYYM | DIQMTQSPSLLSASVGDRVTLSCKASQNIYKY |
| YWIRQAPGKGLEWISSINTDGGSTYYPDSVKGRF | LNWFQQKLGEAPKLLIYYSNSLQTGIPSRFSG |
| AISRDNAENTVYLQMNSLRSEDTATYYCAKSTPN | SGSGTDFTLTISSLQPEDVAIYFCFQYSSGPT |
| IPLAYWGQGTLVTVSS (SEQ ID NO: 107) | FGPGTKLELKRA (SEQ ID NO: 108) |
| EVQLVESGGGLVQPGRSLKLSCAASGFTFSHYYM | DIVLTQSPALAVSLGQRATISCRANQGVSLSR |
| AWVRQAPTKGLEWVASITNSGGSINYRDSVKGRF | YNLMHWYQQKPGQKPKLLIYRSSNLASGIPAR |
| TISRDNAKSTLYLQMDSLRSEDTATYYCTREGGE | FSGSGSGTDFTLTINPVQADDIATYYCQQSRE |
| NYGGYPPFAYWGQGTLVTVSS (SEQ ID NO: | SPFTFGAGTKLELKRA (SEQ ID NO: |
| 109) | 110) |
| EVQLVESGGGLVQPGRSLKLSCAASGFTFSHYYM | DIVLTQSPDSLAVSLGERATINCRANQGVSLS |
| AWVRQAPTKGLEWVASITNSGGSINYRDSVKGRF | RYNLMHWYQQKPGQKPKLLIYRSSNLAQGIPA |
| TISRDNAKSTLYLQMDSLRSEDTATYYCTREGGE | RFSGSGSGTDFTLTISSVQADDIAVYYCQQSR |
| NYGGYPPFAYWGQGTLVTVSS (SEQ ID NO: | ESPFTFGQGTKLELKRA (SEQ ID NO: |
| 109) | 115) |
| EVQLVESGGGLVQPGGSLKLSCAASGFTFSYYAM | DIQMTQSPASLSASLEEIVTITCQASQDIGNW |
| SWVCQAPTKGLEWVASISRGGDNTYYRDTVKGRF | LSWYQQKPGKSPQLLIYGATSLADGVPSRFSG |
| TTSRDNAKNTLYLQMDSLRSEDTATYYCARLNYN | SRSGTQYSLKISRLRVEDIGIYYCLQAYSAPF |
| NYFDYWGQGVMVTVSS (SEQ ID NO: 111) | TFGSGTKLEIKRA (SEQ ID NO: 112) |
| EVQLVESGGGLVQPGRSLKLSCLASGSTFSNYGM | EIVLTQSPTTMAASPGEKVTLNCLASSSVSYM |
| KWIRQAPGKGLEWVASISRSSTYIYYADTVKGRF | TWYQQKSGASPKLWIYGTSNLASGVPNRFSGS |
| TISRDNARNTLYLQMTSLRSEDTALYYCAAAIST | GSGTSYSLAISSMEAEDVATYYCLHLTSYPPY |
| PFYWYFDFWGPGTVVTVSS (SEQ ID NO: | TFGAGTKLELKRA (SEQ ID NO: 114) |
| 113) | |
| EVQLVESGGGLVQPGGSLRLSCAASGFTFSHYYM | DIVMTQSPDSLAVSLGERATINCRANQGVSLS |
| AWVRQAPGKGLEWVASITNSGGSINYRDSVKGRF | RYNLMHWYQQKPGQPPKLLIYRSSNLASGVPD |
| TISRDNSKNTLYLQMNSLRAEDTAVYYCAREGGE | RFSGSGSGTDFTLTISSLQAEDVAVYYCQQSR |
| NYGGYPPFAYWGQGTLVTVSS (SEQ ID NO: | ESPFTFGQGTKVEIKRA (SEQ ID NO: |
| 116) | 118) |
| EVQLVESGGGLVQPGGSLRLSCAASGFTFSHYYM | DIVLTQSPDSLAVSLGERATINCRANQGVSLS |
| AWVRQAPGKGLEWVASITNSGGSINYRDSVKGRF | RYNLMHWYQQKPGQKPKLLIYRSSNLASGIPA |
| TISRDNAKSTLYLQMNSLRAEDTATYYCTREGGE | RFSGSGSGTDFTLTISSVQADDIAVYYCQQSR |
| NYGGYPPFAYWGQGTLVTVSS (SEQ ID NO: | ESPFTFGQGTKLELKRA (SEQ ID NO: |
| 117) | 119) |
| EVQLVESGGGLVQPGGSLRLSCAASGFTFSHYYM | DIVLTQSPDSLAVSLGERATINCRANQGVSLS |
| AWVRQAPGKGLEWVASITNSGGSINYRDSVKGRF | RYNLMHWYQQKPGQKPKLAIYRSSNLASGIPA |
| TISRDNAKSTLYLQMNSLRAEDTATYYCTREGGE | RFSGSGSGTDFTLTISSVQADDIAVYYCQQSR |
| NYGGYPPFAYWGQGTLVTVSS (SEQ ID NO: | ESPFTFGQGTKLELKRA (SEQ ID NO: |
| 131) | 120) |
| EVQLVESGGGLVQPGGSLRLSCAASGSTFSNYGM | EIVLTQSPGTLSLSPGERATLSCLASSSVSYM |
| KWVRQAPGKGLEWVSSISRSSTYIYYADTVKGRF | TWYQQKPGQAPRLLIYGTSNLASGIPDRFSGS |
| TISRDNSKNTLYLQMNSLRAEDTAVYYCARAIST | GSGTDFTLTISRLEPEDFAVYYCLHLTSYPPY |
| PFYWYFDFWGQGTLVTVSS (SEQ ID NO: | TFGQGTKVEIKRA (SEQ ID NO: 126) |
| 121) | |
| EVQLVESGGGLVQPGGSLRLSCAASGSTFSNYGM | EIVLTQSPGTLSLSPGERATLSCLASSSVSYM |
| KWIRQAPGKGLEWVSSISRSSTYIYYADTVKGRF | TWYQQKPGQSPRLWIYGTSNLASGVPDRFSGS |
| TISRDNSKNTLYLQMNSLRAEDTAVYYCAAAIST | GSGTDFTLTISRLEPEDFAVYYCLHLTSYPPY |
| PFYWYFDFWGQGTLVTVSS (SEQ ID NO: | TFGQGTKVEIKRA (SEQ ID NO: 127) |
| 122) | |
| EVQLVESGGGLVQPGGSLRLSCAASGSTFSNYGM | EIVLTQSPGTLSLSPGERATLSCLASSSVSYM |
| KWIRQAPGKGLEWVSSISRSSTYIYYADTVKGRF | TWYQQKPGASPRLWIYGTSNLASGVPDRFSGS |
| TISRDNSKNTLYLQMNSLRAEDTAVYYCAAAIST | GSGTDYTLTISRLEPEDFATYYCLHLTSYPPY |
| PFYWYFDFWGQGTLVTVSS (SEQ ID NO: | TFGAGTKVEIKRA (SEQ ID NO: 128) |
| 132) | |
| EVQLVESGGGLVQPGGSLRLSCAASGSTFSNYGM | EIVLTQSPGTMSASPGERVTLSCLASSSVSYM |
| KWIRQAPGKGLEWVASISRSSTYIYYADTVKGRF | TWYQQKPGASPRLWIYGTSNLASGVPDRFSGS |
| TISRDNSKNTLYLQMNSLRAEDTAVYYCAAAIST | GSGTDYTLTISRMEPEDFATYYCLHLTSYPPY |
| PFYWYFDFWGQGTLVTVSS (SEQ ID NO: | TFGAGTKLELKRA (SEQ ID NO: 129) |
| 123) | |
| EVQLVESGGGLVQPGGSLRLSCAASGSTFSNYGM | EIVLTQSPGTMSASPGERVTLNCLASSSVSYM |
| KWIRQAPGKGLEWVASISRSSTYIYYADTVKGRF | TWYQQKSGASPKLWIYGTSNLASGVPNRFSGS |
| TISRDNSKNTLYLQMNSLRAEDTAVYYCAAAIST | GSGTSYTLTISRMEPEDFATYYCLHLTSYPPY |
| PFYWYFDFWGQGTLVTVSS (SEQ ID NO: | TFGAGTKLELKRA (SEQ ID NO: 130) |
| 133) | |
| EVQLVESGGGLVQPGGSLRLSCAASGSTFSNYGM | DIQMTQSPSSLSASVGDRVTITCKASQNIYKY |
| KWIRQAPGKGLEWVASISRSSTYIYYADTVKGRF | LNWFQQKPGKAPKLLIYYSNSLQTGVPSRFSG |
| TISRDNSKNTLYLQMNSLRAEDTALYYCAAAIST | SGSGTDFTLTISSLQPEDFATYYCFQYSSGPT |
| PFYWYFDFWGPGTLVTVSS (SEQ ID NO: | FGQGTKVEIKRT (SEQ ID NO: 136) |
| 124) | |
| EVQLVESGGGLVQPGGSLRLSCAASGSTFSNYGM | DIQMTQSPSSLSASVGDRVTITCKASQNIYKY |
| KWIRQAPGKGLEWVASISRSSTYIYYADTVKGRF | LNWFQQKPGKAPKLLIYYSNSLQTGIPSRFSG |
| TISRDNAKNTLYLQMNSLRSEDTALYYCAAAIST | SGSGTDFTLTISSLQPEDFATYYCFQYSSGPT |
| PFYWYFDFWGPGTLVTVSS (SEQ ID NO: | FGQGTKVEIKRT (SEQ ID NO: 137) |
| 125) | |
| EVQLVESGGGLVQPGGSLRLSCAASGFTFSNYYM | DIQMTQSPSSLSASVGDRVTISCKASQNIYKY |
| YWIRQAPGKGLEWVSSINTDGGSTYYADSVKGRF | LNWFQQKPGEAPKLLIYYSNSLQTGIPSRFSG |
| TISRDNSKNTVYLQMNSLRAEDTAVYYCAKSTPN | SGSGTDFTLTISSLQPEDFAIYFCFQYSSGPT |
| IPLAYWGQGTLVTVSS (SEQ ID NO: 134) | FGPGTKVEIKRT (SEQ ID NO: 138) |
| EVQLVESGGGLVQPGGSLRLSCAASGFTFSNYYM | DIQMTQSPSSLSASVGDRVTITCQASQDIGNW |
| YWIRQAPGKGLEWISSINTDGGSTYYPDSVKGRF | LSWYQQKPGKAPKLLIYGATSLADGVPSRFSG |
| TISRDNSKNTVYLQMNSLRAEDTAVYYCAKSTPN | SGSGTDFTLTISSLQPEDFATYYCLQAYSAPF |
| IPLAYWGQGTLVTVSS (SEQ ID NO: 135) | TFGQGTKVEIKRT (SEQ ID NO: 141) |
| EVQLVESGGGLVQPGGSLRLSCAASGFTFSYYAM | DIQMTQSPSSLSASVGDRVTITCQASQDIGNW |
| SWVRQAPGKGLEWVASISRGGDNTYYRDSVKGRF | LSWYQQKPGKAPKLLIYGATSLADGVPSRFSG |
| TTSRDNSKNTLYLQMNSLRAEDTAVYYCARLNYN | SRSGTDYTLTISSLQPEDFATYYCLQAYSAPF |
| NYFDYWGQGTLVTVSS (SEQ ID NO: 139) | TFGQGTKVEIKRT (SEQ ID NO: 142) |
| EVQLVESGGGLVQPGGSLRLSCAASGFTFSYYAM | DIQMTQSPSSLSASVGDRVTITCQASQDIGNW |
| SWVRQAPGKGLEWVASISRGGDNTYYRDSVKGRF | LSWYQQKPGKSPKLLIYGATSLADGVPSRFSG |
| TTSRDNSKNTLYLQMNSLRAEDTATYYCARLNYN | SRSGTQYTLTISSLQPEDFATYYCLQAYSAPF |
| NYFDYWGQGVLVTVSS (SEQ ID NO: 140) | TFGSGTKVEIKRT (SEQ ID NO: 143) |
| EVQLQQSGAELVRPGASVRLSCTASGFNIKDSLM | DIQMTQSPASQSASLGESVTFTCLASQTIGTW |
| HWVKQRPEQGLEWIGWIDPEDGETKYAPNFQDKA | LAWYQQKPGKSPQLLIYAATSLADGVPSRFSG |
| TITAVTSSNTAYLQLSSLTSEDSAIYYCARYTSD | SGSGTKFSFKISSLQAEDFASYYCQQLYSTPW |
| YYTMDYWGQGTSVTVSS (SEQ ID NO: 201) | TFGGGTKLEIK (SEQ ID NO: 202) |
| EVQLQQSGAELVRPGASVRLSCTASGFNIKDSLM | DIQMTQSPASQSASLGESVTFTCLASQTIGTW |
| HWVKQRPEQGLEWIGWIDPEDGETKYAPNFQDKA | LAWYQQKPGKSPQLLIYAATSLADGVPSRFSG |
| TITAVTSSNTAYLQLSSLTSEDSAIYYCARYTSD | SGSGTKFSFKISSLQAEDFASYYCQQLYSTPW |
| YYTMDYWGQGTSVTVSS (SEQ ID NO: 203) | TFGGGTKLEIK (SEQ ID NO: 204) |
| EVQLQQSGAELVRPGASVRLSCTASGFNIKDSLM | DIQMTQSPASQSASLGESVTFTCLASQTIGTW |
| HWVKQRPEQGLEWIGWIDPEDGETKYAPNFQSKA | LAWYQQKPGKSPQLLIYAATSLADGVPSRFSG |
| TITAVTSSNTAYLQLSSLTSEDSAIYYCARYTSP | SGSGTKFSFKISSLQAEDFASYYCQQVYSTPW |
| YYTMDYWGQGTSVTVSS (SEQ ID NO: 205) | TFGGGTKLEIK (SEQ ID NO: 206) |
| EVQLQQSGAELVRPGASVRLSCTASGFNIKDSLM | DIQMTQSPASQSASLGESVTFTCLASQTIGTW |
| HWVKQRPEQGLEWIGWIDPEDGETRYAPNFQDKA | LAWYQQKPGKSPQLLIYAATSLADGVPSRFSG |
| TITAVTSSNTAYLQLSSLTSEDSAIYYCARYTSR | SGSGTKFSFKISSLQAEDFASYYCQQLYWTPW |
| YYTMEYWGQGTSVTVSS (SEQ ID NO: 207) | TFGGGTKLEIK (SEQ ID NO: 208) |
| QVHLVESGGGVVQPGRSLRLSCAASGFTFSNYGI | EIVMTQSPATLSVSTGERATLSCRASQNISSK |
| HWVRQVPGKGLEWVAVISYDGNNKYYGDSVKGRF | LAWYQQKPGQAPRLLIYGASTRASGIPARFWG |
| TISRDNSKNTLYLQMNSLRPEDTAIYYCAKDRGL | SGSGTEFTLTISSLQSEDFAVFYCLQYNNWWT |
| DTAGDYFDYWGQGTLVTVSS (SEQ ID NO: | FGQGTKVEIK (SEQ ID NO: 305) |
| 304) | |
| QVQLQQWGAGLLKPSETLSLTCAVYNGSFSSYYW | DIQMTQSPSSLSASVGDRVTITCQASQDINIY |
| NWIRQPPGKGLEWLGEINHRGSTNYNPSLTSRVT | LNWYQQQPGKAPKLLIYDVSNLVPGVPSRFSG |
| ISVDTSKNQFSLKLTSVTAADTAIYYCTAHTRGF | TGSGTHFSFTISSLQPEDIATYYCQHYDDLPL |
| LYWGQGTLVTVSS (SEQ ID NO: 306) | TFGGGTTVAIR (SEQ ID NO: 307) |
| QVQLQQWGAGLLKPSETLSLTCAVYNGSFSSFYW | DIQMTQSPSSLSASVGDRVTITCQASQDINNY |
| SWIRQPPGKGLEWLGEINHRGRTNNNPSLASRVT | LNWYQQKPGQAPNLLIYDVFNLLPGVPSRFSG |
| ISVDTSKSQFSLRLTSVTAADTATYYCTAHTRGF | SGSGTDFSFTISSLQPEDIATYYCQHYDDLPL |
| LYWGQGTRVTVSS (SEQ ID NO: 308) | TFGGGTTVAIR (SEQ ID NO: 309) |
| QVQLQQWGAGLLKPSETLSLTCAVYNGSFSSYYW | DIQMTQSPSSLSASVGDRVTITCQASQDINNY |
| SWIRQPPGKGLEWLGEINHRGRTNYNPSLTSRVT | LNWYQQKPGKAPKLLIYDASNLVPGVPSRFSG |
| ISVDTSKNQFSLKLPSVTAADTAIYYCSAHTRGF | TGSGTDFSFTISSLQPEDIATYYCQHYYDLPL |
| LYWGQGTLVTVSS (SEQ ID NO: 310) | TFGGGTTVAIS (SEQ ID NO: 311) |
| QVQLQQWGAGLLKPSETLSLTCAVYNGSFSSYYW | DIQMTQSPSSLSASVGDRVTITCQASQDINNY |
| NWIRQPPGKGLEWLGEINHRGSTNYNPSLTSRVT | LNWYQQKPGKAPKLLIYDASNLVPGVPSRFSG |
| ISVDTSKNQFSLKLTSVTAADTAIYYCTAHTRGF | TGSGTDFSFTISSLQPEDIATYYCQHYYDLPL |
| LYWGQGTLVTVSS (SEQ ID NO: 306) | TFGGGTTVAIR (SEQ ID NO: 312) |
| EIQLVESGGDLVQPGGSLKLSCAASGFTFSDSAM | DVVMTQSPLSLSATLGQPASISCRSSQSLVYS |
| HWVRQASGKGLEWVGRIRNKANTYATSYAASVKG | DGITYLTWFQQRPGQSPTRLIYQVSNRDSGVP |
| RFTVSTDDSKTTAYLQMRYLKIEDTAIYYCTSDQ | DRFNGSGSGTDFTLNISRVEAEDVGFYYCMEG |
| FDYWGQGTLVTVSS (SEQ ID NO: 313) | THWPWTFGQGTKVELK (SEQ ID NO: |
| | 314) |
| QVQLVESGGGLVKPGGSLRLSCAASGFTFSDYYM | NVVLTQTPISSPVILGQPASISCRSSQSLVHS |
| SWIRQAPGKGLEWISFISRTGSTKYYADSVKGRF | DGDTYLSWLHQRPGQPPRLLIYTVSNRVSGVP |
| TISRDNAKNSLNLQMSSLRAEDTAVYFCAREPLQ | DRFSGSGAGTDFTLKISRVEAEDVGVYYCMQG |
| HWGQGTLVTVSS (SEQ ID NO: 315) | TQFPALTFGGGTKVEIK (SEQ ID NO: |
| | 316) |
| QVQLVESGGGLVKPGGSLRLSCAASGFTFSDYYM | KIVLTQTPLSSPVTLGQPASISCRSSQSLVHS |
| SWIRQAPGKGLEWVSYISRTGSTKYYADSVKGRF | DGDTYLSWLHQRPGQPPRLLIYTISNRLSGVP |
| TISRDNAKNSLNLQMSSLRAEDTAVYFCAREPLQ | DRFSGSGAGTDFTLKISRVEAEDVGVYYCMQG |
| HWGQGTLVTVSS (SEQ ID NO: 317) | TQFPALTFGGGTKVEIK (SEQ ID NO: |
| | 318) |
| EMQLVESGGGLVQPGGSLKVSCAASGFTLSDSSI | DIQMTQSPSTLSASVGDRITITCRASQSISTW |
| HWVRQASGKGLEWIGRIRSKPYSYATAYAASVKG | LAWYQQKPGKAPKLLIYKASSLEGGVPSRFSG |
| RFTISRDDSKNTAFLQMSGLKTEDTAVYYCTGGD | SGSGTDFTLTISSLQPDDFATYYCQQYNTYWT |
| DFWGQGTLVTVSS (SEQ ID NO: 319) | FGQGTKVEIK (SEQ ID NO: 320) |
| EVQLVESGGGLVQPGGSLRLSCAASGFTFSSYAM | EIVMTQSPATLSVSPGKRATLSCRASQSVSSN |
| SWVRQAPGKGLEWVSAISGSGGSTYYADSVKGRF | LAWYQQKPGQAPRLLIYGASTRATGIPARFSG |
| TISRDNSKNTLYLQMNSLRAEDTAVYYCAIPYNW | SGSGTEFTLTISSLQSEDFAVYYCQQYNNWPP |
| NYGGAFDIWGQGTMVTVSS (SEQ ID NO: | YTFGQGTKLEIK (SEQ ID NO: 322) |
| 321) | |
| QVQLVQSGAEVKKPGASVKVSCKASGYTFTGYYM | DIQMTQSPSSLSASVGDRVTITCRASQGINNY |
| HWVRQAPGQGLEWMGWINPNSVGTNFAQKFQGRV | LAWYQQKPGKVPKLLIYAASTLQSGVPSRFSG |
| TMTRDTSISTAYMELSRLRSDDTAVYYCTRDGAA | SGSGTDFTLTISSLQPEDVATYYCQKFNSAPL |
| AGLFDYWGQGTLVTVSS (SEQ ID NO: 323) | TFGGGTKVEIK (SEQ ID NO: 324) |
| EVQLVESGGGLVQPGGSLRLSCAASGFTFSTYAM | DIQMTQSPSSLSASVGDRVTITCRASQSISSY |
| SWVRQAPGKGLEWVSAISGSGGSTYYADSVKGRF | LNWYQQKPGKAPKLLIYAASSLQSGVPSRFSG |
| TISRDNSKNTLYLQMKSLRVEDTAVYYCANSPSW | SGSGTDFTLTISSLQPEDFATYYCQQSYSTPP |
| FDPWGQGTLVTVSS (SEQ ID NO: 325) | ITFGQGTRLEIK (SEQ ID NO: 326) |
| EVQLVESGGGLVQPGRSLRLSCAASGFTFDDYGM | DIVMTQSPDSLAVSLGERATINCKSSQSVLYS |
| HWVRQAPGKGLEWVSVISWNSATIDYADSVKGRF | SNDNNYLAWYQQKPGQPPKLLIYWASTRESGV |
| TISRDNAKNSLFLQMNSLRPEDTALYYCAKKNSL | PDRFSGSGSGTDFTLTISSLQAEDVAVYYCQQ |
| GWFFDYWGQGTLVTVSS (SEQ ID NO: 327) | YYSTPLTFGGGTKVEIK (SEQ ID NO: |
| | 328) |
| QVQLVQSGAEVKKPGASVKVSCKASGYTFTSYGI | DIQMTQSPSSLSASVGDRVTITCRASQGIRND |
| SWVRQAPGQGLEWMGWISAYNGNTIYAQKFQGRV | LGWFQQKPGKAPKRLIYAASSLQSGVPSRFSG |
| TMTTDTSTSTAYMELRSLRSDDTAVYYCARDINW | SGSGTEFTLTISSLQPEDFATYYCLQHNSYPW |
| YFDLWGRGTLVTVSS (SEQ ID NO: 329) | TFGQGTKVEIK (SEQ ID NO: 330) |
| EVQLVESGGGLVQPGRSLRLSCAASGFTFDDYGM | DIQMTQSPSSLSAFVGDRVTITCRASQGIRND |
| HWVRQAPGKGLEWVSVISWNGATIDYADSVKGRF | LGWYQQKPGKTPKRLIYTASSLRSGVPSRFSG |
| TISRDNAKNSLFLQMNSLRPEDTALYYCAKKNSL | SGSGTDFTLTISSLQPEDFATYYCLQHNNYPY |
| GWFFDYWGQGTLVTVSS (SEQ ID NO: 331) | TFGQGTKLEIK (SEQ ID NO: 332) |
| EVQLVESGGNLVQPGGSLRLSCAASGFTFSNYAM | DIQMTQSPSSLSASVGDRVTITCRSSQSVISY |
| SWVRQAPGKGLEWVSGITGNGVNTYYSVSVKGRF | LNWYQQKPGKAPKLLIFGASSLISGVPSRFSG |
| TISRDNSKNTLFLEMNSLRAEDTAIYYCVKERGH | SGSGTDFTLTIVSLQPEDFAVYFCQQNYLPPL |
| SWFGDWFDPWGQGTLVTVSS (SEQ ID NO: | TFGGGTKVEIK (SEQ ID NO: 334) |
| 333) | |
| EVQLVESGGNLVQPGGSLRLSCAASGFTFSNYAM | DIVMTQTPLSSPVTLGQPASISCRSSQGLVNS |
| SWVRQAPGKGLEWVSGITGNGIKTYYSVSAKGRF | DGNTYLSWLHQRPGQPPRLLIYEISNRFSGVP |
| TISRDNSKNTLFLQMNSLRAEDTAIYYCVKERGH | DRFSGSGAGTDFTLKISRVEAEDVGFYYCMQS |
| SWFGDWFDPWGQGTLVTVSS (SEQ ID NO: | TQFPLTFGGGTKVEIK (SEQ ID NO: |
| 335) | 336) |
| EVQLVESGGNLVQPGGSLRLSCAASGFTFSNYAM | DIQMTQSPSSLSASVGDRVTITCRASQGIRND |
| SWVRQAPGKGLEWVSGITGNGINTYYSVSVKGRF | LGWYQQKPGKAPKRLIYAASSLQSGVPSRFSG |
| TISRDNSKNTVFLQMNSLRAEDTAIYYCVKERGH | SGSGTEFTLTISSLQPEDFATYYCLQHNSYPW |
| SWFGDWFDPWGQGTLVTVSS (SEQ ID NO: | TFGQGTKVEIK (SEQ ID NO: 338) |
| 337) | |
| QVQLVESGGGVVQPGRSLRLSCEVSGFTFSNYGM | AIRMTQSPSSFSATTGDRVTITCRASQGISSH |
| HWVRQAPGKGLEWVAVVSYDGSNKYYTDSVKGRF | LAWYQQKPGKAPKFLIYVASTLQSGVPSRFSG |
| TISRDNSKNTLSLQMNSLRAEDTAVYYCAKDRGI | SGSGTDFTLTISSLQSEDFATYYCQQYYSYPP |
| TGTSGGVFDIWGQGTMVTVSS (SEQ ID NO: | TFGQGTKVEIK (SEQ ID NO: 340) |
| 339) | |
| EVQLVESGGGLVQPGGSLRLSCAASGFTSSRYWM | DVVMTQSLLSLSVTLGQPASISCRSSQSLVYS |
| SWVRQAPGKGLEWLANINQDGSEKYYLESLRGRF | DGDTYLSWFQQRPGQSPRRLIYKVSHRDSGVP |
| TISRDNAKNSLYLQMNNLRAEDTAVYYCARDEEG | DRFRGSGSGTDFTLKISRVEAEDVGIYYCMQG |
| YWGQGTLVTVSS (SEQ ID NO: 341) | THWPPTFGQGTKVEIK (SEQ ID NO: |
| | 342) |

In some embodiments, the ALK2 antibody administered to the subject is an ALK2 antibody or an antigen-binding fragment thereof, having a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 6. In some embodiments, the ALK2 antibody or an antigen binding fragment thereof, has a heavy chain and light chain sequence from the same row of Table 6. In some embodiments, the heavy chain and light chain have the sequence of SEQ ID NOs: 144 and 145; 146 and 147; 148 and 149; 150 and 151; 152 and 153; 154 and 155; 156 and 157; 156 and 158; 159 and 157; 159 and 158; 159 and 149; 159 and 160; 161 and 166; 161 and 145; 161 and 167; 162 and 166; 162 and 145; 162 and 167; 163 and 166; 163 and 145; 163 and 167; 163 and 168; 164 and 167; 164 and 168; 164 and 169; 165 and 168; 170 and 168; 171 and 172; 171 and 173; 171 and 147; 146 and 172; 146 and 173; 174 and 175; 174 and 176; 174 and 151; 150 and 175; 150 and 176; 209 and 210; 211 and 212; 213 and 214; 215 and 216; 343 and 344; 345 and 346; 347 and 348; 349 and 350; 345 and 351; 352 and 353; 354 and 355; 356 and 357; 358 and 359; 360 and 361; 362 and 363; 364 and 365; 366 and 367; 368 and 369; 370 and 371; 372 and 373; 374 and 375; 376 and 377; 378 and 379; 380 and 381 (e.g., the heavy chain has at least 90% sequence identity (e.g., 90%, 91%, 92%, 93%, 94%, 95%, 95%, 97%, 98%, 99% or 100% sequence identity) to the sequence of the first SEQ ID NO: in each pair and the light chain has at least 90% sequence identity (e.g., 90%, 91%, 92%, 93%, 94%, 95%, 95%, 97%, 98%, 99% or 100% sequence identity) to the sequence of the second SEQ ID NO: in each pair).

TABLE 6
Exemplary ALK2 antibody heavy and light chain sequences

| Heavy chain | Light chain |
|---|---|
| EVQLVESGGGLVQPGGSLRLSCAASGSTFSNYG | EIVLTQSPGTLSLSPGERATLSCLASSSVSYMT |
| MKWIRQAPGKGLEWVSSISRSSTYIYYADTVKGR | WYQQKPGQSPRLWIYGTSNLASGVPDRFSGS |
| FTISRDNSKNTLYLQMNSLRAEDTAVYYCAAAIST | GSGTDFTLTISRLEPEDFAVYYCLHLTSYPPYT |
| PFYWYFDFWGQGTLVTVSSASTKGPSVFPLAPS | FGQGTKVEIKRAVAAPSVFIFPPSDEQLKSGTA |
| SKSTSGGTAALGCLVKDYFPEPVTVSWNSGALTS | SVVCLLNNFYPREAKVQWKVDNALQSGNSQE |
| GVHTFPAVLQSSGLYSLSSVVTVPSSSLGTQTYIC | SVTEQDSKDSTYSLSSTLTLSKADYEKHKVYA |
| NVNHKPSNTKVDKRVEPKSCDKTHTCPPCPAPE | CEVTHQGLSSPVTKSFNRGEC (SEQ ID NO: |
| AAGGPSVFLFPPKPKDTLMISRTPEVTCVVVDVS | 145) |
| HEDPEVKFNWYVDGVEVHNAKTKPREEQYNSTY | |
| RVVSVLTVLHQDWLNGKEYKCKVSNKALPAPIEK | |
| TISKAKGQPREPQVYTLPPSREEMTKNQVSLTCL | |
| VKGFYPSDIAVEWESNGQPENNYKTTPPVLDSD | |
| GSFFLYSKLTVDKSRWQQGNVFSCSVMHEALHN | |
| HYTQKSLSLSPGK (SEQ ID NO: 144) | |
| EVQLVESGGGLVQPGGSLRLSCAASGFTFSNYY | DIQMTQSPSSLSASVGDRVTISCKASQNIYKYL |
| MYWIRQAPGKGLEWISSINTDGGSTYYPDSVKGR | NWFQQKPGEAPKLLIYYSNSLQTGIPSRFSGS |
| FTISRDNSKNTVYLQMNSLRAEDTAVYYCAKSTP | GSGTDFTLTISSLQPEDFAIYFCFQYSSGPTFG |
| NIPLAYWGQGTLVTVSSASTKGPSVFPLAPSSKS | PGTKVEIKRTVAAPSVFIFPPSDEQLKSGTASV |
| TSGGTAALGCLVKDYFPEPVTVSWNSGALTSGV | VCLLNNFYPREAKVQWKVDNALQSGNSQESV |
| HTFPAVLQSSGLYSLSSVVTVPSSSLGTQTYICNV | TEQDSKDSTYSLSSTLTLSKADYEKHKVYACE |
| NHKPSNTKVDKRVEPKSCDKTHTCPPCPAPELLG | VTHQGLSSPVTKSFNRGEC (SEQ ID NO: 147) |
| GPSVFLFPPKPKDTLMISRTPEVTCVVVDVSHED | |
| PEVKFNWYVDGVEVHNAKTKPREEQYNSTYRVV | |
| SVLTVLHQDWLNGKEYKCKVSNKALPAPIEKTISK | |
| AKGQPREPQVYTLPPSREEMTKNQVSLTCLVKG | |
| FYPSDIAVEWESNGQPENNYKTTPPVLDSDGSFF | |
| LYSKLTVDKSRWQQGNVFSCSVMHEALHNHYTQ | |
| KSLSLSPGK (SEQ ID NO: 146) | |
| EVQLVESGGGLVQPGGSLRLSCAASGFTFSHYY | DIVLTQSPDSLAVSLGERATINCRANQGVSLSR |
| MAWVRQAPGKGLEWVASITNSGGSINYRDSVKG | YNLMHWYQQKPGQKPKLLIYRSSNLAQGIPAR |
| RFTISRDNAKSTLYLQMNSLRAEDTATYYCTREG | FSGSGSGTDFTLTISSVQADDIAVYYCQQSRE |
| GENYGGYPPFAYWGQGTLVTVSSASTKGPSVFP | SPFTFGQGTKLELKRAVAAPSVFIFPPSDEQLK |
| LAPCSRSTSESTAALGCLVKDYFPEPVTVSWNSG | SGTASVVCLLNNFYPREAKVQWKVDNALQSG |
| ALTSGVHTFPAVLQSSGLYSLSSVVTVPSSNFGT | NSQESVTEQDSKDSTYSLSSTLTLSKADYEKH |
| QTYTCNVDHKPSNTKVDKTVERKCCVECPPCPA | KVYACEVTHQGLSSPVTKSFNRGEC (SEQ ID |
| PPVAGPSVFLFPPKPKDTLMISRTPEVTCVVVDVS | NO: 149) |
| HEDPEVQFNWYVDGVEVHNAKTKPREEQFNSTF | |
| RVVSVLTVVHQDWLNGKEYKCKVSNKGLPAPIEK | |
| TISKTKGQPREPQVYTLPPSREEMTKNQVSLTCL | |
| VKGFYPSDIAVEWESNGQPENNYKTTPPMLDSD | |
| GSFFLYSKLTVDKSRWQQGNVFSCSVMHEALHN | |
| HYTQKSLSLSPGK (SEQ ID NO: 148) | |
| EVQLVESGGGLVQPGGSLRLSCAASGFTFSYYA | DIQMTQSPSSLSASVGDRVTITCQASQDIGNW |
| MSWVRQAPGKGLEWVASISRGGDNTYYRDSVK | LSWYQQKPGKSPKLLIYGATSLADGVPSRFSG |
| GRFTTSRDNSKNTLYLQMNSLRAEDTATYYCARL | SRSGTQYTLTISSLQPEDFATYYCLQAYSAPFT |
| NYNNYFDYWGQGVLVTVSSASTKGPSVFPLAPS | FGSGTKVEIKRTVAAPSVFIFPPSDEQLKSGTA |
| SKSTSGGTAALGCLVKDYFPEPVTVSWNSGALTS | SVVCLLNNFYPREAKVQWKVDNALQSGNSQE |
| GVHTFPAVLQSSGLYSLSSVVTVPSSSLGTQTYIC | SVTEQDSKDSTYSLSSTLTLSKADYEKHKVYA |
| NVNHKPSNTKVDKRVEPKSCDKTHTCPPCPAPE | CEVTHQGLSSPVTKSFNRGEC (SEQ ID NO: |
| LLGGPSVFLFPPKPKDTLMISRTPEVTCVVVDVSH | 151) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 150) | |
| EVQLVESGGGLVQPGRSLKLSCAASGFTFSHYY | DIVLTQSPALAVSLGQRATISCRANQGVSLSRY |
| MAWVRQAPTKGLEWVASITNSGGSINYRDSVKG | NLMHWYQQKPGQKPKLLIYRSSNLASGIPARF |
| RFTISRDNAKSTLYLQMDSLRSEDTATYYCTREG | SGSGSGTDFTLTINPVQADDIATYYCQQSRES |
| GENYGGYPPFAYWGQGTLVTVSSASTKGPSVFP | PFTFGAGTKLELKRAVAAPSVFIFPPSDEQLKS |
| LAPSSKSTSGGTAALGCLVKDYFPEPVTVSWNS | GTASVVCLLNNFYPREAKVQWKVDNALQSG |
| GALTSGVHTFPAVLQSSGLYSLSSVVTVPSSSLG | NSQESVTEQDSKDSTYSLSSTLTLSKADYEKH |
| TQTYICNVNHKPSNTKVDKRVEPKSCDKTHTCPP | KVYACEVTHQGLSSPVTKSFNRGEC (SEQ ID |
| CPAPELLGGPSVFLFPPKPKDTLMISRTPEVTCVV | NO: 153) |
| VDVSHEDPEVKFNWYVDGVEVHNAKTKPREEQY | |
| NSTYRVVSVLTVLHQDWLNGKEYKCKVSNKALP | |
| APIEKTISKAKGQPREPQVYTLPPSREEMTKNQV | |
| SLTCLVKGFYPSDIAVEWESNGQPENNYKTTPPV | |
| LDSDGSFFLYSKLTVDKSRWQQGNVFSCSVMHE | |
| ALHNHYTQKSLSLSPGK (SEQ ID NO: 152) | |
| EVQLVESGGGLVQPGRSLKLSCLASGSTFSNYG | EIVLTQSPTTMAASPGEKVTLNCLASSSVSYM |
| MKWIRQAPGKGLEWVASISRSSTYIYYADTVKGR | TWYQQKSGASPKLWIYGTSNLASGVPNRFSG |
| FTISRDNARNTLYLQMTSLRSEDTALYYCAAAIST | SGSGTSYSLAISSMEAEDVATYYCLHLTSYPP |
| PFYWYFDFWGPGTVVTVSSASTKGPSVFPLAPS | YTFGAGTKLELKRAVAAPSVFIFPPSDEQLKSG |
| SKSTSGGTAALGCLVKDYFPEPVTVSWNSGALTS | TASVVCLLNNFYPREAKVQWKVDNALQSGNS |
| GVHTFPAVLQSSGLYSLSSVVTVPSSSLGTQTYIC | QESVTEQDSKDSTYSLSSTLTLSKADYEKHKV |
| NVNHKPSNTKVDKRVEPKSCDKTHTCPPCPAPE | YACEVTHQGLSSPVTKSFNRGEC (SEQ ID |
| LLGGPSVFLFPPKPKDTLMISRTPEVTCVVVDVSH | NO: 155) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 154) | |
| EVQLVESGGGLVQPGGSLRLSCAASGFTFSHYY | DIVMTQSPDSLAVSLGERATINCRANQGVSLS |
| MAWVRQAPGKGLEWVASITNSGGSINYRDSVKG | RYNLMHWYQQKPGQPPKLLIYRSSNLASGVP |
| RFTISRDNSKNTLYLQMNSLRAEDTAVYYCAREG | DRFSGSGSGTDFTLTISSLQAEDVAVYYCQQS |
| GENYGGYPPFAYWGQGTLVTVSSASTKGPSVFP | RESPFTFGQGTKVEIKRAVAAPSVFIFPPSDEQ |
| LAPSSKSTSGGTAALGCLVKDYFPEPVTVSWNS | LKSGTASVVCLLNNFYPREAKVQWKVDNALQ |
| GALTSGVHTFPAVLQSSGLYSLSSVVTVPSSSLG | SGNSQESVTEQDSKDSTYSLSSTLTLSKADYE |
| TQTYICNVNHKPSNTKVDKRVEPKSCDKTHTCPP | KHKVYACEVTHQGLSSPVTKSFNRGEC (SEQ |
| CPAPELLGGPSVFLFPPKPKDTLMISRTPEVTCVV | ID NO: 157) |
| VDVSHEDPEVKFNWYVDGVEVHNAKTKPREEQY | |
| NSTYRVVSVLTVLHQDWLNGKEYKCKVSNKALP | |
| APIEKTISKAKGQPREPQVYTLPPSREEMTKNQV | |
| SLTCLVKGFYPSDIAVEWESNGQPENNYKTTPPV | |
| LDSDGSFFLYSKLTVDKSRWQQGNVFSCSVMHE | |
| ALHNHYTQKSLSLSPGK (SEQ ID NO: 156) | |
| EVQLVESGGGLVQPGGSLRLSCAASGFTFSHYY | DIVLTQSPDSLAVSLGERATINCRANQGVSLSR |
| MAWVRQAPGKGLEWVASITNSGGSINYRDSVKG | YNLMHWYQQKPGQKPKLLIYRSSNLASGIPAR |
| RFTISRDNSKNTLYLQMNSLRAEDTAVYYCAREG | FSGSGSGTDFTLTISSVQADDIAVYYCQQSRE |
| GENYGGYPPFAYWGQGTLVTVSSASTKGPSVFP | SPFTFGQGTKLELKRAVAAPSVFIFPPSDEQLK |
| LAPSSKSTSGGTAALGCLVKDYFPEPVTVSWNS | SGTASVVCLLNNFYPREAKVQWKVDNALQSG |
| GALTSGVHTFPAVLQSSGLYSLSSVVTVPSSSLG | NSQESVTEQDSKDSTYSLSSTLTLSKADYEKH |
| TQTYICNVNHKPSNTKVDKRVEPKSCDKTHTCPP | KVYACEVTHQGLSSPVTKSFNRGEC (SEQ ID |
| CPAPELLGGPSVFLFPPKPKDTLMISRTPEVTCVV | NO: 158) |
| VDVSHEDPEVKFNWYVDGVEVHNAKTKPREEQY | |
| NSTYRVVSVLTVLHQDWLNGKEYKCKVSNKALP | |
| APIEKTISKAKGQPREPQVYTLPPSREEMTKNQV | |
| SLTCLVKGFYPSDIAVEWESNGQPENNYKTTPPV | |
| LDSDGSFFLYSKLTVDKSRWQQGNVFSCSVMHE | |
| ALHNHYTQKSLSLSPGK (SEQ ID NO: 156) | |
| EVQLVESGGGLVQPGGSLRLSCAASGFTFSHYY | DIVMTQSPDSLAVSLGERATINCRANQGVSLS |
| MAWVRQAPGKGLEWVASITNSGGSINYRDSVKG | RYNLMHWYQQKPGQPPKLLIYRSSNLASGVP |
| RFTISRDNAKSTLYLQMNSLRAEDTATYYCTREG | DRFSGSGSGTDFTLTISSLQAEDVAVYYCQQS |
| GENYGGYPPFAYWGQGTLVTVSSASTKGPSVFP | RESPFTFGQGTKVEIKRAVAAPSVFIFPPSDEQ |
| LAPSSKSTSGGTAALGCLVKDYFPEPVTVSWNS | LKSGTASVVCLLNNFYPREAKVQWKVDNALQ |
| GALTSGVHTFPAVLQSSGLYSLSSVVTVPSSSLG | SGNSQESVTEQDSKDSTYSLSSTLTLSKADYE |
| TQTYICNVNHKPSNTKVDKRVEPKSCDKTHTCPP | KHKVYACEVTHQGLSSPVTKSFNRGEC (SEQ |
| CPAPELLGGPSVFLFPPKPKDTLMISRTPEVTCVV | ID NO: 157) |
| VDVSHEDPEVKFNWYVDGVEVHNAKTKPREEQY | |
| NSTYRVVSVLTVLHQDWLNGKEYKCKVSNKALP | |
| APIEKTISKAKGQPREPQVYTLPPSREEMTKNQV | |
| SLTCLVKGFYPSDIAVEWESNGQPENNYKTTPPV | |
| LDSDGSFFLYSKLTVDKSRWQQGNVFSCSVMHE | |
| ALHNHYTQKSLSLSPGK (SEQ ID NO: 159) | |
| EVQLVESGGGLVQPGGSLRLSCAASGFTFSHYY | DIVLTQSPDSLAVSLGERATINCRANQGVSLSR |
| MAWVRQAPGKGLEWVASITNSGGSINYRDSVKG | YNLMHWYQQKPGQKPKLLIYRSSNLASGIPAR |
| RFTISRDNAKSTLYLQMNSLRAEDTATYYCTREG | FSGSGSGTDFTLTISSVQADDIAVYYCQQSRE |
| GENYGGYPPFAYWGQGTLVTVSSASTKGPSVFP | SPFTFGQGTKLELKRAVAAPSVFIFPPSDEQLK |
| LAPSSKSTSGGTAALGCLVKDYFPEPVTVSWNS | SGTASVVCLLNNFYPREAKVQWKVDNALQSG |
| GALTSGVHTFPAVLQSSGLYSLSSVVTVPSSSLG | NSQESVTEQDSKDSTYSLSSTLTLSKADYEKH |
| TQTYICNVNHKPSNTKVDKRVEPKSCDKTHTCPP | KVYACEVTHQGLSSPVTKSFNRGEC (SEQ ID |
| CPAPELLGGPSVFLFPPKPKDTLMISRTPEVTCVV | NO: 158) |
| VDVSHEDPEVKFNWYVDGVEVHNAKTKPREEQY | |
| NSTYRVVSVLTVLHQDWLNGKEYKCKVSNKALP | |
| APIEKTISKAKGQPREPQVYTLPPSREEMTKNQV | |
| SLTCLVKGFYPSDIAVEWESNGQPENNYKTTPPV | |
| LDSDGSFFLYSKLTVDKSRWQQGNVFSCSVMHE | |
| ALHNHYTQKSLSLSPGK (SEQ ID NO: 159) | |
| EVQLVESGGGLVQPGGSLRLSCAASGFTFSHYY | DIVLTQSPDSLAVSLGERATINCRANQGVSLSR |
| MAWVRQAPGKGLEWVASITNSGGSINYRDSVKG | YNLMHWYQQKPGQKPKLLIYRSSNLAQGIPAR |
| RFTISRDNAKSTLYLQMNSLRAEDTATYYCTREG | FSGSGSGTDFTLTISSVQADDIAVYYCQQSRE |
| GENYGGYPPFAYWGQGTLVTVSSASTKGPSVFP | SPFTFGQGTKLELKRAVAAPSVFIFPPSDEQLK |
| LAPSSKSTSGGTAALGCLVKDYFPEPVTVSWNS | SGTASVVCLLNNFYPREAKVQWKVDNALQSG |
| GALTSGVHTFPAVLQSSGLYSLSSVVTVPSSSLG | NSQESVTEQDSKDSTYSLSSTLTLSKADYEKH |
| TQTYICNVNHKPSNTKVDKRVEPKSCDKTHTCPP | KVYACEVTHQGLSSPVTKSFNRGEC (SEQ ID |
| CPAPELLGGPSVFLFPPKPKDTLMISRTPEVTCVV | NO: 149) |
| VDVSHEDPEVKFNWYVDGVEVHNAKTKPREEQY | |
| NSTYRVVSVLTVLHQDWLNGKEYKCKVSNKALP | |
| APIEKTISKAKGQPREPQVYTLPPSREEMTKNQV | |
| SLTCLVKGFYPSDIAVEWESNGQPENNYKTTPPV | |
| LDSDGSFFLYSKLTVDKSRWQQGNVFSCSVMHE | |
| ALHNHYTQKSLSLSPGK (SEQ ID NO: 159) | |
| EVQLVESGGGLVQPGGSLRLSCAASGFTFSHYY | DIVLTQSPDSLAVSLGERATINCRANQGVSLSR |
| MAWVRQAPGKGLEWVASITNSGGSINYRDSVKG | YNLMHWYQQKPGQKPKLAIYRSSNLASGIPAR |
| RFTISRDNAKSTLYLQMNSLRAEDTATYYCTREG | FSGSGSGTDFTLTISSVQADDIAVYYCQQSRE |
| GENYGGYPPFAYWGQGTLVTVSSASTKGPSVFP | SPFTFGQGTKLELKRAVAAPSVFIFPPSDEQLK |
| LAPSSKSTSGGTAALGCLVKDYFPEPVTVSWNS | SGTASVVCLLNNFYPREAKVQWKVDNALQSG |
| GALTSGVHTFPAVLQSSGLYSLSSVVTVPSSSLG | NSQESVTEQDSKDSTYSLSSTLTLSKADYEKH |
| TQTYICNVNHKPSNTKVDKRVEPKSCDKTHTCPP | KVYACEVTHQGLSSPVTKSFNRGEC (SEQ ID |
| CPAPELLGGPSVFLFPPKPKDTLMISRTPEVTCVV | NO: 160) |
| VDVSHEDPEVKFNWYVDGVEVHNAKTKPREEQY | |
| NSTYRVVSVLTVLHQDWLNGKEYKCKVSNKALP | |
| APIEKTISKAKGQPREPQVYTLPPSREEMTKNQV | |
| SLTCLVKGFYPSDIAVEWESNGQPENNYKTTPPV | |
| LDSDGSFFLYSKLTVDKSRWQQGNVFSCSVMHE | |
| ALHNHYTQKSLSLSPGK (SEQ ID NO: 159) | |
| EVQLVESGGGLVQPGGSLRLSCAASGSTFSNYG | EIVLTQSPGTLSLSPGERATLSCLASSSVSYMT |
| MKWVRQAPGKGLEWVSSISRSSTYIYYADTVKG | WYQQKPGQAPRLLIYGTSNLASGIPDRFSGSG |
| RFTISRDNSKNTLYLQMNSLRAEDTAVYYCARAIS | SGTDFTLTISRLEPEDFAVYYCLHLTSYPPYTF |
| TPFYWYFDFWGQGTLVTVSSASTKGPSVFPLAP | GQGTKVEIKRAVAAPSVFIFPPSDEQLKSGTAS |
| SSKSTSGGTAALGCLVKDYFPEPVTVSWNSGALT | VVCLLNNFYPREAKVQWKVDNALQSGNSQ |
| SGVHTFPAVLQSSGLYSLSSVVTVPSSSLGTQTYI | ESVTEQDSKDSTYSLSSTLTLSKADYEKHKVY |
| CNVNHKPSNTKVDKRVEPKSCDKTHTCPPCPAP | ACEVTHQGLSSPVTKSFNRGEC (SEQ ID NO: |
| ELLGGPSVFLFPPKPKDTLMISRTPEVTCVVVDVS | 166) |
| HEDPEVKFNWYVDGVEVHNAKTKPREEQYNSTY | |
| RVVSVLTVLHQDWLNGKEYKCKVSNKALPAPIEK | |
| TISKAKGQPREPQVYTLPPSREEMTKNQVSLTCL | |
| VKGFYPSDIAVEWESNGQPENNYKTTPPVLDSD | |
| GSFFLYSKLTVDKSRWQQGNVFSCSVMHEALHN | |
| HYTQKSLSLSPGK (SEQ ID NO: 161) | |
| EVQLVESGGGLVQPGGSLRLSCAASGSTFSNYG | EIVLTQSPGTLSLSPGERATLSCLASSSVSYMT |
| MKWVRQAPGKGLEWVSSISRSSTYIYYADTVKG | WYQQKPGQSPRLWIYGTSNLASGVPDRFSGS |
| RFTISRDNSKNTLYLQMNSLRAEDTAVYYCARAIS | GSGTDFTLTISRLEPEDFAVYYCLHLTSYPPYT |
| TPFYWYFDFWGQGTLVTVSSASTKGPSVFPLAP | FGQGTKVEIKRAVAAPSVFIFPPSDEQLKSGTA |
| SSKSTSGGTAALGCLVKDYFPEPVTVSWNSGALT | SVVCLLNNFYPREAKVQWKVDNALQSGNSQ |
| SGVHTFPAVLQSSGLYSLSSVVTVPSSSLGTQTYI | ESVTEQDSKDSTYSLSSTLTLSKADYEKHKVY |
| CNVNHKPSNTKVDKRVEPKSCDKTHTCPPCPAP | ACEVTHQGLSSPVTKSFNRGEC (SEQ ID NO: |
| ELLGGPSVFLFPPKPKDTLMISRTPEVTCVVVDVS | 145) |
| HEDPEVKFNWYVDGVEVHNAKTKPREEQYNSTY | |
| RVVSVLTVLHQDWLNGKEYKCKVSNKALPAPIEK | |
| TISKAKGQPREPQVYTLPPSREEMTKNQVSLTCL | |
| VKGFYPSDIAVEWESNGQPENNYKTTPPVLDSD | |
| GSFFLYSKLTVDKSRWQQGNVFSCSVMHEALHN | |
| HYTQKSLSLSPGK (SEQ ID NO: 161) | |
| EVQLVESGGGLVQPGGSLRLSCAASGSTFSNYG | EIVLTQSPGTLSLSPGERATLSCLASSSVSYMT |
| MKWVRQAPGKGLEWVSSISRSSTYIYYADTVKG | WYQQKPGASPRLWIYGTSNLASGVPDRFSGS |
| RFTISRDNSKNTLYLQMNSLRAEDTAVYYCARAIS | GSGTDYTLTISRLEPEDFATYYCLHLTSYPPYT |
| TPFYWYFDFWGQGTLVTVSSASTKGPSVFPLAP | FGAGTKVEIKRAVAAPSVFIFPPSDEQLKSGTA |
| SSKSTSGGTAALGCLVKDYFPEPVTVSWNSGALT | SVVCLLNNFYPREAKVQWKVDNALQSGNSQ |
| SGVHTFPAVLQSSGLYSLSSVVTVPSSSLGTQTYI | ESVTEQDSKDSTYSLSSTLTLSKADYEKHKVY |
| CNVNHKPSNTKVDKRVEPKSCDKTHTCPPCPAP | ACEVTHQGLSSPVTKSFNRGEC (SEQ ID NO: |
| ELLGGPSVFLFPPKPKDTLMISRTPEVTCVVVDVS | 167) |
| HEDPEVKFNWYVDGVEVHNAKTKPREEQYNSTY | |
| RVVSVLTVLHQDWLNGKEYKCKVSNKALPAPIEK | |
| TISKAKGQPREPQVYTLPPSREEMTKNQVSLTCL | |
| VKGFYPSDIAVEWESNGQPENNYKTTPPVLDSD | |
| GSFFLYSKLTVDKSRWQQGNVFSCSVMHEALHN | |
| HYTQKSLSLSPGK (SEQ ID NO: 161) | |
| EVQLVESGGGLVQPGGSLRLSCAASGSTFSNYG | EIVLTQSPGTLSLSPGERATLSCLASSSVSYMT |
| MKWIRQAPGKGLEWVSSISRSSTYIYYADTVKGR | WYQQKPGQAPRLLIYGTSNLASGIPDRFSGSG |
| FTISRDNSKNTLYLQMNSLRAEDTAVYYCAAAIST | SGTDFTLTISRLEPEDFAVYYCLHLTSYPPYTF |
| PFYWYFDFWGQGTLVTVSSASTKGPSVFPLAPS | GQGTKVEIKRAVAAPSVFIFPPSDEQLKSGTAS |
| SKSTSGGTAALGCLVKDYFPEPVTVSWNSGALTS | VVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 166) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 162) | |
| EVQLVESGGGLVQPGGSLRLSCAASGSTFSNYG | EIVLTQSPGTLSLSPGERATLSCLASSSVSYMT |
| MKWIRQAPGKGLEWVSSISRSSTYIYYADTVKGR | WYQQKPGQSPRLWIYGTSNLASGVPDRFSGS |
| FTISRDNSKNTLYLQMNSLRAEDTAVYYCAAAIST | GSGTDFTLTISRLEPEDFAVYYCLHLTSYPPYT |
| PFYWYFDFWGQGTLVTVSSASTKGPSVFPLAPS | FGQGTKVEIKRAVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 145) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 162) | |
| EVQLVESGGGLVQPGGSLRLSCAASGSTFSNYG | EIVLTQSPGTLSLSPGERATLSCLASSSVSYMT |
| MKWIRQAPGKGLEWVSSISRSSTYIYYADTVKGR | WYQQKPGASPRLWIYGTSNLASGVPDRFSGS |
| FTISRDNSKNTLYLQMNSLRAEDTAVYYCAAAIST | GSGTDYTLTISRLEPEDFATYYCLHLTSYPPYT |
| PFYWYFDFWGQGTLVTVSSASTKGPSVFPLAPS | FGAGTKVEIKRAVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 167) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 162) | |
| EVQLVESGGGLVQPGGSLRLSCAASGSTFSNYG | EIVLTQSPGTLSLSPGERATLSCLASSSVSYMT |
| MKWIRQAPGKGLEWVASISRSSTYIYYADTVKGR | WYQQKPGQAPRLLIYGTSNLASGIPDRFSGSG |
| FTISRDNSKNTLYLQMNSLRAEDTAVYYCAAAIST | SGTDFTLTISRLEPEDFAVYYCLHLTSYPPYTF |
| PFYWYFDFWGQGTLVTVSSASTKGPSVFPLAPS | GQGTKVEIKRAVAAPSVFIFPPSDEQLKSGTAS |
| SKSTSGGTAALGCLVKDYFPEPVTVSWNSGALTS | VVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 166) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 163) | |
| EVQLVESGGGLVQPGGSLRLSCAASGSTFSNYG | EIVLTQSPGTLSLSPGERATLSCLASSSVSYMT |
| MKWIRQAPGKGLEWVASISRSSTYIYYADTVKGR | WYQQKPGQSPRLWIYGTSNLASGVPDRFSGS |
| FTISRDNSKNTLYLQMNSLRAEDTAVYYCAAAIST | GSGTDFTLTISRLEPEDFAVYYCLHLTSYPPYT |
| PFYWYFDFWGQGTLVTVSSASTKGPSVFPLAPS | FGQGTKVEIKRAVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 145) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 163) | |
| EVQLVESGGGLVQPGGSLRLSCAASGSTFSNYG | EIVLTQSPGTLSLSPGERATLSCLASSSVSYMT |
| MKWIRQAPGKGLEWVASISRSSTYIYYADTVKGR | WYQQKPGASPRLWIYGTSNLASGVPDRFSGS |
| FTISRDNSKNTLYLQMNSLRAEDTAVYYCAAAIST | GSGTDYTLTISRLEPEDFATYYCLHLTSYPPYT |
| PFYWYFDFWGQGTLVTVSSASTKGPSVFPLAPS | FGAGTKVEIKRAVAAPSVFIFPPSDEQLKSGTA |
| SKSTSGGTAALGCLVKDYFPEPVTVSWNSGALTS | SVVCLLNNFYPREAKVQWKVDNALQSGNSQE |
| GVHTFPAVLQSSGLYSLSSVVTVPSSSLGTQTYIC | SVTEQDSKDSTYSLSSTLTLSKADYEKHKVYA |
| NVNHKPSNTKVDKRVEPKSCDKTHTCPPCPAPE | CEVTHQGLSSPVTKSFNRGEC (SEQ ID NO: |
| LLGGPSVFLFPPKPKDTLMISRTPEVTCVVVDVSH | 167) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 163) | |
| EVQLVESGGGLVQPGGSLRLSCAASGSTFSNYG | EIVLTQSPGTMSASPGERVTLSCLASSSVSYM |
| MKWIRQAPGKGLEWVASISRSSTYIYYADTVKGR | TWYQQKPGASPRLWIYGTSNLASGVPDRFSG |
| FTISRDNSKNTLYLQMNSLRAEDTAVYYCAAAIST | SGSGTDYTLTISRMEPEDFATYYCLHLTSYPP |
| PFYWYFDFWGQGTLVTVSSASTKGPSVFPLAPS | YTFGAGTKLELKRAVAAPSVFIFPPSDEQLKSG |
| SKSTSGGTAALGCLVKDYFPEPVTVSWNSGALTS | TASVVCLLNNFYPREAKVQWKVDNALQSGNS |
| GVHTFPAVLQSSGLYSLSSVVTVPSSSLGTQTYIC | QESVTEQDSKDSTYSLSSTLTLSKADYEKHKV |
| NVNHKPSNTKVDKRVEPKSCDKTHTCPPCPAPE | YACEVTHQGLSSPVTKSFNRGEC (SEQ ID |
| LLGGPSVFLFPPKPKDTLMISRTPEVTCVVVDVSH | NO: 168) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 163) | |
| EVQLVESGGGLVQPGGSLRLSCAASGSTFSNYG | EIVLTQSPGTLSLSPGERATLSCLASSSVSYMT |
| MKWIRQAPGKGLEWVASISRSSTYIYYADTVKGR | WYQQKPGASPRLWIYGTSNLASGVPDRFSGS |
| FTISRDNSKNTLYLQMNSLRAEDTALYYCAAAIST | GSGTDYTLTISRLEPEDFATYYCLHLTSYPPYT |
| PFYWYFDFWGPGTLVTVSSASTKGPSVFPLAPSS | FGAGTKVEIKRAVAAPSVFIFPPSDEQLKSGTA |
| KSTSGGTAALGCLVKDYFPEPVTVSWNSGALTS | SVVCLLNNFYPREAKVQWKVDNALQSGNSQE |
| GVHTFPAVLQSSGLYSLSSVVTVPSSSLGTQTYIC | SVTEQDSKDSTYSLSSTLTLSKADYEKHKVYA |
| NVNHKPSNTKVDKRVEPKSCDKTHTCPPCPAPE | CEVTHQGLSSPVTKSFNRGEC (SEQ ID NO: |
| LLGGPSVFLFPPKPKDTLMISRTPEVTCVVVDVSH | 167) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 164) | |
| EVQLVESGGGLVQPGGSLRLSCAASGSTFSNYG | EIVLTQSPGTMSASPGERVTLSCLASSSVSYM |
| MKWIRQAPGKGLEWVASISRSSTYIYYADTVKGR | TWYQQKPGASPRLWIYGTSNLASGVPDRFSG |
| FTISRDNSKNTLYLQMNSLRAEDTALYYCAAAIST | SGSGTDYTLTISRMEPEDFATYYCLHLTSYPP |
| PFYWYFDFWGPGTLVTVSSASTKGPSVFPLAPSS | YTFGAGTKLELKRAVAAPSVFIFPPSDEQLKSG |
| KSTSGGTAALGCLVKDYFPEPVTVSWNSGALTS | TASVVCLLNNFYPREAKVQWKVDNALQSGNS |
| GVHTFPAVLQSSGLYSLSSVVTVPSSSLGTQTYIC | QESVTEQDSKDSTYSLSSTLTLSKADYEKHKV |
| NVNHKPSNTKVDKRVEPKSCDKTHTCPPCPAPE | YACEVTHQGLSSPVTKSFNRGEC (SEQ ID |
| LLGGPSVFLFPPKPKDTLMISRTPEVTCVVVDVSH | NO: 168) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 164) | |
| EVQLVESGGGLVQPGGSLRLSCAASGSTFSNYG | EIVLTQSPGTMSASPGERVTLNCLASSSVSYM |
| MKWIRQAPGKGLEWVASISRSSTYIYYADTVKGR | TWYQQKSGASPKLWIYGTSNLASGVPNRFSG |
| FTISRDNSKNTLYLQMNSLRAEDTALYYCAAAIST | SGSGTSYTLTISRMEPEDFATYYCLHLTSYPPY |
| PFYWYFDFWGPGTLVTVSSASTKGPSVFPLAPSS | TFGAGTKLELKRAVAAPSVFIFPPSDEQLKSGT |
| KSTSGGTAALGCLVKDYFPEPVTVSWNSGALTS | ASVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 169) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 164) | |
| EVQLVESGGGLVQPGGSLRLSCAASGSTFSNYG | EIVLTQSPGTMSASPGERVTLSCLASSSVSYM |
| MKWIRQAPGKGLEWVASISRSSTYIYYADTVKGR | TWYQQKPGASPRLWIYGTSNLASGVPDRFSG |
| FTISRDNAKNTLYLQMNSLRSEDTALYYCAAAIST | SGSGTDYTLTISRMEPEDFATYYCLHLTSYPP |
| PFYWYFDFWGPGTLVTVSSASTKGPSVFPLAPSS | YTFGAGTKLELKRAVAAPSVFIFPPSDEQLKSG |
| KSTSGGTAALGCLVKDYFPEPVTVSWNSGALTS | TASVVCLLNNFYPREAKVQWKVDNALQSGNS |
| GVHTFPAVLQSSGLYSLSSVVTVPSSSLGTQTYIC | QESVTEQDSKDSTYSLSSTLTLSKADYEKHKV |
| NVNHKPSNTKVDKRVEPKSCDKTHTCPPCPAPE | YACEVTHQGLSSPVTKSFNRGEC (SEQ ID |
| LLGGPSVFLFPPKPKDTLMISRTPEVTCVVVDVSH | NO: 168) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 165) | |
| EVQLVESGGGLVQPGGSLRLSCAASGSTFSNYG | EIVLTQSPGTMSASPGERVTLSCLASSSVSYM |
| MKWIRQAPGKGLEWVASISRSSTYIYYADTVKGR | TWYQQKPGASPRLWIYGTSNLASGVPDRFSG |
| FTISRDNSKNTLYLQMNSLRAEDTAVYYCAAAIST | SGSGTDYTLTISRMEPEDFATYYCLHLTSYPP |
| PFYWYFDFWGQGTLVTVSSASTKGPSVFPLAPS | YTFGAGTKLELKRAVAAPSVFIFPPSDEQLKSG |
| SKSTSGGTAALGCLVKDYFPEPVTVSWNSGALTS | TASVVCLLNNFYPREAKVQWKVDNALQSGNS |
| GVHTFPAVLQSSGLYSLSSVVTVPSSSLGTQTYIC | QESVTEQDSKDSTYSLSSTLTLSKADYEKHKV |
| NVNHKPSNTKVDKRVEPKSCDKTHTCPPCPAPE | YACEVTHQGLSSPVTKSFNRGEC (SEQ ID |
| AAGGPSVFLFPPKPKDTLMISRTPEVTCVVVDVS | NO: 168) |
| HEDPEVKFNWYVDGVEVHNAKTKPREEQYNSTY | |
| RVVSVLTVLHQDWLNGKEYKCKVSNKALPAPIEK | |
| TISKAKGQPREPQVYTLPPSREEMTKNQVSLTCL | |
| VKGFYPSDIAVEWESNGQPENNYKTTPPVLDSD | |
| GSFFLYSKLTVDKSRWQQGNVFSCSVMHEALHN | |
| HYTQKSLSLSPGK (SEQ ID NO: 170) | |
| EVQLVESGGGLVQPGGSLRLSCAASGFTFSNYY | DIQMTQSPSSLSASVGDRVTITCKASQNIYKYL |
| MYWIRQAPGKGLEWVSSINTDGGSTYYADSVKG | NWFQQKPGKAPKLLIYYSNSLQTGVPSRFSGS |
| RFTISRDNSKNTVYLQMNSLRAEDTAVYYCAKST | GSGTDFTLTISSLQPEDFATYYCFQYSSGPTF |
| PNIPLAYWGQGTLVTVSSASTKGPSVFPLAPSSK | GQGTKVEIKRTVAAPSVFIFPPSDEQLKSGTAS |
| STSGGTAALGCLVKDYFPEPVTVSWNSGALTSG | VVCLLNNFYPREAKVQWKVDNALQSGNSQE |
| VHTFPAVLQSSGLYSLSSVVTVPSSSLGTQTYICN | SVTEQDSKDSTYSLSSTLTLSKADYEKHKVYA |
| VNHKPSNTKVDKRVEPKSCDKTHTCPPCPAPELL | CEVTHQGLSSPVTKSFNRGEC (SEQ ID NO: |
| GGPSVFLFPPKPKDTLMISRTPEVTCVVVDVSHE | 172) |
| DPEVKFNWYVDGVEVHNAKTKPREEQYNSTYRV | |
| VSVLTVLHQDWLNGKEYKCKVSNKALPAPIEKTIS | |
| KAKGQPREPQVYTLPPSREEMTKNQVSLTCLVK | |
| GFYPSDIAVEWESNGQPENNYKTTPPVLDSDGSF | |
| FLYSKLTVDKSRWQQGNVFSCSVMHEALHNHYT | |
| QKSLSLSPGK (SEQ ID NO: 171) | |
| EVQLVESGGGLVQPGGSLRLSCAASGFTFSNYY | DIQMTQSPSSLSASVGDRVTITCKASQNIYKYL |
| MYWIRQAPGKGLEWVSSINTDGGSTYYADSVKG | NWFQQKPGKAPKLLIYYSNSLQTGIPSRFSGS |
| RFTISRDNSKNTVYLQMNSLRAEDTAVYYCAKST | GSGTDFTLTISSLQPEDFATYYCFQYSSGPTF |
| PNIPLAYWGQGTLVTVSSASTKGPSVFPLAPSSK | GQGTKVEIKRTVAAPSVFIFPPSDEQLKSGTAS |
| STSGGTAALGCLVKDYFPEPVTVSWNSGALTSG | VVCLLNNFYPREAKVQWKVDNALQSGNSQE |
| VHTFPAVLQSSGLYSLSSVVTVPSSSLGTQTYICN | SVTEQDSKDSTYSLSSTLTLSKADYEKHKVYA |
| VNHKPSNTKVDKRVEPKSCDKTHTCPPCPAPELL | CEVTHQGLSSPVTKSFNRGEC (SEQ ID NO: |
| GGPSVFLFPPKPKDTLMISRTPEVTCVVVDVSHE | 173) |
| DPEVKFNWYVDGVEVHNAKTKPREEQYNSTYRV | |
| VSVLTVLHQDWLNGKEYKCKVSNKALPAPIEKTIS | |
| KAKGQPREPQVYTLPPSREEMTKNQVSLTCLVK | |
| GFYPSDIAVEWESNGQPENNYKTTPPVLDSDGSF | |
| FLYSKLTVDKSRWQQGNVFSCSVMHEALHNHYT | |
| QKSLSLSPGK (SEQ ID NO: 171) | |
| EVQLVESGGGLVQPGGSLRLSCAASGFTFSNYY | DIQMTQSPSSLSASVGDRVTISCKASQNIYKYL |
| MYWIRQAPGKGLEWVSSINTDGGSTYYADSVKG | NWFQQKPGEAPKLLIYYSNSLQTGIPSRFSGS |
| RFTISRDNSKNTVYLQMNSLRAEDTAVYYCAKST | GSGTDFTLTISSLQPEDFAIYFCFQYSSGPTFG |
| PNIPLAYWGQGTLVTVSSASTKGPSVFPLAPSSK | PGTKVEIKRTVAAPSVFIFPPSDEQLKSGTASV |
| STSGGTAALGCLVKDYFPEPVTVSWNSGALTSG | VCLLNNFYPREAKVQWKVDNALQSGNSQESV |
| VHTFPAVLQSSGLYSLSSVVTVPSSSLGTQTYICN | TEQDSKDSTYSLSSTLTLSKADYEKHKVYACE |
| VNHKPSNTKVDKRVEPKSCDKTHTCPPCPAPELL | VTHQGLSSPVTKSFNRGEC (SEQ ID NO: 147) |
| GGPSVFLFPPKPKDTLMISRTPEVTCVVVDVSHE | |
| DPEVKFNWYVDGVEVHNAKTKPREEQYNSTYRV | |
| VSVLTVLHQDWLNGKEYKCKVSNKALPAPIEKTIS | |
| KAKGQPREPQVYTLPPSREEMTKNQVSLTCLVK | |
| GFYPSDIAVEWESNGQPENNYKTTPPVLDSDGSF | |
| FLYSKLTVDKSRWQQGNVFSCSVMHEALHNHYT | |
| QKSLSLSPGK (SEQ ID NO: 171) | |
| EVQLVESGGGLVQPGGSLRLSCAASGFTFSNYY | DIQMTQSPSSLSASVGDRVTITCKASQNIYKYL |
| MYWIRQAPGKGLEWISSINTDGGSTYYPDSVKGR | NWFQQKPGKAPKLLIYYSNSLQTGVPSRFSGS |
| FTISRDNSKNTVYLQMNSLRAEDTAVYYCAKSTP | GSGTDFTLTISSLQPEDFATYYCFQYSSGPTF |
| NIPLAYWGQGTLVTVSSASTKGPSVFPLAPSSKS | GQGTKVEIKRTVAAPSVFIFPPSDEQLKSGTAS |
| TSGGTAALGCLVKDYFPEPVTVSWNSGALTSGV | VVCLLNNFYPREAKVQWKVDNALQSGNSQE |
| HTFPAVLQSSGLYSLSSVVTVPSSSLGTQTYICNV | SVTEQDSKDSTYSLSSTLTLSKADYEKHKVYA |
| NHKPSNTKVDKRVEPKSCDKTHTCPPCPAPELLG | CEVTHQGLSSPVTKSFNRGEC (SEQ ID NO: |
| GPSVFLFPPKPKDTLMISRTPEVTCVVVDVSHED | 172) |
| PEVKFNWYVDGVEVHNAKTKPREEQYNSTYRVV | |
| SVLTVLHQDWLNGKEYKCKVSNKALPAPIEKTISK | |
| AKGQPREPQVYTLPPSREEMTKNQVSLTCLVKG | |
| FYPSDIAVEWESNGQPENNYKTTPPVLDSDGSFF | |
| LYSKLTVDKSRWQQGNVFSCSVMHEALHNHYTQ | |
| KSLSLSPGK (SEQ ID NO: 146) | |
| EVQLVESGGGLVQPGGSLRLSCAASGFTFSNYY | DIQMTQSPSSLSASVGDRVTITCKASQNIYKYL |
| MYWIRQAPGKGLEWISSINTDGGSTYYPDSVKGR | NWFQQKPGKAPKLLIYYSNSLQTGIPSRFSGS |
| FTISRDNSKNTVYLQMNSLRAEDTAVYYCAKSTP | GSGTDFTLTISSLQPEDFATYYCFQYSSGPTF |
| NIPLAYWGQGTLVTVSSASTKGPSVFPLAPSSKS | GQGTKVEIKRTVAAPSVFIFPPSDEQLKSGTAS |
| TSGGTAALGCLVKDYFPEPVTVSWNSGALTSGV | VVCLLNNFYPREAKVQWKVDNALQSGNSQE |
| HTFPAVLQSSGLYSLSSVVTVPSSSLGTQTYICNV | SVTEQDSKDSTYSLSSTLTLSKADYEKHKVYA |
| NHKPSNTKVDKRVEPKSCDKTHTCPPCPAPELLG | CEVTHQGLSSPVTKSFNRGEC (SEQ ID NO: |
| GPSVFLFPPKPKDTLMISRTPEVTCVVVDVSHED | 173) |
| PEVKFNWYVDGVEVHNAKTKPREEQYNSTYRVV | |
| SVLTVLHQDWLNGKEYKCKVSNKALPAPIEKTISK | |
| AKGQPREPQVYTLPPSREEMTKNQVSLTCLVKG | |
| FYPSDIAVEWESNGQPENNYKTTPPVLDSDGSFF | |
| LYSKLTVDKSRWQQGNVFSCSVMHEALHNHYTQ | |
| KSLSLSPGK (SEQ ID NO: 146) | |
| EVQLVESGGGLVQPGGSLRLSCAASGFTFSYYA | DIQMTQSPSSLSASVGDRVTITCQASQDIGNW |
| MSWVRQAPGKGLEWVASISRGGDNTYYRDSVK | LSWYQQKPGKAPKLLIYGATSLADGVPSRFSG |
| GRFTTSRDNSKNTLYLQMNSLRAEDTAVYYCARL | SGSGTDFTLTISSLQPEDFATYYCLQAYSAPFT |
| NYNNYFDYWGQGTLVTVSSASTKGPSVFPLAPS | FGQGTKVEIKRTVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 175) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 174) | |
| EVQLVESGGGLVQPGGSLRLSCAASGFTFSYYA | DIQMTQSPSSLSASVGDRVTITCQASQDIGNW |
| MSWVRQAPGKGLEWVASISRGGDNTYYRDSVK | LSWYQQKPGKAPKLLIYGATSLADGVPSRFSG |
| GRFTTSRDNSKNTLYLQMNSLRAEDTAVYYCARL | SRSGTDYTLTISSLQPEDFATYYCLQAYSAPFT |
| NYNNYFDYWGQGTLVTVSSASTKGPSVFPLAPS | FGQGTKVEIKRTVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 176) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 174) | |
| EVQLVESGGGLVQPGGSLRLSCAASGFTFSYYA | DIQMTQSPSSLSASVGDRVTITCQASQDIGNW |
| MSWVRQAPGKGLEWVASISRGGDNTYYRDSVK | LSWYQQKPGKSPKLLIYGATSLADGVPSRFSG |
| GRFTTSRDNSKNTLYLQMNSLRAEDTAVYYCARL | SRSGTQYTLTISSLQPEDFATYYCLQAYSAPFT |
| NYNNYFDYWGQGTLVTVSSASTKGPSVFPLAPS | FGSGTKVEIKRTVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 151) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 174) | |
| EVQLVESGGGLVQPGGSLRLSCAASGFTFSYYA | DIQMTQSPSSLSASVGDRVTITCQASQDIGNW |
| MSWVRQAPGKGLEWVASISRGGDNTYYRDSVK | LSWYQQKPGKAPKLLIYGATSLADGVPSRFSG |
| GRFTTSRDNSKNTLYLQMNSLRAEDTATYYCARL | SGSGTDFTLTISSLQPEDFATYYCLQAYSAPFT |
| NYNNYFDYWGQGVLVTVSSASTKGPSVFPLAPS | FGQGTKVEIKRTVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 175) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 150) | |
| EVQLVESGGGLVQPGGSLRLSCAASGFTFSYYA | DIQMTQSPSSLSASVGDRVTITCQASQDIGNW |
| MSWVRQAPGKGLEWVASISRGGDNTYYRDSVK | LSWYQQKPGKAPKLLIYGATSLADGVPSRFSG |
| GRFTTSRDNSKNTLYLQMNSLRAEDTATYYCARL | SRSGTDYTLTISSLQPEDFATYYCLQAYSAPFT |
| NYNNYFDYWGQGVLVTVSSASTKGPSVFPLAPS | FGQGTKVEIKRTVAAPSVFIFPPSDEQLKSGTA |
| SKSTSGGTAALGCLVKDYFPEPVTVSWNSGALTS | SVVCLLNNFYPREAKVQWKVDNALQSGNSQ |
| GVHTFPAVLQSSGLYSLSSVVTVPSSSLGTQTYIC | ESVTEQDSKDSTYSLSSTLTLSKADYEKHKVY |
| NVNHKPSNTKVDKRVEPKSCDKTHTCPPCPAPE | ACEVTHQGLSSPVTKSFNRGEC (SEQ ID NO: |
| LLGGPSVFLFPPKPKDTLMISRTPEVTCVVVDVSH | 176) |
| EDPEVKFNWYVDGVEVHNAKTKPREEQYNSTYR | |
| VVSVLTVLHQDWLNGKEYKCKVSNKALPAPIEKTI | |
| SKAKGQPREPQVYTLPPSREEMTKNQVSLTCLV | |
| KGFYPSDIAVEWESNGQPENNYKTTPPVLDSDG | |
| SFFLYSKLTVDKSRWQQGNVFSCSVMHEALHNH | |
| YTQKSLSLSPGK (SEQ ID NO: 150) | |
| EVQLQQSGAELVRPGASVRLSCTASGFNIKDSLM | DIQMTQSPASQSASLGESVTFTCLASQTIGTW |
| HWVKQRPEQGLEWIGWIDPEDGETKYAPNFQDK | LAWYQQKPGKSPQLLIYAATSLADGVPSRFSG |
| ATITAVTSSNTAYLQLSSLTSEDSAIYYCARYTSDY | SGSGTKFSFKISSLQAEDFASYYCQQLYSTPW |
| YTMDYWGQGTSVTVSSAKTTPPSVYPLAPGFAA | TFGGGTKLEIKRADAAPTVSIFPPSSEQLTSGG |
| QTNSMVTLGCLVKGYFPEPVTVTWNSGSLSSGV | ASVVCFLNNFYPKDINVKWKIDGSERQNGVLN |
| HTFPAVLQSDLYTLSSSVTVPSSTWPSQTVTCNV | SWTDQDSKDSTYSMSSTLTLTKDEYERHNSY |
| AHPASSTKVDKKIVPRDCGCKPCICTVPEVSSVFI | TCEATHKTSTSPIVKSFNRNEC (SEQ ID NO: |
| FPPKPKDVLTITLTPKVTCVVVDISKDDPEVQFSW | 210) |
| FVDDVEVHTAQTKPREEQINSTFRSVSELPIMHQ | |
| DWLNGKEFKCRVNSAAFPAPIEKTISKTKGRPKA | |
| PQVYTIPPPKEQMAKDKVSLTCMITNFFPEDITVE | |
| WQWNGQPAENYKNTQPIMDTDGSYFVYSKLNV | |
| QKSNWEAGNTFTCSVLHEGLHNHHTEKSLSHSP | |
| GK (SEQ ID NO: 209) | |
| EVQLQQSGAELVRPGASVRLSCTASGFNIKDSLM | DIQMTQSPASQSASLGESVTFTCLASQTIGTW |
| HWVKQRPEQGLEWIGWIDPEDGETKYAPNFQDK | LAWYQQKPGKSPQLLIYAATSLADGVPSRFSG |
| ATITAVTSSNTAYLQLSSLTSEDSAIYYCARYTSDY | SGSGTKFSFKISSLQAEDFASYYCQQLYSTPW |
| YTMDYWGQGTSVTVSSAKTTPPSVYPLAPGSAA | TFGGGTKLEIKRADAAPTVSIFPPSSEQLTSGG |
| QTNSMVTLGCLVKGYFPEPVTVTWNSGSLSSGV | ASVVCFLNNFYPKDINVKWKIDGSERQNGVLN |
| HTFPAVLQSDLYTLSSSVTVPSSTWPSQTVTCNV | SWTDQDSKDSTYSMSSTLTLTKDEYERHNSY |
| AHPASSTKVDKKIVPRDCGCKPCICTVPEVSSVFI | TCEATHKTSTSPIVKSFNRNEC (SEQ ID NO: |
| FPPKPKDVLTITLTPKVTCVVVDISKDDPEVQFSW | 212) |
| FVDDVEVHTAQTKPREEQINSTFRSVSELPIMHQ | |
| DWLNGKEFKCRVNSAAFPAPIEKTISKTKGRPKA | |
| PQVYTIPPPKEQMAKDKVSLTCMITNFFPEDITVE | |
| WQWNGQPAENYKNTQPIMDTDGSYFVYSKLNV | |
| QKSNWEAGNTFTCSVLHEGLHNHHTEKSLSHSP | |
| GK (SEQ ID NO: 211) | |
| EVQLQQSGAELVRPGASVRLSCTASGFNIKDSLM | DIQMTQSPASQSASLGESVTFTCLASQTIGTW |
| HWVKQRPEQGLEWIGWIDPEDGETKYAPNFQSK | LAWYQQKPGKSPQLLIYAATSLADGVPSRFSG |
| ATITAVTSSNTAYLQLSSLTSEDSAIYYCARYTSPY | SGSGTKFSFKISSLQAEDFASYYCQQVYSTPW |
| YTMDYWGQGTSVTVSSAKTTPPSVYPLAPGSAA | TFGGGTKLEIKRADAAPTVSIFPPSSEQLTSGG |
| QTNSMVTLGCLVKGYFPEPVTVTWNSGSLSSGV | ASVVCFLNNFYPKDINVKWKIDGSERQNGVLN |
| HTFPAVLQSDLYTLSSSVTVPSSTWPSQTVTCNV | SWTDQDSKDSTYSMSSTLTLTKDEYERHNSY |
| AHPASSTKVDKKIVPRDCGCKPCICTVPEVSSVFI | TCEATHKTSTSPIVKSFNRNEC (SEQ ID NO: |
| FPPKPKDVLTITLTPKVTCVVVDISKDDPEVQFSW | 214) |
| FVDDVEVHTAQTKPREEQINSTFRSVSELPIMHQ | |
| DWLNGKEFKCRVNSAAFPAPIEKTISKTKGRPKA | |
| PQVYTIPPPKEQMAKDKVSLTCMITNFFPEDITVE | |
| WQWNGQPAENYKNTQPIMDTDGSYFVYSKLNV | |
| QKSNWEAGNTFTCSVLHEGLHNHHTEKSLSHSP | |
| GK (SEQ ID NO: 213) | |
| EVQLQQSGAELVRPGASVRLSCTASGFNIKDSLM | DIQMTQSPASQSASLGESVTFTCLASQTIGTW |
| HWVKQRPEQGLEWIGWIDPEDGETRYAPNFQDK | LAWYQQKPGKSPQLLIYAATSLADGVPSRFSG |
| ATITAVTSSNTAYLQLSSLTSEDSAIYYCARYTSRY | SGSGTKFSFKISSLQAEDFASYYCQQLYWTP |
| YTMEYWGQGTSVTVSSAKTTPPSVYPLAPGSAA | WTFGGGTKLEIKRADAAPTVSIFPPSSEQLTS |
| QTNSMVTLGCLVKGYFPEPVTVTWNSGSLSSGV | GGASVVCFLNNFYPKDINVKWKIDGSERQNG |
| HTFPAVLQSDLYTLSSSVTVPSSTWPSQTVTCNV | VLNSWTDQDSKDSTYSMSSTLTLTKDEYERH |
| AHPASSTKVDKKIVPRDCGCKPCICTVPEVSSVFI | NSYTCEATHKTSTSPIVKSFNRNEC (SEQ ID |
| FPPKPKDVLTITLTPKVTCVVVDISKDDPEVQFSW | NO: 216) |
| FVDDVEVHTAQTKPREEQINSTFRSVSELPIMHQ | |
| DWLNGKEFKCRVNSAAFPAPIEKTISKTKGRPKA | |
| PQVYTIPPPKEQMAKDKVSLTCMITNFFPEDITVE | |
| WQWNGQPAENYKNTQPIMDTDGSYFVYSKLNV | |
| QKSNWEAGNTFTCSVLHEGLHNHHTEKSLSHSP | |
| GK (SEQ ID NO: 215) | |
| QVHLVESGGGVVQPGRSLRLSCAASGFTFSNYGI | EIVMTQSPATLSVSTGERATLSCRASQNISSKL |
| HWVRQVPGKGLEWVAVISYDGNNKYYGDSVKG | AWYQQKPGQAPRLLIYGASTRASGIPARFWG |
| RFTISRDNSKNTLYLQMNSLRPEDTAIYYCAKDRG | SGSGTEFTLTISSLQSEDFAVFYCLQYNNWWT |
| LDTAGDYFDYWGQGTLVTVSSASTKGPSVFPLAP | FGQGTKVEIKRTVAAPSVFIFPPSDEQLKSGTA |
| CSRSTSESTAALGCLVKDYFPEPVTVSWNSGALT | SVVCLLNNFYPREAKVQWKVDNALQSGNSQE |
| SGVHTFPAVLQSSGLYSLSSVVTVPSSSLGTKTY | SVTEQDSKDSTYSLSSTLTLSKADYEKHKVYA |
| TCNVDHKPSNTKVDKRVESKYGPPCPPCPAPEF | CEVTHQGLSSPVTKSFNRGEC (SEQ ID NO: |
| LGGPSVFLFPPKPKDTLMISRTPEVTCVVVDVSQ | 344) |
| EDPEVQFNWYVDGVEVHNAKTKPREEQFNSTYR | |
| VVSVLTVLHQDWLNGKEYKCKVSNKGLPSSIEKTI | |
| SKAKGQPREPQVYTLPPSQEEMTKNQVSLTCLV | |
| KGFYPSDIAVEWESNGQPENNYKTTPPVLDSDG | |
| SFFLYSRLTVDKSRWQEGNVFSCSVMHEALHNH | |
| YTQKSLSLSLGK (SEQ ID NO: 343) | |
| QVQLQQWGAGLLKPSETLSLTCAVYNGSFSSYY | DIQMTQSPSSLSASVGDRVTITCQASQDINIYL |
| WNWIRQPPGKGLEWLGEINHRGSTNYNPSLTSR | NWYQQQPGKAPKLLIYDVSNLVPGVPSRFSG |
| VTISVDTSKNQFSLKLTSVTAADTAIYYCTAHTRG | TGSGTHFSFTISSLQPEDIATYYCQHYDDLPLT |
| FLYWGQGTLVTVSSASTKGPSVFPLAPCSRSTSE | FGGGTTVAIRRTVAAPSVFIFPPSDEQLKSGTA |
| STAALGCLVKDYFPEPVTVSWNSGALTSGVHTFP | SVVCLLNNFYPREAKVQWKVDNALQSGNSQE |
| AVLQSSGLYSLSSVVTVPSSSLGTKTYTCNVDHK | SVTEQDSKDSTYSLSSTLTLSKADYEKHKVYA |
| PSNTKVDKRVESKYGPPCPPCPAPEFLGGPSVFL | CEVTHQGLSSPVTKSFNRGEC (SEQ ID NO: |
| FPPKPKDTLMISRTPEVTCVVVDVSQEDPEVQFN | 346) |
| WYVDGVEVHNAKTKPREEQFNSTYRVVSVLTVL | |
| HQDWLNGKEYKCKVSNKGLPSSIEKTISKAKGQP | |
| REPQVYTLPPSQEEMTKNQVSLTCLVKGFYPSDI | |
| AVEWESNGQPENNYKTTPPVLDSDGSFFLYSRLT | |
| VDKSRWQEGNVFSCSVMHEALHNHYTQKSLSLS | |
| LGK (SEQ ID NO: 345) | |
| QVQLQQWGAGLLKPSETLSLTCAVYNGSFSSFY | DIQMTQSPSSLSASVGDRVTITCQASQDINNYL |
| WSWIRQPPGKGLEWLGEINHRGRTNNNPSLASR | NWYQQKPGQAPNLLIYDVFNLLPGVPSRFSG |
| VTISVDTSKSQFSLRLTSVTAADTATYYCTAHTRG | SGSGTDFSFTISSLQPEDIATYYCQHYDDLPLT |
| FLYWGQGTRVTVSSASTKGPSVFPLAPCSRSTSE | FGGGTTVAIRRTVAAPSVFIFPPSDEQLKSGTA |
| STAALGCLVKDYFPEPVTVSWNSGALTSGVHTFP | SVVCLLNNFYPREAKVQWKVDNALQSGNSQE |
| AVLQSSGLYSLSSVVTVPSSSLGTKTYTCNVDHK | SVTEQDSKDSTYSLSSTLTLSKADYEKHKVYA |
| PSNTKVDKRVESKYGPPCPPCPAPEFLGGPSVFL | CEVTHQGLSSPVTKSFNRGEC (SEQ ID NO: |
| FPPKPKDTLMISRTPEVTCVVVDVSQEDPEVQFN | 348) |
| WYVDGVEVHNAKTKPREEQFNSTYRVVSVLTVL | |
| HQDWLNGKEYKCKVSNKGLPSSIEKTISKAKGQP | |
| REPQVYTLPPSQEEMTKNQVSLTCLVKGFYPSDI | |
| AVEWESNGQPENNYKTTPPVLDSDGSFFLYSRLT | |
| VDKSRWQEGNVFSCSVMHEALHNHYTQKSLSLS | |
| LGK (SEQ ID NO: 347) | |
| QVQLQQWGAGLLKPSETLSLTCAVYNGSFSSYY | DIQMTQSPSSLSASVGDRVTITCQASQDINNYL |
| WSWIRQPPGKGLEWLGEINHRGRTNYNPSLTSR | NWYQQKPGKAPKLLIYDASNLVPGVPSRFSGT |
| VTISVDTSKNQFSLKLPSVTAADTAIYYCSAHTRG | GSGTDFSFTISSLQPEDIATYYCQHYYDLPLTF |
| FLYWGQGTLVTVSSASTKGPSVFPLAPCSRSTSE | GGGTTVAISRTVAAPSVFIFPPSDEQLKSGTAS |
| STAALGCLVKDYFPEPVTVSWNSGALTSGVHTFP | VVCLLNNFYPREAKVQWKVDNALQSGNSQES |
| AVLQSSGLYSLSSVVTVPSSSLGTKTYTCNVDHK | VTEQDSKDSTYSLSSTLTLSKADYEKHKVYAC |
| PSNTKVDKRVESKYGPPCPPCPAPEFLGGPSVFL | EVTHQGLSSPVTKSFNRGEC (SEQ ID NO: |
| FPPKPKDTLMISRTPEVTCVVVDVSQEDPEVQFN | 350) |
| WYVDGVEVHNAKTKPREEQFNSTYRVVSVLTVL | |
| HQDWLNGKEYKCKVSNKGLPSSIEKTISKAKGQP | |
| REPQVYTLPPSQEEMTKNQVSLTCLVKGFYPSDI | |
| AVEWESNGQPENNYKTTPPVLDSDGSFFLYSRLT | |
| VDKSRWQEGNVFSCSVMHEALHNHYTQKSLSLS | |
| LGK (SEQ ID NO: 349) | |
| QVQLQQWGAGLLKPSETLSLTCAVYNGSFSSYY | DIQMTQSPSSLSASVGDRVTITCQASQDINNYL |
| WNWIRQPPGKGLEWLGEINHRGSTNYNPSLTSR | NWYQQKPGKAPKLLIYDASNLVPGVPSRFSGT |
| VTISVDTSKNQFSLKLTSVTAADTAIYYCTAHTRG | GSGTDFSFTISSLQPEDIATYYCQHYYDLPLTF |
| FLYWGQGTLVTVSSASTKGPSVFPLAPCSRSTSE | GGGTTVAIRRTVAAPSVFIFPPSDEQLKSGTAS |
| STAALGCLVKDYFPEPVTVSWNSGALTSGVHTFP | VVCLLNNFYPREAKVQWKVDNALQSGNSQES |
| AVLQSSGLYSLSSVVTVPSSSLGTKTYTCNVDHK | VTEQDSKDSTYSLSSTLTLSKADYEKHKVYAC |
| PSNTKVDKRVESKYGPPCPPCPAPEFLGGPSVFL | EVTHQGLSSPVTKSFNRGEC (SEQ ID NO: |
| FPPKPKDTLMISRTPEVTCVVVDVSQEDPEVQFN | 351) |
| WYVDGVEVHNAKTKPREEQFNSTYRVVSVLTVL | |
| HQDWLNGKEYKCKVSNKGLPSSIEKTISKAKGQP | |
| REPQVYTLPPSQEEMTKNQVSLTCLVKGFYPSDI | |
| AVEWESNGQPENNYKTTPPVLDSDGSFFLYSRLT | |
| VDKSRWQEGNVFSCSVMHEALHNHYTQKSLSLS | |
| LGK (SEQ ID NO: 345) | |
| EIQLVESGGDLVQPGGSLKLSCAASGFTFSDSAM | DVVMTQSPLSLSATLGQPASISCRSSQSLVYS |
| HWVRQASGKGLEWVGRIRNKANTYATSYAASVK | DGITYLTWFQQRPGQSPTRLIYQVSNRDSGVP |
| GRFTVSTDDSKTTAYLQMRYLKIEDTAIYYCTSDQ | DRFNGSGSGTDFTLNISRVEAEDVGFYYCME |
| FDYWGQGTLVTVSSASTKGPSVFPLAPCSRSTSE | GTHWPWTFGQGTKVELKRTVAAPSVFIFPPS |
| STAALGCLVKDYFPEPVTVSWNSGALTSGVHTFP | DEQLKSGTASVVCLLNNFYPREAKVQWKVDN |
| AVLQSSGLYSLSSVVTVPSSSLGTKTYTCNVDHK | ALQSGNSQESVTEQDSKDSTYSLSSTLTLSKA |
| PSNTKVDKRVESKYGPPCPPCPAPEFLGGPSVFL | DYEKHKVYACEVTHQGLSSPVTKSFNRGEC |
| FPPKPKDTLMISRTPEVTCVVVDVSQEDPEVQFN | (SEQ ID NO: 353) |
| WYVDGVEVHNAKTKPREEQFNSTYRVVSVLTVL | |
| HQDWLNGKEYKCKVSNKGLPSSIEKTISKAKGQP | |
| REPQVYTLPPSQEEMTKNQVSLTCLVKGFYPSDI | |
| AVEWESNGQPENNYKTTPPVLDSDGSFFLYSRLT | |
| VDKSRWQEGNVFSCSVMHEALHNHYTQKSLSLS | |
| LGK (SEQ ID NO: 352) | |
| QVQLVESGGGLVKPGGSLRLSCAASGFTFSDYY | NVVLTQTPISSPVILGQPASISCRSSQSLVHSD |
| MSWIRQAPGKGLEWISFISRTGSTKYYADSVKGR | GDTYLSWLHQRPGQPPRLLIYTVSNRVSGVP |
| FTISRDNAKNSLNLQMSSLRAEDTAVYFCAREPL | DRFSGSGAGTDFTLKISRVEAEDVGVYYCMQ |
| QHWGQGTLVTVSSASTKGPSVFPLAPCSRSTSE | GTQFPALTFGGGTKVEIKRTVAAPSVFIFPPSD |
| STAALGCLVKDYFPEPVTVSWNSGALTSGVHTFP | EQLKSGTASVVCLLNNFYPREAKVQWKVDNA |
| AVLQSSGLYSLSSVVTVPSSSLGTKTYTCNVDHK | LQSGNSQESVTEQDSKDSTYSLSSTLTLSKAD |
| PSNTKVDKRVESKYGPPCPPCPAPEFLGGPSVFL | YEKHKVYACEVTHQGLSSPVTKSFNRGEC |
| FPPKPKDTLMISRTPEVTCVVVDVSQEDPEVQFN | (SEQ ID NO: 355) |
| WYVDGVEVHNAKTKPREEQFNSTYRVVSVLTVL | |
| HQDWLNGKEYKCKVSNKGLPSSIEKTISKAKGQP | |
| REPQVYTLPPSQEEMTKNQVSLTCLVKGFYPSDI | |
| AVEWESNGQPENNYKTTPPVLDSDGSFFLYSRLT | |
| VDKSRWQEGNVFSCSVMHEALHNHYTQKSLSLS | |
| LGK (SEQ ID NO: 354) | |
| QVQLVESGGGLVKPGGSLRLSCAASGFTFSDYY | KIVLTQTPLSSPVTLGQPASISCRSSQSLVHSD |
| MSWIRQAPGKGLEWVSYISRTGSTKYYADSVKG | GDTYLSWLHQRPGQPPRLLIYTISNRLSGVPD |
| RFTISRDNAKNSLNLQMSSLRAEDTAVYFCAREP | RFSGSGAGTDFTLKISRVEAEDVGVYYCMQG |
| LQHWGQGTLVTVSSASTKGPSVFPLAPCSRSTS | TQFPALTFGGGTKVEIKRTVAAPSVFIFPPSDE |
| ESTAALGCLVKDYFPEPVTVSWNSGALTSGVHTF | QLKSGTASVVCLLNNFYPREAKVQWKVDNAL |
| PAVLQSSGLYSLSSVVTVPSSSLGTKTYTCNVDH | QSGNSQESVTEQDSKDSTYSLSSTLTLSKADY |
| KPSNTKVDKRVESKYGPPCPPCPAPEFLGGPSV | EKHKVYACEVTHQGLSSPVTKSFNRGEC |
| FLFPPKPKDTLMISRTPEVTCVVVDVSQEDPEVQ | (SEQ ID NO: 357) |
| FNWYVDGVEVHNAKTKPREEQFNSTYRVVSVLT | |
| VLHQDWLNGKEYKCKVSNKGLPSSIEKTISKAKG | |
| QPREPQVYTLPPSQEEMTKNQVSLTCLVKGFYP | |
| SDIAVEWESNGQPENNYKTTPPVLDSDGSFFLYS | |
| RLTVDKSRWQEGNVFSCSVMHEALHNHYTQKSL | |
| SLSLGK (SEQ ID NO: 356) | |
| EMQLVESGGGLVQPGGSLKVSCAASGFTLSDSSI | DIQMTQSPSTLSASVGDRITITCRASQSISTWL |
| HWVRQASGKGLEWIGRIRSKPYSYATAYAASVK | AWYQQKPGKAPKLLIYKASSLEGGVPSRFSGS |
| GRFTISRDDSKNTAFLQMSGLKTEDTAVYYCTGG | GSGTDFTLTISSLQPDDFATYYCQQYNTYWTF |
| DDFWGQGTLVTVSSASTKGPSVFPLAPCSRSTS | GQGTKVEIKRTVAAPSVFIFPPSDEQLKSGTAS |
| ESTAALGCLVKDYFPEPVTVSWNSGALTSGVHTF | VVCLLNNFYPREAKVQWKVDNALQSGNSQES |
| PAVLQSSGLYSLSSVVTVPSSSLGTKTYTCNVDH | VTEQDSKDSTYSLSSTLTLSKADYEKHKVYAC |
| KPSNTKVDKRVESKYGPPCPPCPAPEFLGGPSV | EVTHQGLSSPVTKSFNRGEC (SEQ ID NO: |
| FLFPPKPKDTLMISRTPEVTCVVVDVSQEDPEVQ | 359) |
| FNWYVDGVEVHNAKTKPREEQFNSTYRVVSVLT | |
| VLHQDWLNGKEYKCKVSNKGLPSSIEKTISKAKG | |
| QPREPQVYTLPPSQEEMTKNQVSLTCLVKGFYP | |
| SDIAVEWESNGQPENNYKTTPPVLDSDGSFFLYS | |
| RLTVDKSRWQEGNVFSCSVMHEALHNHYTQKSL | |
| SLSLGK (SEQ ID NO: 358) | |
| EVQLVESGGGLVQPGGSLRLSCAASGFTFSSYA | EIVMTQSPATLSVSPGKRATLSCRASQSVSSN |
| MSWVRQAPGKGLEWVSAISGSGGSTYYADSVK | LAWYQQKPGQAPRLLIYGASTRATGIPARFSG |
| GRFTISRDNSKNTLYLQMNSLRAEDTAVYYCAIPY | SGSGTEFTLTISSLQSEDFAVYYCQQYNNWPP |
| NWNYGGAFDIWGQGTMVTVSSASTKGPSVFPLA | YTFGQGTKLEIKRTVAAPSVFIFPPSDEQLKSG |
| PCSRSTSESTAALGCLVKDYFPEPVTVSWNSGAL | TASVVCLLNNFYPREAKVQWKVDNALQSGNS |
| TSGVHTFPAVLQSSGLYSLSSVVTVPSSSLGTKT | QESVTEQDSKDSTYSLSSTLTLSKADYEKHKV |
| YTCNVDHKPSNTKVDKRVESKYGPPCPPCPAPE | YACEVTHQGLSSPVTKSFNRGEC (SEQ ID |
| FLGGPSVFLFPPKPKDTLMISRTPEVTCVVVDVS | NO: 361) |
| QEDPEVQFNWYVDGVEVHNAKTKPREEQFNSTY | |
| RVVSVLTVLHQDWLNGKEYKCKVSNKGLPSSIEK | |
| TISKAKGQPREPQVYTLPPSQEEMTKNQVSLTCL | |
| VKGFYPSDIAVEWESNGQPENNYKTTPPVLDSD | |
| GSFFLYSRLTVDKSRWQEGNVFSCSVMHEALHN | |
| HYTQKSLSLSLGK (SEQ ID NO: 360) | |
| QVQLVQSGAEVKKPGASVKVSCKASGYTFTGYY | DIQMTQSPSSLSASVGDRVTITCRASQGINNYL |
| MHWVRQAPGQGLEWMGWINPNSVGTNFAQKFQ | AWYQQKPGKVPKLLIYAASTLQSGVPSRFSGS |
| GRVTMTRDTSISTAYMELSRLRSDDTAVYYCTRD | GSGTDFTLTISSLQPEDVATYYCQKFNSAPLTF |
| GAAAGLFDYWGQGTLVTVSSASTKGPSVFPLAP | GGGTKVEIKRTVAAPSVFIFPPSDEQLKSGTAS |
| CSRSTSESTAALGCLVKDYFPEPVTVSWNSGALT | VVCLLNNFYPREAKVQWKVDNALQSGNSQES |
| SGVHTFPAVLQSSGLYSLSSVVTVPSSSLGTKTY | VTEQDSKDSTYSLSSTLTLSKADYEKHKVYAC |
| TCNVDHKPSNTKVDKRVESKYGPPCPPCPAPEF | EVTHQGLSSPVTKSFNRGEC (SEQ ID NO: |
| LGGPSVFLFPPKPKDTLMISRTPEVTCVVVDVSQ | 363) |
| EDPEVQFNWYVDGVEVHNAKTKPREEQFNSTYR | |
| VVSVLTVLHQDWLNGKEYKCKVSNKGLPSSIEKTI | |
| SKAKGQPREPQVYTLPPSQEEMTKNQVSLTCLV | |
| KGFYPSDIAVEWESNGQPENNYKTTPPVLDSDG | |
| SFFLYSRLTVDKSRWQEGNVFSCSVMHEALHNH | |
| YTQKSLSLSLGK (SEQ ID NO: 362) | |
| EVQLVESGGGLVQPGGSLRLSCAASGFTFSTYA | DIQMTQSPSSLSASVGDRVTITCRASQSISSYL |
| MSWVRQAPGKGLEWVSAISGSGGSTYYADSVK | NWYQQKPGKAPKLLIYAASSLQSGVPSRFSG |
| GRFTISRDNSKNTLYLQMKSLRVEDTAVYYCANS | SGSGTDFTLTISSLQPEDFATYYCQQSYSTPPI |
| PSWFDPWGQGTLVTVSSASTKGPSVFPLAPCSR | TFGQGTRLEIKRTVAAPSVFIFPPSDEQLKSGT |
| STSESTAALGCLVKDYFPEPVTVSWNSGALTSGV | ASVVCLLNNFYPREAKVQWKVDNALQSGNSQ |
| HTFPAVLQSSGLYSLSSVVTVPSSSLGTKTYTQN | ESVTEQDSKDSTYSLSSTLTLSKADYEKHKVY |
| VDHKPSNTKVDKRVESKYGPPCPPCPAPEFLGG | ACEVTHQGLSSPVTKSFNRGEC (SEQ ID NO: |
| PSVFLFPPKPKDTLMISRTPEVTCVVVDVSQEDP | 365) |
| EVQFNWYVDGVEVHNAKTKPREEQFNSTYRVVS | |
| VLTVLHQDWLNGKEYKCKVSNKGLPSSIEKTISKA | |
| KGQPREPQVYTLPPSQEEMTKNQVSLTCLVKGF | |
| YPSDIAVEWESNGQPENNYKTTPPVLDSDGSFFL | |
| YSRLTVDKSRWQEGNVFSCSVMHEALHNHYTQK | |
| SLSLSLGK (SEQ ID NO: 364) | |
| EVQLVESGGGLVQPGRSLRLSCAASGFTFDDYG | DIVMTQSPDSLAVSLGERATINCKSSQSVLYSS |
| MHWVRQAPGKGLEWVSVISWNSATIDYADSVKG | NDNNYLAWYQQKPGQPPKLLIYWASTRESGV |
| RFTISRDNAKNSLFLQMNSLRPEDTALYYCAKKN | PDRFSGSGSGTDFTLTISSLQAEDVAVYYCQQ |
| SLGWFFDYWGQGTLVTVSSASTKGPSVFPLAPC | YYSTPLTFGGGTKVEIKRTVAAPSVFIFPPSDE |
| SRSTSESTAALGCLVKDYFPEPVTVSWNSGALTS | QLKSGTASVVCLLNNFYPREAKVQWKVDNAL |
| GVHTFPAVLQSSGLYSLSSVVTVPSSSLGTKTYT | QSGNSQESVTEQDSKDSTYSLSSTLTLSKADY |
| CNVDHKPSNTKVDKRVESKYGPPCPPCPAPEFL | EKHKVYACEVTHQGLSSPVTKSFNRGEC |
| GGPSVFLFPPKPKDTLMISRTPEVTCVVVDVSQE | (SEQ ID NO: 367) |
| DPEVQFNWYVDGVEVHNAKTKPREEQFNSTYRV | |
| VSVLTVLHQDWLNGKEYKCKVSNKGLPSSIEKTIS | |
| KAKGQPREPQVYTLPPSQEEMTKNQVSLTCLVK | |
| GFYPSDIAVEWESNGQPENNYKTTPPVLDSDGSF | |
| FLYSRLTVDKSRWQEGNVFSCSVMHEALHNHYT | |
| QKSLSLSLGK (SEQID NO: 366) | |
| QVQLVQSGAEVKKPGASVKVSCKASGYTFTSYGI | DIQMTQSPSSLSASVGDRVTITCRASQGIRND |
| SWVRQAPGQGLEWMGWISAYNGNTIYAQKFQG | LGWFQQKPGKAPKRLIYAASSLQSGVPSRFS |
| RVTMTTDTSTSTAYMELRSLRSDDTAVYYCARDI | GSGSGTEFTLTISSLQPEDFATYYCLQHNSYP |
| NWYFDLWGRGTLVTVSSASTKGPSVFPLAPCSR | WTFGQGTKVEIKRTVAAPSVFIFPPSDEQLKS |
| STSESTAALGCLVKDYFPEPVTVSWNSGALTSGV | GTASVVCLLNNFYPREAKVQWKVDNALQSGN |
| HTFPAVLQSSGLYSLSSVVTVPSSSLGTKTYTQN | SQESVTEQDSKDSTYSLSSTLTLSKADYEKHK |
| VDHKPSNTKVDKRVESKYGPPCPPCPAPEFLGG | VYACEVTHQGLSSPVTKSFNRGEC (SEQ ID |
| PSVFLFPPKPKDTLMISRTPEVTCVVVDVSQEDP | NO: 369) |
| EVQFNWYVDGVEVHNAKTKPREEQFNSTYRVVS | |
| VLTVLHQDWLNGKEYKCKVSNKGLPSSIEKTISKA | |
| KGQPREPQVYTLPPSQEEMTKNQVSLTCLVKGF | |
| YPSDIAVEWESNGQPENNYKTTPPVLDSDGSFFL | |
| YSRLTVDKSRWQEGNVFSCSVMHEALHNHYTQK | |
| SLSLSLGK (SEQ ID NO: 368) | |
| EVQLVESGGGLVQPGRSLRLSCAASGFTFDDYG | DIQMTQSPSSLSAFVGDRVTITCRASQGIRNDL |
| MHWVRQAPGKGLEWVSVISWNGATIDYADSVKG | GWYQQKPGKTPKRLIYTASSLRSGVPSRFSG |
| RFTISRDNAKNSLFLQMNSLRPEDTALYYCAKKN | SGSGTDFTLTISSLQPEDFATYYCLQHNNYPY |
| SLGWFFDYWGQGTLVTVSSASTKGPSVFPLAPC | TFGQGTKLEIKRTVAAPSVFIFPPSDEQLKSGT |
| SRSTSESTAALGCLVKDYFPEPVTVSWNSGALTS | ASVVCLLNNFYPREAKVQWKVDNALQSGNSQ |
| GVHTFPAVLQSSGLYSLSSVVTVPSSSLGTKTYT | ESVTEQDSKDSTYSLSSTLTLSKADYEKHKVY |
| CNVDHKPSNTKVDKRVESKYGPPCPPCPAPEFL | ACEVTHQGLSSPVTKSFNRGEC (SEQ ID NO: |
| GGPSVFLFPPKPKDTLMISRTPEVTCVVVDVSQE | 371) |
| DPEVQFNWYVDGVEVHNAKTKPREEQFNSTYRV | |
| VSVLTVLHQDWLNGKEYKCKVSNKGLPSSIEKTIS | |
| KAKGQPREPQVYTLPPSQEEMTKNQVSLTCLVK | |
| GFYPSDIAVEWESNGQPENNYKTTPPVLDSDGSF | |
| FLYSRLTVDKSRWQEGNVFSCSVMHEALHNHYT | |
| QKSLSLSLGK (SEQ ID NO: 370) | |
| EVQLVESGGNLVQPGGSLRLSCAASGFTFSNYA | DIQMTQSPSSLSASVGDRVTITCRSSQSVISYL |
| MSWVRQAPGKGLEWVSGITGNGVNTYYSVSVK | NWYQQKPGKAPKLLIFGASSLISGVPSRFSGS |
| GRFTISRDNSKNTLFLEMNSLRAEDTAIYYCVKER | GSGTDFTLTIVSLQPEDFAVYFCQQNYLPPLTF |
| GHSWFGDWFDPWGQGTLVTVSSASTKGPSVFP | GGGTKVEIKRTVAAPSVFIFPPSDEQLKSGTAS |
| LAPCSRSTSESTAALGCLVKDYFPEPVTVSWNSG | VVCLLNNFYPREAKVQWKVDNALQSGNSQES |
| ALTSGVHTFPAVLQSSGLYSLSSVVTVPSSSLGT | VTEQDSKDSTYSLSSTLTLSKADYEKHKVYAC |
| KTYTCNVDHKPSNTKVDKRVESKYGPPCPPCPA | EVTHQGLSSPVTKSFNRGEC (SEQ ID NO: |
| PEFLGGPSVFLFPPKPKDTLMISRTPEVTCVVVDV | 373) |
| SQEDPEVQFNWYVDGVEVHNAKTKPREEQFNST | |
| YRVVSVLTVLHQDWLNGKEYKCKVSNKGLPSSIE | |
| KTISKAKGQPREPQVYTLPPSQEEMTKNQVSLTC | |
| LVKGFYPSDIAVEWESNGQPENNYKTTPPVLDSD | |
| GSFFLYSRLTVDKSRWQEGNVFSCSVMHEALHN | |
| HYTQKSLSLSLGK (SEQ ID NO: 372) | |
| EVQLVESGGNLVQPGGSLRLSCAASGFTFSNYA | DIVMTQTPLSSPVTLGQPASISCRSSQGLVNS |
| MSWVRQAPGKGLEWVSGITGNGIKTYYSVSAKG | DGNTYLSWLHQRPGQPPRLLIYEISNRFSGVP |
| RFTISRDNSKNTLFLQMNSLRAEDTAIYYCVKERG | DRFSGSGAGTDFTLKISRVEAEDVGFYYCMQ |
| HSWFGDWFDPWGQGTLVTVSSASTKGPSVFPLA | STQFPLTFGGGTKVEIKRTVAAPSVFIFPPSDE |
| PCSRSTSESTAALGCLVKDYFPEPVTVSWNSGAL | QLKSGTASVVCLLNNFYPREAKVQWKVDNAL |
| TSGVHTFPAVLQSSGLYSLSSVVTVPSSSLGTKT | QSGNSQESVTEQDSKDSTYSLSSTLTLSKADY |
| YTCNVDHKPSNTKVDKRVESKYGPPCPPCPAPE | EKHKVYACEVTHQGLSSPVTKSFNRGEC |
| FLGGPSVFLFPPKPKDTLMISRTPEVTCVVVDVS | (SEQ ID NO: 375) |
| QEDPEVQFNWYVDGVEVHNAKTKPREEQFNSTY | |
| RVVSVLTVLHQDWLNGKEYKCKVSNKGLPSSIEK | |
| TISKAKGQPREPQVYTLPPSQEEMTKNQVSLTCL | |
| VKGFYPSDIAVEWESNGQPENNYKTTPPVLDSD | |
| GSFFLYSRLTVDKSRWQEGNVFSCSVMHEALHN | |
| HYTQKSLSLSLGK (SEQ ID NO: 374) | |
| EVQLVESGGNLVQPGGSLRLSCAASGFTFSNYA | DIQMTQSPSSLSASVGDRVTITCRASQGIRND |
| MSWVRQAPGKGLEWVSGITGNGINTYYSVSVKG | LGWYQQKPGKAPKRLIYAASSLQSGVPSRFS |
| RFTISRDNSKNTVFLQMNSLRAEDTAIYYCVKERG | GSGSGTEFTLTISSLQPEDFATYYCLQHNSYP |
| HSWFGDWFDPWGQGTLVTVSSASTKGPSVFPLA | WTFGQGTKVEIKRTVAAPSVFIFPPSDEQLKS |
| PCSRSTSESTAALGCLVKDYFPEPVTVSWNSGAL | GTASVVCLLNNFYPREAKVQWKVDNALQSGN |
| TSGVHTFPAVLQSSGLYSLSSVVTVPSSSLGTKT | SQESVTEQDSKDSTYSLSSTLTLSKADYEKHK |
| YTCNVDHKPSNTKVDKRVESKYGPPCPPCPAPE | VYACEVTHQGLSSPVTKSFNRGEC (SEQ ID |
| FLGGPSVFLFPPKPKDTLMISRTPEVTCVVVDVS | NO: 377) |
| QEDPEVQFNWYVDGVEVHNAKTKPREEQFNSTY | |
| RVVSVLTVLHQDWLNGKEYKCKVSNKGLPSSIEK | |
| TISKAKGQPREPQVYTLPPSQEEMTKNQVSLTCL | |
| VKGFYPSDIAVEWESNGQPENNYKTTPPVLDSD | |
| GSFFLYSRLTVDKSRWQEGNVFSCSVMHEALHN | |
| HYTQKSLSLSLGK (SEQ ID NO: 376) | |
| QVQLVESGGGVVQPGRSLRLSCEVSGFTFSNYG | AIRMTQSPSSFSATTGDRVTITCRASQGISSHL |
| MHWVRQAPGKGLEWVAVVSYDGSNKYYTDSVK | AWYQQKPGKAPKFLIYVASTLQSGVPSRFSGS |
| GRFTISRDNSKNTLSLQMNSLRAEDTAVYYCAKD | GSGTDFTLTISSLQSEDFATYYCQQYYSYPPT |
| RGITGTSGGVFDIWGQGTMVTVSSASTKGPSVFP | FGQGTKVEIKRTVAAPSVFIFPPSDEQLKSGTA |
| LAPCSRSTSESTAALGCLVKDYFPEPVTVSWNSG | SVVCLLNNFYPREAKVQWKVDNALQSGNSQE |
| ALTSGVHTFPAVLQSSGLYSLSSVVTVPSSSLGT | SVTEQDSKDSTYSLSSTLTLSKADYEKHKVYA |
| KTYTCNVDHKPSNTKVDKRVESKYGPPCPPCPA | CEVTHQGLSSPVTKSFNRGEC (SEQ ID NO: |
| PEFLGGPSVFLFPPKPKDTLMISRTPEVTCVVVDV | 379) |
| SQEDPEVQFNWYVDGVEVHNAKTKPREEQFNST | |
| YRVVSVLTVLHQDWLNGKEYKCKVSNKGLPSSIE | |
| KTISKAKGQPREPQVYTLPPSQEEMTKNQVSLTC | |
| LVKGFYPSDIAVEWESNGQPENNYKTTPPVLDSD | |
| GSFFLYSRLTVDKSRWQEGNVFSCSVMHEALHN | |
| HYTQKSLSLSLGK (SEQ ID NO: 378) | |
| EVQLVESGGGLVQPGGSLRLSCAASGFTSSRYW | DVVMTQSLLSLSVTLGQPASISCRSSQSLVYS |
| MSWVRQAPGKGLEWLANINQDGSEKYYLESLRG | DGDTYLSWFQQRPGQSPRRLIYKVSHRDSGV |
| RFTISRDNAKNSLYLQMNNLRAEDTAVYYCARDE | PDRFRGSGSGTDFTLKISRVEAEDVGIYYCMQ |
| EGYWGQGTLVTVSSASTKGPSVFPLAPCSRSTS | GTHWPPTFGQGTKVEIKRTVAAPSVFIFPPSD |
| ESTAALGCLVKDYFPEPVTVSWNSGALTSGVHTF | EQLKSGTASVVCLLNNFYPREAKVQWKVDNA |
| PAVLQSSGLYSLSSVVTVPSSSLGTKTYTCNVDH | LQSGNSQESVTEQDSKDSTYSLSSTLTLSKAD |
| KPSNTKVDKRVESKYGPPCPPCPAPEFLGGPSV | YEKHKVYACEVTHQGLSSPVTKSFNRGEC |
| FLFPPKPKDTLMISRTPEVTCVVVDVSQEDPEVQ | (SEQ ID NO: 381) |
| FNWYVDGVEVHNAKTKPREEQFNSTYRVVSVLT | |
| VLHQDWLNGKEYKCKVSNKGLPSSIEKTISKAKG | |
| QPREPQVYTLPPSQEEMTKNQVSLTCLVKGFYP | |
| SDIAVEWESNGQPENNYKTTPPVLDSDGSFFLYS | |
| RLTVDKSRWQEGNVFSCSVMHEALHNHYTQKSL | |
| SLSLGK (SEQ ID NO: 380) | |

In some embodiments, the subject may be administered an isolated ALK3 antibody, or antigen-binding fragment thereof. The ALK3 antibody or antigen binding fragment thereof can contain an antigen binding fragment (Fab) described in Harth et al., PLoS ONE 5: e13049, 2010, such as AbD1556 or AbD1564, both of which were found to have high nanomolar affinities for BMPR1 and to neutralize BMP2 activity.

In some embodiments, the ALK3 antibody or an antigen binding fragment thereof specifically binds to an extracellular domain of human ALK3 (BMPR1A) and contains: (a) a heavy chain CDR1 including TGYYMK (SEQ ID NO: 79); (b) a heavy chain CDR2 including RINPDNGGRTYNQIFKDK (SEQ ID NO: 80); and (c) a heavy chain CDR3 including RERGQYGNYGGFSD (SEQ ID NO: 81).

In some embodiments, the ALK3 antibody or an antigen binding fragment thereof contains a heavy chain variable region having at least 90% (e.g., at least 91%, 92%, 93%, 94%, 95%, 96%, 97%, 98%, 99%, or more) sequence identity to SEQ ID NO: 77 or SEQ ID NO: 78, shown below:

(SEQ ID NO: 77)
MEWSWIFLFLLSGTAGVLSEVQLQQSGPELVKPGTSVKISCKASGYSFTG
YYMHWVKQSQVKSLEWIGRINPDNGGRTYNQIFKDKASLTVHKSSSTAYM
ELHSLTSDDSAVYYCTRERGQYGNYGGFSDWGQGTLVT
(SEQ ID NO: 78)
EVQLQQSGPELVKPGTSVKISCKASGYSFTGYYMHWVKQSQVKSLEWIGR
INPDNGGRTYNQIFKDKASLTVHKSSSTAYMELHSLTSDDSAVYYCTRER
GQYGNYGGFSDWGQGTLVT

In some embodiments, the ALK3 antibody or an antigen binding fragment thereof contains a heavy chain variable region having at least 95% (e.g., at 95%, 96%, 97%, 98%, 99%, or more), at least 97% (e.g., at least 97%, 98%, 99%, or more), or at least 99% sequence identity to SEQ ID NO: 77 or SEQ ID NO: 78. In some embodiments, the antibody contains a heavy chain variable region having the sequence of SEQ ID NO: 77 or SEQ ID NO: 78. Such antibodies are described in U.S. Patent Application Publication No. US20130089560A1, which is incorporated herein by reference.

### Vectors, Host Cells, and Protein Production

The ALK2 and ALK3 antibodies and antigen binding fragments thereof (e.g., ALK2 or ALK3 binding fragments) described herein can be produced from a host cell. A host cell refers to a vehicle that includes the necessary cellular components, e.g., organelles, needed to express the antibodies and antigen binding fragments thereof, described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Generally, preferred host cells are of either eukaryotic (e.g., mammalian) or prokaryotic (e.g., bacterial) origin.

### Nucleic Acid Vector Construction and Host Cells

A nucleic acid sequence encoding the amino acid sequence of an ALK2 or ALK3 antibody or antigen binding fragment thereof (e.g., an ALK2 or ALK3 binding fragment), described herein may be prepared by a variety of methods known in the art. These methods include, but are not limited to, oligonucleotide-mediated (or site-directed) mutagenesis and PCR mutagenesis. A nucleic acid molecule encoding an ALK2 or ALK3 antibody, or antigen binding fragment thereof, described herein may be obtained using standard techniques, e.g., gene synthesis. Alternatively, a nucleic acid molecule encoding an ALK2 or ALK3 antibody or antigen binding fragment thereof, described herein may be mutated to include specific amino acid substitutions using standard techniques in the art, e.g., QuikChange™ mutagenesis. Nucleic acid molecules can be synthesized using a nucleotide synthesizer or PCR techniques.

A nucleic acid sequence encoding an ALK2 or ALK3 antibody or antigen binding fragment thereof (e.g., an ALK2 or ALK3 binding fragment), described herein may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding the protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7O3O, and HsS78Bst cells. In some embodiments, E. coli cells may also be used as host cells for the invention. Examples of E. coli strains include, but are not limited to, E. coli 294 (ATCC®31,446), E. coli A 1776 (ATCC®31,537, E. coli BL21 (DE3) (ATCC® BAA-1025), and E. coli RV308 (ATCC®31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the ALK2 or ALK3 antibodies or antigen binding fragments (e.g., ALK2 or ALK3 binding fragments) thereof, described herein may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector of the invention, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. An ALK2 or ALK3 antibody or antigen binding fragment thereof (e.g., an ALK2 or ALK3 binding fragment) described herein may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultrafiltration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the ALK2 or ALK3 antibodies or antigen binding fragments thereof (e.g., ALK2 or ALK3 binding fragments), described herein can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding an antibody or antigen binding fragment thereof, described herein. The vector, once inside a cell of the subject (e.g., by transformation, transfection, electroporation, calcium phosphate precipitation, direct microinjection, infection, etc.) will promote expression of the antibody or antigen binding fragment thereof, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions and Preparations

The ALK2 or ALK3 antibodies or antigen binding fragments thereof (e.g., ALK2 or ALK3 binding fragments), described herein can be incorporated into a vehicle for administration into a patient, such as a human patient suffering from bone disease, low red blood cell levels (e.g., low hemoglobin levels or low red blood cell count, e.g., anemia), heterotopic ossification (e.g., heterotopic ossification resulting from FOP), Sjogren's syndrome (e.g., dry eye associated with Sjogren's syndrome), MO, DIPG, PCO, or cardiac hypertrophy and/or cardiac fibrosis. In some embodiments, a pharmaceutical composition including an ALK2 or ALK3 antibody or an antigen binding fragment thereof (e.g., an ALK2 or ALK3 binding fragment), described herein may be used in combination with other agents (e.g., therapeutic biologics and/or small molecules) or compositions in a therapy. Pharmaceutical compositions containing ALK2 or ALK3 antibodies or antigen binding fragments thereof, can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of lyophilized formulations or aqueous solutions. In some embodiments, a pharmaceutical composition of the invention includes a nucleic acid molecule (DNA or RNA, e.g., mRNA) encoding an antibody or ALK2 or ALK3 binding fragment thereof, described herein, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEMI

Mixtures of ALK2 or ALK3 antibodies or antigen binding fragments thereof (e.g., ALK2 or ALK3 binding fragments), may be prepared in water suitably mixed with one or more excipients, carriers, or diluents. Dispersions may also be prepared in glycerol, liquid polyethylene glycols, and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated.

The pharmaceutical compositions of the invention may be prepared in microcapsules, such as hydroxylmethylcellulose or gelatin-microcapsule and poly-(methylmethacrylate) microcapsule. The pharmaceutical compositions of the invention may also be prepared in other drug delivery systems such as liposomes, albumin microspheres, microemulsions, nanoparticles, and nanocapsules. Such techniques are described in Remington: The Science and Practice of Pharmacology 22nd edition, Allen, L. Ed. (2013). The pharmaceutical compositions to be used for in vivo administration must be sterile. This is readily accomplished by filtration through sterile filtration membranes.

The pharmaceutical compositions of the invention may also be prepared as a sustained-release formulation. Suitable examples of sustained-release preparations include semipermeable matrices of solid hydrophobic polymers containing the polypeptides of the invention. Examples of sustained release matrices include polyesters, hydrogels, polylactides, copolymers of L-glutamic acid and y ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions of the invention for shorter time periods, e.g., days to weeks.

The pharmaceutical composition may be formed in a unit dose form as needed. The amount of active component, e.g., an ALK2 or ALK3 antibody or an antigen binding fragment thereof (e.g., an ALK2 or ALK3 binding fragment), described herein, included in the pharmaceutical preparations is such that a suitable dose within the designated range is provided (e.g., a dose within the range of 0.002 mg/kg to 3 mg/kg of body weight).

The pharmaceutical composition for gene therapy can be in an acceptable diluent or can include a slow-release matrix in which the gene delivery vehicle is imbedded. If hydrodynamic injection is used as the delivery method, the pharmaceutical composition containing a nucleic acid molecule encoding an antibody or ALK2 or ALK3 binding fragment thereof, described herein or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### Routes, Dosage, and Administration

Pharmaceutical compositions that include the ALK2 or ALK3 antibodies or antigen binding fragments thereof (e.g., ALK2 or ALK3 binding fragments), of the invention as the therapeutic agents may be by a variety of routes, such as intravenous, parenteral, intradermal, transdermal, intramuscular, intranasal, subcutaneous, percutaneous, topical, intratracheal, intraperitoneal, intraarterial, intravascular, intrathecal, intracerebroventricular, inhalation, perfusion, lavage, and oral administration. The pharmaceutical composition may also be formulated for, or administered via, oral, ocular, nasal, spray, aerosol, rectal, or vaginal administration. In specific embodiments, the pharmaceutical composition is administered subcutaneously. For injectable formulations, various effective pharmaceutical carriers are known in the art. See, e.g., ASHP Handbook on Injectable Drugs, Toissel, 18th ed. (2014). For ocular administration, the formulation may be delivered systemically, by injection (e.g., intraocular injection), or topically (e.g., as a solution, suspension, or ointment, such as by instillation (e.g., an eye drop)).

In some embodiments, a pharmaceutical composition that includes a nucleic acid molecule encoding an ALK2 or ALK3 antibody or an ALK2 or ALK3 binding fragment thereof, described herein or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding polypeptides of the invention may be administered directly to a subject.

In some embodiments of the present invention, nucleic acid molecules encoding a polypeptide described herein or vectors containing such nucleic acid molecules may be administered using a hydrodynamic injection platform. In the hydrodynamic injection method, a nucleic acid molecule encoding an antibody or ALK2 or ALK3 binding fragment thereof, described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding an antibody or ALK2 or ALK3 binding fragment thereof, described herein may be administered using hydrodynamic injection. The most suitable route and dosage for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. A pharmaceutical composition of the invention may include a dosage of an ALK2 or ALK3 antibody or an antigen binding fragment thereof (e.g., an ALK2 or ALK3 binding fragment), of the invention ranging from 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg), and, in a more specific embodiment, 0.01 mg/kg to 2 mg/kg (e.g., 0.05 mg/kg to 2 mg/kg, 0.1 mg/kg to 2 mg/kg, 0.5 mg/kg to 2 mg/kg, 1 mg/kg to 2 mg/kg, 1.5 mg/kg to 2 mg/kg, 0.01 mg/kg to 1.5 mg/kg, 0.01 mg/kg to 1 mg/kg, 0.01 mg/kg to 0.5 mg/kg, 0.01 mg/kg to 0.25 mg/kg, 0.01 mg/kg to 0.1 mg/kg, or 0.01 mg/kg to 0.05 mg/kg), and, in a more specific embodiment, 0.01 mg/kg to 1 mg/kg (e.g., 0.05 mg/kg to 1 mg/kg, 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.01 mg/kg to 0.9 mg/kg, 0.01 mg/kg to 0.8 mg/kg, 0.01 mg/kg to 0.7 mg/kg, 0.01 mg/kg to 0.6 mg/kg, 0.01 mg/kg to 0.5 mg/kg, 0.01 mg/kg to 0.4 mg/kg, 0.01 mg/kg to 0.3 mg/kg, 0.01 mg/kg to 0.2 mg/kg, 0.01 mg/kg to 0.1 mg/kg, or 0.01 mg/kg to 0.05 mg/kg), and, in a more specific embodiment, 0.05 mg/kg to 1 mg/kg (e.g., 0.1 mg/kg to 1 mg/kg, 0.2 mg/kg to 1 mg/kg, 0.3 mg/kg to 1 mg/kg, 0.4 mg/kg to 1 mg/kg, 0.5 mg/kg to 1 mg/kg, 0.6 mg/kg to 1 mg/kg, 0.7 mg/kg to 1 mg/kg, 0.8 mg/kg to 1 mg/kg, 0.9 mg/kg to 1 mg/kg, 0.05 mg/kg to 0.9 mg/kg, 0.05 mg/kg to 0.08 mg/kg, 0.05 mg/kg to 0.8 mg/kg, 0.05 mg/kg to 0.7 mg/kg, 0.05 mg/kg to 0.6 mg/kg, 0.05 mg/kg to 0.5 mg/kg, 0.05 mg/kg to 0.4 mg/kg, 0.05 mg/kg to 0.3 mg/kg, 0.05 mg/kg to 0.2 mg/kg, or 0.05 mg/kg to 0.1 mg/kg) or 0.01 mg/kg to 0.3 mg/kg (e.g., 0.01 mg/kg to 3 mg/kg, 0.025 mg/kg to 0.3 mg/kg, 0.05 mg/kg to 0.3 mg/kg, 0.075 mg/kg to 0.3 mg/kg, 0.1 mg/kg to 0.3 mg/kg, 0.125 mg/kg to 0.3 mg/kg, 0.15 mg/kg to 0.3 mg/kg, 0.175 mg/kg to 0.3 mg/kg, 0.2 mg/kg to 0.3 mg/kg, 0.225 mg/kg to 0.3 mg/kg, 0.25 mg/kg to 0.3 mg/kg, 0.275 mg/kg to 0.3 mg/kg, 0.01 mg/kg to 0.275 mg/kg, 0.01 mg/kg to 0.25 mg/kg, 0.01 mg/kg to 0.225 mg/kg, 0.01 mg/kg to 0.2 mg/kg, 0.01 mg/kg to 0.225 mg/kg, 0.01 mg/kg to 0.2 mg/kg, 0.01 mg/kg to 0.175 mg/kg, 0.01 mg/kg to 0.15 mg/kg, 0.01 mg/kg to 0.125 mg/kg, 0.01 mg/kg to 0.1 mg/kg, 0.01 mg/kg to 0.075 mg/kg, 0.01 mg/kg to 0.05 mg/kg, or 0.01 mg/kg to 0.025 mg/kg), and, in a more specific embodiment, 0.1 mg/kg to 0.3 mg/kg (e.g., 0.1 mg/kg to 0.25 mg/kg, 0.1 mg/kg to 0.2 mg/kg, 0.1 mg/kg to 0.15 mg/kg, 0.15 mg/kg to 0.3 mg/kg, 0.2 mg/kg to 0.3 mg/kg, and 0.25 mg/kg to 0.3 mg/kg). The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). In some embodiments, the pharmaceutical composition is administered subcutaneously. In some embodiments, the pharmaceutical composition is administered intravenously. Pharmaceutical compositions that include an ALK2 or ALK3 antibody or an antigen binding fragment thereof (e.g., an ALK2 or ALK3 binding fragment), of the invention may be administered to a subject in need thereof, at a frequency that is weekly to semi-annually. For example, the antibody or antigen binding fragment thereof may be administered once a week, once every 14 days, once a month, once every two months, once every three months, once every four months, once every five months, or once every six months. In some embodiments, the antibody may be administered to the subject at a frequency of once every 6 months. In some embodiments, the antibody may be administered to the subject at a frequency of once every 4 months. In some embodiments, the antibody may be administered to the subject at a frequency of once every 2 months. In some embodiments, the antibody may be administered to the subject at a frequency of once every month. In some embodiments, the antibody may be administered to the subject at a frequency of once biweekly. In some embodiments, the frequency is once every 10 to 14 days (e.g., once every 10 days, once every 11 days, once every 12 days, once every 13 days, or once every 14 days). In some embodiments, the frequency is once a week.

In some embodiments, the ALK2 or ALK3 antibody or an antigen binding fragment thereof is administered using a microneedle injector. In some embodiments, the ALK2 or ALK3 antibody or an antigen binding fragment thereof is administered using needle-free injection technology. Such technology is described in Ravi et al., Int J Pharm Investig. 5:192-199, 2015. For example, an ALK2 or ALK3 antibody or an antigen binding fragment thereof can be administered subcutaneously using any one of the Biojector® 2000, Vitajet™ 3, Tev-Tropin®, Sumavel®DosePro™, Bioject® ZetaJet™, PharmaJet Stratis®, or Jupiter Jet™, devices or other similar needle-free injection technology that is designed for subcutaneous administration of a small volume of a pharmaceutical composition.

In some embodiments, pharmaceutical compositions that include an ALK2 or ALK3 antibody or an antigen binding fragment thereof (e.g., an ALK2 or ALK3 binding fragment), of the invention may be administered to a subject in need thereof weekly, biweekly, monthly, bimonthly, or quarterly. Dosages may be provided in either a single or multiple dosage regimens. The timing between administrations may increase as the medical condition improves or decrease as the health of the patient declines.

### Methods of Treatment

The compositions and methods described herein can be used to treat and/or prevent (e.g., prevent the development of or treat a subject diagnosed with) medical conditions, e.g., bone disease, anemia or low red blood cell levels (e.g., low hemoglobin levels or low red blood cell count), heterotopic ossification (e.g., heterotopic ossification resulting from FOP), Sjogren's syndrome (e.g., dry eye associated with Sjogren's syndrome), DIPG, MO, PCO, or cardiac hypertrophy and/or cardiac fibrosis. In some embodiments, the ALK2 or ALK3 antibodies (e.g., neutralizing antibodies) or antigen binding fragments thereof (e.g., ALK2 or ALK3 binding fragments), described herein are administered to increase bone mineral density, increase bone formation, increase bone strength, reduce bone resorption (e.g., bone loss), or reduce the risk of bone fracture in a subject in need thereof. The ALK2 or ALK3 antibodies or antigen binding fragments thereof (e.g., ALK2 or ALK3 binding fragments), described herein may increase bone mineral density, increase bone formation, increase bone strength, reduce bone resorption (e.g., bone loss), or reduce the risk of bone fracture compared to measurements obtained prior to treatment or compared to bone mineral density, bone strength, bone formation, bone resorption, or risk of bone fracture typically observed in untreated subjects. In some embodiments, the subject may have or be at risk of developing a disease that results in bone damage (e.g., osteoporosis or osteopenia). In some embodiments of the methods described herein, the subject has or is at risk of developing a disease or condition involving bone damage (e.g., primary osteoporosis, secondary osteoporosis, osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, and immobility-related bone loss). The invention also includes methods of treating a subject having or at risk of developing primary osteoporosis, secondary osteoporosis, osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, and immobility-related bone loss, by administering to the subject an effective amount of an ALK2 or ALK3 antibody or an antigen binding fragment thereof (e.g., an ALK2 or ALK3 binding fragment), described herein.

In any of the methods described herein, a subject having or at risk of developing bone disease (e.g., bone damage) has or is at risk of developing a disease or condition including osteoporosis, osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, or immobility-related bone loss. The osteoporosis may be primary osteoporosis or secondary osteoporosis. In some embodiments, the primary osteoporosis is age-related or hormone-related osteoporosis (e.g., related to a decline in estrogen). In some embodiments, the secondary osteoporosis is immobilization-induced or glucocorticoid-induced osteoporosis. In some embodiments, the bone cancer is multiple myeloma, or the cancer metastasis-related bone loss is caused by multiple myeloma. In some embodiments, the treatment-related bone loss occurs due to treatment with FGF-21 or GLP-1, due to treatment with an FGF-21 or GLP-1 containing therapeutic, due to treatment of Type-2 diabetes and/or obesity, or due to cancer therapy (e.g., chemotherapy or radiation). In some embodiments, the diet-related bone loss is rickets (e.g., vitamin D deficiency). In some embodiments, the low-gravity related bone loss is lack of load-related bone loss. In some embodiments, the methods described herein increase bone mineral density (e.g., increase bone mass). In some embodiments, the methods described herein reduce bone resorption (e.g., reduce bone catabolic activity or reduce bone loss), e.g., reduce bone resorption compared to measurements obtained prior to treatment or compared to bone resorption typically observed in untreated subjects. In some embodiments, the methods described herein increase bone formation (e.g., increase bone anabolic activity or increase osteogenesis), e.g., increase bone formation compared to measurements obtained prior to treatment or compared to bone formation typically observed in untreated subjects. In some embodiments, the methods described herein increase osteoblast activity or osteoblastogenesis, e.g., increase osteoblast activity or osteoblastogenesis compared to measurements obtained prior to treatment or compared to osteoblast activity or osteoblastogenesis typically observed in untreated subjects. In some embodiments, the methods described herein decrease osteoclast activity or osteoclastogenesis, e.g., decrease osteoclast activity or osteoclastogenesis compared to measurements obtained prior to treatment or compared to osteoclast activity or osteoclastogenesis typically observed in untreated subjects. In some embodiments, the bone is cortical or trabecular bone. In some embodiments, a subject having or at risk of developing a bone disease may be administered an ALK2 or ALK3 antibody, or an antigen-binding fragment thereof, once a month, every two months, every three months, every four months, every five months, or every six months. In an embodiment, the ALK2 or ALK3 antibody, or an antigen-binding fragment thereof, may be administered to the subject every 4 months. In an embodiment, the ALK2 or ALK3 antibody, or an antigen-binding fragment thereof, may be administered to the subject every 6 months.

In some embodiments, the ALK2 or ALK3 antibodies or antigen binding fragments thereof (e.g., ALK2 or ALK3 binding fragments), described herein are administered to increase red blood cell levels (e.g., increase hemoglobin levels, increase red blood cell count, or increase red blood cell formation or production) in a subject in need thereof. The ALK2 or ALK3 antibodies or antigen binding fragments thereof (e.g., ALK2 or ALK3 binding fragments), described herein may increase red blood cell levels (e.g., increase hemoglobin levels, red blood cell count, or red blood cell formation) compared to measurements obtained prior to treatment or compared to red blood cell levels typically observed in untreated subjects. In some embodiments, the subject may have a disease or condition associated with low red blood cell levels (e.g., anemia or blood loss). In some embodiments of the methods described herein, the subject has or is at risk of developing a disease or condition involving low red blood cell levels (e.g., anemia or blood loss, such as aplastic anemia, iron deficiency anemia, iron-refractory iron deficiency anemia (IRIDA), or anemia of inflammation (also called anemia of chronic disease, e.g., anemia caused by inflammatory diseases or conditions, such as infection (e.g., chronic infection, such as HIV/AIDS or tuberculosis), autoimmune disease (e.g., rheumatoid arthritis or lupus), cancer (e.g., cancer or cancer treatment), inflammatory bowel disease (e.g., Crohn's disease or ulcerative colitis), and chronic kidney disease). The invention also includes methods of treating a subject having or at risk of developing low red blood cell levels (e.g., low hemoglobin levels or low red blood cell count, e.g., anemia) by administering to the subject an effective amount of an ALK2 or ALK3 antibody or an antigen binding fragment thereof (e.g., an ALK2 or ALK3 binding fragment), described herein. The methods described herein may include a step of screening a subject for one or more mutations in genes known to be associated with anemia (e.g., mutations in TMPRSS6, which are associated with IRIDA) prior to treatment with or administration of the compositions described herein. A subject can be screened for a genetic mutation using standard methods known to those of skill in the art (e.g., genetic testing).

The invention also includes methods of treating a subject having or at risk of developing anemia or blood loss by administering to the subject an effective amount of an ALK2 or ALK3 antibody or an antigen binding fragment thereof (e.g., an ALK2 or ALK3 binding fragment), described herein. In any of the methods described herein, a subject having or at risk of developing low red blood cell levels (e.g., low hemoglobin levels, low hematocrit, or low red blood cell counts) has or is at risk of developing anemia or blood loss In some embodiments, the anemia is associated with a nutritional deficit (e.g., a vitamin deficiency, such as vitamin B-12 deficiency, folate deficiency), a bone marrow defect (e.g., paroxysmal nocturnal hemoglobinuria), adverse reaction to medication (e.g., anti-retroviral HIV drugs), a myelodysplastic syndrome, bone marrow transplantation, myelofibrosis, ineffective hematopoiesis, cancer (e.g., a solid tumor, such as breast cancer, lung cancer, colon cancer; a tumor of the lymphatic system, such as chronic lymphocytic leukemia, non-Hodgkin's lymphoma, Hodgkin's lymphoma; or a tumor of the hematopoietic system, such as leukemia or multiple myeloma), cancer treatment (e.g., radiation or chemotherapy, e.g., chemotherapy with platinum-containing agents), myelofibrosis treatment (e.g., treatment with a JAK inhibitor, such as ruxolitinib or fedratinib), an inflammatory or autoimmune disease (e.g., rheumatoid arthritis, other inflammatory arthritides, systemic lupus erythematosus (SLE), an acute or chronic skin disease (e.g. psoriasis), or inflammatory bowel disease (e.g., Crohn's disease or ulcerative colitis), cystitis, gastritis), acute or chronic renal disease or failure (e.g., chronic kidney disease) including idiopathic or congenital conditions, diabetes, acute or chronic liver disease, acute or chronic bleeding, infection (e.g., malaria, osteomyelitis), splenomegaly, porphyria, vasculitis, hemolysis, urinary tract infection, hemoglobinopathy (e.g., sickle cell disease), thalassemia (e.g., α- or β-thalassemia), Churg-Strauss syndrome, Felty syndrome, Pearson syndrome, dyskeratosis congenita, graft versus host disease, hematopoietic stem cell transplantation, osteomyelofibrosis, pancytopenia, pure red-cell aplasia, purpura Schoenlein-Henoch, Shwachman syndrome (e.g., Shwachman-Diamond syndrome), drug use or abuse (e.g., alcohol abuse), or contraindication to transfusion (e.g., patients of advanced age, patients with allo- or auto-antibodies, pediatric patients, patients with cardiopulmonary disease, patients who object to transfusion for religious reasons (e.g., some Jehovah's Witnesses)). The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (MDS with isolated del(5q)), myelodysplastic syndrome with excess blasts (MDS-EB; which includes myelodysplastic syndrome with excess blasts—type 1 (MDS-EB-1) and myelodysplastic syndrome with excess blasts—type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk MDS as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations, such a subject may have increased erythroid progenitor cells in the bone marrow relative to a healthy subject). In some embodiments, the MDS is associated with a defect in early-stage hematopoiesis (e.g., early-stage erythroid cell development, such as commitment or early differentiation, such a subject may have fewer erythroid progenitor cells in the bone marrow compared to a healthy subject or to a subject with a defect in terminal maturation). In some embodiments, the MDS is associated with elevated endogenous erythropoietin levels. In some embodiments, the myelodysplastic syndrome is associated with hypocellular bone marrow (e.g., a subject with MDS has hypocellular bone marrow). The subject may have a low transfusion burden or a high transfusion burden. In some embodiments, the subject has a low transfusion burden and received 1-3 RBC units in the eight weeks prior to treatment with an antibody described herein. In some embodiments, the subject has a low transfusion burden and did not receive a transfusion (received 0 RBC units) in the eight weeks prior to treatment with an antibody described herein. In some embodiments, the anemia is aplastic anemia, iron deficiency anemia, iron-refractory iron deficiency anemia (IRIDA), vitamin deficiency anemia, anemia of inflammation (also called anemia of chronic disease, e.g., anemia caused by an inflammatory disease or condition, such as an infection (e.g., a chronic infection, such as HIV/AIDS or tuberculosis), an autoimmune disease (e.g., rheumatoid arthritis or lupus), cancer, inflammatory bowel disease (e.g., Crohn's disease or ulcerative colitis), and chronic kidney disease), anemia associated with bone marrow disease, hemolytic anemia, sickle cell anemia, microcytic anemia, hypochromic anemia, sideroblastic anemia, congenital dyserythropoietic anemia, Diamond Blackfan anemia, Fanconi anemia, or refractory anemia with excess of blasts. The sideroblastic anemia can be acquired sideroblastic anemia or congenital sideroblastic anemia. In some embodiments, the congenital sideroblastic anemia is associated with a mutation in ALAS2, SLC25A38, FECH, GLRX5, HSPA9, HSCB, SLC25A38, or ABCB7. In some embodiments, the congenital sideroblastic anemia is associated with a mutation in PUS1, YARS2, LARS2, TRNT1, MT-ATP6, NDUFB11, or SLC19A2, or with an mtDNA mutation. In some embodiments, the anemia is associated with elevated hepcidin levels (e.g., elevated compared to hepcidin levels in a subject that does not have anemia). The compositions and methods described herein can also be used to treat subjects that do not respond well to erythropoietin (EPO) or that are susceptible to adverse effects of EPO (e.g., hypertension, headaches, vascular thrombosis, influenza-like syndrome, obstruction of shunts, and myocardial infarction) or to treat subjects that do not respond to an erythroid maturation agent. In some embodiments, the subject has previously been treated with an ESA. In some embodiments, the subject has not previously been treated with an ESA. In some embodiments, the blood loss is due to surgery, trauma, a wound, an ulcer, urinary tract bleeding, digestive tract bleeding, frequent blood donation, or heavy menstrual bleeding (e.g., menorrhagia). In some embodiments, the methods described herein increase red blood cell levels (e.g., hemoglobin levels or red blood cell counts) compared to measurements obtained prior to treatment or compared to red blood cell levels typically observed in untreated subjects. In some embodiments, the methods described herein increase or induce red blood cell formation compared to measurements obtained prior to treatment or compared to red blood cell formation typically observed in untreated subjects. In some embodiments, the compositions and methods described herein reduce the need of a subject for a blood transfusion (e.g., the subject no longer needs blood transfusions, or the subject needs less frequent blood transfusion than before treatment with the compositions and methods described herein). Subjects with normal red blood cell levels can also be treated using the methods and compositions described herein to increase red blood cell levels so that blood can be drawn and stored for later use in transfusions. In some embodiments, a subject having or at risk of developing anemia may be administered an ALK2 or ALK3 antibody, or an antigen-binding fragment thereof, once a month, every two months, every three months, every four months, every five months, or every six months. In an embodiment, the ALK2 or ALK3 antibody, or an antigen-binding fragment thereof, may be administered to the subject every 4 months. In an embodiment, the ALK2 or ALK3 antibody, or an antigen-binding fragment thereof, may be administered to the subject every 6 months.

In some embodiments, the ALK2 or ALK3 antibodies or antigen binding fragments thereof (e.g., ALK2 or ALK3 binding fragments), described herein may be administered to a subject to treat FOP, to slow or stop the progression of FOP, to delay the onset of FOP (e.g., delay the development of heterotopic ossification), or to prevent or reduce heterotopic ossification (e.g., the formation of bone in muscle, tendons, ligaments, or other connective tissues, e.g., prevent or reduce heterotopic ossification in a subject with FOP who has already experienced heterotopic ossification, or prevent the development of heterotopic ossification in a subject who has not yet exhibited heterotopic ossification), such as heterotopic ossification in a subject with FOP. The FOP can be inherited FOP (e.g., FOP related to germline transmission of a mutation, such as an autosomal dominant mutation) or sporadic FOP (e.g., FOP related to a spontaneous, non-inherited mutation). The ALK2 or ALK3 antibodies or antigen binding fragments thereof (e.g., ALK2 or ALK3 binding fragments), described herein may reduce the amount of heterotopic bone, reduce the formation of heterotopic bone, or reduce the recurrence of heterotopic bone (e.g., recurrence after surgical resection) compared to the amount of heterotopic bone, formation of heterotopic bone, or recurrence of heterotopic bone observed in untreated or control treated subjects. The ALK2 or ALK3 antibodies or antigen binding fragments thereof (e.g., ALK2 or ALK3 binding fragments), described herein may prevent the formation of heterotopic bone (e.g., in a subject at risk of developing heterotopic ossification, such as a subject with FOP, or prevent the recurrence of heterotopic bone, such as recurrence after surgical resection). In some embodiments, the subject is at risk of developing heterotopic ossification (e.g., the subject has a genetic mutation associated with FOP but has not yet exhibited symptoms of FOP, e.g., has not yet exhibited heterotopic ossification). The methods described herein may include a step of screening a subject for one or more mutations in genes known to be associated with FOP (e.g., mutations in ALK2, also known as Activin A type I receptor (ACVR1)) prior to treatment with or administration of the compositions described herein. A subject can be screened for a genetic mutation using standard methods known to those of skill in the art (e.g., genetic testing). In some embodiments, a subject having FOP may be administered an ALK2 or ALK3 antibody, or an antigen-binding fragment thereof, once a week, every two weeks, every three weeks, or once a month. In an embodiment, the ALK2 or ALK3 antibody, or an antigen-binding fragment thereof, may be administered to the subject once a week. In an embodiment, the ALK2 or ALK3 antibody, or an antigen-binding fragment thereof, may be administered to the subject once every 10 to 14 days. In an embodiment, the ALK2 or ALK3 antibody, or an antigen-binding fragment thereof, may be administered to the subject once a month.

In some embodiments, the ALK2 or ALK3 antibodies or antigen binding fragments thereof (e.g., ALK2 or ALK3 binding fragments), described herein may be administered to a subject to treat Sjogren's syndrome (e.g., to treat dry eye associated with Sjogren's syndrome) or to reduce one or more symptom of Sjogren's syndrome (e.g., to reduce dry eye or dry mouth associated with Sjogren's syndrome). The ALK2 or ALK3 antibodies or antigen binding fragments thereof (e.g., ALK2 or ALK3 binding fragments), described herein may reduce eye irritation, reduce fibrosis of the lacrimal gland, reduce inflammation of the lacrimal gland, improve vision, increase the production of saliva, increase the production of tears (e.g., increase tear volume production), reduce systemic inflammation, reduce joint pain, or reduce fatigue in a subject with Sjogren's syndrome, or reduce the severity or occurrence of one or more symptom of Sjogren's syndrome (e.g., reduce dry eye, dry mouth, or joint pain associated with Sjogren's syndrome) compared to eye irritation, vision, inflammation or fibrosis of the lacrimal gland, the production of saliva, the production of tears, systemic inflammation, joint pain, fatigue, or the severity or occurrence of symptoms of Sjogren's syndrome in the subject prior to treatment or in an untreated subject with Sjogren's syndrome. In some embodiments, a subject having Sjogren's syndrome may be administered an ALK2 or ALK3 antibody, or an antigen-binding fragment thereof, once a week, every two weeks, every three weeks, or once a month. In an embodiment, the ALK2 or ALK3 antibody, or an antigen-binding fragment thereof, may be administered to the subject once a week. In an embodiment, the ALK2 or ALK3 antibody, or an antigen-binding fragment thereof, may be administered to the subject once every 10 to 14 days. In an embodiment, the ALK2 or ALK3 antibody, or an antigen-binding fragment thereof, may be administered to the subject once a month.

In some embodiments, the ALK2 or ALK3 antibodies or antigen binding fragments thereof (e.g., ALK2 or ALK3 binding fragments), described herein may be administered to a subject to treat DIPG, increase the survival (e.g., survival time, e.g., lifespan) of a subject having DIPG, increase progression free survival of a subject having DIPG, reduce DIPG tumor growth, reduce DIPG tumor size or volume, or prevent or reduce DIPG tumor metastasis. The ALK2 or ALK3 antibodies or antigen binding fragments thereof (e.g., ALK2 or ALK3 binding fragments), described herein may increase the survival (e.g., survival time, e.g., lifespan) of a subject having DIPG, increase progression free survival of a subject having DIPG, reduce DIPG tumor growth, reduce DIPG tumor size or volume, or prevent or reduce DIPG tumor metastasis compared survival time, progression free survival, tumor growth, tumor size or volume, or tumor metastasis compared to untreated or control treated subjects or compared to tumor growth, tumor size or volume, or tumor metastasis in the subject prior to treatment. In some embodiments, the subject has an activating mutation in ALK2 (ACVR1). The methods described herein may include a step of screening a subject for a mutation in ALK2 (ACVR1) prior to treatment with or administration of the compositions described herein. A subject can be screened for a genetic mutation using standard methods known to those of skill in the art (e.g., genetic testing). In some embodiments, a subject having DIPG may be administered an ALK2 or ALK3 antibody, or an antigen-binding fragment thereof, once a week, every two weeks, every three weeks, or once a month. In an embodiment, the ALK2 or ALK3 antibody, or an antigen-binding fragment thereof, may be administered to the subject once a week. In an embodiment, the ALK2 or ALK3 antibody, or an antigen-binding fragment thereof, may be administered to the subject once every 10 to 14 days. In an embodiment, the ALK2 or ALK3 antibody, or an antigen-binding fragment thereof, may be administered to the subject once a month.

In some embodiments, the ALK2 or ALK3 antibodies or antigen binding fragments thereof (e.g., ALK2 or ALK3 binding fragments), described herein may be administered to a subject to treat MO, reduce or prevent the formation of an osteochondroma in a subject with MO, reduce the number of osteochondromas in a subject with MO, reduce the size of an osteochondroma in a subject with MO, or slow the growth of an osteochondroma in a subject with MO. The osteochondroma can be formed on the growing end (metaphysis) of a bone, on a long bone (e.g., a long bone of the leg, arm, or digit), and/or on a flat bone (e.g., a hip bone (pelvic bone), rib bone, or shoulder blade). The ALK2 or ALK3 antibodies or antigen binding fragments thereof (e.g., ALK2 or ALK3 binding fragments), described herein may reduce or prevent the formation of an osteochondroma (e.g., prevent the formation of an osteochondroma in a subject with MO who has not yet developed osteochondroma or reduce or prevent the formation of osteochondroma in a subject with MO who has already developed one or more osteochondroma), reduce the number of osteochondromas, reduce the size of an osteochondroma, or slow the growth of an osteochondroma compared to the formation of an osteochondroma, the number of osteochondromas, the size of an osteochondroma, or the growth of an osteochondroma in the subject prior to treatment or in an untreated subject with MO. The methods described herein may include a step of screening a subject for a mutation in EXT1 or EXT2 prior to treatment with or administration of the compositions described herein. A subject can be screened for a genetic mutation using standard methods known to those of skill in the art (e.g., genetic testing). In some embodiments, a subject having MO may be administered an ALK2 or ALK3 antibody, or an antigen-binding fragment thereof, once a week, every two weeks, every three weeks, or once a month. In an embodiment, the ALK2 or ALK3 antibody, or an antigen-binding fragment thereof, may be administered to the subject once a week. In an embodiment, the ALK2 or ALK3 antibody, or an antigen-binding fragment thereof, may be administered to the subject once every 10 to 14 days. In an embodiment, the ALK2 or ALK3 antibody, or an antigen-binding fragment thereof, may be administered to the subject once a month.

In some embodiments, the ALK2 or ALK3 antibodies or antigen binding fragments thereof (e.g., ALK2 or ALK3 binding fragments), described herein may be administered to a subject to treat PCO, reduce or prevent the development of PCO (e.g., to prevent the development of PCO in a subject who has undergone cataract surgery but has not yet developed PCTO or in a subject who is soon to undergo cataract surgery, e.g., a subject who will undergo cataract surgery in 6 months, 5 months, 4 months, 3, months, 2 months, 1 month, 2 weeks, 1 week, or less, e.g., a subject at risk of developing PCO), improve visual acuity (e.g., reduce blurry or cloudy vision) in a subject with PCO, reduce light sensitivity or glare in a subject with PCO, reduce or prevent fibrosis (e.g., fibrosis near the implanted lens, e.g., fibrosis on or near the posterior capsule), reduce or inhibit lens epithelial cell proliferation, reduce or prevent lens fiber differentiation, or reduce or prevent inflammation in the eye in a subject having PCO or at risk of developing PCO (e.g., in a subject who has undergone or is soon to undergo cataract surgery e.g., a subject who will undergo cataract surgery in 6 months, 5 months, 4 months, 3, months, 2 months, 1 month, 2 weeks, 1 week, or less). The ALK2 or ALK3 antibodies or antigen binding fragments thereof (e.g., ALK2 or ALK3 binding fragments), described herein may reduce or prevent the development of PCO, reduce or prevent fibrosis (e.g., fibrosis near the implanted lens, e.g., fibrosis on or near the posterior capsule), reduce or prevent lens fiber differentiation, reduce or inhibit lens epithelial cell proliferation, or reduce or prevent eye inflammation in a subject at risk of developing PCO compared the development of PCO, fibrosis (e.g., fibrosis near the implanted lens, e.g., fibrosis on or near the posterior capsule), lens fiber differentiation, lens epithelial cell proliferation, or eye inflammation in an untreated subject, or may improve visual acuity (e.g., reduce blurry or cloudy vision) or reduce light sensitivity or glare, reduce fibrosis, reduce eye inflammation, reduce lens epithelial cell proliferation, or reduce lens fiber differentiation compared to visual acuity, light sensitivity or glare, fibrosis, eye inflammation, lens epithelial cell proliferation, or lens fiber differentiation in the subject prior to treatment.

In some embodiments, the ALK2 or ALK3 antibodies or antigen binding fragments thereof (e.g., ALK2 or ALK3 binding fragments), described herein may be administered to a subject to treat cardiac hypertrophy, treat cardiac fibrosis, slow or prevent the development of cardiac hypertrophy (e.g., in a subject at risk of developing cardiac hypertrophy, such as a subject with hypertension or valvular disease), slow or prevent the development of cardiac fibrosis (e.g., in a subject at risk of developing cardiac fibrosis, such as a subject with hypertension, a subject who has had a myocardial infarction, a subject with diabetic hypertrophic cardiomyopathy, or a subject with idiopathic dilated cardiomyopathy), reverse cardiac fibrosis, reduce or inhibit cardiac scar formation, increase or induce cardiac regeneration, or improve one or more symptoms of cardiac hypertrophy or cardiac fibrosis (e.g., increase exercise capacity, increase blood ejection volume, reduce left ventricular end diastolic pressure, reduce pulmonary capillary wedge pressure, increase cardiac output, increase cardiac index, reduce pulmonary artery pressures, reduce left ventricular end systolic and diastolic dimensions, reduce left and right ventricular wall stress, reduce wall tension and/or wall thickness, increase myocardial contractility, reduce cardiomyocyte area, reduce extracellular matrix deposition in the cardiac muscle, improve quality of life, and/or reduce disease-related morbidity and mortality). The ALK2 or ALK3 antibodies or antigen binding fragments thereof (e.g., ALK2 or ALK3 binding fragments), described herein slow or prevent the development of cardiac hypertrophy, slow or prevent the development of cardiac fibrosis, reverse cardiac fibrosis, reduce or inhibit cardiac scar formation, increase or induce cardiac regeneration, or improve one or more symptoms of cardiac hypertrophy or cardiac fibrosis in a subject having or risk of developing cardiac hypertrophy or cardiac fibrosis compared to the development of cardiac hypertrophy, the development of cardiac fibrosis, the extent of cardiac fibrosis, cardiac scar formation, cardiac regeneration, or one or more symptoms of cardiac hypertrophy or cardiac fibrosis in the subject prior to treatment or in an untreated or control treated subject. In some embodiments, an ALK2 or ALK3 antibody, or an antigen-binding fragment thereof, may be administered to a subject to treat hypertrophy, treat cardiac fibrosis, slow or prevent the development of cardiac hypertrophy once a week, every two weeks, every three weeks, or once a month. In an embodiment, the ALK2 or ALK3 antibody, or an antigen-binding fragment thereof, may be administered to the subject once a week. In an embodiment, the ALK2 or ALK3 antibody, or an antigen-binding fragment thereof, may be administered to the subject once every 10 to 14 days. In an embodiment, the ALK2 or ALK3 antibody, or an antigen-binding fragment thereof, may be administered to the subject once a month.

The compositions described herein are administered in an amount sufficient to increase bone mineral density, increase bone strength, density, reduce bone resorption (e.g., bone loss), reduce the rate of bone resorption (e.g., bone loss), increase bone formation, increase the rate of bone formation, reduce osteoclast activity, increase osteoblast activity, reduce the risk of bone fracture, increase red blood cell levels, increase hemoglobin levels, reduce the need for a transfusion, increase red blood cell formation, or increase red blood cell count, treat anemia, increase iron levels, reduce iron deficiency, decrease elevated hepcidin levels, treat FOP, prevent heterotopic ossification (e.g., prevent the initial formation of ectopic bone in a subject or prevent the recurrence of ectopic bone after surgical resection), slow or stop the progression of FOP, delay the onset of FOP (e.g., delay the onset of heterotopic ossification), reduce heterotopic ossification (e.g., reduce the amount of ectopic bone in a subject or reduce the formation of ectopic bone, such as the initial formation of ectopic bone or the recurrence of ectopic bone after surgical resection), treat Sjogren's syndrome, reduce dry eye associated with Sjogren's syndrome, reduce dry mouth associated with Sjogren's syndrome, reduce eye inflammation, increase the production of saliva, increase the production of tears, reduce systemic inflammation, reduce joint pain, reduce fatigue, reduce the severity or occurrence of symptoms of Sjogren's syndrome (e.g., reduce dry eye, dry mouth, or joint pain associated with Sjogren's syndrome), treat DIPG, increase the survival (e.g., survival time, e.g., lifespan) of a subject having DIPG, increase progression free survival of a subject having DIPG, reduce DIPG tumor growth, reduce DIPG tumor size or volume, prevent or reduce DIPG tumor metastasis, treat MO, reduce or prevent the formation of an osteochondroma in a subject with MO, reduce the number of osteochondromas in a subject with MO, reduce the size of an osteochondroma in a subject with MO, slow the growth of an osteochondroma in a subject with MO, reduce or prevent the development of PCO, improve visual acuity (e.g., reduce blurry or cloudy vision) in a subject with PCO, reduce light sensitivity or glare in a subject with PCO, reduce or prevent fibrosis (e.g., fibrosis near the implanted lens, e.g., fibrosis on or near the posterior capsule), reduce or inhibit lens epithelial cell proliferation, reduce or prevent lens fiber differentiation, or reduce or prevent inflammation in the eye of a subject with PCO, treat cardiac hypertrophy, treat cardiac fibrosis, slow or prevent the development of cardiac hypertrophy, slow or prevent the development of cardiac fibrosis, reverse cardiac fibrosis, reduce or inhibit cardiac scar formation, increase or induce cardiac regeneration, or improve one or more symptoms of cardiac hypertrophy or cardiac fibrosis (e.g., increase exercise capacity, increase blood ejection volume, reduce left ventricular end diastolic pressure, reduce pulmonary capillary wedge pressure, increase cardiac output, increase cardiac index, reduce pulmonary artery pressures, reduce left ventricular end systolic and diastolic dimensions, reduce left and right ventricular wall stress, reduce wall tension and/or wall thickness, increase myocardial contractility, reduce extracellular matrix deposition in the cardiac muscle, improve quality of life, and/or reduce disease-related morbidity and mortality). Bone mineral density can be evaluated using well-established clinical techniques known to one of skill in the art (e.g., by dual-energy x-ray absorptiometry). Red blood cell levels can be assessed using a standard blood test, which measures red blood cell counts and hemoglobin levels. FOP symptoms (e.g., heterotopic ossification) and MO (e.g., osteochondromas) can be evaluated using standard imaging methods, such as radiographs (e.g., X-rays), CT (computed tomography), and/or MRI (magnetic resonance imaging). DIPG tumor size, growth, volume, or metastasis, may be assessed using imaging methods, such as MRI. Symptoms of cardiac hypertrophy and/or cardiac fibrosis may be evaluated using clinical approaches to evaluate heart function and morphology (e.g., a stress test, echocardiogram, CT, MRI, cardiac magnetic resonance imaging, single photon emission computed tomography, positron emission tomography, a balloon catheter-based approach, or histological analysis of a biopsy). Sjogren's syndrome and PCO symptoms may be assessed by evaluating visual function and imaging the eye to assess inflammation and/or fibrosis (e.g., using slit lamp imaging to evaluate fibrosis forming on the posterior capsule). The methods described herein may also include a step of assessing bone mineral density, red blood cell levels, or symptoms of FOP, DIPG, MO, PCO, Sjogren's syndrome, or cardiac hypertrophy and/or cardiac fibrosis in a subject prior to treatment with or administration of the compositions described herein or after administration of or treatment with the compositions described herein. The subject may be evaluated 1 month, 2 months, 3 months, 4 months, 5 months, 6 months or more following administration of the ALK2 or ALK3 antibody or an antigen binding fragment thereof (e.g., an ALK2 or ALK3 binding fragment), or pharmaceutical composition depending on the dose and route of administration used for treatment. Depending on the outcome of the evaluation, the subject may receive additional treatments.

Nucleic acids encoding the ALK2 or ALK3 antibodies or antigen binding fragments thereof (e.g., ALK2 or ALK3 binding fragments), described herein, or expression vectors containing said nucleic acids can also be administered according to any of the methods described herein. In any of the methods described herein, the polypeptide, nucleic acid, or vector can be administered as part of a pharmaceutical composition. Compositions that can be administered to a subject according to the methods described herein are provided in Table 2.

### Kits

The compositions described herein can be provided in a kit for use in treating bone disease, anemia, FOP, Sjogren's syndrome (e.g., dry eye associated with Sjogren's syndrome), MO, DIPG, PCO, or cardiac hypertrophy and/or cardiac fibrosis. Compositions may include an ALK2 antibody or antigen binding fragment thereof (e.g., an ALK2 binding fragment), described herein, and may be provided in unit dosage form, optionally in a pharmaceutically acceptable excipient (e.g., saline), in an amount sufficient to treat bone disease, anemia, FOP, Sjogren's syndrome (e.g., dry eye associated with Sjogren's syndrome), MO, DIPG, PCO, or cardiac hypertrophy and/or cardiac fibrosis. The kit can further include a package insert that instructs a user of the kit, such as a physician, to perform the methods described herein. The kit may optionally include a syringe or other device for administering the composition.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Effect of ALK2 Antibodies in a Non-Human Primate

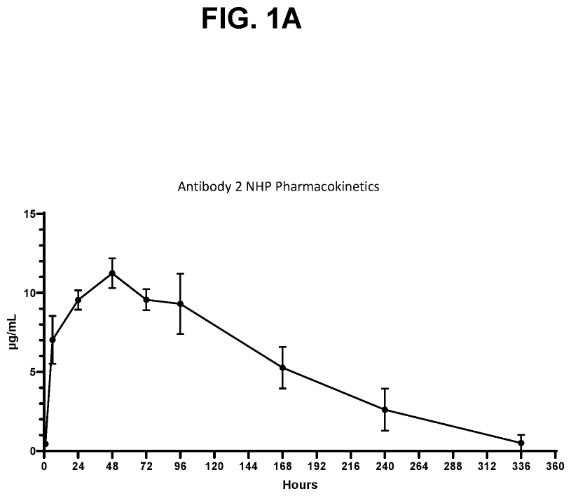

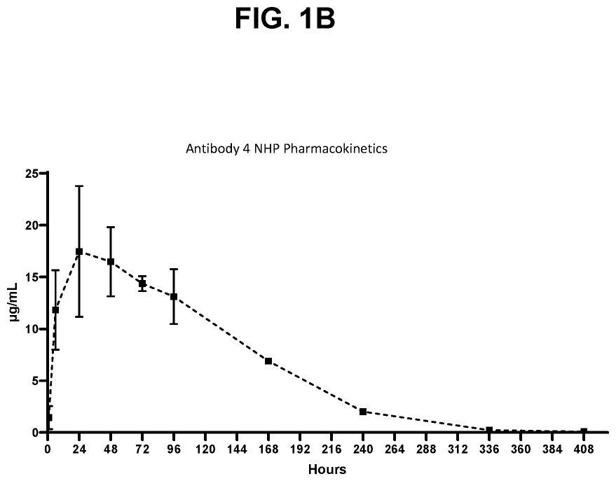

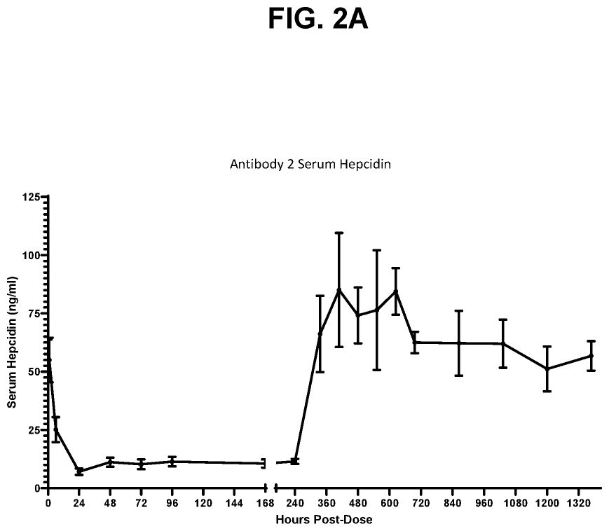

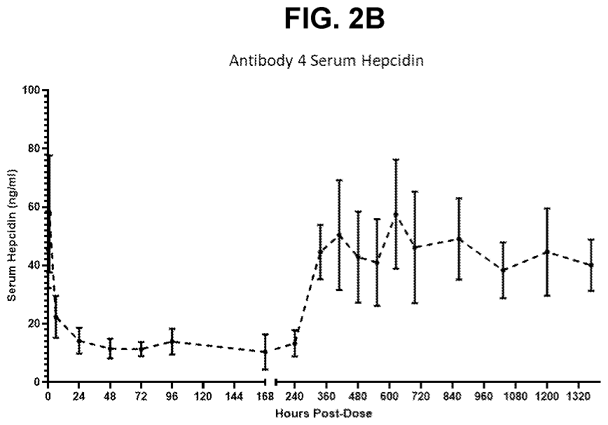

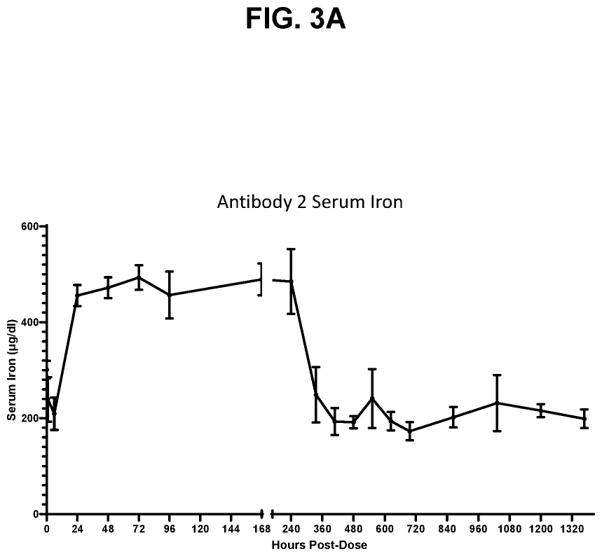

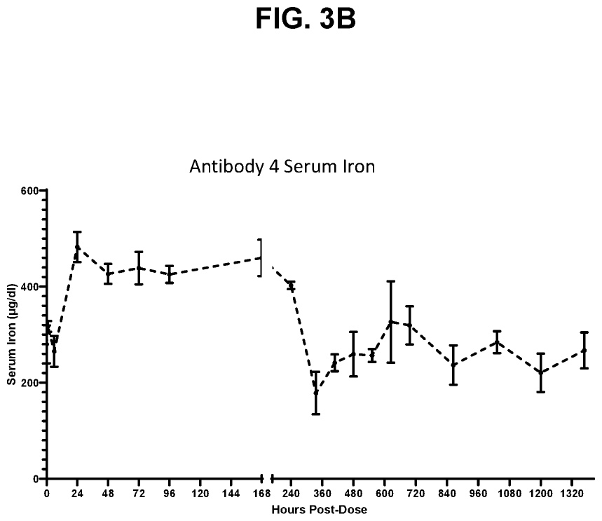

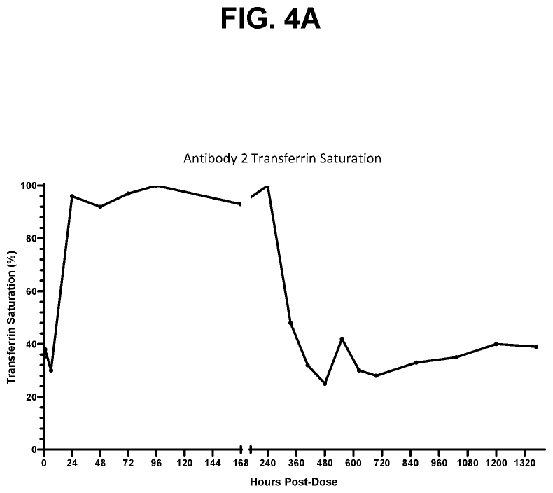

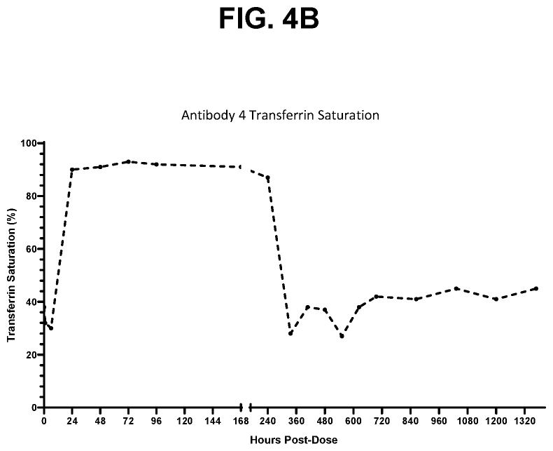

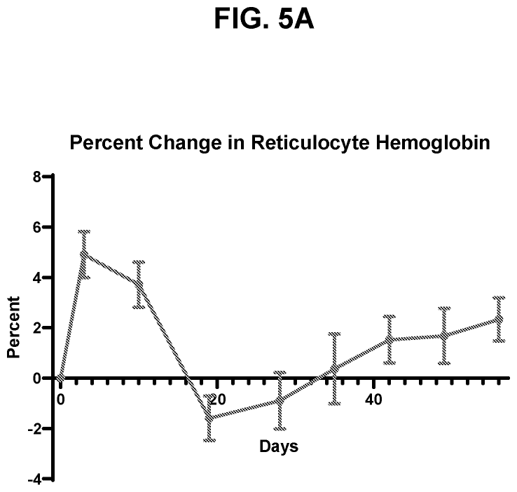

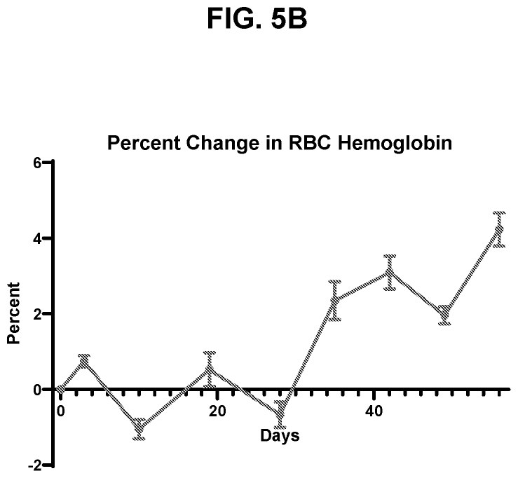

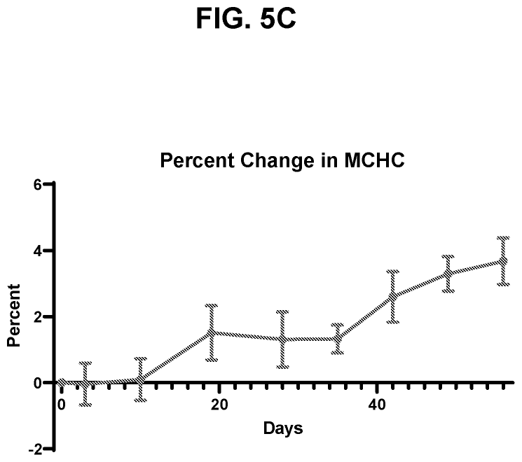

To determine the pharmacokinetic and pharmacodynamic properties of Antibody 2 and Antibody 4 in cynomolgus monkeys, a preclinical model highly representative of human biology, ad libitum-fed, female cynomolgus monkeys ranging in age from 2-4 years old were randomly assigned to receive Antibody 2 or Antibody 4 administered as a single subcutaneous dose of 3 mg/kg. Serum was sampled intermittently over a 56-day period and assessed for drug exposure (FIGS. 1A and 1B). Analysis of the exposure data using Excel PK Solver resulted in the parameters summarized in Table 7. Serum sampled intermittently over a 56-day period was also assessed for serum hepcidin levels (FIGS. 2A and 2B) measured using Intrinsic LifeSciences Primate-Hepcidin Compete ELISA kit as per manufacturer's instructions, serum iron levels (FIGS. 3A and 3B) which were measured using BioAssay Systems Iron Assay Kit, and transferrin saturation (FIGS. 4A and 4B) measured at IDEXX. At a subset of timepoints, blood was sampled and assessed for reticulocyte (FIG. 5A) and red blood cell hemoglobin content (FIG. 5B) (RET-Hgb and RBC-Hgb, respectively) and mean corpuscular hemoglobin concentration (FIG. 5C) with the use of a IDEXX Procyte analyzer. The hematological data was combined across the therapeutic groups that were administered Antibody 2 or Antibody 4.

TABLE 7
Summary of pharmacokinetic parameters of drug exposure

| | | Antibody 2 | Antibody 4 |
|---|---|---|---|
| Parameter | Unit | Value | Value |
| Lambda_z | 1/h | 0.014113837 | 0.020451 |
| t½ | h | 49.11117886 | 33.893021 |
| Tmax | h | 48 | 24 |
| Cmax | μg/ml | 11.24 | 17.466667 |
| Tlag | h | 0 | 0 |
| Clast_obs/Cmax | | 0.045077106 | 0.0129771 |
| AUC 0-t | μg/ml*h | 1852.255 | 2551.89 |
| AUC 0-inf_obs | μg/ml*h | 1888.153577 | 2562.9734 |
| AUC 0-t/0-inf_obs | | 0.98098747 | 0.9956756 |
| AUMC 0-inf_obs | μg/ml*h\hat{ }2 | 216906.6341 | 245569.71 |
| MRT 0-inf_obs | h | 114.8776438 | 95.81438 |
| Vz/F_obs | (mg/kg)/(μg/ml) | 0.112574192 | 0.057235 |
| Cl/F_obs | (mg/kg)/(μg/ml)/h | 0.001588854 | 0.0011705 |

Antibody 2 and Antibody 4 were rapidly absorbed and reached Cₘₐₓ within 48 hrs and had half-lives of 33.9 hours and 49.1 hours, respectively (FIGS. 1A and 1B). Robust and profound effects on hepcidin and serum iron were observed with both antibodies. Within 6 hours of administration of Antibody 2 or Antibody 4, serum hepcidin was reduced by 55.6% and 50.3%, respectively (FIGS. 2A and 2B). The peak effect was observed beginning after 48 hours with reductions of 77.2% in the Antibody 2 treated group, and 77.8% in the Antibody 4 group and continued through day 10 before returning to baseline by day 14. The reduction in hepcidin corresponded to increased circulating iron with maximal changes occurring within 24 hours of antibody administration. A 63.3% and 54.2% increase in iron was achieved with Antibody 2 and Antibody 4, respectively (FIGS. 3A and 3B). Similar to hepcidin, the response was sustained through 10 days, returning to baseline by day 14. RET-Hgb increased by 4.9% at 3 days post dose and remained elevated for 10 days (FIG. 5A). Increases in RBC-Hgb content were observed initially 35 days post dose and remained 4.2% higher at study termination at day 56 (FIG. 5B).

These data demonstrate that both Antibody 2 and Antibody 4 are efficacious at reducing serum hepcidin and increasing circulating iron in non-human primates, a preclinical model highly representative of human biology. Furthermore, the mobilized iron resulted in increased RET-Hgb and RBC-Hgb. These results provide evidence that treatment with Antibody 2 and Antibody 4 may be a viable approach to treating anemias such as IRIDA and inflammation of anemia that arise from elevated hepcidin.

### Example 2—Effect of ALK2 Antibodies in Wild-Type Mice

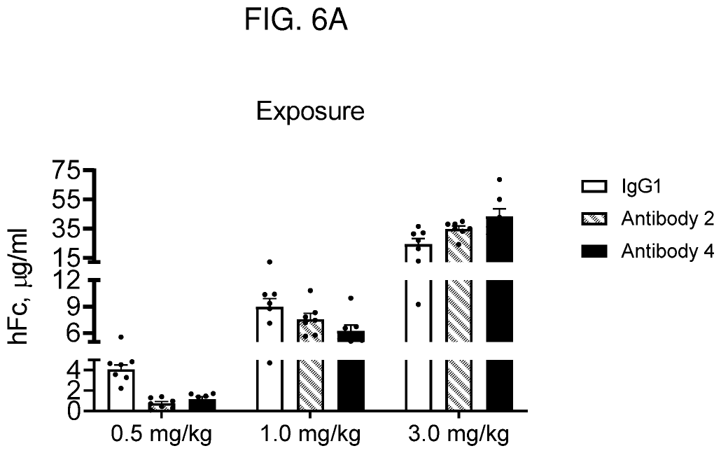

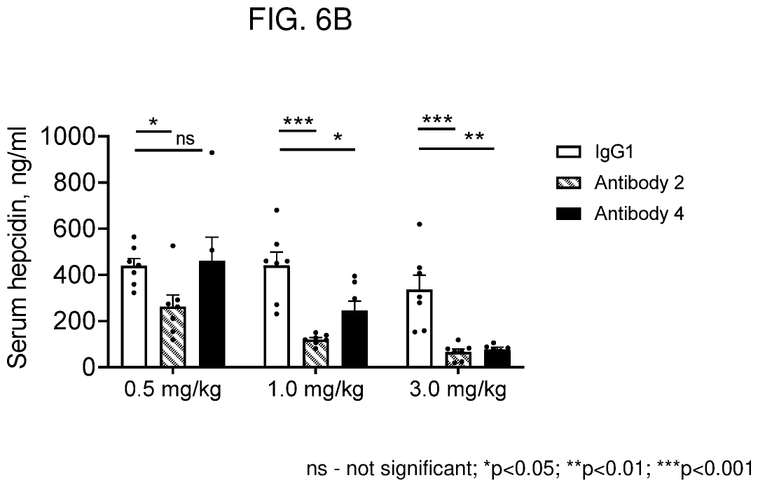

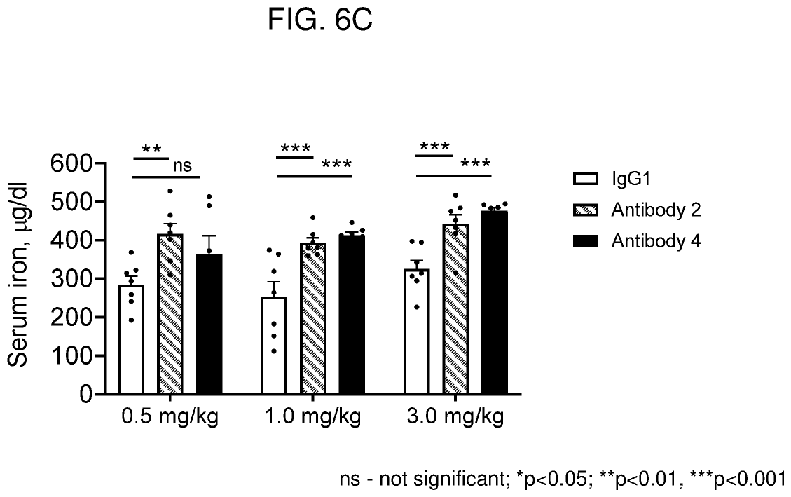

To determine the pharmacokinetic and pharmacodynamic properties of Antibody 2 and Antibody 4 in mice, ad libitum-fed, 8-week-old, male CD1 mice were randomly assigned to receive IgG1 isotype control, Antibody 2, or Antibody 4 administered as a single subcutaneous dose of 0.5 mg/kg, 1.0 mg/kg, or 3 mg/kg. Serum was collected from whole blood 24 hours post-dose. Serum was assessed for drug exposure, hepcidin levels (Intrinsic LifeSciences Hepcidin Murine-Compete ELISA kit as per manufacturer's instructions), and iron levels (Pointe Scientific Iron/TIBC Reagent Set). Results are shown in FIGS. 6A-6C. ns—not significant; *p<0.05; **p<0.01; ***p<0.001.

### Example 3—Treatment of Anemia by Administration of an ALK2 or ALK3 Antibody

According to the methods disclosed herein, a physician of skill in the art can treat a subject, such as a human patient, having anemia (e.g., IRIDA or anemia of inflammation) so as to increase hemoglobin levels, increase red blood cell counts, or reduce iron deficiency. The method of treatment can include diagnosing or identifying a subject as a candidate for treatment based on a blood test measuring hematological parameters. To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 or ALK3 binding fragment), may be administered to the subject, for example, subcutaneously to treat anemia. The ALK2 or ALK3 antibody or an antigen binding fragment thereof (e.g., an ALK2 or ALK3 binding fragment), is administered in a therapeutically effective amount, such as from 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 or ALK3 antibody or an antigen binding fragment thereof (e.g., an ALK2 or ALK3 binding fragment), is administered once a month, once every two months, once every three months, once every four months, once every five months, or once every six months. The ALK2 or ALK3 antibody or an antigen binding fragment thereof (e.g., an ALK2 or ALK3 binding fragment), is administered in an amount sufficient to increase hemoglobin levels, increase red blood cell counts, or reduce iron deficiency.

Following administration of the composition to a patient, a practitioner of skill in the art can monitor the patient's improvement in response to the therapy by a variety of methods. For example, a physician can monitor the patient's hemoglobin levels, red blood cell counts, or iron deficiency by performing a blood test. A finding that the patient exhibits improved hemoglobin levels, red blood cell counts, or iron deficiency following administration of the composition compared to test results prior to administration of the composition indicates that the patient is responding favorably to the treatment. Subsequent doses can be determined and administered as needed.

### Example 4—Treatment of Bone Disease by Administration of an ALK2 or ALK3 Antibody

According to the methods disclosed herein, a physician of skill in the art can treat a subject, such as a human patient, having bone disease (e.g., osteoporosis or osteopenia) so as to increase bone mineral density, increase bone formation, reduce bone resorption (e.g., bone loss), or reduce the risk of bone fracture. The method of treatment can include diagnosing or identifying a subject as a candidate for treatment based on standard clinical tests for bone mineral density (e.g., dual X-ray absorptiometry). To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 binding fragment), may be administered to the subject, for example, by parenteral injection (e.g., intravenous injection) to treat bone disease. The ALK2 or ALK3 antibody or an antigen binding fragment thereof (e.g., an ALK2 or ALK3 binding fragment), is administered in a therapeutically effective amount, such as from 0.002 mg/kg to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 antibody or an antigen binding fragment thereof (e.g., an ALK2 or ALK3 binding fragment), is administered once a month, once every two months, once every three months, once every four months, once every five months, or once every six months. The ALK2 or ALK3 antibody or an antigen binding fragment thereof (e.g., an ALK2 or ALK3 binding fragment), is administered in an amount sufficient to increase bone mineral density, increase bone formation, reduce bone resorption (e.g., bone loss), or reduce the risk of bone fracture.

Following administration of the composition to a patient, a practitioner of skill in the art can monitor the patient's improvement in response to the therapy by a variety of methods. For example, a physician can monitor the patient's bone mineral density by performing dual X-ray absorptiometry. A finding that the patient exhibits increased bone mineral density, increased bone formation, reduced bone resorption (e.g., bone loss), or a reduced risk of bone fracture following administration of the composition compared to test results prior to administration of the composition indicates that the patient is responding favorably to the treatment. Subsequent doses can be determined and administered as needed.

### Example 5—Treatment of FOP by Administration of an ALK2 Antibody

According to the methods disclosed herein, a physician of skill in the art can treat a subject, such as a human patient, having FOP (e.g., a patient with a gain of function mutation in ALK2) so as to prevent or reduce heterotopic ossification, slow the progression of FOP, or delay the onset of FOP. The method of treatment can include diagnosing or identifying a subject as a candidate for treatment based on genetic testing for a mutation in ALK2 or performing radiographic imaging, a CT, or an MRI. To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 or ALK3 binding fragment), may be administered to the subject, for example, by parenteral injection (e.g., intravenous injection) to treat FOP. The ALK2 or ALK3 antibody or an antigen binding fragment thereof (e.g., an ALK2 or ALK3 binding fragment), is administered in a therapeutically effective amount, such as from 0.002 to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 or ALK3 antibody or an antigen binding fragment thereof (e.g., an ALK2 or ALK3 binding fragment), is administered once a week, once every two weeks, once every three weeks, or once a month. The ALK2 or ALK3 antibody or an antigen binding fragment thereof (e.g., an ALK2 or ALK3 binding fragment), is administered in an amount sufficient to reduce heterotopic ossification, slow the progression of FOP, or delay the onset of FOP.

Following administration of the composition to a patient, a practitioner of skill in the art can monitor the patient's improvement in response to the therapy by a variety of methods. For example, a physician can monitor the patient's heterotopic ossification or disease progression using radiography, CT, and/or MRI. A finding that the patient exhibits reduced heterotopic ossification (e.g., a reduction in the amount of heterotopic ossification or the formation of additional heterotopic bone), a failure to develop heterotopic ossification, or delayed onset or slowed progression of FOP following administration of the composition compared to test results prior to administration of the composition or compared to an untreated subject indicates that the patient is responding favorably to the treatment. Subsequent doses can be determined and administered as needed.

### Example 6—Treatment of Sjogren's Syndrome by Administration of an ALK2 or ALK3 Antibody

According to the methods disclosed herein, a physician of skill in the art can treat a subject, such as a human patient, having Sjogren's syndrome so as to ameliorate or reduce one or more symptom of Sjogren's syndrome (e.g., dry eye, dry mouth, eye irritation, blurred vision, lacrimal gland fibrosis, lacrimal gland inflammation, joint pain, or fatigue). To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 or ALK3 binding fragment), may be administered to the subject, for example, by parenteral injection (e.g., intravenous injection and subcutaneous injection) or by administration to the eye (e.g., intraocular injection or topical administration) to treat Sjogren's syndrome. The ALK2 or ALK3 antibody or an antigen binding fragment thereof (e.g., an ALK2 or ALK3 binding fragment), is administered in a therapeutically effective amount, such as from 0.002 to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 or ALK3 antibody or an antigen binding fragment thereof (e.g., an ALK2 or ALK3 binding fragment), is administered once a week, once every two weeks, once every three weeks, or once a month. The ALK2 or ALK3 antibody or an antigen binding fragment thereof (e.g., an ALK2 or ALK3 binding fragment), is administered in an amount sufficient to ameliorate or reduce one or more symptom of Sjogren's syndrome (e.g., dry eye, dry mouth, eye irritation, blurred vision, lacrimal gland fibrosis, lacrimal gland inflammation, joint pain, or fatigue, e.g., by increasing the production of tears or saliva).

Following administration of the composition to a patient, a practitioner of skill in the art can monitor the patient's improvement in response to the therapy by a variety of methods. For example, a physician can monitor the patient's symptoms of Sjogren's syndrome by evaluating the production of tears (e.g., tear volume production), the production of saliva, vision, eye irritation, inflammation or fibrosis of the lacrimal gland, or the patient's reports of joint pain or fatigue. A finding that the patient exhibits a reduction in one or more symptom of Sjogren's syndrome (e.g., dry eye, dry mouth, eye irritation, blurred vision, lacrimal gland fibrosis, lacrimal gland inflammation, joint pain, or fatigue) following administration of the composition compared to the symptom prior to administration of the composition or compared to an untreated subject indicates that the patient is responding favorably to the treatment. Subsequent doses can be determined and administered as needed.

### Example 7—Treatment of MO by Administration of an ALK2 or ALK3 Antibody

According to the methods disclosed herein, a physician of skill in the art can treat a subject, such as a human patient, having MO so as to prevent or reduce the formation of an osteochondroma, reduce the size of an osteochondroma, reduce the number of osteochondromas, or slow the growth of an osteochondroma. The method of treatment can include diagnosing or identifying a subject as a candidate for treatment based on genetic testing for a mutation in EXT1 or EXT2 or performing radiographic imaging, a CT, or an MRI. To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 or ALK3 binding fragment), may be administered to the subject, for example, by parenteral injection (e.g., intravenous injection and subcutaneous injection) to treat MO. The ALK2 on ALK3 antibody or an antigen binding fragment thereof (e.g., an ALK2 or ALK3 binding fragment), is administered in a therapeutically effective amount, such as from 0.002 to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 of ALK3 antibody or an antigen binding fragment thereof (e.g., an ALK2 or ALK3 binding fragment), is administered once a week, once every two weeks, once every three weeks, or once a month. The ALK2 or ALK3 antibody or an antigen binding fragment thereof (e.g., an ALK2 or ALK3 binding fragment), is administered in an amount sufficient to reduce the formation of an osteochondroma, reduce the size of an osteochondroma, reduce the number of osteochondromas, or slow the growth of an osteochondroma.

Following administration of the composition to a patient, a practitioner of skill in the art can monitor the patient's improvement in response to the therapy by a variety of methods. For example, a physician can monitor the growth, number, or size of osteochondromas in the patient using radiography, CT, and/or MRI. A finding that the patient exhibits reduced osteochondroma formation (e.g., does not form new osteochondromas or forms fewer osteochondromas compared to an untreated subject or osteochondroma formation prior to treatment), reduced osteochondroma size, reduced osteochondroma number, or slower osteochondroma growth following administration of the composition compared to test results prior to administration of the composition or compared to an untreated subject indicates that the patient is responding favorably to the treatment. Subsequent doses can be determined and administered as needed.

### Example 8—Treatment of DIPG by Administration of an ALK2 or ALK3 Antibody

According to the methods disclosed herein, a physician of skill in the art can treat a subject, such as a human patient, having DIPG so as to improve survival time, reduce tumor growth, reduce tumor size or volume, or prevent or reduce tumor metastasis. The method of treatment can include diagnosing or identifying a subject as a candidate for treatment based on genetic testing for an activating mutation in ALK2. To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 or ALK3 binding fragment), may be administered to the subject, for example, by parenteral injection (e.g., intravenous or subcutaneous injection) or by local administration to the central nervous system (e.g., intracerebroventricular injection, intrathecal injection, or intra-cisternal injection). The ALK2 or ALK3 antibody or an antigen binding fragment thereof (e.g., an ALK2 or ALK3 binding fragment), is administered in a therapeutically effective amount, such as from 0.002 to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 or ALK3 antibody or an antigen binding fragment thereof (e.g., an ALK2 or ALK3 binding fragment), is administered once a week, once every two weeks, once every three weeks, or once a month. The ALK2 or ALK3 antibody or an antigen binding fragment thereof (e.g., an ALK2 or ALK3 binding fragment), is administered in an amount sufficient to improve survival time, reduce tumor growth, reduce tumor size or volume, or prevent or reduce tumor metastasis.

Following administration of the composition to a patient, a practitioner of skill in the art can monitor the patient's improvement in response to the therapy by a variety of methods. For example, a physician can monitor the patient's survival time or tumor size, growth, or metastasis using imaging (e.g., MRI). A finding that the patient exhibits reduced tumor growth, reduced tumor size or volume, reduced tumor metastasis, or increased survival time compared to an untreated subject or compared to tumor growth, size, or metastasis in the subject prior to administration indicates that the patient is responding favorably to the treatment. Subsequent doses can be determined and administered as needed.

### Example 9—Treatment or Prevention of PCO by Administration of an ALK2 or ALK3 Antibody

According to the methods disclosed herein, a physician of skill in the art can treat a subject, such as a human patient, having or at risk of developing PCO (e.g., a patient having PCO or a patient that has undergone cataract surgery or is soon to undergo cataract surgery, e.g., a patient that will undergo cataract surgery in 6 months, 5 months, 4 months, 3 months, 2 months, 1 month, 2 weeks, 1 week, or less) so as to prevent the development of PCO, reduce PCO, improve visual acuity (e.g., reduce blurry or cloudy vision), reduce light sensitivity or glare, reduce or prevent fibrosis (e.g., fibrosis on or near the posterior capsule, e.g., near the implanted lens), reduce or prevent lens fiber differentiation, reduce or inhibit lens epithelial cell proliferation, or reduce or prevent inflammation in the eye. To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 or ALK3 binding fragment), may be administered to the subject, for example, by parenteral injection (e.g., intravenous or subcutaneous injection) or by administration to the eye (e.g., intraocular injection or topical administration) to treat PCO. The ALK2 or ALK3 antibody or an antigen binding fragment thereof (e.g., an ALK2 or ALK3 binding fragment) is administered in a therapeutically effective amount, such as from 0.002 to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 or ALK3 antibody or an antigen binding fragment thereof (e.g., an ALK2 or ALK3 binding fragment), is administered once a week, once every two weeks, once every three weeks, or once a month. The ALK2 or ALK3 antibody or an antigen binding fragment thereof (e.g., an ALK2 or ALK3 binding fragment), is administered in an amount sufficient to prevent the development of PCO, reduce PCO, improve visual acuity (e.g., reduce blurry or cloudy vision), reduce light sensitivity or glare, reduce or prevent fibrosis (e.g., fibrosis on or near the posterior capsule, e.g., near the implanted lens), reduce or prevent lens fiber differentiation, reduce or inhibit lens epithelial cell proliferation, or reduce or prevent inflammation in the eye.

Following administration of the composition to a patient, a practitioner of skill in the art can monitor the patient's improvement in response to the therapy by a variety of methods. For example, a physician can monitor the patient's visual acuity, sensitivity to light or glare, opacity of the posterior capsule, or eye inflammation. A finding that the patient does not develop PCO or exhibits improved visual acuity, reduced sensitivity to light or glare, reduced fibrosis, or reduced inflammation in the eye following administration of the composition compared to an untreated subject or compared to measurements from the patient prior to treatment indicates that the patient is responding favorably to the treatment. Subsequent doses can be determined and administered as needed.

### Example 10—Treatment of Cardiac Hypertrophy and/or Cardiac Fibrosis by Administration of an ALK2 or ALK3 Antibody

According to the methods disclosed herein, a physician of skill in the art can treat a subject, such as a human patient, having cardiac hypertrophy and/or cardiac fibrosis so as to slow or prevent the development of cardiac hypertrophy or cardiac fibrosis, reverse cardiac fibrosis, reduce or inhibit cardiac scar formation, increase or induce cardiac regeneration, or improve one or more symptoms of cardiac hypertrophy or cardiac fibrosis (e.g., increase exercise capacity, increase blood ejection volume, reduce left ventricular end diastolic pressure, reduce pulmonary capillary wedge pressure, increase cardiac output, increase cardiac index, reduce pulmonary artery pressures, reduce left ventricular end systolic and diastolic dimensions, reduce left and right ventricular wall stress, reduce wall tension and/or wall thickness, increase myocardial contractility, reduce extracellular matrix deposition in the cardiac muscle, or reduce fibrosis). To treat the subject, a physician of skill in the art can administer to the subject a composition containing an ALK2 or ALK3 antibody or an antigen binding fragment thereof (e.g., an ALK2 or ALK3 binding fragment). The composition containing the ALK2 or ALK3 antibody or an antigen binding fragment thereof (e.g., an ALK2 or ALK3 binding fragment), may be administered to the subject, for example, by parenteral injection (e.g., intravenous or subcutaneous injection) to treat cardiac hypertrophy and/or cardiac fibrosis. The ALK2 or ALK3 antibody or an antigen binding fragment thereof (e.g., an ALK2 or ALK3 binding fragment), is administered in a therapeutically effective amount, such as from 0.002 to 3 mg/kg (e.g., 0.005 mg/kg to 3 mg/kg, 0.01 mg/kg to 3 mg/kg, 0.05 mg/kg to 3 mg/kg, 0.1 mg/kg to 3 mg/kg, 0.5 mg/kg to 3 mg/kg, 1 mg/kg to 3 mg/kg, 1.5 mg/kg to 3 mg/kg, 2 mg/kg to 3 mg/kg, 2.5 mg/kg to 3 mg/kg, 0.002 mg/kg to 2.5 mg/kg, 0.002 mg/kg to 2 mg/kg, 0.002 mg/kg to 1.5 mg/kg, 0.002 mg/kg to 1 mg/kg, 0.002 mg/kg to 0.5 mg/kg, 0.002 mg/kg to 0.25 mg/kg, 0.002 mg/kg to 0.1 mg/kg, 0.002 mg/kg to 0.05 mg/kg, 0.002 mg/kg to 0.01 mg/kg, 0.002 mg/kg to 0.005 mg/kg, 0.1 mg/kg to 0.3 mg/kg, or 0.1 mg/kg to 1 mg/kg). In some embodiments, the ALK2 or ALK3 antibody or an antigen binding fragment thereof (e.g., an ALK2 or ALK3 binding fragment), is administered once a week, once every two weeks, once every three weeks, or once a month. The ALK2 or ALK3 antibody or an antigen binding fragment thereof (e.g., an ALK2 or ALK3 binding fragment), is administered in an amount sufficient to slow or prevent the development of cardiac hypertrophy or cardiac fibrosis, reverse cardiac fibrosis, reduce or inhibit cardiac scar formation, increase or induce cardiac regeneration, or improve one or more symptoms of cardiac hypertrophy or cardiac fibrosis (e.g., increase exercise capacity, increase blood ejection volume, reduce left ventricular end diastolic pressure, reduce pulmonary capillary wedge pressure, increase cardiac output, increase cardiac index, reduce pulmonary artery pressures, reduce left ventricular end systolic and diastolic dimensions, reduce left and right ventricular wall stress, reduce wall tension and/or wall thickness, increase myocardial contractility, reduce extracellular matrix deposition in the cardiac muscle, or reduce fibrosis).

Following administration of the composition to a patient, a practitioner of skill in the art can monitor the patient's improvement in response to the therapy by a variety of methods. For example, a physician can monitor the patient's symptoms of cardiac hypertrophy or cardiac fibrosis (e.g., exercise capacity, blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, wall tension and/or wall thickness, or myocardial contractility) using a stress test, echocardiogram, MRI, or other approaches. A finding that the patient exhibits reduced cardiac fibrosis, increased cardiac regeneration, or an improvement of one or more symptoms of cardiac hypertrophy or cardiac fibrosis (e.g., increase exercise capacity, increase blood ejection volume, reduce left ventricular end diastolic pressure, reduce pulmonary capillary wedge pressure, increase cardiac output, increase cardiac index, reduce pulmonary artery pressures, reduce left ventricular end systolic and diastolic dimensions, reduce left and right ventricular wall stress, reduce wall tension and/or wall thickness, increase myocardial contractility, reduce extracellular matrix deposition in the cardiac muscle, or reduce fibrosis) following administration of the composition compared to test results prior to administration of the composition or compared to an untreated subject indicates that the patient is responding favorably to the treatment. Subsequent doses can be determined and administered as needed.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent, or patent application was specifically and individually indicated to be incorporated by reference in its entirety.

Other embodiments are within the following claims.

1. A method of treating a subject having a disease or condition that can be treated with an ALK2 or ALK3 inhibitor, the method comprising administering to the subject an isolated ALK2 or ALK3 antibody, or an antigen-binding fragment thereof, in an amount of 0.002 mg/kg to 3 mg/kg.

2. The method of claim 1, wherein the subject is administered an ALK2 antibody, or an antigen-binding fragment thereof.

3. The method of claim 2, wherein the ALK2 antibody, or antigen-binding fragment thereof, is a composition of Table 2.

4. The method of claim 1, wherein the subject is administered an ALK3 antibody, or an antigen-binding fragment thereof.

5. The method of claim 1, wherein the antibody is administered in an amount of 0.01 mg/kg to 2 mg/kg.

6. The method of claim 5, wherein the antibody is administered in an amount of 0.05 mg/kg to 1 mg/kg.

7. The method of claim 6, wherein the antibody is administered in amount of 0.1 mg/kg to 0.3 mg/kg.

8. The method of claim 1, wherein the antibody is administered to the subject at a frequency that is weekly to semi-annually.

9. The method of claim 8, wherein the frequency is once every 6 months, once every 4 months, or once every 2 months.

10-11. (canceled)

12. The method of claim 8, wherein the frequency is once every month.

13. The method of claim 8, wherein the frequency is once biweekly, once every 10 to 14 days, or once a week.

14-15. (canceled)

16. The method of claim 1, wherein the antibody, or antigen-binding fragment thereof, is administered subcutaneously.

17. The method of claim 1, wherein the disease or condition that can be treated with an ALK2 or ALK3 inhibitor is anemia.

18. The method of claim 17, wherein the anemia is associated with cancer, cancer treatment, myelofibrosis treatment, chronic kidney disease, acute renal disease or failure, chronic renal disease or failure, a myelodysplastic syndrome, thalassemia, a nutritional deficit, adverse reaction to medication, ineffective hematopoiesis, an inflammatory or autoimmune disease, splenomegaly, porphyria, vasculitis, hemolysis, a bone marrow defect, bone marrow transplantation, myelofibrosis, diabetes, acute liver disease, chronic liver disease, acute bleeding, chronic bleeding, an infection, hemoglobinopathy, drug use, alcohol abuse, Churg-Strauss syndrome, Felty syndrome, graft versus host disease, hematopoietic stem cell transplantation, osteomyelofibrosis, pancytopenia, pure red-cell aplasia, purpura Schoenlein-Henoch, Shwachman Diamond syndrome, advanced age, contraindication to transfusion, surgery, trauma, a wound, an ulcer, urinary tract bleeding, digestive tract bleeding, frequent blood donation, or heavy menstrual bleeding.

19. The method of claim 17, wherein the anemia is aplastic anemia, iron deficiency anemia, vitamin deficiency anemia, anemia of inflammation, IRIDA, anemia associated with a bone marrow disease, hemolytic anemia, sickle cell anemia, microcytic anemia, hypochromic anemia, sideroblastic anemia, Diamond Blackfan anemia, Fanconi anemia, or refractory anemia with excess of blasts.

20-21. (canceled)

22. The method of claim 17, wherein the anemia is associated with elevated hepcidin levels.

23. The method of claim 1, wherein the disease or condition that can be treated with an ALK2 or ALK3 inhibitor is fibrodysplasia ossificans progressiva (FOP).

24. (canceled)

25. The method of claim 1, wherein the disease or condition that can be treated with an ALK2 or ALK3 inhibitor is a bone disease.

26. The method of claim 25, wherein the bone disease is osteoporosis, osteopenia, osteopetrosis, osteogenesis imperfecta, bone fracture, bone cancer or cancer metastasis-related bone loss, Paget's disease, renal osteodystrophy, treatment-related bone loss, neuromuscular disease-related bone loss, burn-induced bone loss, anorexia-related bone loss, diet-related bone loss, bone loss associated with the treatment of obesity, low gravity-related bone loss, or immobility-related bone loss.

27. The method of claim 1, wherein the disease or condition that can be treated with an ALK2 or ALK3 inhibitor is Sjogren's syndrome, multiple osteochondroma, diffuse intrinsic pontine glioma, posterior capsule opacification, cardiac hypertrophy, or cardiac fibrosis.

28. (canceled)


SEQUENCE LISTING (382 sequences)

SEQ ID NO: 1 - protein, 13 aa, Artificial Sequence
  Synthetic Construct
SGSSSNIGSNYVS

SEQ ID NO: 2 - protein, 11 aa, Artificial Sequence
  Synthetic Construct
  MISC_FEATURE (4)..(4): Xaa is Ser or Asn
  MISC_FEATURE (5)..(5): Xaa is Ile or Leu
  MISC_FEATURE (6)..(6): Xaa is Pro, Gly, or Arg
  MISC_FEATURE (7)..(7): Xaa is Ser, Thr, or Lys
  MISC_FEATURE (8)..(8): Xaa is Phe, Lys, or Tyr
  MISC_FEATURE (9)..(9): Xaa is Phe, Tyr, or Ser
  MISC_FEATURE (10)..(10): Xaa is Ala or Val
  MISC_FEATURE (11)..(11): Xaa is Ser, Tyr, or His
SGDXXXXXXXX

SEQ ID NO: 3 - protein, 11 aa, Artificial Sequence
  Synthetic Construct
  MISC_FEATURE (1)..(2): Xaa is Val or Leu
  MISC_FEATURE (5)..(5): Xaa is Lys, Arg, Gly, or Tyr
  MISC_FEATURE (6)..(6): Xaa is Asn or Asp
  MISC_FEATURE (7)..(7): Xaa is Asn or Ser
  MISC_FEATURE (8)..(8): Xaa is His, Asn, Asp, or Lys
XXIYXXXXRPS

SEQ ID NO: 4 - protein, 11 aa, Artificial Sequence
  Synthetic Construct
ASWDHSDRFYV

SEQ ID NO: 5 - protein, 10 aa, Artificial Sequence
  Synthetic Construct
YVTAPWKSIW

SEQ ID NO: 6 - protein, 10 aa, Artificial Sequence
  Synthetic Construct
YSADAQQMKA

SEQ ID NO: 7 - protein, 9 aa, Artificial Sequence
  Synthetic Construct
QVYASVHRM

SEQ ID NO: 8 - protein, 10 aa, Artificial Sequence
  Synthetic Construct
QTYDWSHFGW

SEQ ID NO: 9 - protein, 10 aa, Artificial Sequence
  Synthetic Construct
  MISC_FEATURE (2)..(2): Xaa is Gly or Phe
  MISC_FEATURE (5)..(5): Xaa is Ser or Asn
  MISC_FEATURE (7)..(7): Xaa is Tyr, His, Ser, or Ala
  MISC_FEATURE (8)..(8): Xaa is Gly or Ala
  MISC_FEATURE (9)..(9): Xaa is Val, Met, or Ile
  MISC_FEATURE (10)..(10): Xaa is Ser or His
GXTFXSXXXX

SEQ ID NO: 10 - protein, 20 aa, Artificial Sequence
  Synthetic Construct
  MISC_FEATURE (4)..(4): Xaa is Gly or Arg
  MISC_FEATURE (8)..(8): Xaa is His or Asp
  MISC_FEATURE (11)..(11): Xaa is Ile or Thr
WMGXIIPXFGXANYAQKFQG

SEQ ID NO: 11 - protein, 22 aa, Artificial Sequence
  Synthetic Construct
  MISC_FEATURE (9)..(9): Xaa is Ala or Arg
  MISC_FEATURE (11)..(11): Xaa is Ser or Gly
  MISC_FEATURE (12)..(12): Xaa is Gly or Tyr
WVGRIKSKXDXXTTDYAAPVKG

SEQ ID NO: 12 - protein, 20 aa, Artificial Sequence
  Synthetic Construct
WVSVISSDGGSTYYADSVKG

SEQ ID NO: 13 - protein, 7 aa, Artificial Sequence
  Synthetic Construct
EIGSLDI

SEQ ID NO: 14 - protein, 8 aa, Artificial Sequence
  Synthetic Construct
DYGVAFAY

SEQ ID NO: 15 - protein, 9 aa, Artificial Sequence
  Synthetic Construct
DYGGLKFDY

SEQ ID NO: 16 - protein, 11 aa, Artificial Sequence
  Synthetic Construct
GPTQAIHYFAY

SEQ ID NO: 17 - protein, 15 aa, Artificial Sequence
  Synthetic Construct
AGFILGSLGVAWMDV

SEQ ID NO: 18 - protein, 11 aa, Artificial Sequence
  Synthetic Construct
SGDSIPSFFAS

SEQ ID NO: 19 - protein, 11 aa, Artificial Sequence
  Synthetic Construct
SGDNIGTKYAY

SEQ ID NO: 20 - protein, 11 aa, Artificial Sequence
  Synthetic Construct
SGDNLRKYSAH

SEQ ID NO: 21 - protein, 11 aa, Artificial Sequence
  Synthetic Construct
SGDSLGSKSVH

SEQ ID NO: 22 - protein, 11 aa, Artificial Sequence
  Synthetic Construct
  MISC_FEATURE (5)..(5): Xaa is Arg, Gly, or Tyr
  MISC_FEATURE (7)..(7): Xaa is Ser or Asn
  MISC_FEATURE (8)..(8): Xaa is Asn, Asp, or Lys
LVIYXDXXRPS

SEQ ID NO: 23 - protein, 11 aa, Artificial Sequence
  Synthetic Construct
  MISC_FEATURE (8)..(8): Xaa is Asn or Lys
LVIYRDSXRPS

SEQ ID NO: 24 - protein, 11 aa, Artificial Sequence
  Synthetic Construct
VLIYKNNHRPS

SEQ ID NO: 25 - protein, 11 aa, Artificial Sequence
  Synthetic Construct
LVIYRDSNRPS

SEQ ID NO: 26 - protein, 11 aa, Artificial Sequence
  Synthetic Construct
LVIYGDSDRPS

SEQ ID NO: 27 - protein, 11 aa, Artificial Sequence
  Synthetic Construct
LVIYYDNKRPS

SEQ ID NO: 28 - protein, 11 aa, Artificial Sequence
  Synthetic Construct
LVIYRDSKRPS

SEQ ID NO: 29 - protein, 10 aa, Artificial Sequence
  Synthetic Construct
  MISC_FEATURE (7)..(7): Xaa is His or Ala
  MISC_FEATURE (10)..(10): Xaa is Ser or His
GFTFSSXAMX

SEQ ID NO: 30 - protein, 10 aa, Artificial Sequence
  Synthetic Construct
  MISC_FEATURE (5)..(5): Xaa is Ser or Asn
  MISC_FEATURE (7)..(7): Xaa His or Ser
GFTFXSXAMS

SEQ ID NO: 31 - protein, 10 aa, Artificial Sequence
  Synthetic Construct
GGTFSSYGVS

SEQ ID NO: 32 - protein, 10 aa, Artificial Sequence
  Synthetic Construct
GFTFSSHAMS

SEQ ID NO: 33 - protein, 10 aa, Artificial Sequence
  Synthetic Construct
GFTFNSSAMS

SEQ ID NO: 34 - protein, 10 aa, Artificial Sequence
  Synthetic Construct
GGTFSSYAIH

SEQ ID NO: 35 - protein, 10 aa, Artificial Sequence
  Synthetic Construct
GFTFSSAAMH

SEQ ID NO: 36 - protein, 20 aa, Artificial Sequence
  Synthetic Construct
WMGGIIPHFGIANYAQKFQG

SEQ ID NO: 37 - protein, 22 aa, Artificial Sequence
  Synthetic Construct
WVGRIKSKADSGTTDYAAPVKG

SEQ ID NO: 38 - protein, 22 aa, Artificial Sequence
  Synthetic Construct
WVGRIKSKRDGYTTDYAAPVKG

SEQ ID NO: 39 - protein, 20 aa, Artificial Sequence
  Synthetic Construct
WMGRIIPDFGTANYAQKFQG

SEQ ID NO: 40 - protein, 12 aa, Artificial Sequence
  Synthetic Construct
RASQGISGNWLT

SEQ ID NO: 41 - protein, 11 aa, Artificial Sequence
  Synthetic Construct
LLIYDASNLQS

SEQ ID NO: 42 - protein, 8 aa, Artificial Sequence
  Synthetic Construct
HQSYRGPM

SEQ ID NO: 43 - protein, 10 aa, Artificial Sequence
  Synthetic Construct
GFTFGRFVMH

SEQ ID NO: 44 - protein, 20 aa, Artificial Sequence
  Synthetic Construct
WVSVIGYSGSSTYYADSVKG

SEQ ID NO: 45 - protein, 20 aa, Artificial Sequence
  Synthetic Construct
EPGYYYPSGYYRGPGYWMDV

SEQ ID NO: 46 - protein, 11 aa, Artificial Sequence
  Synthetic Construct
SGDNIRKKYVH

SEQ ID NO: 47 - protein, 11 aa, Artificial Sequence
  Synthetic Construct
LVIYRDSNRPS

SEQ ID NO: 48 - protein, 8 aa, Artificial Sequence
  Synthetic Construct
SSAGRDNY

SEQ ID NO: 49 - protein, 10 aa, Artificial Sequence
  Synthetic Construct
GFTFSSSAMH

SEQ ID NO: 50 - protein, 20 aa, Artificial Sequence
  Synthetic Construct
WVSVIHYDSSETYYADSVKG

SEQ ID NO: 51 - protein, 7 aa, Artificial Sequence
  Synthetic Construct
DRYFFDV

SEQ ID NO: 52 - protein, 11 aa, Artificial Sequence
  Synthetic Construct
SGDALRYYIAH

SEQ ID NO: 53 - protein, 11 aa, Artificial Sequence
  Synthetic Construct
LVIYYNNNRPS

SEQ ID NO: 54 - protein, 8 aa, Artificial Sequence
  Synthetic Construct
QSYGPGSV

SEQ ID NO: 55 - protein, 10 aa, Artificial Sequence
  Synthetic Construct
GFTFSDYAMH

SEQ ID NO: 56 - protein, 20 aa, Artificial Sequence
  Synthetic Construct
WVSSIFYSGSNTYYADSVKG

SEQ ID NO: 57 - protein, 11 aa, Artificial Sequence
  Synthetic Construct
PKSYASGPFAY

SEQ ID NO: 58 - protein, 13 aa, Artificial Sequence
  Synthetic Construct
SGSSSNIGQNYVS

SEQ ID NO: 59 - protein, 11 aa, Artificial Sequence
  Synthetic Construct
LLIYDNSKRPS

SEQ ID NO: 60 - protein, 10 aa, Artificial Sequence
  Synthetic Construct
SSWDLLSKSR

SEQ ID NO: 61 - protein, 10 aa, Artificial Sequence
  Synthetic Construct
GGTFSTHAIS

SEQ ID NO: 62 - protein, 20 aa, Artificial Sequence
  Synthetic Construct
WMGLIQPRFGTANYAQKFQR

SEQ ID NO: 63 - protein, 8 aa, Artificial Sequence
  Synthetic Construct
DYYGGMAY

SEQ ID NO: 64 - protein, 11 aa, Artificial Sequence
  Synthetic Construct
  MISC_FEATURE (4)..(4): Xaa is Asn or Ala
  MISC_FEATURE (5)..(5): Xaa is Ile or Leu
  MISC_FEATURE (7)..(8): Xaa is Lys or Tyr
  MISC_FEATURE (9)..(9): Xaa is Tyr or Ile
  MISC_FEATURE (10)..(10): Xaa is Val or Ala
SGDXXRXXXXH

SEQ ID NO: 65 - protein, 11 aa, Artificial Sequence
  Synthetic Construct
  MISC_FEATURE (2)..(2): Xaa is Val or Leu
  MISC_FEATURE (5)..(5): Xaa is Asp, Arg, or Tyr
  MISC_FEATURE (6)..(6): Xaa is Ala, Asp, or Asn
  MISC_FEATURE (7)..(7): Xaa is Ser or Asn
  MISC_FEATURE (8)..(8): Xaa is Lys or Asn
  MISC_FEATURE (9)..(9): Xaa is Leu or Arg
  MISC_FEATURE (10)..(10): Xaa is Gln or Pro
LXIYXXXXXXS

SEQ ID NO: 66 - protein, 10 aa, Artificial Sequence
  Synthetic Construct
  MISC_FEATURE (2)..(2): Xaa is Phe or Gly
  MISC_FEATURE (5)..(5): Xaa is Gly or Ser
  MISC_FEATURE (6)..(6): Xaa is Arg, Ser, Asp, or Thr
  MISC_FEATURE (7)..(7): Xaa is Phe, Ser, Tyr, or His
  MISC_FEATURE (8)..(8): Xaa is Val or Ala
  MISC_FEATURE (9)..(9): Xaa is Met or Ile
  MISC_FEATURE (10)..(10): Xaa is His or Ser
GXTFXXXXXX

SEQ ID NO: 67 - protein, 452 aa, Artificial Sequence
  Synthetic Construct
DIVLTQPPSVSGAPGQRVTISCSGSSSNIGSNYVSWYQQLPGTAPKVLIYKNNHRPSGVPDRFSGSKSGTSASLAITGLQAEDEADYYCASWDHSDRFYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAQVQLVQSGAEVKKPGSSVKVSCKASGGTFSSYGVSWVRQAPGQGLEWMGGIIPHFGIANYAQKFQGRVTITADESTSTAYMELSSLRSEDTAVYYCAREIGSLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 68 - protein, 453 aa, Artificial Sequence
  Synthetic Construct
DIELTQPPSVSVSPGQTASITCSGDSIPSFFASWYQQKPGQAPVLVIYRDSNRPSGIPERFSGSNSGNTATLTISGTQAEDEADYYCYVTAPWKSIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAEVQLVESGGGLVKPGGSLRLSCAASGFTFSSHAMSWVRQAPGKGLEWVGRIKSKADSGTTDYAAPVKGRFTISRDDSKNTLYLQMNSLKTEDTAVYYCARDYGVAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 69 - protein, 454 aa, Artificial Sequence
  Synthetic Construct
DIELTQPPSVSVSPGQTASITCSGDNIGTKYAYWYQQKPGQAPVLVIYGDSDRPSGIPERFSGSNSGNTATLTISGTQAEDEADYYCYSADAQQMKAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAEVQLVESGGGLVKPGGSLRLSCAASGFTFNSSAMSWVRQAPGKGLEWVGRIKSKRDGYTTDYAAPVKGRFTISRDDSKNTLYLQMNSLKTEDTAVYYCARDYGGLKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 70 - protein, 453 aa, Artificial Sequence
  Synthetic Construct
DIELTQPPSVSVSPGQTASITCSGDNLRKYSAHWYQQKPGQAPVLVIYYDNKRPSGIPERFSGSNSGNTATLTISGTQAEDEADYYCQVYASVHRMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAQVQLVQSGAEVKKPGSSVKVSCKASGGTFSSYAIHWVRQAPGQGLEWMGRIIPDFGTANYAQKFQGRVTITADESTSTAYMELSSLRSEDTAVYYCARGPTQAIHYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 71 - protein, 458 aa, Artificial Sequence
  Synthetic Construct
DIELTQPPSVSVSPGQTASITCSGDSLGSKSVHWYQQKPGQAPVLVIYRDSKRPSGIPERFSGSNSGNTATLTISGTQAEDEADYYCQTYDWSHFGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAEVQLLESGGGLVQPGGSLRLSCAASGFTFSSAAMHWVRQAPGKGLEWVSVISSDGGSTYYADSVKGRFTISRDNSKNTLYLQMNSLRAEDTAVYYCARAGFILGSLGVAWMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 72 - protein, 465 aa, Artificial Sequence
  Synthetic Construct
DIQMTQSPSSLSASVGDRVTITCRASQGISGNWLTWYQQKPGKAPKLLIYDASNLQSGVPSRFSGSGSGTDFTLTISSLQPEDFATYYCHQSYRGPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEAEVQLLESGGGLVQPGGSLRLSCAASGFTFGRFVMHWVRQAPGKGLEWVSVIGYSGSSTYYADSVKGRFTISRDNSKNTLYLQMNSLRAEDTAVYYCAREPGYYYPSGYYRGPGYWMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 73 - protein, 448 aa, Artificial Sequence
  Synthetic Construct
DIELTQPPSVSVSPGQTASITCSGDNIRKKYVHWYQQKPGQAPVLVIYRDSNRPSGIPERFSGSNSGNTATLTISGTQAEDEADYYCSSAGRDNYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAEVQLLESGGGLVQPGGSLRLSCAASGFTFSSSAMHWVRQAPGKGLEWVSVIHYDSSETYYADSVKGRFTISRDNSKNTLYLQMNSLRAEDTAVYYCARDRYFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 74 - protein, 452 aa, Artificial Sequence
  Synthetic Construct
DIELTQPPSVSVSPGQTASITCSGDALRYYIAHWYQQKPGQAPVLVIYYNNNRPSGIPERFSGSNSGNTATLTISGTQAEDEADYYCQSYGPGSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAEVQLLESGGGLVQPGGSLRLSCAASGFTFSDYAMHWVRQAPGKGLEWVSSIFYSGSNTYYADSVKGRFTISRDNSKNTLYLQMNSLRAEDTAVYYCARPKSYASGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 75 - protein, 453 aa, Artificial Sequence
  Synthetic Construct
DIVLTQPPSVSGAPGQRVTISCSGSSSNIGQNYVSWYQQLPGTAPKLLIYDNSKRPSGVPDRFSGSKSGTSASLAITGLQAEDEADYYCSSWDLLSKSRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEAQVQLVQSGAEVKKPGSSVKVSCKASGGTFSTHAISWVRQAPGQGLEWMGLIQPRFGTANYAQKFQGRVTITADESTSTAYMELSSLRSEDTAVYYCARDYYGGMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 76 - protein, 20 aa, Artificial Sequence
  Synthetic Construct
  MISC_FEATURE (4)..(4): Xaa is Val or Ser
  MISC_FEATURE (6)..(6): Xaa is Gly, His, or Phe
  MISC_FEATURE (8)..(8): Xaa is Ser or Asp
  MISC_FEATURE (9)..(9): Xaa is Gly or Ser
  MISC_FEATURE (11)..(11): Xaa is Ser, Glu, or Asn
WVSXIXYXXSXTYYADSVKG

SEQ ID NO: 77 - protein, 138 aa, Artificial Sequence
  Synthetic Construct
MEWSWIFLFLLSGTAGVLSEVQLQQSGPELVKPGTSVKISCKASGYSFTGYYMHWVKQSQVKSLEWIGRINPDNGGRTYNQIFKDKASLTVHKSSSTAYMELHSLTSDDSAVYYCTRERGQYGNYGGFSDWGQGTLVT

SEQ ID NO: 78 - protein, 119 aa, Artificial Sequence
  Synthetic Construct
EVQLQQSGPELVKPGTSVKISCKASGYSFTGYYMHWVKQSQVKSLEWIGRINPDNGGRTYNQIFKDKASLTVHKSSSTAYMELHSLTSDDSAVYYCTRERGQYGNYGGFSDWGQGTLVT

SEQ ID NO: 79 - protein, 6 aa, Artificial Sequence
  Synthetic Construct
TGYYMK

SEQ ID NO: 80 - protein, 18 aa, Artificial Sequence
  Synthetic Construct
RINPDNGGRTYNQIFKDK

SEQ ID NO: 81 - protein, 14 aa, Artificial Sequence
  Synthetic Construct
RERGQYGNYGGFSD

SEQ ID NO: 82 - protein, 10 aa, Rattus norvegicus
GFTFSHYYMA

SEQ ID NO: 83 - protein, 17 aa, Rattus norvegicus
SITNSGGSINYRDSVKG

SEQ ID NO: 84 - protein, 14 aa, Rattus norvegicus
EGGENYGGYPPFAY

SEQ ID NO: 85 - protein, 15 aa, Rattus norvegicus
RANQGVSLSRYNLMH

SEQ ID NO: 86 - protein, 7 aa, Rattus norvegicus
RSSNLAS

SEQ ID NO: 87 - protein, 9 aa, Rattus norvegicus
QQSRESPFT

SEQ ID NO: 88 - protein, 10 aa, Rattus norvegicus
GSTFSNYGMK

SEQ ID NO: 89 - protein, 17 aa, Rattus norvegicus
SISRSSTYIYYADTVKG

SEQ ID NO: 90 - protein, 12 aa, Rattus norvegicus
AISTPFYWYFDF

SEQ ID NO: 91 - protein, 10 aa, Rattus norvegicus
LASSSVSYMT

SEQ ID NO: 92 - protein, 7 aa, Rattus norvegicus
GTSNLAS

SEQ ID NO: 93 - protein, 10 aa, Rattus norvegicus
LHLTSYPPYT

SEQ ID NO: 94 - protein, 7 aa, Rattus norvegicus
RSSNLAQ

SEQ ID NO: 95 - protein, 10 aa, Rattus norvegicus
GFTFSNYYMY

SEQ ID NO: 96 - protein, 17 aa, Rattus norvegicus
SINTDGGSTYYPDSVKG

SEQ ID NO: 97 - protein, 9 aa, Rattus norvegicus
STPNIPLAY

SEQ ID NO: 98 - protein, 11 aa, Rattus norvegicus
KASQNIYKYLN

SEQ ID NO: 99 - protein, 7 aa, Rattus norvegicus
YSNSLQT

SEQ ID NO: 100 - protein, 8 aa, Rattus norvegicus
FQYSSGPT

SEQ ID NO: 101 - protein, 10 aa, Rattus norvegicus
GFTFSYYAMS

SEQ ID NO: 102 - protein, 17 aa, Rattus norvegicus
SISRGGDNTYYRDTVKG

SEQ ID NO: 103 - protein, 9 aa, Rattus norvegicus
LNYNNYFDY

SEQ ID NO: 104 - protein, 11 aa, Rattus norvegicus
QASQDIGNWLS

SEQ ID NO: 105 - protein, 7 aa, Rattus norvegicus
GATSLAD

SEQ ID NO: 106 - protein, 9 aa, Rattus norvegicus
LQAYSAPFT

SEQ ID NO: 107 - protein, 118 aa, Rattus norvegicus
EVQLVETGGGLVQPGRSLKLSCVASGFTFSNYYMYWIRQAPGKGLEWISSINTDGGSTYYPDSVKGRFAISRDNAENTVYLQMNSLRSEDTATYYCAKSTPNIPLAYWGQGTLVTVSS

SEQ ID NO: 108 - protein, 108 aa, Rattus norvegicus
DIQMTQSPSLLSASVGDRVTLSCKASQNIYKYLNWFQQKLGEAPKLLIYYSNSLQTGIPSRFSGSGSGTDFTLTISSLQPEDVAIYFCFQYSSGPTFGPGTKLELKRA

SEQ ID NO: 109 - protein, 123 aa, Rattus norvegicus
EVQLVESGGGLVQPGRSLKLSCAASGFTFSHYYMAWVRQAPTKGLEWVASITNSGGSINYRDSVKGRFTISRDNAKSTLYLQMDSLRSEDTATYYCTREGGENYGGYPPFAYWGQGTLVTVSS

SEQ ID NO: 110 - protein, 112 aa, Rattus norvegicus
DIVLTQSPALAVSLGQRATISCRANQGVSLSRYNLMHWYQQKPGQKPKLLIYRSSNLASGIPARFSGSGSGTDFTLTINPVQADDIATYYCQQSRESPFTFGAGTKLELKRA

SEQ ID NO: 111 - protein, 118 aa, Rattus norvegicus
EVQLVESGGGLVQPGGSLKLSCAASGFTFSYYAMSWVCQAPTKGLEWVASISRGGDNTYYRDTVKGRFTTSRDNAKNTLYLQMDSLRSEDTATYYCARLNYNNYFDYWGQGVMVTVSS

SEQ ID NO: 112 - protein, 109 aa, Rattus norvegicus
DIQMTQSPASLSASLEEIVTITCQASQDIGNWLSWYQQKPGKSPQLLIYGATSLADGVPSRFSGSRSGTQYSLKISRLRVEDIGIYYCLQAYSAPFTFGSGTKLEIKRA

SEQ ID NO: 113 - protein, 121 aa, Rattus norvegicus
EVQLVESGGGLVQPGRSLKLSCLASGSTFSNYGMKWIRQAPGKGLEWVASISRSSTYIYYADTVKGRFTISRDNARNTLYLQMTSLRSEDTALYYCAAAISTPFYWYFDFWGPGTVVTVSS

SEQ ID NO: 114 - protein, 109 aa, Rattus norvegicus
EIVLTQSPTTMAASPGEKVTLNCLASSSVSYMTWYQQKSGASPKLWIYGTSNLASGVPNRFSGSGSGTSYSLAISSMEAEDVATYYCLHLTSYPPYTFGAGTKLELKRA

SEQ ID NO: 115 - protein, 113 aa, Artificial Sequence
  Synthetic Construct
DIVLTQSPDSLAVSLGERATINCRANQGVSLSRYNLMHWYQQKPGQKPKLLIYRSSNLAQGIPARFSGSGSGTDFTLTISSVQADDIAVYYCQQSRESPFTFGQGTKLELKRA

SEQ ID NO: 116 - protein, 123 aa, Artificial Sequence
  Synthetic Construct
EVQLVESGGGLVQPGGSLRLSCAASGFTFSHYYMAWVRQAPGKGLEWVASITNSGGSINYRDSVKGRFTISRDNSKNTLYLQMNSLRAEDTAVYYCAREGGENYGGYPPFAYWGQGTLVTVSS

SEQ ID NO: 117 - protein, 123 aa, Artificial Sequence
  Synthetic Construct
EVQLVESGGGLVQPGGSLRLSCAASGFTFSHYYMAWVRQAPGKGLEWVASITNSGGSINYRDSVKGRFTISRDNAKSTLYLQMNSLRAEDTATYYCTREGGENYGGYPPFAYWGQGTLVTVSS

SEQ ID NO: 118 - protein, 113 aa, Artificial Sequence
  Synthetic Construct
DIVMTQSPDSLAVSLGERATINCRANQGVSLSRYNLMHWYQQKPGQPPKLLIYRSSNLASGVPDRFSGSGSGTDFTLTISSLQAEDVAVYYCQQSRESPFTFGQGTKVEIKRA

SEQ ID NO: 119 - protein, 113 aa, Artificial Sequence
  Synthetic Construct
DIVLTQSPDSLAVSLGERATINCRANQGVSLSRYNLMHWYQQKPGQKPKLLIYRSSNLASGIPARFSGSGSGTDFTLTISSVQADDIAVYYCQQSRESPFTFGQGTKLELKRA

SEQ ID NO: 120 - protein, 113 aa, Artificial Sequence
  Synthetic Construct
DIVLTQSPDSLAVSLGERATINCRANQGVSLSRYNLMHWYQQKPGQKPKLAIYRSSNLASGIPARFSGSGSGTDFTLTISSVQADDIAVYYCQQSRESPFTFGQGTKLELKRA

SEQ ID NO: 121 - protein, 121 aa, Artificial Sequence
  Synthetic Construct
EVQLVESGGGLVQPGGSLRLSCAASGSTFSNYGMKWVRQAPGKGLEWVSSISRSSTYIYYADTVKGRFTISRDNSKNTLYLQMNSLRAEDTAVYYCARAISTPFYWYFDFWGQGTLVTVSS

SEQ ID NO: 122 - protein, 121 aa, Artificial Sequence
  Synthetic Construct
EVQLVESGGGLVQPGGSLRLSCAASGSTFSNYGMKWIRQAPGKGLEWVSSISRSSTYIYYADTVKGRFTISRDNSKNTLYLQMNSLRAEDTAVYYCAAAISTPFYWYFDFWGQGTLVTVSS

SEQ ID NO: 123 - protein, 121 aa, Artificial Sequence
  Synthetic Construct
EVQLVESGGGLVQPGGSLRLSCAASGSTFSNYGMKWIRQAPGKGLEWVASISRSSTYIYYADTVKGRFTISRDNSKNTLYLQMNSLRAEDTAVYYCAAAISTPFYWYFDFWGQGTLVTVSS

SEQ ID NO: 124 - protein, 121 aa, Artificial Sequence
  Synthetic Construct
EVQLVESGGGLVQPGGSLRLSCAASGSTFSNYGMKWIRQAPGKGLEWVASISRSSTYIYYADTVKGRFTISRDNSKNTLYLQMNSLRAEDTALYYCAAAISTPFYWYFDFWGPGTLVTVSS

SEQ ID NO: 125 - protein, 121 aa, Artificial Sequence
  Synthetic Construct
EVQLVESGGGLVQPGGSLRLSCAASGSTFSNYGMKWIRQAPGKGLEWVASISRSSTYIYYADTVKGRFTISRDNAKNTLYLQMNSLRSEDTALYYCAAAISTPFYWYFDFWGPGTLVTVSS

SEQ ID NO: 126 - protein, 109 aa, Artificial Sequence
  Synthetic Construct
EIVLTQSPGTLSLSPGERATLSCLASSSVSYMTWYQQKPGQAPRLLIYGTSNLASGIPDRFSGSGSGTDFTLTISRLEPEDFAVYYCLHLTSYPPYTFGQGTKVEIKRA

SEQ ID NO: 127 - protein, 109 aa, Artificial Sequence
  Synthetic Construct
EIVLTQSPGTLSLSPGERATLSCLASSSVSYMTWYQQKPGQSPRLWIYGTSNLASGVPDRFSGSGSGTDFTLTISRLEPEDFAVYYCLHLTSYPPYTFGQGTKVEIKRA

SEQ ID NO: 128 - protein, 109 aa, Artificial Sequence
  Synthetic Construct
EIVLTQSPGTLSLSPGERATLSCLASSSVSYMTWYQQKPGASPRLWIYGTSNLASGVPDRFSGSGSGTDYTLTISRLEPEDFATYYCLHLTSYPPYTFGAGTKVEIKRA

SEQ ID NO: 129 - protein, 109 aa, Artificial Sequence
  Synthetic Construct
EIVLTQSPGTMSASPGERVTLSCLASSSVSYMTWYQQKPGASPRLWIYGTSNLASGVPDRFSGSGSGTDYTLTISRMEPEDFATYYCLHLTSYPPYTFGAGTKLELKRA

SEQ ID NO: 130 - protein, 109 aa, Artificial Sequence
  Synthetic Construct
EIVLTQSPGTMSASPGERVTLNCLASSSVSYMTWYQQKSGASPKLWIYGTSNLASGVPNRFSGSGSGTSYTLTISRMEPEDFATYYCLHLTSYPPYTFGAGTKLELKRA

SEQ ID NO: 131 - protein, 123 aa, Artificial Sequence; identical to SEQ ID NO: 117
  Synthetic Construct

SEQ ID NO: 132 - protein, 121 aa, Artificial Sequence; identical to SEQ ID NO: 122
  Synthetic Construct

SEQ ID NO: 133 - protein, 121 aa, Artificial Sequence; identical to SEQ ID NO: 123
  Synthetic Construct

SEQ ID NO: 134 - protein, 118 aa, Artificial Sequence
  Synthetic Construct
EVQLVESGGGLVQPGGSLRLSCAASGFTFSNYYMYWIRQAPGKGLEWVSSINTDGGSTYYADSVKGRFTISRDNSKNTVYLQMNSLRAEDTAVYYCAKSTPNIPLAYWGQGTLVTVSS

SEQ ID NO: 135 - protein, 118 aa, Artificial Sequence
  Synthetic Construct
EVQLVESGGGLVQPGGSLRLSCAASGFTFSNYYMYWIRQAPGKGLEWISSINTDGGSTYYPDSVKGRFTISRDNSKNTVYLQMNSLRAEDTAVYYCAKSTPNIPLAYWGQGTLVTVSS

SEQ ID NO: 136 - protein, 108 aa, Artificial Sequence
  Synthetic Construct
DIQMTQSPSSLSASVGDRVTITCKASQNIYKYLNWFQQKPGKAPKLLIYYSNSLQTGVPSRFSGSGSGTDFTLTISSLQPEDFATYYCFQYSSGPTFGQGTKVEIKRT

SEQ ID NO: 137 - protein, 108 aa, Artificial Sequence
  Synthetic Construct
DIQMTQSPSSLSASVGDRVTITCKASQNIYKYLNWFQQKPGKAPKLLIYYSNSLQTGIPSRFSGSGSGTDFTLTISSLQPEDFATYYCFQYSSGPTFGQGTKVEIKRT

SEQ ID NO: 138 - protein, 108 aa, Artificial Sequence
  Synthetic Construct
DIQMTQSPSSLSASVGDRVTISCKASQNIYKYLNWFQQKPGEAPKLLIYYSNSLQTGIPSRFSGSGSGTDFTLTISSLQPEDFAIYFCFQYSSGPTFGPGTKVEIKRT

SEQ ID NO: 139 - protein, 118 aa, Artificial Sequence
  Synthetic Construct
EVQLVESGGGLVQPGGSLRLSCAASGFTFSYYAMSWVRQAPGKGLEWVASISRGGDNTYYRDSVKGRFTTSRDNSKNTLYLQMNSLRAEDTAVYYCARLNYNNYFDYWGQGTLVTVSS

SEQ ID NO: 140 - protein, 118 aa, Artificial Sequence
  Synthetic Construct
EVQLVESGGGLVQPGGSLRLSCAASGFTFSYYAMSWVRQAPGKGLEWVASISRGGDNTYYRDSVKGRFTTSRDNSKNTLYLQMNSLRAEDTATYYCARLNYNNYFDYWGQGVLVTVSS

SEQ ID NO: 141 - protein, 109 aa, Artificial Sequence
  Synthetic Construct
DIQMTQSPSSLSASVGDRVTITCQASQDIGNWLSWYQQKPGKAPKLLIYGATSLADGVPSRFSGSGSGTDFTLTISSLQPEDFATYYCLQAYSAPFTFGQGTKVEIKRT

SEQ ID NO: 142 - protein, 109 aa, Artificial Sequence
  Synthetic Construct
DIQMTQSPSSLSASVGDRVTITCQASQDIGNWLSWYQQKPGKAPKLLIYGATSLADGVPSRFSGSRSGTDYTLTISSLQPEDFATYYCLQAYSAPFTFGQGTKVEIKRT

SEQ ID NO: 143 - protein, 109 aa, Artificial Sequence
  Synthetic Construct
DIQMTQSPSSLSASVGDRVTITCQASQDIGNWLSWYQQKPGKSPKLLIYGATSLADGVPSRFSGSRSGTQYTLTISSLQPEDFATYYCLQAYSAPFTFGSGTKVEIKRT

SEQ ID NO: 144 - protein, 451 aa, Artificial Sequence
  Synthetic Construct
EVQLVESGGGLVQPGGSLRLSCAASGSTFSNYGMKWIRQAPGKGLEWVSSISRSSTYIYYADTVKGRFTISRDNSKNTLYLQMNSLRAEDTAVYYCAAAISTPFYWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 214 aa, Artificial Sequence
  Synthetic Construct
EIVLTQSPGTLSLSPGERATLSCLASSSVSYMTWYQQKPGQSPRLWIYGTSNLASGVPDRFSGSGSGTDFTLTISRLEPEDFAVYYCLHLTSYPPYTFGQGTKVEIKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 448 aa, Artificial Sequence
  Synthetic Construct
EVQLVESGGGLVQPGGSLRLSCAASGFTFSNYYMYWIRQAPGKGLEWISSINTDGGSTYYPDSVKGRFTISRDNSKNTVYLQMNSLRAEDTAVYYCAKSTPNIPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 213 aa, Artificial Sequence
  Synthetic Construct
DIQMTQSPSSLSASVGDRVTISCKASQNIYKYLNWFQQKPGEAPKLLIYYSNSLQTGIPSRFSGSGSGTDFTLTISSLQPEDFAIYFCFQYSSGPTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 449 aa, Artificial Sequence
  Synthetic Construct
EVQLVESGGGLVQPGGSLRLSCAASGFTFSHYYMAWVRQAPGKGLEWVASITNSGGSINYRDSVKGRFTISRDNAKSTLYLQMNSLRAEDTATYYCTREGGENYGGYPPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 149 - protein, 218 aa, Artificial Sequence
  Synthetic Construct
DIVLTQSPDSLAVSLGERATINCRANQGVSLSRYNLMHWYQQKPGQKPKLLIYRSSNLAQGIPARFSGSGSGTDFTLTISSVQADDIAVYYCQQSRESPFTFGQGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 448 aa, Artificial Sequence
  Synthetic Construct
EVQLVESGGGLVQPGGSLRLSCAASGFTFSYYAMSWVRQAPGKGLEWVASISRGGDNTYYRDSVKGRFTTSRDNSKNTLYLQMNSLRAEDTATYYCARLNYNNYFDYWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 214 aa, Artificial Sequence
  Synthetic Construct
DIQMTQSPSSLSASVGDRVTITCQASQDIGNWLSWYQQKPGKSPKLLIYGATSLADGVPSRFSGSRSGTQYTLTISSLQPEDFATYYCLQAYSAPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 453 aa, Artificial Sequence
  Synthetic Construct
EVQLVESGGGLVQPGRSLKLSCAASGFTFSHYYMAWVRQAPTKGLEWVASITNSGGSINYRDSVKGRFTISRDNAKSTLYLQMDSLRSEDTATYYCTREGGENYGGYPPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 217 aa, Artificial Sequence
  Synthetic Construct
DIVLTQSPALAVSLGQRATISCRANQGVSLSRYNLMHWYQQKPGQKPKLLIYRSSNLASGIPARFSGSGSGTDFTLTINPVQADDIATYYCQQSRESPFTFGAGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 451 aa, Artificial Sequence
  Synthetic Construct
EVQLVESGGGLVQPGRSLKLSCLASGSTFSNYGMKWIRQAPGKGLEWVASISRSSTYIYYADTVKGRFTISRDNARNTLYLQMTSLRSEDTALYYCAAAISTPFYWYFDFWGPGTVVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 214 aa, Artificial Sequence
  Synthetic Construct
EIVLTQSPTTMAASPGEKVTLNCLASSSVSYMTWYQQKSGASPKLWIYGTSNLASGVPNRFSGSGSGTSYSLAISSMEAEDVATYYCLHLTSYPPYTFGAGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 453 aa, Artificial Sequence
  Synthetic Construct
EVQLVESGGGLVQPGGSLRLSCAASGFTFSHYYMAWVRQAPGKGLEWVASITNSGGSINYRDSVKGRFTISRDNSKNTLYLQMNSLRAEDTAVYYCAREGGENYGGYPPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 218 aa, Artificial Sequence
  Synthetic Construct
DIVMTQSPDSLAVSLGERATINCRANQGVSLSRYNLMHWYQQKPGQPPKLLIYRSSNLASGVPDRFSGSGSGTDFTLTISSLQAEDVAVYYCQQSRESPFTFGQGTKVEIKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 218 aa, Artificial Sequence
  Synthetic Construct
DIVLTQSPDSLAVSLGERATINCRANQGVSLSRYNLMHWYQQKPGQKPKLLIYRSSNLASGIPARFSGSGSGTDFTLTISSVQADDIAVYYCQQSRESPFTFGQGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 453 aa, Artificial Sequence
  Synthetic Construct
EVQLVESGGGLVQPGGSLRLSCAASGFTFSHYYMAWVRQAPGKGLEWVASITNSGGSINYRDSVKGRFTISRDNAKSTLYLQMNSLRAEDTATYYCTREGGENYGGYPPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 218 aa, Artificial Sequence
  Synthetic Construct
DIVLTQSPDSLAVSLGERATINCRANQGVSLSRYNLMHWYQQKPGQKPKLAIYRSSNLASGIPARFSGSGSGTDFTLTISSVQADDIAVYYCQQSRESPFTFGQGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 451 aa, Artificial Sequence
  Synthetic Construct
EVQLVESGGGLVQPGGSLRLSCAASGSTFSNYGMKWVRQAPGKGLEWVSSISRSSTYIYYADTVKGRFTISRDNSKNTLYLQMNSLRAEDTAVYYCARAISTPFYWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 451 aa, Artificial Sequence
  Synthetic Construct
EVQLVESGGGLVQPGGSLRLSCAASGSTFSNYGMKWIRQAPGKGLEWVSSISRSSTYIYYADTVKGRFTISRDNSKNTLYLQMNSLRAEDTAVYYCAAAISTPFYWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 451 aa, Artificial Sequence
  Synthetic Construct
EVQLVESGGGLVQPGGSLRLSCAASGSTFSNYGMKWIRQAPGKGLEWVASISRSSTYIYYADTVKGRFTISRDNSKNTLYLQMNSLRAEDTAVYYCAAAISTPFYWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 451 aa, Artificial Sequence
  Synthetic Construct
EVQLVESGGGLVQPGGSLRLSCAASGSTFSNYGMKWIRQAPGKGLEWVASISRSSTYIYYADTVKGRFTISRDNSKNTLYLQMNSLRAEDTALYYCAAAISTPFYWYFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 451 aa, Artificial Sequence
  Synthetic Construct
EVQLVESGGGLVQPGGSLRLSCAASGSTFSNYGMKWIRQAPGKGLEWVASISRSSTYIYYADTVKGRFTISRDNAKNTLYLQMNSLRSEDTALYYCAAAISTPFYWYFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 214 aa, Artificial Sequence
  Synthetic Construct
EIVLTQSPGTLSLSPGERATLSCLASSSVSYMTWYQQKPGQAPRLLIYGTSNLASGIPDRFSGSGSGTDFTLTISRLEPEDFAVYYCLHLTSYPPYTFGQGTKVEIKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 214 aa, Artificial Sequence
  Synthetic Construct
EIVLTQSPGTLSLSPGERATLSCLASSSVSYMTWYQQKPGASPRLWIYGTSNLASGVPDRFSGSGSGTDYTLTISRLEPEDFATYYCLHLTSYPPYTFGAGTKVEIKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 214 aa, Artificial Sequence
  Synthetic Construct
EIVLTQSPGTMSASPGERVTLSCLASSSVSYMTWYQQKPGASPRLWIYGTSNLASGVPDRFSGSGSGTDYTLTISRMEPEDFATYYCLHLTSYPPYTFGAGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 214 aa, Artificial Sequence
  Synthetic Construct
EIVLTQSPGTMSASPGERVTLNCLASSSVSYMTWYQQKSGASPKLWIYGTSNLASGVPNRFSGSGSGTSYTLTISRMEPEDFATYYCLHLTSYPPYTFGAGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 451 aa, Artificial Sequence
  Synthetic Construct
EVQLVESGGGLVQPGGSLRLSCAASGSTFSNYGMKWIRQAPGKGLEWVASISRSSTYIYYADTVKGRFTISRDNSKNTLYLQMNSLRAEDTAVYYCAAAISTPFYWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 448 aa, Artificial Sequence
  Synthetic Construct
EVQLVESGGGLVQPGGSLRLSCAASGFTFSNYYMYWIRQAPGKGLEWVSSINTDGGSTYYADSVKGRFTISRDNSKNTVYLQMNSLRAEDTAVYYCAKSTPNIPLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 213 aa, Artificial Sequence
  Synthetic Construct
DIQMTQSPSSLSASVGDRVTITCKASQNIYKYLNWFQQKPGKAPKLLIYYSNSLQTGVPSRFSGSGSGTDFTLTISSLQPEDFATYYCFQYSSGPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 213 aa, Artificial Sequence
  Synthetic Construct
DIQMTQSPSSLSASVGDRVTITCKASQNIYKYLNWFQQKPGKAPKLLIYYSNSLQTGIPSRFSGSGSGTDFTLTISSLQPEDFATYYCFQYSSGPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 448 aa, Artificial Sequence
  Synthetic Construct
EVQLVESGGGLVQPGGSLRLSCAASGFTFSYYAMSWVRQAPGKGLEWVASISRGGDNTYYRDSVKGRFTTSRDNSKNTLYLQMNSLRAEDTAVYYCARLNYNNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 214 aa, Artificial Sequence
  Synthetic Construct
DIQMTQSPSSLSASVGDRVTITCQASQDIGNWLSWYQQKPGKAPKLLIYGATSLADGVPSRFSGSGSGTDFTLTISSLQPEDFATYYCLQAYSAPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 214 aa, Artificial Sequence
  Synthetic Construct
DIQMTQSPSSLSASVGDRVTITCQASQDIGNWLSWYQQKPGKAPKLLIYGATSLADGVPSRFSGSRSGTDYTLTISSLQPEDFATYYCLQAYSAPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 10 aa, Artificial Sequence
  Synthetic Construct
GFNIKDSLMH

SEQ ID NO: 178 - protein, 16 aa, Artificial Sequence
  Synthetic Construct
IDPEDGETKYAPNFQD

SEQ ID NO: 179 - protein, 10 aa, Artificial Sequence
  Synthetic Construct
YTSDYYTMDY

SEQ ID NO: 180 - protein, 11 aa, Artificial Sequence
  Synthetic Construct
LASQTIGTWLA

SEQ ID NO: 181 - protein, 7 aa, Artificial Sequence
  Synthetic Construct
AATSLAD

SEQ ID NO: 182 - protein, 9 aa, Artificial Sequence
  Synthetic Construct
QQLYSTPWT

SEQ ID NO: 183 - protein, 10 aa, Artificial Sequence
  Synthetic Construct
GFNIKDSLMH

SEQ ID NO: 184 - protein, 16 aa, Artificial Sequence
  Synthetic Construct
IDPEDGETKYAPNFQD

SEQ ID NO: 185 - protein, 10 aa, Artificial Sequence
  Synthetic Construct
YTSDYYTMDY

SEQ ID NO: 186 - protein, 11 aa, Artificial Sequence
  Synthetic Construct
LASQTIGTWLA

SEQ ID NO: 187 - protein, 7 aa, Artificial Sequence
  Synthetic Construct
AATSLAD

SEQ ID NO: 188 - protein, 9 aa, Artificial Sequence
  Synthetic Construct
QQLYSTPWT

SEQ ID NO: 189 - protein, 10 aa, Artificial Sequence
  Synthetic Construct
GFNIKDSLMH

SEQ ID NO: 190 - protein, 16 aa, Artificial Sequence
  Synthetic Construct
IDPEDGETKYAPNFQS

SEQ ID NO: 191 - protein, 10 aa, Artificial Sequence
  Synthetic Construct
YTSPYYTMDY

SEQ ID NO: 192 - protein, 11 aa, Artificial Sequence
  Synthetic Construct
LASQTIGTWLA

SEQ ID NO: 193 - protein, 7 aa, Artificial Sequence
  Synthetic Construct
AATSLAD

SEQ ID NO: 194 - protein, 9 aa, Artificial Sequence
  Synthetic Construct
QQVYSTPWT

SEQ ID NO: 195 - protein, 10 aa, Artificial Sequence
  Synthetic Construct
GFNIKDSLMH

SEQ ID NO: 196 - protein, 16 aa, Artificial Sequence
  Synthetic Construct
IDPEDGETRYAPNFQD

SEQ ID NO: 197 - protein, 10 aa, Artificial Sequence
  Synthetic Construct
YTSRYYTMEY

SEQ ID NO: 198 - protein, 11 aa, Artificial Sequence
  Synthetic Construct
LASQTIGTWLA

SEQ ID NO: 199 - protein, 7 aa, Artificial Sequence
  Synthetic Construct
AATSLAD

SEQ ID NO: 200 - protein, 9 aa, Artificial Sequence
  Synthetic Construct
QQLYWTPWT

SEQ ID NO: 201 - protein, 119 aa, Artificial Sequence
  Synthetic Construct
EVQLQQSGAELVRPGASVRLSCTASGFNIKDSLMHWVKQRPEQGLEWIGWIDPEDGETKYAPNFQDKATITAVTSSNTAYLQLSSLTSEDSAIYYCARYTSDYYTMDYWGQGTSVTVSS

SEQ ID NO: 202 - protein, 107 aa, Artificial Sequence
  Synthetic Construct
DIQMTQSPASQSASLGESVTFTCLASQTIGTWLAWYQQKPGKSPQLLIYAATSLADGVPSRFSGSGSGTKFSFKISSLQAEDFASYYCQQLYSTPWTFGGGTKLEIK

SEQ ID NO: 203 - protein, 119 aa, Artificial Sequence; identical to SEQ ID NO: 201
  Synthetic Construct

SEQ ID NO: 204 - protein, 107 aa, Artificial Sequence; identical to SEQ ID NO: 202
  Synthetic Construct

SEQ ID NO: 205 - protein, 119 aa, Artificial Sequence
  Synthetic Construct
EVQLQQSGAELVRPGASVRLSCTASGFNIKDSLMHWVKQRPEQGLEWIGWIDPEDGETKYAPNFQSKATITAVTSSNTAYLQLSSLTSEDSAIYYCARYTSPYYTMDYWGQGTSVTVSS

SEQ ID NO: 206 - protein, 107 aa, Artificial Sequence
  Synthetic Construct
DIQMTQSPASQSASLGESVTFTCLASQTIGTWLAWYQQKPGKSPQLLIYAATSLADGVPSRFSGSGSGTKFSFKISSLQAEDFASYYCQQVYSTPWTFGGGTKLEIK

SEQ ID NO: 207 - protein, 119 aa, Artificial Sequence
  Synthetic Construct
EVQLQQSGAELVRPGASVRLSCTASGFNIKDSLMHWVKQRPEQGLEWIGWIDPEDGETRYAPNFQDKATITAVTSSNTAYLQLSSLTSEDSAIYYCARYTSRYYTMEYWGQGTSVTVSS

SEQ ID NO: 208 - protein, 107 aa, Artificial Sequence
  Synthetic Construct
DIQMTQSPASQSASLGESVTFTCLASQTIGTWLAWYQQKPGKSPQLLIYAATSLADGVPSRFSGSGSGTKFSFKISSLQAEDFASYYCQQLYWTPWTFGGGTKLEIK

SEQ ID NO: 209 - protein, 443 aa, Artificial Sequence
  Synthetic Construct
EVQLQQSGAELVRPGASVRLSCTASGFNIKDSLMHWVKQRPEQGLEWIGWIDPEDGETKYAPNFQDKATITAVTSSNTAYLQLSSLTSEDSAIYYCARYTSDYYTMDYWGQGTSVTVSSAKTTPPSVYPLAPGFAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 210 - protein, 214 aa, Artificial Sequence
  Synthetic Construct
DIQMTQSPASQSASLGESVTFTCLASQTIGTWLAWYQQKPGKSPQLLIYAATSLADGVPSRFSGSGSGTKFSFKISSLQAEDFASYYCQQLYSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 211 - protein, 443 aa, Artificial Sequence
  Synthetic Construct
EVQLQQSGAELVRPGASVRLSCTASGFNIKDSLMHWVKQRPEQGLEWIGWIDPEDGETKYAPNFQDKATITAVTSSNTAYLQLSSLTSEDSAIYYCARYTSDYYTMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 212 - protein, 214 aa, Artificial Sequence; identical to SEQ ID NO: 210
  Synthetic Construct

SEQ ID NO: 213 - protein, 443 aa, Artificial Sequence
  Synthetic Construct
EVQLQQSGAELVRPGASVRLSCTASGFNIKDSLMHWVKQRPEQGLEWIGWIDPEDGETKYAPNFQSKATITAVTSSNTAYLQLSSLTSEDSAIYYCARYTSPYYTMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 214 - protein, 214 aa, Artificial Sequence
  Synthetic Construct
DIQMTQSPASQSASLGESVTFTCLASQTIGTWLAWYQQKPGKSPQLLIYAATSLADGVPSRFSGSGSGTKFSFKISSLQAEDFASYYCQQVYSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 215 - protein, 443 aa, Artificial Sequence
  Synthetic Construct
EVQLQQSGAELVRPGASVRLSCTASGFNIKDSLMHWVKQRPEQGLEWIGWIDPEDGETRYAPNFQDKATITAVTSSNTAYLQLSSLTSEDSAIYYCARYTSRYYTMEYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 216 - protein, 214 aa, Artificial Sequence
  Synthetic Construct
DIQMTQSPASQSASLGESVTFTCLASQTIGTWLAWYQQKPGKSPQLLIYAATSLADGVPSRFSGSGSGTKFSFKISSLQAEDFASYYCQQLYWTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 217 - protein, 8 aa, Artificial Sequence
  Synthetic Construct
GFTFSNYG

SEQ ID NO: 218 - protein, 8 aa, Artificial Sequence
  Synthetic Construct
ISYDGNNK

SEQ ID NO: 219 - protein, 15 aa, Artificial Sequence
  Synthetic Construct
AKDRGLDTAGDYFDY

SEQ ID NO: 220 - protein, 6 aa, Artificial Sequence
  Synthetic Construct
QNISSK

SEQ ID NO: 221 - protein, 3 aa, Artificial Sequence
  Synthetic Construct
GAS

SEQ ID NO: 222 - protein, 8 aa, Artificial Sequence
  Synthetic Construct
LQYNNWWT

SEQ ID NO: 223 - protein, 8 aa, Artificial Sequence
  Synthetic Construct
NGSFSSYY

SEQ ID NO: 224 - protein, 7 aa, Artificial Sequence
  Synthetic Construct
INHRGST

SEQ ID NO: 225 - protein, 9 aa, Artificial Sequence
  Synthetic Construct
TAHTRGFLY

SEQ ID NO: 226 - protein, 6 aa, Artificial Sequence
  Synthetic Construct
QDINIY

SEQ ID NO: 227 - protein, 3 aa, Artificial Sequence
  Synthetic Construct
DVS

SEQ ID NO: 228 - protein, 9 aa, Artificial Sequence
  Synthetic Construct
QHYDDLPLT

SEQ ID NO: 229 - protein, 8 aa, Artificial Sequence
  Synthetic Construct
NGSFSSFY

SEQ ID NO: 230 - protein, 7 aa, Artificial Sequence
  Synthetic Construct
INHRGRT

SEQ ID NO: 231 - protein, 6 aa, Artificial Sequence
  Synthetic Construct
QDINNY

SEQ ID NO: 232 - protein, 3 aa, Artificial Sequence
  Synthetic Construct
DVF

SEQ ID NO: 233 - protein, 9 aa, Artificial Sequence
  Synthetic Construct
SAHTRGFLY

SEQ ID NO: 234 - protein, 3 aa, Artificial Sequence
  Synthetic Construct
DAS

SEQ ID NO: 235 - protein, 9 aa, Artificial Sequence
  Synthetic Construct
QHYYDLPLT

SEQ ID NO: 236 - protein, 8 aa, Artificial Sequence
  Synthetic Construct
GFTFSDSA

SEQ ID NO: 237 - protein, 10 aa, Artificial Sequence
  Synthetic Construct
IRNKANTYAT

SEQ ID NO: 238 - protein, 6 aa, Artificial Sequence
  Synthetic Construct
TSDQFD

SEQ ID NO: 239 - protein, 11 aa, Artificial Sequence
  Synthetic Construct
QSLVYSDGITY

SEQ ID NO: 240 - protein, 3 aa, Artificial Sequence
  Synthetic Construct
QVS

SEQ ID NO: 241 - protein, 9 aa, Artificial Sequence
  Synthetic Construct
MEGTHWPWT

SEQ ID NO: 242 - protein, 8 aa, Artificial Sequence
  Synthetic Construct
GFTFSDYY

SEQ ID NO: 243 - protein, 8 aa, Artificial Sequence
  Synthetic Construct
ISRTGSTK

SEQ ID NO: 244 - protein, 7 aa, Artificial Sequence
  Synthetic Construct
AREPLQH

SEQ ID NO: 245 - protein, 11 aa, Artificial Sequence
  Synthetic Construct
QSLVHSDGDTY

SEQ ID NO: 246 - protein, 3 aa, Artificial Sequence
  Synthetic Construct
TVS

SEQ ID NO: 247 - protein, 10 aa, Artificial Sequence
  Synthetic Construct
MQGTQFPALT

SEQ ID NO: 248 - protein, 3 aa, Artificial Sequence
  Synthetic Construct
TIS

SEQ ID NO: 249 - protein, 8 aa, Artificial Sequence
  Synthetic Construct
GFTLSDSS

SEQ ID NO: 250 - protein, 10 aa, Artificial Sequence
  Synthetic Construct
IRSKPYSYAT

SEQ ID NO: 251 - protein, 6 aa, Artificial Sequence
  Synthetic Construct
TGGDDF

SEQ ID NO: 252 - protein, 6 aa, Artificial Sequence
  Synthetic Construct
QSISTW

SEQ ID NO: 253 - protein, 3 aa, Artificial Sequence
  Synthetic Construct
KAS

SEQ ID NO: 254 - protein, 8 aa, Artificial Sequence
  Synthetic Construct
QQYNTYWT

SEQ ID NO: 255 - protein, 8 aa, Artificial Sequence
  Synthetic Construct
GFTFSSYA

SEQ ID NO: 256 - protein, 8 aa, Artificial Sequence
  Synthetic Construct
ISGSGGST

SEQ ID NO: 257 - protein, 6 aa, Artificial Sequence
  Synthetic Construct
QSVSSN

SEQ ID NO: 258 - protein, 10 aa, Artificial Sequence
  Synthetic Construct
QQYNNWPPYT

SEQ ID NO: 259 - protein, 8 aa, Artificial Sequence
  Synthetic Construct
GYTFTGYY

SEQ ID NO: 260 - protein, 8 aa, Artificial Sequence
  Synthetic Construct
INPNSVGT

SEQ ID NO: 261 - protein, 12 aa, Artificial Sequence
  Synthetic Construct
TRDGAAAGLFDY

SEQ ID NO: 262 - protein, 6 aa, Artificial Sequence
  Synthetic Construct
QGINNY

SEQ ID NO: 263 - protein, 3 aa, Artificial Sequence
  Synthetic Construct
AAS

SEQ ID NO: 264 - protein, 9 aa, Artificial Sequence
  Synthetic Construct
QKFNSAPLT

SEQ ID NO: 265 - protein, 8 aa, Artificial Sequence
  Synthetic Construct
GFTFSTYA

SEQ ID NO: 266 - protein, 9 aa, Artificial Sequence
  Synthetic Construct
ANSPSWFDP

SEQ ID NO: 267 - protein, 6 aa, Artificial Sequence
  Synthetic Construct
QSISSY

SEQ ID NO: 268 - protein, 10 aa, Artificial Sequence
  Synthetic Construct
QQSYSTPPIT

SEQ ID NO: 269 - protein, 8 aa, Artificial Sequence
  Synthetic Construct
GFTFDDYG

SEQ ID NO: 270 - protein, 8 aa, Artificial Sequence
  Synthetic Construct
ISWNSATI

SEQ ID NO: 271 - protein, 12 aa, Artificial Sequence
  Synthetic Construct
AKKNSLGWFFDY

SEQ ID NO: 272 - protein, 12 aa, Artificial Sequence
  Synthetic Construct
QSVLYSSNDNNY

SEQ ID NO: 273 - protein, 3 aa, Artificial Sequence
  Synthetic Construct
WAS

SEQ ID NO: 274 - protein, 9 aa, Artificial Sequence
  Synthetic Construct
QQYYSTPLT

SEQ ID NO: 275 - protein, 8 aa, Artificial Sequence
  Synthetic Construct
GYTFTSYG

SEQ ID NO: 276 - protein, 8 aa, Artificial Sequence
  Synthetic Construct
ISAYNGNT

SEQ ID NO: 277 - protein, 10 aa, Artificial Sequence
  Synthetic Construct
ARDINWYFDL

SEQ ID NO: 278 - protein, 6 aa, Artificial Sequence
  Synthetic Construct
QGIRND

SEQ ID NO: 279 - protein, 9 aa, Artificial Sequence
  Synthetic Construct
LQHNSYPWT

SEQ ID NO: 280 - protein, 8 aa, Artificial Sequence
  Synthetic Construct
ISWNGATI

SEQ ID NO: 281 - protein, 3 aa, Artificial Sequence
  Synthetic Construct
TAS

SEQ ID NO: 282 - protein, 9 aa, Artificial Sequence
  Synthetic Construct
LQHNNYPYT

SEQ ID NO: 283 - protein, 8 aa, Artificial Sequence
  Synthetic Construct
GFTFSNYA

SEQ ID NO: 284 - protein, 8 aa, Artificial Sequence
  Synthetic Construct
ITGNGVNT

SEQ ID NO: 285 - protein, 15 aa, Artificial Sequence
  Synthetic Construct
VKERGHSWFGDWFDP

SEQ ID NO: 286 - protein, 6 aa, Artificial Sequence
  Synthetic Construct
QSVISY

SEQ ID NO: 287 - protein, 9 aa, Artificial Sequence
  Synthetic Construct
QQNYLPPLT

SEQ ID NO: 288 - protein, 8 aa, Artificial Sequence
  Synthetic Construct
ITGNGIKT

SEQ ID NO: 289 - protein, 11 aa, Artificial Sequence
  Synthetic Construct
QGLVNSDGNTY

SEQ ID NO: 290 - protein, 3 aa, Artificial Sequence
  Synthetic Construct
EIS

SEQ ID NO: 291 - protein, 9 aa, Artificial Sequence
  Synthetic Construct
MQSTQFPLT

SEQ ID NO: 292 - protein, 8 aa, Artificial Sequence
  Synthetic Construct
ITGNGINT

SEQ ID NO: 293 - protein, 8 aa, Artificial Sequence
  Synthetic Construct
VSYDGSNK

SEQ ID NO: 294 - protein, 15 aa, Artificial Sequence
  Synthetic Construct
AKDRGITGTSGGVFD

SEQ ID NO: 295 - protein, 6 aa, Artificial Sequence
  Synthetic Construct
QGISSH

SEQ ID NO: 296 - protein, 3 aa, Artificial Sequence
  Synthetic Construct
VAS

SEQ ID NO: 297 - protein, 9 aa, Artificial Sequence
  Synthetic Construct
QQYYSYPPT

SEQ ID NO: 298 - protein, 8 aa, Artificial Sequence
  Synthetic Construct
GFTSSRYW

SEQ ID NO: 299 - protein, 8 aa, Artificial Sequence
  Synthetic Construct
INQDGSEK

SEQ ID NO: 300 - protein, 7 aa, Artificial Sequence
  Synthetic Construct
ARDEEGY

SEQ ID NO: 301 - protein, 11 aa, Artificial Sequence
  Synthetic Construct
QSLVYSDGDTY

SEQ ID NO: 302 - protein, 3 aa, Artificial Sequence
  Synthetic Construct
KVS

SEQ ID NO: 303 - protein, 9 aa, Artificial Sequence
  Synthetic Construct
MQGTHWPPT

SEQ ID NO: 304 - protein, 122 aa, Artificial Sequence
  Synthetic Construct
QVHLVESGGGVVQPGRSLRLSCAASGFTFSNYGIHWVRQVPGKGLEWVAVISYDGNNKYYGDSVKGRFTISRDNSKNTLYLQMNSLRPEDTAIYYCAKDRGLDTAGDYFDYWGQGTLVTVSS

SEQ ID NO: 305 - protein, 106 aa, Artificial Sequence
  Synthetic Construct
EIVMTQSPATLSVSTGERATLSCRASQNISSKLAWYQQKPGQAPRLLIYGASTRASGIPARFWGSGSGTEFTLTISSLQSEDFAVFYCLQYNNWWTFGQGTKVEIK

SEQ ID NO: 306 - protein, 115 aa, Artificial Sequence
  Synthetic Construct
QVQLQQWGAGLLKPSETLSLTCAVYNGSFSSYYWNWIRQPPGKGLEWLGEINHRGSTNYNPSLTSRVTISVDTSKNQFSLKLTSVTAADTAIYYCTAHTRGFLYWGQGTLVTVSS

SEQ ID NO: 307 - protein, 107 aa, Artificial Sequence
  Synthetic Construct
DIQMTQSPSSLSASVGDRVTITCQASQDINIYLNWYQQQPGKAPKLLIYDVSNLVPGVPSRFSGTGSGTHFSFTISSLQPEDIATYYCQHYDDLPLTFGGGTTVAIR

SEQ ID NO: 308 - protein, 115 aa, Artificial Sequence
  Synthetic Construct
QVQLQQWGAGLLKPSETLSLTCAVYNGSFSSFYWSWIRQPPGKGLEWLGEINHRGRTNNNPSLASRVTISVDTSKSQFSLRLTSVTAADTATYYCTAHTRGFLYWGQGTRVTVSS

SEQ ID NO: 309 - protein, 107 aa, Artificial Sequence
  Synthetic Construct
DIQMTQSPSSLSASVGDRVTITCQASQDINNYLNWYQQKPGQAPNLLIYDVFNLLPGVPSRFSGSGSGTDFSFTISSLQPEDIATYYCQHYDDLPLTFGGGTTVAIR

SEQ ID NO: 310 - protein, 115 aa, Artificial Sequence
  Synthetic Construct
QVQLQQWGAGLLKPSETLSLTCAVYNGSFSSYYWSWIRQPPGKGLEWLGEINHRGRTNYNPSLTSRVTISVDTSKNQFSLKLPSVTAADTAIYYCSAHTRGFLYWGQGTLVTVSS

SEQ ID NO: 311 - protein, 107 aa, Artificial Sequence
  Synthetic Construct
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAIS

SEQ ID NO: 312 - protein, 107 aa, Artificial Sequence
  Synthetic Construct
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAIR

SEQ ID NO: 313 - protein, 116 aa, Artificial Sequence
  Synthetic Construct
EIQLVESGGDLVQPGGSLKLSCAASGFTFSDSAMHWVRQASGKGLEWVGRIRNKANTYATSYAASVKGRFTVSTDDSKTTAYLQMRYLKIEDTAIYYCTSDQFDYWGQGTLVTVSS

SEQ ID NO: 314 - protein, 112 aa, Artificial Sequence
  Synthetic Construct
DVVMTQSPLSLSATLGQPASISCRSSQSLVYSDGITYLTWFQQRPGQSPTRLIYQVSNRDSGVPDRFNGSGSGTDFTLNISRVEAEDVGFYYCMEGTHWPWTFGQGTKVELK

SEQ ID NO: 315 - protein, 114 aa, Artificial Sequence
  Synthetic Construct
QVQLVESGGGLVKPGGSLRLSCAASGFTFSDYYMSWIRQAPGKGLEWISFISRTGSTKYYADSVKGRFTISRDNAKNSLNLQMSSLRAEDTAVYFCAREPLQHWGQGTLVTVSS

SEQ ID NO: 316 - protein, 113 aa, Artificial Sequence
  Synthetic Construct
NVVLTQTPISSPVILGQPASISCRSSQSLVHSDGDTYLSWLHQRPGQPPRLLIYTVSNRVSGVPDRFSGSGAGTDFTLKISRVEAEDVGVYYCMQGTQFPALTFGGGTKVEIK

SEQ ID NO: 317 - protein, 114 aa, Artificial Sequence
  Synthetic Construct
QVQLVESGGGLVKPGGSLRLSCAASGFTFSDYYMSWIRQAPGKGLEWVSYISRTGSTKYYADSVKGRFTISRDNAKNSLNLQMSSLRAEDTAVYFCAREPLQHWGQGTLVTVSS

SEQ ID NO: 318 - protein, 113 aa, Artificial Sequence
  Synthetic Construct
KIVLTQTPLSSPVTLGQPASISCRSSQSLVHSDGDTYLSWLHQRPGQPPRLLIYTISNRLSGVPDRFSGSGAGTDFTLKISRVEAEDVGVYYCMQGTQFPALTFGGGTKVEIK

SEQ ID NO: 319 - protein, 115 aa, Artificial Sequence
  Synthetic Construct
EMQLVESGGGLVQPGGSLKVSCAASGFTLSDSSIHWVRQASGKGLEWIGRIRSKPYSYATAYAASVKGRFTISRDDSKNTAFLQMSGLKTEDTAVYYCTGGDDFWGQGTLVTVSS

SEQ ID NO: 320 - protein, 106 aa, Artificial Sequence
  Synthetic Construct
DIQMTQSPSTLSASVGDRITITCRASQSISTWLAWYQQKPGKAPKLLIYKASSLEGGVPSRFSGSGSGTDFTLTISSLQPDDFATYYCQQYNTYWTFGQGTKVEIK

SEQ ID NO: 321 - protein, 121 aa, Artificial Sequence
  Synthetic Construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAIPYNWNYGGAFDIWGQGTMVTVSS

SEQ ID NO: 322 - protein, 108 aa, Artificial Sequence
  Synthetic Construct
EIVMTQSPATLSVSPGKRATLSCRASQSVSSNLAWYQQKPGQAPRLLIYGASTRATGIPARFSGSGSGTEFTLTISSLQSEDFAVYYCQQYNNWPPYTFGQGTKLEIK

SEQ ID NO: 323 - protein, 119 aa, Artificial Sequence
  Synthetic Construct
QVQLVQSGAEVKKPGASVKVSCKASGYTFTGYYMHWVRQAPGQGLEWMGWINPNSVGTNFAQKFQGRVTMTRDTSISTAYMELSRLRSDDTAVYYCTRDGAAAGLFDYWGQGTLVTVSS

SEQ ID NO: 324 - protein, 107 aa, Artificial Sequence
  Synthetic Construct
DIQMTQSPSSLSASVGDRVTITCRASQGINNYLAWYQQKPGKVPKLLIYAASTLQSGVPSRFSGSGSGTDFTLTISSLQPEDVATYYCQKFNSAPLTFGGGTKVEIK

SEQ ID NO: 325 - protein, 116 aa, Artificial Sequence
  Synthetic Construct
EVQLVESGGGLVQPGGSLRLSCAASGFTFSTYAMSWVRQAPGKGLEWVSAISGSGGSTYYADSVKGRFTISRDNSKNTLYLQMKSLRVEDTAVYYCANSPSWFDPWGQGTLVTVSS

SEQ ID NO: 326 - protein, 108 aa, Artificial Sequence
  Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 327 - protein, 119 aa, Artificial Sequence
  Synthetic Construct
EVQLVESGGGLVQPGRSLRLSCAASGFTFDDYGMHWVRQAPGKGLEWVSVISWNSATIDYADSVKGRFTISRDNAKNSLFLQMNSLRPEDTALYYCAKKNSLGWFFDYWGQGTLVTVSS

SEQ ID NO: 328 - protein, 113 aa, Artificial Sequence
  Synthetic Construct
DIVMTQSPDSLAVSLGERATINCKSSQSVLYSSNDNNYLAWYQQKPGQPPKLLIYWASTRESGVPDRFSGSGSGTDFTLTISSLQAEDVAVYYCQQYYSTPLTFGGGTKVEIK

SEQ ID NO: 329 - protein, 117 aa, Artificial Sequence
  Synthetic Construct
QVQLVQSGAEVKKPGASVKVSCKASGYTFTSYGISWVRQAPGQGLEWMGWISAYNGNTIYAQKFQGRVTMTTDTSTSTAYMELRSLRSDDTAVYYCARDINWYFDLWGRGTLVTVSS

SEQ ID NO: 330 - protein, 107 aa, Artificial Sequence
  Synthetic Construct
DIQMTQSPSSLSASVGDRVTITCRASQGIRNDLGWFQQKPGKAPKRLIYAASSLQSGVPSRFSGSGSGTEFTLTISSLQPEDFATYYCLQHNSYPWTFGQGTKVEIK

SEQ ID NO: 331 - protein, 119 aa, Artificial Sequence
  Synthetic Construct
EVQLVESGGGLVQPGRSLRLSCAASGFTFDDYGMHWVRQAPGKGLEWVSVISWNGATIDYADSVKGRFTISRDNAKNSLFLQMNSLRPEDTALYYCAKKNSLGWFFDYWGQGTLVTVSS

SEQ ID NO: 332 - protein, 107 aa, Artificial Sequence
  Synthetic Construct
DIQMTQSPSSLSAFVGDRVTITCRASQGIRNDLGWYQQKPGKTPKRLIYTASSLRSGVPSRFSGSGSGTDFTLTISSLQPEDFATYYCLQHNNYPYTFGQGTKLEIK

SEQ ID NO: 333 - protein, 122 aa, Artificial Sequence
  Synthetic Construct
EVQLVESGGNLVQPGGSLRLSCAASGFTFSNYAMSWVRQAPGKGLEWVSGITGNGVNTYYSVSVKGRFTISRDNSKNTLFLEMNSLRAEDTAIYYCVKERGHSWFGDWFDPWGQGTLVTVSS

SEQ ID NO: 334 - protein, 107 aa, Artificial Sequence
  Synthetic Construct
DIQMTQSPSSLSASVGDRVTITCRSSQSVISYLNWYQQKPGKAPKLLIFGASSLISGVPSRFSGSGSGTDFTLTIVSLQPEDFAVYFCQQNYLPPLTFGGGTKVEIK

SEQ ID NO: 335 - protein, 122 aa, Artificial Sequence
  Synthetic Construct
EVQLVESGGNLVQPGGSLRLSCAASGFTFSNYAMSWVRQAPGKGLEWVSGITGNGIKTYYSVSAKGRFTISRDNSKNTLFLQMNSLRAEDTAIYYCVKERGHSWFGDWFDPWGQGTLVTVSS

SEQ ID NO: 336 - protein, 112 aa, Artificial Sequence
  Synthetic Construct
DIVMTQTPLSSPVTLGQPASISCRSSQGLVNSDGNTYLSWLHQRPGQPPRLLIYEISNRFSGVPDRFSGSGAGTDFTLKISRVEAEDVGFYYCMQSTQFPLTFGGGTKVEIK

SEQ ID NO: 337 - protein, 122 aa, Artificial Sequence
  Synthetic Construct
EVQLVESGGNLVQPGGSLRLSCAASGFTFSNYAMSWVRQAPGKGLEWVSGITGNGINTYYSVSVKGRFTISRDNSKNTVFLQMNSLRAEDTAIYYCVKERGHSWFGDWFDPWGQGTLVTVSS

SEQ ID NO: 338 - protein, 107 aa, Artificial Sequence
  Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 339 - protein, 123 aa, Artificial Sequence
  Synthetic Construct
QVQLVESGGGVVQPGRSLRLSCEVSGFTFSNYGMHWVRQAPGKGLEWVAVVSYDGSNKYYTDSVKGRFTISRDNSKNTLSLQMNSLRAEDTAVYYCAKDRGITGTSGGVFDIWGQGTMVTVSS

SEQ ID NO: 340 - protein, 107 aa, Artificial Sequence
  Synthetic Construct
AIRMTQSPSSFSATTGDRVTITCRASQGISSHLAWYQQKPGKAPKFLIYVASTLQSGVPSRFSGSGSGTDFTLTISSLQSEDFATYYCQQYYSYPPTFGQGTKVEIK

SEQ ID NO: 341 - protein, 114 aa, Artificial Sequence
  Synthetic Construct
EVQLVESGGGLVQPGGSLRLSCAASGFTSSRYWMSWVRQAPGKGLEWLANINQDGSEKYYLESLRGRFTISRDNAKNSLYLQMNNLRAEDTAVYYCARDEEGYWGQGTLVTVSS

SEQ ID NO: 342 - protein, 112 aa, Artificial Sequence
  Synthetic Construct
DVVMTQSLLSLSVTLGQPASISCRSSQSLVYSDGDTYLSWFQQRPGQSPRRLIYKVSHRDSGVPDRFRGSGSGTDFTLKISRVEAEDVGIYYCMQGTHWPPTFGQGTKVEIK

SEQ ID NO: 343 - protein, 449 aa, Artificial Sequence
  Synthetic Construct
QVHLVESGGGVVQPGRSLRLSCAASGFTFSNYGIHWVRQVPGKGLEWVAVISYDGNNKYYGDSVKGRFTISRDNSKNTLYLQMNSLRPEDTAIYYCAKDRGLDTAGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 344 - protein, 213 aa, Artificial Sequence
  Synthetic Construct
EIVMTQSPATLSVSTGERATLSCRASQNISSKLAWYQQKPGQAPRLLIYGASTRASGIPARFWGSGSGTEFTLTISSLQSEDFAVFYCLQYNNWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 442 aa, Artificial Sequence
  Synthetic Construct
QVQLQQWGAGLLKPSETLSLTCAVYNGSFSSYYWNWIRQPPGKGLEWLGEINHRGSTNYNPSLTSRVTISVDTSKNQFSLKLTSVTAADTAIYYCTAHTRGFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 346 - protein, 214 aa, Artificial Sequence
  Synthetic Construct
DIQMTQSPSSLSASVGDRVTITCQASQDINIYLNWYQQQPGKAPKLLIYDVSNLVPGVPSRFSGTGSGTHFSFTISSLQPEDIATYYCQHYDDLPLTFGGGTTVAIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 442 aa, Artificial Sequence
  Synthetic Construct
QVQLQQWGAGLLKPSETLSLTCAVYNGSFSSFYWSWIRQPPGKGLEWLGEINHRGRTNNNPSLASRVTISVDTSKSQFSLRLTSVTAADTATYYCTAHTRGFLYWGQGTRVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 348 - protein, 214 aa, Artificial Sequence
  Synthetic Construct
DIQMTQSPSSLSASVGDRVTITCQASQDINNYLNWYQQKPGQAPNLLIYDVFNLLPGVPSRFSGSGSGTDFSFTISSLQPEDIATYYCQHYDDLPLTFGGGTTVAIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 442 aa, Artificial Sequence
  Synthetic Construct
QVQLQQWGAGLLKPSETLSLTCAVYNGSFSSYYWSWIRQPPGKGLEWLGEINHRGRTNYNPSLTSRVTISVDTSKNQFSLKLPSVTAADTAIYYCSAHTRGFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 350 - protein, 214 aa, Artificial Sequence
  Synthetic Construct
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 214 aa, Artificial Sequence
  Synthetic Construct
DIQMTQSPSSLSASVGDRVTITCQASQDINNYLNWYQQKPGKAPKLLIYDASNLVPGVPSRFSGTGSGTDFSFTISSLQPEDIATYYCQHYYDLPLTFGGGTTVAIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 352 - protein, 443 aa, Artificial Sequence
  Synthetic Construct
EIQLVESGGDLVQPGGSLKLSCAASGFTFSDSAMHWVRQASGKGLEWVGRIRNKANTYATSYAASVKGRFTVSTDDSKTTAYLQMRYLKIEDTAIYYCTSDQFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 353 - protein, 219 aa, Artificial Sequence
  Synthetic Construct
DVVMTQSPLSLSATLGQPASISCRSSQSLVYSDGITYLTWFQQRPGQSPTRLIYQVSNRDSGVPDRFNGSGSGTDFTLNISRVEAEDVGFYYCMEGTHWPWTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 354 - protein, 441 aa, Artificial Sequence
  Synthetic Construct
QVQLVESGGGLVKPGGSLRLSCAASGFTFSDYYMSWIRQAPGKGLEWISFISRTGSTKYYADSVKGRFTISRDNAKNSLNLQMSSLRAEDTAVYFCAREPLQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 355 - protein, 220 aa, Artificial Sequence
  Synthetic Construct
NVVLTQTPISSPVILGQPASISCRSSQSLVHSDGDTYLSWLHQRPGQPPRLLIYTVSNRVSGVPDRFSGSGAGTDFTLKISRVEAEDVGVYYCMQGTQFPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 441 aa, Artificial Sequence
  Synthetic Construct
QVQLVESGGGLVKPGGSLRLSCAASGFTFSDYYMSWIRQAPGKGLEWVSYISRTGSTKYYADSVKGRFTISRDNAKNSLNLQMSSLRAEDTAVYFCAREPLQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 357 - protein, 220 aa, Artificial Sequence
  Synthetic Construct
KIVLTQTPLSSPVTLGQPASISCRSSQSLVHSDGDTYLSWLHQRPGQPPRLLIYTISNRLSGVPDRFSGSGAGTDFTLKISRVEAEDVGVYYCMQGTQFPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 442 aa, Artificial Sequence
  Synthetic Construct
EMQLVESGGGLVQPGGSLKVSCAASGFTLSDSSIHWVRQASGKGLEWIGRIRSKPYSYATAYAASVKGRFTISRDDSKNTAFLQMSGLKTEDTAVYYCTGGDDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 213 aa, Artificial Sequence
  Synthetic Construct
DIQMTQSPSTLSASVGDRITITCRASQSISTWLAWYQQKPGKAPKLLIYKASSLEGGVPSRFSGSGSGTDFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - protein, 448 aa, Artificial Sequence
  Synthetic Construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAIPYNWNYGGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 361 - protein, 215 aa, Artificial Sequence
  Synthetic Construct
EIVMTQSPATLSVSPGKRATLSCRASQSVSSNLAWYQQKPGQAPRLLIYGASTRATGIPARFSGSGSGTEFTLTISSLQSEDFAVYYCQQYNNWPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - protein, 446 aa, Artificial Sequence
  Synthetic Construct
QVQLVQSGAEVKKPGASVKVSCKASGYTFTGYYMHWVRQAPGQGLEWMGWINPNSVGTNFAQKFQGRVTMTRDTSISTAYMELSRLRSDDTAVYYCTRDGAAAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 214 aa, Artificial Sequence
  Synthetic Construct
DIQMTQSPSSLSASVGDRVTITCRASQGINNYLAWYQQKPGKVPKLLIYAASTLQSGVPSRFSGSGSGTDFTLTISSLQPEDVATYYCQKFNSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 443 aa, Artificial Sequence
  Synthetic Construct
EVQLVESGGGLVQPGGSLRLSCAASGFTFSTYAMSWVRQAPGKGLEWVSAISGSGGSTYYADSVKGRFTISRDNSKNTLYLQMKSLRVEDTAVYYCANSPSWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 365 - protein, 215 aa, Artificial Sequence
  Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 366 - protein, 446 aa, Artificial Sequence
  Synthetic Construct
EVQLVESGGGLVQPGRSLRLSCAASGFTFDDYGMHWVRQAPGKGLEWVSVISWNSATIDYADSVKGRFTISRDNAKNSLFLQMNSLRPEDTALYYCAKKNSLGWFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 367 - protein, 220 aa, Artificial Sequence
  Synthetic Construct
DIVMTQSPDSLAVSLGERATINCKSSQSVLYSSNDNNYLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368 - protein, 444 aa, Artificial Sequence
  Synthetic Construct
QVQLVQSGAEVKKPGASVKVSCKASGYTFTSYGISWVRQAPGQGLEWMGWISAYNGNTIYAQKFQGRVTMTTDTSTSTAYMELRSLRSDDTAVYYCARDINWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 369 - protein, 214 aa, Artificial Sequence
  Synthetic Construct
DIQMTQSPSSLSASVGDRVTITCRASQGIRNDLGWFQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - protein, 446 aa, Artificial Sequence
  Synthetic Construct
EVQLVESGGGLVQPGRSLRLSCAASGFTFDDYGMHWVRQAPGKGLEWVSVISWNGATIDYADSVKGRFTISRDNAKNSLFLQMNSLRPEDTALYYCAKKNSLGWFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 371 - protein, 214 aa, Artificial Sequence
  Synthetic Construct
DIQMTQSPSSLSAFVGDRVTITCRASQGIRNDLGWYQQKPGKTPKRLIYTASSLRSGVPSRFSGSGSGTDFTLTISSLQPEDFATYYCLQHNNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 372 - protein, 449 aa, Artificial Sequence
  Synthetic Construct
EVQLVESGGNLVQPGGSLRLSCAASGFTFSNYAMSWVRQAPGKGLEWVSGITGNGVNTYYSVSVKGRFTISRDNSKNTLFLEMNSLRAEDTAIYYCVKERGHSWFGDWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 373 - protein, 214 aa, Artificial Sequence
  Synthetic Construct
DIQMTQSPSSLSASVGDRVTITCRSSQSVISYLNWYQQKPGKAPKLLIFGASSLISGVPSRFSGSGSGTDFTLTIVSLQPEDFAVYFCQQNYLPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 374 - protein, 449 aa, Artificial Sequence
  Synthetic Construct
EVQLVESGGNLVQPGGSLRLSCAASGFTFSNYAMSWVRQAPGKGLEWVSGITGNGIKTYYSVSAKGRFTISRDNSKNTLFLQMNSLRAEDTAIYYCVKERGHSWFGDWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 375 - protein, 219 aa, Artificial Sequence
  Synthetic Construct
DIVMTQTPLSSPVTLGQPASISCRSSQGLVNSDGNTYLSWLHQRPGQPPRLLIYEISNRFSGVPDRFSGSGAGTDFTLKISRVEAEDVGFYYCMQSTQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 376 - protein, 449 aa, Artificial Sequence
  Synthetic Construct
EVQLVESGGNLVQPGGSLRLSCAASGFTFSNYAMSWVRQAPGKGLEWVSGITGNGINTYYSVSVKGRFTISRDNSKNTVFLQMNSLRAEDTAIYYCVKERGHSWFGDWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 377 - protein, 214 aa, Artificial Sequence
  Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 378 - protein, 450 aa, Artificial Sequence
  Synthetic Construct
QVQLVESGGGVVQPGRSLRLSCEVSGFTFSNYGMHWVRQAPGKGLEWVAVVSYDGSNKYYTDSVKGRFTISRDNSKNTLSLQMNSLRAEDTAVYYCAKDRGITGTSGGVFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 379 - protein, 214 aa, Artificial Sequence
  Synthetic Construct
AIRMTQSPSSFSATTGDRVTITCRASQGISSHLAWYQQKPGKAPKFLIYVASTLQSGVPSRFSGSGSGTDFTLTISSLQSEDFATYYCQQYYSYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 380 - protein, 441 aa, Artificial Sequence
  Synthetic Construct
EVQLVESGGGLVQPGGSLRLSCAASGFTSSRYWMSWVRQAPGKGLEWLANINQDGSEKYYLESLRGRFTISRDNAKNSLYLQMNNLRAEDTAVYYCARDEEGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 381 - protein, 219 aa, Artificial Sequence
  Synthetic Construct
DVVMTQSLLSLSVTLGQPASISCRSSQSLVYSDGDTYLSWFQQRPGQSPRRLIYKVSHRDSGVPDRFRGSGSGTDFTLKISRVEAEDVGIYYCMQGTHWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 382 - protein, 14 aa, Artificial Sequence
  Synthetic Construct
AIPYNWNYGGAFDI